(19) United States
(12) Patent Application Publication
(10) Pub. No.: US 2024/0026374 A1
(43) Pub. Date: Jan. 25, 2024

(54) CLOSED-ENDED DNA VECTORS AND USES THEREOF FOR EXPRESSING PHENYLALANINE HYDROXYLASE (PAH)

(71) Applicant: Generation Bio Co., Cambridge, MA (US)

(72) Inventors: Leah Yu Liu, Cambridge, MA (US); Russell Monds, Cambridge, MA (US); Matthew James Chiocco, Cambridge, MA (US); Nathaniel Silver, Cambridge, MA (US); Phillip Samayoa, Cambridge, MA (US); Douglas Anthony Kerr, Cambridge, MA (US); Jessica Lynn Keenan, Cambridge, MA (US)

(21) Appl. No.: 18/025,926

(22) PCT Filed: Sep. 16, 2021
(86) PCT No.: PCT/US2021/050695

§ 371 Date: Mar. 13, 2023

Related U.S. Application Data
(60) Provisional application No. 63/078,954, filed on Sep. 16, 2020.

(51) Int. Cl. C12N 15/85; C12N 9/02; A61K 48/00; A61P 3/00

(52) U.S. Cl. CPC C12N 15/85; C12N 9/0071; C12Y 114/16001; A61K 48/0066; A61P 3/00; A61K 48/0016; C12N 2830/50

(57) ABSTRACT

Described herein are ceDNA vectors having linear and continuous structure for delivery and expression of a transgene. ceDNA vectors comprise an expression cassette flanked by two ITR sequences, where the expression cassette comprises a codon optimized nucleic acid sequence encoding a PAH protein, in combination with particular promoter sequences and cis-regulatory elements. Further provided herein are methods and cell lines for reliable gene expression of PAH protein in vitro, ex vivo and in vivo using the ceDNA vectors. Also provided herein are methods and compositions comprising ceDNA vectors useful for the expression of PAH protein in a cell, tissue or subject, and methods of treatment of diseases with said ceDNA vectors expressing PAH protein. Such PAH protein can be expressed for treating disease, e.g., Phenylketonuria (PKU).

### RELATED APPLICATION

This application claims priority to U.S. Provisional Application No. 63/078,954, filed on Sep. 16, 2020, the entire contents of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Sep. 16, 2021, is named 131698-08120_SL.txt and is 633,329 bytes in size.

### TECHNICAL FIELD

The present disclosure relates to the field of gene therapy, including non-viral vectors for expressing a transgene or isolated polynucleotides in a subject or cell. The disclosure also relates to nucleic acid constructs, promoters, vectors, and host cells including the polynucleotides as well as methods of delivering exogenous DNA sequences to a target cell, tissue, organ or organism. For example, the present disclosure provides methods for using non-viral closed-ended DNA (ceDNA) vectors to express phenylalanine hydroxylase (PAH) for treating disease by expressing PAH in a cell or tissue of a subject in need thereof.

### BACKGROUND

Gene therapy aims to improve clinical outcomes for patients suffering from either genetic mutations or acquired diseases caused by an aberration in the gene expression profile. Gene therapy includes the treatment or prevention of medical conditions resulting from defective genes or abnormal regulation or expression, e.g., underexpression or overexpression, that can result in a disorder, disease, malignancy, etc. For example, a disease or disorder caused by a defective gene might be treated, prevented or ameliorated by delivery of a corrective genetic material to a patient, or might be treated, prevented or ameliorated by altering or silencing a defective gene, e.g., with a corrective genetic material to a patient resulting in the therapeutic expression of the genetic material within the patient.

The basis of gene therapy is to supply a transcription cassette with an active gene product (sometimes referred to as a transgene), e.g., that can result in a positive gain-of-function effect, a negative loss-of-function effect, or another outcome. Such outcomes can be attributed to expression of a therapeutic protein such as an antibody, a functional enzyme, or a fusion protein. Gene therapy can also be used to treat a disease or malignancy caused by other factors. Human monogenic disorders can be treated by the delivery and expression of a normal gene to the target cells. Delivery and expression of a corrective gene in the patient's target cells can be carried out via numerous methods, including the use of engineered viruses and viral gene delivery vectors. Among the many virus-derived vectors available (e.g., recombinant retrovirus, recombinant lentivirus, recombinant adenovirus, and the like), recombinant adeno-associated virus (rAAV) is gaining popularity as a versatile vector in gene therapy.

Adeno-associated viruses (AAV) belong to the Parvoviridae family and more specifically constitute the dependoparvovirus genus. Vectors derived from AAV (i.e., recombinant AAV (rAVV) or AAV vectors) are attractive for delivering genetic material because (i) they are able to infect (transduce) a wide variety of non-dividing and dividing cell types including myocytes and neurons; (ii) they are devoid of the virus structural genes, thereby diminishing the host cell responses to virus infection, e.g., interferon-mediated responses; (iii) wild-type viruses are considered non-pathologic in humans; (iv) in contrast to wild type AAV, which are capable of integrating into the host cell genome, replication-deficient AAV vectors lack the rep gene and generally persist as episomes, thus limiting the risk of insertional mutagenesis or genotoxicity; and (v) in comparison to other vector systems, AAV vectors are generally considered to be relatively poor immunogens and therefore do not trigger a significant immune response (see ii), thus gaining persistence of the vector DNA and potentially, long-term expression of the therapeutic transgenes.

However, there are several major deficiencies in using AAV particles as a gene delivery vector. One major drawback associated with rAAV is its limited viral packaging capacity of about 4.5 kb of heterologous DNA (Dong et al., 1996; Athanasopoulos et al., 2004; Lai et al., 2010), and as a result, use of AAV vectors has been limited to less than 150,000 Da protein coding capacity. The second drawback is that as a result of the prevalence of wild-type AAV infection in the population, candidates for rAAV gene therapy have to be screened for the presence of neutralizing antibodies that eliminate the vector from the patient. A third drawback is related to the capsid immunogenicity that prevents re-administration to patients that were not excluded from an initial treatment. The immune system in the patient can respond to the vector which effectively acts as a “booster” shot to stimulate the immune system generating high titer anti-AAV antibodies that preclude future treatments. Some recent reports indicate concerns with immunogenicity in high dose situations. Another notable drawback is that the onset of AAV-mediated gene expression is relatively slow, given that single-stranded AAV DNA must be converted to double-stranded DNA prior to heterologous gene expression.

Additionally, conventional AAV virions with capsids are produced by introducing a plasmid or plasmids containing the AAV genome, rep genes, and cap genes (Grimm et al., 1998). However, such encapsidated AAV virus vectors were found to inefficiently transduce certain cell and tissue types and the capsids also induce an immune response.

Accordingly, use of adeno-associated virus (AAV) vectors for gene therapy is limited due to the single administration to patients (owing to the patient immune response), the limited range of transgene genetic material suitable for delivery in AAV vectors due to minimal viral packaging capacity (about 4.5 kb), and slow AAV-mediated gene expression.

Phenylketonuria (PKU) is a rare, inherited inborn error of metabolism caused by a mutation in the PAH gene. Phenylketonuria (PKU) results in decreased metabolism of the amino acid phenylalanine. Untreated, PKU can lead to intellectual disability, seizures, behavioral problems, and mental disorders. It may also result in a musty smell and lighter skin. Babies born to mothers who have poorly treated PKU may have heart problems, a small head, and low birth weight. PKU is due to mutations in the PAH gene, which results in low levels of the enzyme phenylalanine hydroxylase (PAH), i.e. subjects with PKU have mutations in PAH that render its enzymatic activity deficient. PKU is autosomal recessive, meaning that both copies of the gene must be mutated for the condition to develop. There are two main types, classic PKU and variant PKU, depending on if any enzyme function remains. Those with one copy of a mutated PAH gene typically do not have symptoms.

PAH is an enzyme that is normally expressed in the liver and is necessary to metabolize dietary phenylalanine (phe) into tyrosine, an amino acid responsible for the production of neurotransmitters. PAH catalyzes the hydroxylation of phenylalanine to tyrosine. Defective PAH enzyme results in the buildup of dietary phenylalanine to potentially toxic levels.

PKU can be caused by a single-gene defect in the enzyme phenylalanine hydroxylase (PAH), which results in elevated serum phe levels. PAH converts phe to tyrosine in vertebrates. In the absence of PAH, the only other mechanisms to remove Phe are protein synthesis and a minor degradative path involving the deamination and oxidative decarboxylation of the alanine side chain, which yields the characteristic phenyllactate and phenylacetate seen in urine of PKU patients. Unfortunately, a typical diet contains more Phe than can be eliminated in the absence of PAH. The resulting accumulation of Phe in PKU patients leads to a number of symptoms including abnormal brain development and severe mental retardation. (Kaufman, Proc Nat'l Acad Sci USA 96: 3160-3164, 1999).

The current standard of care is a highly restrictive diet (restriction of phenylalanine (Phe)), but it is not always effective, as such dietary restriction is difficult to maintain and does not correct the underlying defect. Current therapy for PKU is with a diet low in foods that contain phenylalanine and special supplements. The strict diet must begin as soon as possible after birth and be continued for at least 10 years, if not for the duration of life. If left untreated, PKU can result in progressive and severe neurological impairment. There are approximately 16,500 people living in the United States with PKU, and to date there are no treatments available that address the genetic defect in PKU.

Despite the tremendous advances in understanding the biochemistry, molecular biology, and genetics of PKU, little progress has been made in developing new treatments for the disorder. There is large unmet need for disease-modifying therapies in PKU. First, current therapies are not disease modifying and are only effective in a subset of patients, and still require strict dietary restrictions, and non-compliance can lead to neuronal damage. Second, there are no approved gene therapies for PKU, and AAV based therapies cannot be used by 25% to 40% of patients due to pre-existing antibodies. Further, AAV can only be administered once, and the resulting PAH levels might not be high enough to be efficacious, or may be supranormal, dose levels cannot be titrated.

Accordingly, there is need in the field for a technology that permits expression of a therapeutic PAH protein in a cell, tissue or subject for the treatment of PKU.

### BRIEF DESCRIPTION

The technology described herein relates to methods and compositions for treatment of Phenylketonuria (PKU) by expression of enzyme phenylalanine hydroxylase (PAH) from a capsid-free (e.g., non-viral) DNA vector with covalently-closed ends (referred to herein as a “closed-ended DNA vector” or a “ceDNA vector”), where the ceDNA vector comprises a PAH nucleic acid sequence that has been codon optimized and combined with particular cis-elements (e.g., specific promoters, specific enhancers and specific promoter and enhancer combinations), and that have been tested for optimal correction of phenylalanine level (e.g., expression and duration) in a mouse model of PKU. According to some embodiments, particular codon optimized PAH nucleic acid sequences perform better when combined with a specific promoter sequence and/or a specific enhancer sequence, compared to the same codon optimized PAH nucleic acid sequence combined with another promoter sequence and/or a specific enhancer sequence. As described by the present disclosure, the constructs comprising codon optimized sequences performed considerably better than native hPAH cDNA sequence, and certain constructs comprising codon optimized sequences and particular cis-acting elements showed extended correction through the duration of 28 days of the study, demonstrating durability of expression and efficacy. Surprisingly, it was found that constructs comprising certain promoters (e.g., hAAT CpG minimized promoter) performed better with certain open reading frames (ORFs) (ceDNA412 codop 2 ORF) in vivo in the PAH^{enu2} mouse model, while hAAT promoter was generally outcompeted by VD Promoter (VD) or 3×VD in vitro or with other ORFs (e.g., luciferase).

These ceDNA vectors can be used to produce PAH proteins for treatment, monitoring, and diagnosis. The application of ceDNA vectors expressing PAH to a subject for the treatment of PKU is useful to: (i) provide disease modifying levels of PAH enzyme, (ii) be minimally invasive in delivery, (iii) be repeatable and dosed-to-effect, (iv) have rapid onset of therapeutic effect, (v) result in sustained expression of corrective PAH enzyme in the liver, (vi) restoring urea cycle function, phenylalanine metabolism, and/or (vii) be titratable to achieve the appropriate pharmacologic levels of the defective enzyme.

Accordingly, the disclosure described herein relates to a capsid-free (e.g., non-viral) DNA vector with covalently-closed ends (referred to herein as a “closed-ended DNA vector” or a “ceDNA vector”) comprising a PAH nucleic acid sequence that has been codon optimized and combined with particular cis-acting elements (e.g., specific promoters, specific enhancers and specific promoter and enhancer combinations), to permit expression of the PAH therapeutic protein in a cell.

In one aspect, disclosed herein is a closed-ended DNA (ceDNA) vector comprising at least one nucleic acid sequence that encodes at least one PAH protein, wherein the at least one nucleic acid sequence is selected from a sequence having at least 90% identity to any of the sequences listed in Table 1A, wherein the at least one nucleic acid sequence is codon optimized, and wherein the at least one nucleic acid sequence is located between flanking inverted terminal repeats (ITRs); a promoter operatively linked to the least one nucleic acid sequence that encodes the at least one PAH protein, wherein the promoter is selected from the group consisting of the VD promoter, the human alpha 1-antitrypsin (hAAT) promoter (including a sequence having at least 96%, 97%, 98%, 99% or 100% identity to the hAAT(979) promoter (hAAT_core_C10) or other CpG minimized (CpGmin)_hAAT promoters like hAAT_core_C06; hAAT_core_C07; hAAT_core_C08; and hAAT_core_C09) and the transthyretin (TTR) liver specific promoter.

In one embodiment, the at least one nucleic acid sequence encoding the at least one PAH protein is selected from a sequence having at least 95% identity to any one of the sequences set forth in Table 1A. In one embodiment, the at least one nucleic acid sequence encoding the at least one PAH protein is selected from a sequence having at least 96% identity to any one of the sequences set forth in Table 1A. In one embodiment, the at least one nucleic acid sequence encoding the at least one PAH protein is selected from a sequence having at least 97% identity to any one of the sequences set forth in Table 1A. In one embodiment, the at least one nucleic acid sequence encoding the at least one PAH protein is selected from a sequence having at least 98% identity to any one of the sequences set forth in Table 1A. In one embodiment, the at least one nucleic acid sequence encoding the at least one PAH protein is selected from a sequence having at least 99% identity to any one of the sequences set forth in Table 1A. In one embodiment, the at least one nucleic acid sequence encoding the at least one PAH protein is selected from a sequence comprising any one of the sequences set forth in Table 1A.

According to another aspect, the disclosure provides a closed-ended DNA (ceDNA) vector comprising a nucleic acid sequence that encodes at least one PAH protein, wherein the nucleic acid sequence is selected from a sequence having at least 95% identity to any of the sequences listed in Table 1A, wherein the at least one nucleic acid sequence is located between flanking inverted terminal repeats (ITRs); and a promoter operatively linked to the nucleic acid sequence that encodes the at least one PAH protein, wherein the promoter is selected from the group consisting of the VD promoter, the human alpha 1-antitrypsin (hAAT) promoter and the transthyretin (TTR) liver specific promoter.

According to another aspect, the disclosure provides a closed-ended DNA (ceDNA) vector comprising a nucleic acid sequence that encodes at least one PAH protein, wherein the nucleic acid sequence is selected from a sequence having at least 96% identity to any of the sequences listed in Table 1A, wherein the at least one nucleic acid sequence is located between flanking inverted terminal repeats (ITRs); and a promoter operatively linked to the nucleic acid sequence that encodes the at least one PAH protein, wherein the promoter is selected from the group consisting of the VD promoter, the human alpha 1-antitrypsin (hAAT) promoter and the transthyretin (TTR) liver specific promoter.

According to another aspect, the disclosure provides a closed-ended DNA (ceDNA) vector comprising a nucleic acid sequence that encodes at least one PAH protein, wherein the nucleic acid sequence is selected from a sequence having at least 97% identity to any of the sequences listed in Table 1A, wherein the at least one nucleic acid sequence is located between flanking inverted terminal repeats (ITRs); and a promoter operatively linked to the nucleic acid sequence that encodes the at least one PAH protein, wherein the promoter is selected from the group consisting of the VD promoter, the human alpha 1-antitrypsin (hAAT) promoter and the transthyretin (TTR) liver specific promoter.

According to another aspect, the disclosure provides a closed-ended DNA (ceDNA) vector comprising a nucleic acid sequence that encodes at least one PAH protein, wherein the nucleic acid sequence is selected from a sequence having at least 98% identity to any of the sequences listed in Table 1A, wherein the at least one nucleic acid sequence is located between flanking inverted terminal repeats (ITRs); and a promoter operatively linked to the nucleic acid sequence that encodes the at least one PAH protein, wherein the promoter is selected from the group consisting of the VD promoter, the human alpha 1-antitrypsin (hAAT) promoter and the transthyretin (TTR) liver specific promoter.

According to another aspect, the disclosure provides a closed-ended DNA (ceDNA) vector comprising a nucleic acid sequence that encodes at least one PAH protein, wherein the nucleic acid sequence is selected from a sequence having at least 99% identity to any of the sequences listed in Table 1A, wherein the at least one nucleic acid sequence is located between flanking inverted terminal repeats (ITRs); and a promoter operatively linked to the nucleic acid sequence that encodes the at least one PAH protein, wherein the promoter is selected from the group consisting of the VD promoter, the human alpha 1-antitrypsin (hAAT) promoter and the transthyretin (TTR) liver specific promoter.

In one embodiment, the at least one nucleic acid sequence encoding the at least one PAH protein is a sequence having at least 98% identity to the sequence set forth as SEQ ID NO:382. In one embodiment, the at least one nucleic acid sequence encoding the at least one PAH protein is a sequence having at least 99% identity to the sequence set forth as SEQ ID NO:382. In one embodiment, the at least one nucleic acid sequence encoding the at least one PAH protein comprises SEQ ID NO:382 or consists of SEQ ID NO:382.

In one embodiment, the at least one nucleic acid sequence encoding the at least one PAH protein is a sequence having at least 99% identity to the sequence set forth as SEQ ID NO:425. In one embodiment, the at least one nucleic acid sequence encoding the at least one PAH protein is set forth as SEQ ID NO:425. In one embodiment, the at least one nucleic acid sequence encoding the at least one PAH protein is a sequence having at least 99% identity to the sequence set forth as SEQ ID NO:431. In one embodiment, the at least one nucleic acid sequence encoding the at least one PAH protein is set forth as SEQ ID NO:431. In one embodiment, the at least one nucleic acid sequence encoding the at least one PAH protein is a sequence having at least 99% identity to the sequence set forth as SEQ ID NO:435. In one embodiment, the at least one nucleic acid sequence encoding the at least one PAH protein is set forth as SEQ ID NO:435.

In one embodiment, the promoter comprises a sequence that is at least 85%, 90%, 95%, 96%, 97%, 98%, or 99% identical to, comprises, or consists of any one of SEQ ID NOs:441-448 and/or a sequence set forth in Table 7A.

In one embodiment, the promoter comprises a nucleic acid sequence having at least 85% identity, at least 90% identity, at least 95% identity, at least 96% identity, at least 97% identity, at least 98% identity, at least 99% identity or comprises SEQ ID NO: 191.

In one embodiment of the aspects and embodiments herein, the promoter comprises a nucleic acid sequence having at least 98% identity to SEQ ID NO: 443. In one embodiment of the aspects and embodiments herein, the promoter comprises a nucleic acid sequence having at least 99% identity to SEQ ID NO: 444. In one embodiment of the aspects and embodiments herein, the promoter comprises a nucleic acid sequence having at least 99% identity to SEQ ID NO: 445. In one embodiment of the aspects and embodiments herein, the promoter comprises a nucleic acid sequence having at least 99% identity to SEQ ID NO: 446. In one embodiment of the aspects and embodiments herein, the promoter comprises a nucleic acid sequence having at least 96% identity to SEQ ID NO: 447. In one embodiment of the aspects and embodiments herein, the promoter is a promoter set that comprises a nucleic acid sequence having at least 85% identity to SEQ ID NO: 462. In one embodiment of the aspects and embodiments herein, the promoter is a promoter set that comprises a nucleic acid sequence having at least 85% identity to SEQ ID NO: 467. In one embodiment of the aspects and embodiments herein, the promoter is a promoter set that comprises a nucleic acid sequence having at least 85% identity to SEQ ID NO: 470. In one embodiment of the aspects and embodiments herein, the promoter is a promoter set that comprises a nucleic acid sequence having at least 90% identity to SEQ ID NO: 470. In one embodiment of the aspects and embodiments herein, the promoter is a promoter set that comprises a nucleic acid sequence having at least 95% identity to SEQ ID NO: 470.

In one embodiment, the ceDNA vector further comprises an enhancer. In one embodiment, the enhancer comprises a sequence that is at least 85%, 90%, 95%, 96%, 97%, 98%, or 99% identical to, comprises, or consists of any one of SEQ ID NOs:449-461 and/or a sequence set forth in Table 8A. In one embodiment, the enhancer is selected from the group consisting of a serpin enhancer, a 3×HNF1-4_ProEnh_10mer, and a 5×HNF1_ProEnh_10mer. In one embodiment, the enhancer comprises a nucleic acid sequence having at least 85% identity to SEQ ID NO: 450. In one embodiments, the enhancer comprises a nucleic acid sequence having at least 85% identity to SEQ ID NO: 586. In one embodiment, the enhancer comprises a nucleic acid sequence having at least 85% identity to SEQ ID NO: 587.

In one embodiment, the ceDNA vector further comprises one or more introns. In one embodiment, the one or more introns comprises a sequence that is at least 85%, 90%, 95%, 96%, 97%, 98%, or 99% identical to, comprises, or consists of any one of SEQ ID NOs:509-516 and 1000 and/or a sequence set forth in Table 11A. In one embodiment, the one or more introns is the minute virus of mice (MVM).

In one embodiment, the ceDNA vector comprises a 3′ untranslated region (3′ UTR). In one embodiment, the 3′ UTR comprises a sequence that is at least 85%, 90%, 95%, 96%, 97%, 98%, or 99% identical to, comprises, or consists of any one of SEQ ID NOs:517-525 and/or a sequence set forth in Table 12.

In one embodiment, the ceDNA vector comprises a 5′ untranslated region (5′ UTR). In one embodiment, the 5′ UTR comprises a sequence that is at least 85%, 90%, 95%, 96%, 97%, 98%, or 99% identical to, comprises, or consists of any one of SEQ ID NOs:482-508 and/or a sequence set forth in Table 10.

In one embodiment, the ceDNA vector comprises at least one polyA sequence.

In one embodiment, the VD promoter comprises a SERP enhancer. In one embodiment, the VD promoter comprises a 3×SERP enhancer.

In one embodiment, the promoter is the TTR liver promoter and the ceDNA further comprises an MVM intron.

In one embodiment, the ceDNA vector comprises a nucleic acid sequence that is at least 90%, at least 95%, at least 96%, at least 97%, at least 98%, or at least 99% identical to, comprises, or consists of a nucleic acid sequence selected from the group consisting of: SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 570, SEQ ID NO: 571, SEQ ID NO: 572, SEQ ID NO: 573, SEQ ID NO: 574, SEQ ID NO: 575, SEQ ID NO: 576, SEQ ID NO: 577, SEQ ID NO: 578, SEQ ID NO: 579, SEQ ID NO: 580, SEQ ID NO: 581, SEQ ID NO: 582, SEQ ID NO: 583, and SEQ ID NO: 584.

In one embodiment, the at least one nucleic acid sequence is cDNA for PAH.

In one embodiment, at least one ITR comprises a functional terminal resolution site (TRS) and a Rep binding site. In one embodiment, one or both of the ITRs are from a virus selected from a parvovirus, a dependovirus, and an adeno-associated virus (AAV). In one embodiment, the flanking ITRs are symmetric or asymmetric. In one embodiment, the flanking ITRs are symmetrical or substantially symmetrical. In one embodiment, the flanking ITRs are asymmetric. In one embodiment, one or both of the ITRs are wild type, or wherein both of the ITRs are wild-type. In one embodiment, the flanking ITRs are from different viral serotypes. In one embodiment, the flanking ITRs are from a pair of viral serotypes shown in Table 2. In one embodiment, one or both of the ITRs comprises a sequence selected from the sequences in Table 3, Table 5A, Table 5B, or Table 6.

In one embodiment, at least one of the ITRs is altered from a wild-type AAV ITR sequence by a deletion, addition, or substitution that affects the overall three-dimensional conformation of the ITR. In one embodiment, one or both of the ITRs are derived from an AAV serotype selected from AAV1, AAV2, AAV3, AAV4, AAV5, AAV6, AAV7, AAV8, AAV9, AAV10, AAV11, and AAV12.

In one embodiment, one or both of the ITRs are synthetic. In one embodiment, one or both of the ITRs is not a wild type ITR, or wherein both of the ITRs are not wild-type.

In one embodiment, one or both of the ITRs is modified by a deletion, insertion, and/or substitution in at least one of the ITR regions selected from A, A′, B, B′, C, C′, D, and D′. In one embodiment, the deletion, insertion, and/or substitution results in the deletion of all or part of a stem-loop structure normally formed by the A, A′, B, B′ C, or C′ regions.

In one embodiment, one or both of the ITRs are modified by a deletion, insertion, and/or substitution that results in the deletion of all or part of a stem-loop structure normally formed by the B and B′ regions. In one embodiment, one or both of the ITRs are modified by a deletion, insertion, and/or substitution that results in the deletion of all or part of a stem-loop structure normally formed by the C and C′ regions. In one embodiment, one or both of the ITRs are modified by a deletion, insertion, and/or substitution that results in the deletion of part of a stem-loop structure normally formed by the B and B′ regions and/or part of a stem-loop structure normally formed by the C and C′ regions. In one embodiment, one or both of the ITRs comprise a single stem-loop structure in the region that normally comprises a first stem-loop structure formed by the B and B′ regions and a second stem-loop structure formed by the C and C′ regions. In one embodiment, one or both of the ITRs comprise a single stem and two loops in the region that normally comprises a first stem-loop structure formed by the B and B′ regions and a second stem-loop structure formed by the C and C′ regions.

In one embodiment, one or both of the ITRs comprise a single stem and a single loop in the region that normally comprises a first stem-loop structure formed by the B and B′ regions and a second stem-loop structure formed by the C and C′ regions.

In one embodiment, both ITRs are altered in a manner that results in an overall three-dimensional symmetry when the ITRs are inverted relative to each other.

In one embodiment, the ceDNA vector comprises a nucleic acid sequence that is at least 90% identical to SEQ ID NOs: 382-440 or SEQ ID NOs: 1011-1015. In one embodiment, the ceDNA vector comprises a nucleic acid sequence that is at least 95% identical to SEQ ID NOs: 382-440, or SEQ ID NOs: 1011-1015. In one embodiment, the ceDNA vector comprises a nucleic acid sequence that is at least 96% identical to SEQ ID NOs: 382-440 or SEQ ID NOs: 1011-1015. In one embodiment, the ceDNA vector comprises a nucleic acid sequence that is at least 97% identical to SEQ ID NOs: 382-440 or SEQ ID NOs: 1011-1015. In one embodiment, the ceDNA vector comprises a nucleic acid sequence that is at least 98% identical to SEQ ID NOs: 382-440 or SEQ ID NOs: 1011-1015. In one embodiment, the ceDNA vector comprises a nucleic acid sequence that is at least 99% identical to SEQ ID NOs: 382-440 or SEQ ID NOs: 1011-1015.

In another aspect, disclosed herein is a method of expressing a PAH protein in a cell, the method comprising contacting the cell with a ceDNA vector disclosed herein. In one embodiment, the cell is a photoreceptor or a RPE cell. In one embodiment, the cell is contacted in vitro or in vivo.

In another aspect, disclosed herein is a method of treating a subject with phenylketonuria (PKU), the method comprising administering to the subject a ceDNA vector disclosed herein. In one embodiment, the at least one nucleic acid sequence that encodes at least one PAH protein is selected from a sequence having at least 90% identity with any of the sequences set forth in Table 1A. In one embodiment, the subject exhibits at least about a 50% decrease in level of serum phenylalanine as compared to a level of serum phenylalanine in the subject prior to administration. In one embodiment, the subject exhibits at least about a 10% increase in PAH activity after administration as compared to a level of PAH activity prior to administration.

In one embodiment, the ceDNA vector is formulated in lipid nanoparticles. In one embodiment, the ceDNA vector is administered intravenously. In one embodiment, the ceDNA vector is adminstered intramuscularly. In one embodiment, the ceDNA vector is administered by infusion.

In another aspect, disclosed herein is a pharmaceutical composition comprising a ceDNA vector.

In another aspect, disclosed herein is a composition comprising a ceDNA vector and a lipid. In one embodiment, the lipid is a lipid nanoparticle (LNP).

In another aspect, disclosed herein is a kit comprising a ceDNA vector disclosed herein, a pharmaceutical composition disclosed herein, or a composition disclosed herein. In one embodiment, the kit comprises instructions for use.

These and other aspects of the disclosure are described in further detail below.

### DESCRIPTION OF DRAWINGS

The patent or application file contains at least one drawing executed in color. Copies of this patent or patent application publication with color drawing(s) will be provided by the Office upon request and payment of the necessary fee.

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1A provides the T-shaped stem-loop structure of a wild-type left ITR of AAV2 (SEQ ID NO: 52) with identification of A-A′ arm, B-B′ arm, C-C′ arm, two Rep binding sites (RBE and RBE′) and also shows the terminal resolution site (TRS). The RBE contains a series of 4 duplex tetramers that are believed to interact with either Rep 78 or Rep 68. In addition, the RBE′ is also believed to interact with Rep complex assembled on the wild-type ITR or mutated ITR in the construct. The D and D′ regions contain transcription factor binding sites and other conserved structure. FIG. 1B shows proposed Rep-catalyzed nicking and ligating activities in a wild-type left ITR (SEQ ID NO: 53), including the T-shaped stem-loop structure of the wild-type left ITR of AAV2 with identification of A-A′ arm, B-B′ arm, C-C′ arm, two Rep Binding sites (RBE and RBE′) and also shows the terminal resolution site (TRS), and the D and D′ region comprising several transcription factor binding sites and other conserved structure.

FIG. 2A provides the primary structure (polynucleic acid sequence) (left) and the secondary structure (right) of the RBE-containing portions of the A-A′ arm, and the C-C′ and B-B′ arm of the wild type left AAV2 ITR (SEQ ID NO: 54). FIG. 2B shows an exemplary mutated ITR (also referred to as a modified ITR) sequence for the left ITR. Shown is the primary structure (left) and the predicted secondary structure (right) of the RBE portion of the A-A′ arm, the C arm and B-B′ arm of an exemplary mutated left ITR (ITR-1, left) (SEQ ID NO: 113). FIG. 2C shows the primary structure (left) and the secondary structure (right) of the RBE-containing portion of the A-A′ loop, and the B-B′ and C-C′ arms of wild type right AAV2 ITR (SEQ ID NO: 55). FIG. 2D shows an exemplary right modified ITR. Shown is the primary structure (left) and the predicted secondary structure (right) of the RBE containing portion of the A-A′ arm, the B-B′ and the C arm of an exemplary mutant right ITR (ITR-1, right) (SEQ ID NO: 114). Any combination of left and right ITR (e.g., AAV2 ITRs or other viral serotype or synthetic ITRs) can be used as taught herein. Each of FIGS. 2A-2D polynucleotide sequences refer to the sequence used in the plasmid or bacmid/baculovirus genome used to produce the ceDNA as described herein. Also included in each of FIGS. 2A-2D are corresponding ceDNA secondary structures inferred from the ceDNA vector configurations in the plasmid or bacmid/baculovirus genome and the predicted Gibbs free energy values.

FIG. 3A is a schematic illustrating an upstream process for making baculovirus infected insect cells (BIICs) that are useful in the production of a ceDNA vector for expression of the PAH as disclosed herein in the process described in the schematic in FIG. 3B. FIG. 3B is a schematic of an exemplary method of ceDNA production and FIG. 3C illustrates a biochemical method and process to confirm ceDNA vector production. FIG. 3D and FIG. 3E are schematic illustrations describing a process for identifying the presence of ceDNA in DNA harvested from cell pellets obtained during the ceDNA production processes in FIG. 3B. FIG. 3D shows schematic expected bands for an exemplary ceDNA either left uncut or digested with a restriction endonuclease and then subjected to electrophoresis on either a native gel or a denaturing gel. The leftmost schematic is a native gel, and shows multiple bands suggesting that in its duplex and uncut form ceDNA exists in at least monomeric and dimeric states, visible as a faster-migrating smaller monomer and a slower-migrating dimer that is twice the size of the monomer. The schematic second from the left shows that when ceDNA is cut with a restriction endonuclease, the original bands are gone and faster-migrating (e.g., smaller) bands appear, corresponding to the expected fragment sizes remaining after the cleavage. Under denaturing conditions, the original duplex DNA is single-stranded and migrates as a species twice as large as observed on native gel because the complementary strands are covalently linked. Thus, in the second schematic from the right, the digested ceDNA shows a similar banding distribution to that observed on native gel, but the bands migrate as fragments twice the size of their native gel counterparts. The rightmost schematic shows that uncut ceDNA under denaturing conditions migrates as a single-stranded open circle, and thus the observed bands are twice the size of those observed under native conditions where the circle is not open. In this figure “kb” is used to indicate relative size of nucleotide molecules based, depending on context, on either nucleotide chain length (e.g., for the single stranded molecules observed in denaturing conditions) or number of basepairs (e.g., for the double-stranded molecules observed in native conditions). FIG. 3E shows DNA having a non-continuous structure. The ceDNA can be cut by a restriction endonuclease, having a single recognition site on the ceDNA vector, and generate two DNA fragments with different sizes (1 kb and 2 kb) in both neutral and denaturing conditions. FIG. 3E also shows a ceDNA having a linear and continuous structure. The ceDNA vector can be cut by the restriction endonuclease, and generate two DNA fragments that migrate as 1 kb and 2 kb in neutral conditions, but in denaturing conditions, the stands remain connected and produce single strands that migrate as 2 kb and 4 kb.

FIG. 4 is an exemplary picture of a denaturing gel running examples of ceDNA vectors with (+) or without (−) digestion with endonucleases (EcoRI for ceDNA construct 1 and 2; BamH1 for ceDNA construct 3 and 4; SpeI for ceDNA construct 5 and 6; and XhoI for ceDNA construct 7 and 8) Constructs 1-8 are described in Example 1 of International Patent Application No. PCT/US18/49996, which is incorporated herein in its entirety by reference. Sizes of bands highlighted with an asterisk were determined and provided on the bottom of the picture.

FIGS. 5A-5E are graphs depicting the results of the experiment described in Example 6. The effect of ceDNA vector comprising a PAH nucleic acid sequence that has been codon optimized (ceDNA412; hPAH_codop_ORF_v2) or a ceDNA vector comprising a PAH nucleic acid sequence that has been codon optimized (ceDNA1530; hPAH-r5-s29) with a 3×HS-CRM8_SERP_Enhancer, a TTR-promoter-d5pUTR and MVM_intron on correction of phenylalanine concentration (“PHE μM”) was assessed in individual mice at both a 0.5 μg and 5 μg hydrodynamic dose, over 21 days.

FIGS. 6A-6B are graphs depicting the results of the experiment described in Example 7. The effect of ceDNA vectors comprising a PAH nucleic acid sequence that has been codon optimized (ceDNA412, ceDNA1132, ceDNA1274 and ceDNA1527) on correction of phenylalanine concentration (“PHE μM”) was assessed in individual mice at both a 0.5 μg and 5 μg hydrodynamic dose, and the mean correction for all five mice in each group are shown in the graphs of FIGS. 6A-6B. PHE concentration did not decrease in the control animal (PAH^{enu2}: vehicle).

FIGS. 7A-7G are graphs depicting the results of the experiment described in Example 7 for individual mice.

FIGS. 8A-8B are graphs depicting the results of the experiment described in Example 8. The effect of ceDNA vectors comprising a PAH nucleic acid sequence that has been codon optimized (ceDNA1416, ceDNA1428, and ceDNA1528, ceDNA1414) on correction of phenylalanine concentration (“PHE μM”) was assessed in individual mice at both a 0.5 μg and 5 μg hydrodynamic dose, after 7 days, and the mean correction for all five mice in each group are shown in the graphs of FIGS. 8A-8B. PHE concentration did not decrease in the control animal (PAHenu2: vehicle).

FIGS. 9A-9E are graphs depicting the results of the experiment described in Example 8. The effect of ceDNA vectors comprising a PAH nucleic acid sequence that has been codon optimized (ceDNA1416, ceDNA1428, ceDNA1414 and ceDNA1528) on correction of phenylalanine concentration (“PHE μM”) was assessed in individual mice at a 0.5 μg hydrodynamic dose, after 7 days.

FIGS. 10A-10E are graphs depicting the results of the experiment described in Example 8. The effect of ceDNA vectors comprising a PAH nucleic acid sequence that has been codon optimized (ceDNA1416, ceDNA1428, ceDNA1414 and ceDNA1528) on correction of phenylalanine concentration (“PHE μM”) was assessed in individual mice at a 5 μg hydrodynamic dose, after 7 days.

FIGS. 11A-11B are graphs depicting the results of the experiment described in Example 8. The effect of ceDNA vectors comprising a PAH nucleic acid sequence that has been codon optimized (ceDNA1416, ceDNA1428, ceDNA1414 and ceDNA1528) on correction of phenylalanine concentration (“PHE μM”) was assessed in individual mice at both a 0.5 μg and 5 μg hydrodynamic dose, after 28 days, and the mean correction for all five mice in each group are shown in the graphs of FIGS. 11A-11B. PHE concentration did not decrease in the control animal (PAH^{enu2}: vehicle).

FIGS. 12A-12E are graphs depicting the results of the experiment described in Example 8. The effect of ceDNA vectors comprising a PAH nucleic acid sequence that has been codon optimized (ceDNA1416, ceDNA1428, ceDNA1414 and ceDNA1528) on correction of phenylalanine concentration (“PHE μM”) was assessed in individual mice at a 0.5 μg hydrodynamic dose, after 28 days.

FIGS. 13A-13E are graphs depicting the results of the experiment described in Example 8. The effect of ceDNA vectors comprising a PAH nucleic acid sequence that has been codon optimized (ceDNA1416, ceDNA1428, ceDNA1414 and ceDNA1528) on correction of phenylalanine concentration (“PHE μM”) was assessed in individual mice at a 5 μg hydrodynamic dose, after 28 days.

FIGS. 14A-14I are graphs depicting the results of the experiment described in Example 8. The effect of ceDNA vectors comprising a PAH nucleic acid sequence that has been codon optimized (ceDNA412, ceDNA1430, ceDNA1432, ceDNA1473, ceDNA1474, ceDNA1436, ceDNA1471, ceDNA1472) on correction of phenylalanine concentration (“PHE μM”) was assessed in individual mice at a 5 μg hydrodynamic dose, after 7 days.

FIGS. 15A-15I are graphs depicting the results of the experiment described in Example 9. The effect of ceDNA vectors comprising a PAH nucleic acid sequence that has been codon optimized (ceDNA412, ceDNA1476, ceDNA1479, ceDNA1939, ceDNA1940, ceDNA1941, ceDNA1942, ceDNA1943, ceDNA1944) on correction of serum phenylalanine levels (“PHE μM”) was assessed in individual mice at a 0.5 μg hydrodynamic dose, after 28 days.

FIGS. 16A-16D are graphs depicting the results of the experiment described in Example 10. The effect of ceDNA vectors comprising a PAH nucleic acid sequence that has been codon optimized (ceDNA412, ceDNA1939, ceDNA1955, ceDNA62) on correction of serum phenylalanine levels (“PHE μM”) was assessed in individual mice at a 0.5 μg hydrodynamic dose, after 28 days.

FIGS. 17A-17H are graphs depicting the results of the experiment described in Example 11. The effect of ceDNA vectors comprising a PAH nucleic acid sequence that has been codon optimized (ceDNA412, ceDNA2409, ceDNA2410, ceDNA2415, ceDNA2418, ceDNA2416, ceDNA2419, ceDNA2420) on correction of serum phenylalanine levels (“PHE μM”) was assessed in individual mice at a 0.5 μg hydrodynamic dose, after 28 days.

FIGS. 18A-18D are graphs depicting the results of the experiment described in Example 11. The effect of ceDNA vectors comprising a PAH nucleic acid sequence that has been codon optimized (ceDNA2415, ceDNA2418) on correction of serum phenylalanine levels (“PHE μM”) was assessed in individual mice at a 0.1 μg hydrodynamic dose, after 28 days.

FIGS. 19A-19F are graphs depicting the results of the experiment described in Example 12. The effect of ceDNA vectors comprising a PAH nucleic acid sequence that has been codon optimized (ceDNA412, ceDNA34, ceDNA36, ceDNA41, ceDNA42, ceDNA43) on correction of serum phenylalanine levels (“PHE μM”) was assessed in individual mice at a 0.5 μg hydrodynamic dose, after 28 days.

FIGS. 20A and 20B are schematic diagrams for an exemplary insertion of introns into PAH CDS. Chimeric PAH intron with functional splice donor and acceptor sites was inserted at the native position of intron 1 of PAH CDS. Intron flanking regions (33 bp) derived from PAH cDNA sequence were substituted for codon optimized sequence in PAH CDS. Figure discloses SEQ ID NO: 1022

FIG. 21 is a schematic for an exemplary insertion of introns into PAH CDS. Chimeric PAH intron with functional splice donor and acceptor sites was inserted at the native position of intron 1 of PAH CDS. The sequence of the regions flanking intron splice sites were altered to better match consensus sequences. Figure discloses SEQ ID NOS: 1023 and 1024, respectively, in order of appearance.

### DETAILED DESCRIPTION

Provided herein is a method for treating phenylketonuria (PKU) using a ceDNA vector comprising one or more codon optimized nucleic acids that encode an PAH therapeutic protein or fragment thereof. Also provided herein are ceDNA vectors for expression of PAH protein as described herein comprising one or more codon optimized nucleic acids that encode for the PAH protein or fragment thereof. It is a surprising finding of the present disclosure that codon optimized nucleic acids that encode a PAH therapeutic protein or fragment thereof, when combined with particular cis-acting elements (e.g., specific promoters and/or regulatory elements), provide optimal correction of phenylalanine level (e.g., expression and duration) in a subject.

According to some embodiments, an optimal correction of phenylalanine level is a level that is therapeutically effective to treat a disease or disorder resulting from a deficiency in phenylalanine hydroxylase (PAH). As described by the present disclosure, the constructs comprising codon optimized sequences performed considerably better than native hPAH cDNA sequence, and certain constructs comprising codon optimized sequences and particular cis-acting elements showed extended expression and correction of phenylalanine levels. In some embodiments, the expression of PAH protein can comprise secretion of the therapeutic protein out of the cell in which it is expressed or alternatively in some embodiments, the expressed PAH protein can act or function (e.g., exert its effect) within the cell in which it is expressed. In some embodiments, the ceDNA vector expresses PAH protein in the liver, a muscle (e.g., skeletal muscle) of a subject, or other body part, which can act as a depot for PAH therapeutic protein production and secretion to many systemic compartments.

### I. Definitions

Unless otherwise defined herein, scientific and technical terms used in connection with the present application shall have the meanings that are commonly understood by those of ordinary skill in the art to which this disclosure belongs. It should be understood that this disclosure is not limited to the particular methodology, protocols, and reagents, etc., described herein and as such can vary. The terminology used herein is for the purpose of describing particular embodiments only, and is not intended to limit the scope of the present disclosure, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), Fields Virology, 6^{th} Edition, published by Lippincott Williams & Wilkins, Philadelphia, PA, USA (2013), Knipe, D. M. and Howley, P. M. (ed.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the terms, “administration,” “administering” and variants thereof refers to introducing a composition or agent (e.g., a therapeutic nucleic acid or an immunosuppressant as described herein) into a subject and includes concurrent and sequential introduction of one or more compositions or agents. “Administration” can refer, e.g., to therapeutic, pharmacokinetic, diagnostic, research, placebo, and experimental methods. “Administration” also encompasses in vitro and ex vivo treatments. The introduction of a composition or agent into a subject is by any suitable route, including orally, pulmonarily, intranasally, parenterally (intravenously, intramuscularly, intraperitoneally, or subcutaneously), rectally, intralymphatically, intratumorally, or topically. The introduction of a composition or agent into a subject is by electroporation. Administration includes self-administration and the administration by another. Administration can be carried out by any suitable route. A suitable route of administration allows the composition or the agent to perform its intended function. For example, if a suitable route is intravenous, the composition is administered by introducing the composition or agent into a vein of the subject.

As used herein, the phrases “nucleic acid therapeutic”, “therapeutic nucleic acid” and “TNA” are used interchangeably and refer to any modality of therapeutic using nucleic acids as an active component of therapeutic agent to treat a disease or disorder. As used herein, these phrases refer to RNA-based therapeutics and DNA-based therapeutics. Non-limiting examples of RNA-based therapeutics include mRNA, antisense RNA and oligonucleotides, ribozymes, aptamers, interfering RNAs (RNAi), Dicer-substrate dsRNA, small hairpin RNA (shRNA), asymmetrical interfering RNA (aiRNA), guide RNA (gRNA), and microRNA (miRNA). Non-limiting examples of DNA-based therapeutics include minicircle DNA, minigene, viral DNA (e.g., Lentiviral or AAV genome) or non-viral synthetic DNA vectors, closed-ended linear duplex DNA (ceDNA/CELiD), plasmids, bacmids, doggybone (dbDNA™) DNA vectors, minimalistic immunological-defined gene expression (MIDGE)-vector, nonviral ministring DNA vector (linear-covalently closed DNA vector), or dumbbell-shaped DNA minimal vector (“dumbbell DNA”).

As used herein, an “effective amount” or “therapeutically effective amount” of a therapeutic agent, such as a PAH therapeutic protein or fragment thereof, is an amount sufficient to produce the desired effect, e.g., provide disease modifying levels of PAH enzyme, result in sustained expression of corrective PAH enzyme in the liver, restored urea cycle function, phenylalanine metabolism, and/or achieve the appropriate pharmacologic levels of the defective enzyme. Suitable assays for measuring expression of a target gene or target sequence include, e.g., examination of protein or RNA levels using techniques known to those of skill in the art such as dot blots, northern blots, in situ hybridization, ELISA, immunoprecipitation, enzyme function, as well as phenotypic assays known to those of skill in the art. However, dosage levels are based on a variety of factors, including the type of injury, the age, weight, sex, medical condition of the patient, the severity of the condition, the route of administration, and the particular active agent employed. Thus, the dosage regimen may vary widely, but can be determined routinely by a physician using standard methods. Additionally, the terms “therapeutic amount”, “therapeutically effective amounts” and “pharmaceutically effective amounts” include prophylactic or preventative amounts of the compositions of the described disclosure. In prophylactic or preventative applications of the described disclosure, pharmaceutical compositions or medicaments are administered to a patient susceptible to, or otherwise at risk of, a disease, disorder or condition in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition. It is generally preferred that a maximum dose be used, that is, the highest safe dose according to some medical judgment. According to some embodiments, the disease, disorder or condition is PKU. The terms “dose” and “dosage” are used interchangeably herein.

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

For any therapeutic agent described herein therapeutically effective amount may be initially determined from preliminary in vitro studies and/or animal models. A therapeutically effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10^{th} Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to therapeutic window, additional guidance for dosage modification can be obtained.

As used herein, the terms “heterologous nucleotide sequence” and “transgene” are used interchangeably and refer to a nucleic acid of interest (other than a nucleic acid encoding a capsid polypeptide) that is incorporated into and may be delivered and expressed by a ceDNA vector as disclosed herein. In one embodiment, the at least one nucleic acid sequence encoding the at least one PAH protein is a heterologous nucleic acid sequence.

As used herein, the terms “expression cassette” and “transcription cassette” are used interchangeably and refer to a linear stretch of nucleic acids that includes a transgene that is operably linked to one or more promoters or other regulatory sequences sufficient to direct transcription of the transgene, but which does not comprise capsid-encoding sequences, other vector sequences or inverted terminal repeat regions. An expression cassette may additionally comprise one or more cis-acting sequences (e.g., promoters, enhancers, or repressors), one or more introns, and one or more post-transcriptional regulatory elements.

The terms “polynucleotide” and “nucleic acid,” used interchangeably herein, refer to a polymeric form of nucleotides of any length, either ribonucleotides or deoxyribonucleotides. Thus, this term includes single, double, or multi-stranded DNA or RNA, genomic DNA, cDNA, DNA-RNA hybrids, or a polymer including purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides. DNA may be in the form of, e.g., antisense molecules, plasmid DNA, DNA-DNA duplexes, pre-condensed DNA, PCR products, vectors (P1, PAC, BAC, YAC, artificial chromosomes), expression cassettes, chimeric sequences, chromosomal DNA, or derivatives and combinations of these groups. DNA may be in the form of minicircle, plasmid, bacmid, minigene, ministring DNA (linear covalently closed DNA vector), closed-ended linear duplex DNA (CELiD or ceDNA), doggybone (dbDNA™) DNA, dumbbell shaped DNA, minimalistic immunological-defined gene expression (MIDGE)-vector, viral vector or nonviral vectors. RNA may be in the form of small interfering RNA (siRNA), Dicer-substrate dsRNA, small hairpin RNA (shRNA), asymmetrical interfering RNA (aiRNA), microRNA (miRNA), mRNA, gRNA, rRNA, tRNA, viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs and/or modified residues include, without limitation, phosphorothioates, phosphorodiamidate morpholino oligomer (morpholino), phosphoramidates, methyl phosphonates, chiral-methyl phosphonates, 2′-O-methyl ribonucleotides, locked nucleic acid (LNA™), and peptide nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated.

“Nucleotides” contain a sugar deoxyribose (DNA) or ribose (RNA), a base, and a phosphate group. Nucleotides are linked together through the phosphate groups.

“Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

As used herein, the term “interfering RNA” or “RNAi” or “interfering RNA sequence” includes single-stranded RNA (e.g., mature miRNA, ssRNAi oligonucleotides, ssDNAi oligonucleotides), double-stranded RNA (i.e., duplex RNA such as siRNA, Dicer-substrate dsRNA, shRNA, aiRNA, or pre-miRNA), a DNA-RNA hybrid (see, e.g., International Patent Application Publication No. WO 2004/078941), or a DNA-DNA hybrid (see, e.g., PCT Publication No. WO 2004/104199) that is capable of reducing or inhibiting the expression of a target gene or sequence (e.g., by mediating the degradation or inhibiting the translation of mRNAs which are complementary to the interfering RNA sequence) when the interfering RNA is in the same cell as the target gene or sequence. Interfering RNA thus refers to the single-stranded RNA that is complementary to a target mRNA sequence or to the double-stranded RNA formed by two complementary strands or by a single, self-complementary strand. Interfering RNA may have substantial or complete identity to the target gene or sequence, or may comprise a region of mismatch (i.e., a mismatch motif). The sequence of the interfering RNA can correspond to the full-length target gene, or a subsequence thereof. Preferably, the interfering RNA molecules are chemically synthesized. The disclosures of each of the above patent documents are herein incorporated by reference in their entirety for all purposes.

Interfering RNA includes “small-interfering RNA” or “siRNA,” e.g., interfering RNA of about 15-60, 15-50, or 15-40 (duplex) nucleotides in length, more typically about 15-30, 15-25, or 19-25 (duplex) nucleotides in length, and is preferably about 20-24, 21-22, or 21-23 (duplex) nucleotides in length (e.g., each complementary sequence of the double-stranded siRNA is 15-60, 15-50, 15-40, 15-30, 15-25, or 19-25 nucleotides in length, preferably about 20-24, 21-22, or 21-23 nucleotides in length, and the double-stranded siRNA is about 15-60, 15-50, 15-40, 15-30, 15-25, or 19-25 base pairs in length, preferably about 18-22, 19-20, or 19-21 base pairs in length). siRNA duplexes may comprise 3′ overhangs of about 1 to about 4 nucleotides or about 2 to about 3 nucleotides and 5′ phosphate termini. Examples of siRNA include, without limitation, a double-stranded polynucleotide molecule assembled from two separate stranded molecules, wherein one strand is the sense strand and the other is the complementary antisense strand; a double-stranded polynucleotide molecule assembled from a single stranded molecule, where the sense and antisense regions are linked by a nucleic acid-based or non-nucleic acid-based linker; a double-stranded polynucleotide molecule with a hairpin secondary structure having self-complementary sense and antisense regions; and a circular single-stranded polynucleotide molecule with two or more loop structures and a stem having self-complementary sense and antisense regions, where the circular polynucleotide can be processed in vivo or in vitro to generate an active double-stranded siRNA molecule. As used herein, the term “siRNA” includes RNA-RNA duplexes as well as DNA-RNA hybrids (see, e.g., PCT Publication No. WO 2004/078941, incorporated by reference in its entirety herein).

The term “nucleic acid construct” as used herein refers to a nucleic acid molecule, either single- or double-stranded, which is isolated from a naturally occurring gene or which is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present disclosure. An “expression cassette” includes a DNA coding sequence operably linked to a promoter.

By “hybridizable” or “complementary” or “substantially complementary” it is meant that a nucleic acid (e.g., RNA) includes a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C). In addition, it is also known in the art that for hybridization between two RNA molecules (e.g., dsRNA), guanine (G) base pairs with uracil (U). For example, G/U base-pairing is partially responsible for the degeneracy (i.e., redundancy) of the genetic code in the context of tRNA anti-codon base-pairing with codons in mRNA. In the context of this disclosure, a guanine (G) of a protein-binding segment (dsRNA duplex) of a subject DNA-targeting RNA molecule is considered complementary to an uracil (U), and vice versa. As such, when a G/U base-pair can be made at a given nucleotide position a protein-binding segment (dsRNA duplex) of a subject DNA-targeting RNA molecule, the position is not considered to be non-complementary, but is instead considered to be complementary.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

A DNA sequence that “encodes” a particular PAH protein is a DNA nucleic acid sequence that is transcribed into the particular RNA and/or protein. A DNA polynucleotide may encode an RNA (mRNA) that is translated into protein, or a DNA polynucleotide may encode an RNA that is not translated into protein (e.g., tRNA, rRNA, or a DNA-targeting RNA; also called “non-coding” RNA or “ncRNA”).

As used herein, the term “fusion protein” as used herein refers to a polypeptide which comprises protein domains from at least two different proteins. For example, a fusion protein may comprise (i) PAH or fragment thereof and (ii) at least one non-GOI protein. Fusion proteins encompassed herein include, but are not limited to, an antibody, or Fc or antigen-binding fragment of an antibody fused to a PAH protein, e.g., an extracellular domain of a receptor, ligand, enzyme or peptide. The PAH protein or fragment thereof that is part of a fusion protein can be a monospecific antibody or a bispecific or multispecific antibody.

As used herein, the term “genomic safe harbor gene” or “safe harbor gene” refers to a gene or loci that a nucleic acid sequence can be inserted such that the sequence can integrate and function in a predictable manner (e.g., express a protein of interest) without significant negative consequences to endogenous gene activity, or the promotion of cancer. In some embodiments, a safe harbor gene is also a loci or gene where an inserted nucleic acid sequence can be expressed efficiently and at higher levels than a non-safe harbor site.

As used herein, the term “gene delivery” means a process by which foreign DNA is transferred to host cells for applications of gene therapy.

As used herein, the term “terminal repeat” or “TR” includes any viral terminal repeat or synthetic sequence that comprises at least one minimal required origin of replication and a region comprising a palindrome hairpin structure. A Rep-binding sequence (“RBS”) (also referred to as RBE (Rep-binding element)) and a terminal resolution site (“TRS”) together constitute a “minimal required origin of replication” and thus the TR comprises at least one RBS and at least one TRS. TRs that are the inverse complement of one another within a given stretch of polynucleotide sequence are typically each referred to as an “inverted terminal repeat” or “ITR”. In the context of a virus, ITRs mediate replication, virus packaging, integration and provirus rescue. As was unexpectedly found in the disclosure herein, TRs that are not inverse complements across their full length can still perform the traditional functions of ITRs, and thus the term ITR is used herein to refer to a TR in a ceDNA genome or ceDNA vector that is capable of mediating replication of ceDNA vector. It will be understood by one of ordinary skill in the art that in complex ceDNA vector configurations more than two ITRs or asymmetric ITR pairs may be present. The ITR can be an AAV ITR or a non-AAV ITR, or can be derived from an AAV ITR or a non-AAV ITR. For example, the ITR can be derived from the family Parvoviridae, which encompasses parvoviruses and dependoviruses (e.g., canine parvovirus, bovine parvovirus, mouse parvovirus, porcine parvovirus, human parvovirus B-19), or the SV40 hairpin that serves as the origin of SV40 replication can be used as an ITR, which can further be modified by truncation, substitution, deletion, insertion and/or addition. Parvoviridae family viruses consist of two subfamilies: Parvovirinae, which infect vertebrates, and Densovirinae, which infect invertebrates. Dependoparvoviruses include the viral family of the adeno-associated viruses (AAV) which are capable of replication in vertebrate hosts including, but not limited to, human, primate, bovine, canine, equine and ovine species. For convenience herein, an ITR located 5′ to (upstream of) an expression cassette in a ceDNA vector is referred to as a “5′ ITR” or a “left ITR”, and an ITR located 3′ to (downstream of) an expression cassette in a ceDNA vector is referred to as a “3′ ITR” or a “right ITR”.

A “wild-type ITR” or “WT-ITR” refers to the sequence of a naturally occurring ITR sequence in an AAV or other Dependovirus that retains, e.g., Rep binding activity and Rep nicking ability. The nucleotide sequence of a WT-ITR from any AAV serotype may slightly vary from the canonical naturally occurring sequence due to degeneracy of the genetic code or drift, and therefore WT-ITR sequences encompassed for use herein include WT-ITR sequences as result of naturally occurring changes taking place during the production process (e.g., a replication error).

As used herein, the term “substantially symmetrical WT-ITRs” or a “substantially symmetrical WT-ITR pair” refers to a pair of WT-ITRs within a single ceDNA genome or ceDNA vector that are both wild type ITRs that have an inverse complement sequence across their entire length. For example, an ITR can be considered to be a wild-type sequence, even if it has one or more nucleotides that deviate from the canonical naturally occurring sequence, so long as the changes do not affect the properties and overall three-dimensional structure of the sequence. In some aspects, the deviating nucleotides represent conservative sequence changes. As one non-limiting example, a sequence that has at least 95%, 96%, 97%, 98%, or 99% sequence identity to the canonical sequence (as measured, e.g., using BLAST at default settings), and also has a symmetrical three-dimensional spatial organization to the other WT-ITR such that their 3D structures are the same shape in geometrical space. The substantially symmetrical WT-ITR has the same A, C-C′ and B-B′ loops in 3D space. A substantially symmetrical WT-ITR can be functionally confirmed as WT by determining that it has an operable Rep binding site (RBE or RBE′) and terminal resolution site (TRS) that pairs with the appropriate Rep protein. One can optionally test other functions, including transgene expression under permissive conditions.

As used herein, the phrases of “modified ITR” or “mod-ITR” or “mutant ITR” are used interchangeably herein and refer to an ITR that has a mutation in at least one or more nucleotides as compared to the WT-ITR from the same serotype. The mutation can result in a change in one or more of A, C, C′, B, B′ regions in the ITR, and can result in a change in the three-dimensional spatial organization (i.e. its 3D structure in geometric space) as compared to the 3D spatial organization of a WT-ITR of the same serotype.

As used herein, the term “asymmetric ITRs” also referred to as “asymmetric ITR pairs” refers to a pair of ITRs within a single ceDNA genome or ceDNA vector that are not inverse complements across their full length. As one non-limiting example, an asymmetric ITR pair does not have a symmetrical three-dimensional spatial organization to their cognate ITR such that their 3D structures are different shapes in geometrical space. Stated differently, an asymmetrical ITR pair have the different overall geometric structure, i.e., they have different organization of their A, C-C′ and B-B′ loops in 3D space (e.g., one ITR may have a short C-C′ arm and/or short B-B′ arm as compared to the cognate ITR). The difference in sequence between the two ITRs may be due to one or more nucleotide addition, deletion, truncation, or point mutation. In one embodiment, one ITR of the asymmetric ITR pair may be a wild-type AAV ITR sequence and the other ITR a modified ITR as defined herein (e.g., a non-wild-type or synthetic ITR sequence). In another embodiment, neither ITRs of the asymmetric ITR pair is a wild-type AAV sequence and the two ITRs are modified ITRs that have different shapes in geometrical space (i.e., a different overall geometric structure). In some embodiments, one mod-ITRs of an asymmetric ITR pair can have a short C-C′ arm and the other ITR can have a different modification (e.g., a single arm, or a short B-B′ arm etc.) such that they have different three-dimensional spatial organization as compared to the cognate asymmetric mod-ITR.

As used herein, the term “symmetric ITRs” refers to a pair of ITRs within a single ceDNA genome or ceDNA vector that are wild-type or mutated (e.g., modified relative to wild-type) dependoviral ITR sequences and are inverse complements across their full length. In one non-limiting example, both ITRs are wild type ITRs sequences from AAV2. In another example, neither ITRs are wild type ITR AAV2 sequences (i.e., they are a modified ITR, also referred to as a mutant ITR), and can have a difference in sequence from the wild type ITR due to nucleotide addition, deletion, substitution, truncation, or point mutation. For convenience herein, an ITR located 5′ to (upstream of) an expression cassette in a ceDNA vector is referred to as a “5′ ITR” or a “left ITR”, and an ITR located 3′ to (downstream of) an expression cassette in a ceDNA vector is referred to as a “3′ ITR” or a “right ITR”.

As used herein, the terms “substantially symmetrical modified-ITRs” or a “substantially symmetrical mod-ITR pair” refers to a pair of modified-ITRs within a single ceDNA genome or ceDNA vector that are both that have an inverse complement sequence across their entire length. For example, the modified ITR can be considered substantially symmetrical, even if it has some nucleotide sequences that deviate from the inverse complement sequence so long as the changes do not affect the properties and overall shape. As one non-limiting example, a sequence that has at least 85%, 90%, 95%, 96%, 97%, 98%, or 99% sequence identity to the canonical sequence (as measured using BLAST at default settings), and also has a symmetrical three-dimensional spatial organization to their cognate modified ITR such that their 3D structures are the same shape in geometrical space. Stated differently, a substantially symmetrical modified-ITR pair have the same A, C-C′ and B-B′ loops organized in 3D space. In some embodiments, the ITRs from a mod-ITR pair may have different reverse complement nucleotide sequences but still have the same symmetrical three-dimensional spatial organization—that is both ITRs have mutations that result in the same overall 3D shape. For example, one ITR (e.g., 5′ ITR) in a mod-ITR pair can be from one serotype, and the other ITR (e.g., 3′ ITR) can be from a different serotype, however, both can have the same corresponding mutation (e.g., if the 5′ITR has a deletion in the C region, the cognate modified 3′ITR from a different serotype has a deletion at the corresponding position in the C′ region), such that the modified ITR pair has the same symmetrical three-dimensional spatial organization. In such embodiments, each ITR in a modified ITR pair can be from different serotypes (e.g., AAV1, 2, 3, 4, 5, 6, 7, 8, 9, 10, 11, and 12) such as the combination of AAV2 and AAV6, with the modification in one ITR reflected in the corresponding position in the cognate ITR from a different serotype. In one embodiment, a substantially symmetrical modified ITR pair refers to a pair of modified ITRs (mod-ITRs) so long as the difference in nucleotide sequences between the ITRs does not affect the properties or overall shape and they have substantially the same shape in 3D space. As a non-limiting example, a mod-ITR that has at least 95%, 96%, 97%, 98% or 99% sequence identity to the canonical mod-ITR as determined by standard means well known in the art such as BLAST (Basic Local Alignment Search Tool), or BLASTN at default settings, and also has a symmetrical three-dimensional spatial organization such that their 3D structure is the same shape in geometric space. A substantially symmetrical mod-ITR pair has the same A, C-C′ and B-B′ loops in 3D space, e.g., if a modified ITR in a substantially symmetrical mod-ITR pair has a deletion of a C-C′ arm, then the cognate mod-ITR has the corresponding deletion of the C-C′ loop and also has a similar 3D structure of the remaining A and B-B′ loops in the same shape in geometric space of its cognate mod-ITR.

The term “flanking” refers to a relative position of one nucleic acid sequence with respect to another nucleic acid sequence. Generally, in the sequence ABC, B is flanked by A and C. The same is true for the arrangement A×B×C. Thus, a flanking sequence precedes or follows a flanked sequence but need not be contiguous with, or immediately adjacent to the flanked sequence. In one embodiment, the term flanking refers to terminal repeats at each end of the linear duplex ceDNA vector.

As used herein, the terms “treat,” “treating,” and/or “treatment” include abrogating, substantially inhibiting, slowing or reversing the progression of a condition, substantially ameliorating clinical symptoms of a condition, or substantially preventing the appearance of clinical symptoms of a condition, obtaining beneficial or desired clinical results. According to some embodiments, the condition is PKU. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s). Beneficial or desired clinical results, such as pharmacologic and/or physiologic effects include, but are not limited to, preventing the disease, disorder or condition from occurring in a subject that may be predisposed to the disease, disorder or condition but does not yet experience or exhibit symptoms of the disease (prophylactic treatment), alleviation of symptoms of the disease, disorder or condition, diminishment of extent of the disease, disorder or condition, stabilization (i.e., not worsening) of the disease, disorder or condition, preventing spread of the disease, disorder or condition, delaying or slowing of the disease, disorder or condition progression, amelioration or palliation of the disease, disorder or condition, and combinations thereof, as well as prolonging survival as compared to expected survival if not receiving treatment.

As used herein, the term “increase,” “enhance,” “raise” (and like terms) generally refers to the act of increasing, either directly or indirectly, a concentration, level, function, activity, or behavior relative to the natural, expected, or average, or relative to a control condition.

As used herein, the term “minimize”, “reduce”, “decrease,” and/or “inhibit” (and like terms) generally refers to the act of reducing, either directly or indirectly, a concentration, level, function, activity, or behavior relative to the natural, expected, or average, or relative to a control condition.

As used herein, the term “ceDNA genome” refers to an expression cassette that further incorporates at least one inverted terminal repeat region. A ceDNA genome may further comprise one or more spacer regions. In some embodiments the ceDNA genome is incorporated as an intermolecular duplex polynucleotide of DNA into a plasmid or viral genome.

As used herein, the term “ceDNA spacer region” refers to an intervening sequence that separates functional elements in the ceDNA vector or ceDNA genome. In some embodiments, ceDNA spacer regions keep two functional elements at a desired distance for optimal functionality. In some embodiments, ceDNA spacer regions provide or add to the genetic stability of the ceDNA genome within e.g., a plasmid or baculovirus. In some embodiments, ceDNA spacer regions facilitate ready genetic manipulation of the ceDNA genome by providing a convenient location for cloning sites and the like. For example, in certain aspects, an oligonucleotide “polylinker” containing several restriction endonuclease sites, or a non-open reading frame sequence designed to have no known protein (e.g., transcription factor) binding sites can be positioned in the ceDNA genome to separate the cis-acting factors, e.g., inserting a 2mer, 3mer, 5mer, 6mer, 10mer, 11mer, 12mer, 18mer, 24mer, 30mer, 48mer, 86mer, 176mer, etc. between the terminal resolution site and the upstream transcriptional regulatory element. Similarly, the spacer may be incorporated between the polyadenylation signal sequence and the 3′-terminal resolution site.

As used herein, the terms “Rep binding site, “Rep binding element, “RBE” and “RBS” are used interchangeably and refer to a binding site for Rep protein (e.g., AAV Rep 78 or AAV Rep 68) which upon binding by a Rep protein permits the Rep protein to perform its site-specific endonuclease activity on the sequence incorporating the RBS. An RBS sequence and its inverse complement together form a single RBS. RBS sequences are known in the art, and include, for example, 5′-GCGCGCTCGCTCGCTC-3′ (SEQ ID NO: 60), an RBS sequence identified in AAV2. Any known RBS sequence may be used in the embodiments of the disclosure, including other known AAV RBS sequences and other naturally known or synthetic RBS sequences. Without being bound by theory it is thought that he nuclease domain of a Rep protein binds to the duplex nucleotide sequence GCTC, and thus the two known AAV Rep proteins bind directly to and stably assemble on the duplex oligonucleotide, 5′-(GCGC)(GCTC)(GCTC)(GCTC)-3′ (SEQ ID NO: 60). In addition, soluble aggregated conformers (i.e., undefined number of inter-associated Rep proteins) dissociate and bind to oligonucleotides that contain Rep binding sites. Each Rep protein interacts with both the nitrogenous bases and phosphodiester backbone on each strand. The interactions with the nitrogenous bases provide sequence specificity whereas the interactions with the phosphodiester backbone are non- or less-sequence specific and stabilize the protein-DNA complex.

As used herein, the terms “terminal resolution site” and “TRS” are used interchangeably herein and refer to a region at which Rep forms a tyrosine-phosphodiester bond with the 5′ thymidine generating a 3′ OH that serves as a substrate for DNA extension via a cellular DNA polymerase, e.g., DNA pol delta or DNA pol epsilon. Alternatively, the Rep-thymidine complex may participate in a coordinated ligation reaction. In some embodiments, a TRS minimally encompasses a non-base-paired thymidine. In some embodiments, the nicking efficiency of the TRS can be controlled at least in part by its distance within the same molecule from the RBS. When the acceptor substrate is the complementary ITR, then the resulting product is an intramolecular duplex. TRS sequences are known in the art, and include, for example, 5′-GGTTGA-3′ (SEQ ID NO: 61), the hexanucleotide sequence identified in AAV2. Any known TRS sequence may be used in the embodiments of the disclosure, including other known AAV TRS sequences and other naturally known or synthetic TRS sequences such as AGTT (SEQ ID NO: 62), GGTTGG (SEQ ID NO: 63), AGTTGG (SEQ ID NO: 64), AGTTGA (SEQ ID NO: 65), and other motifs such as RRTTRR (SEQ ID NO: 66).

As used herein, the term “ceDNA-plasmid” refers to a plasmid that comprises a ceDNA genome as an intermolecular duplex.

As used herein, the term “ceDNA-bacmid” refers to an infectious baculovirus genome comprising a ceDNA genome as an intermolecular duplex that is capable of propagating in E. coli as a plasmid, and so can operate as a shuttle vector for baculovirus.

As used herein, the term “ceDNA-baculovirus” refers to a baculovirus that comprises a ceDNA genome as an intermolecular duplex within the baculovirus genome.

As used herein, the terms “ceDNA-baculovirus infected insect cell” and “ceDNA-BIIC” are used interchangeably, and refer to an invertebrate host cell (including, but not limited to an insect cell (e.g., an Sf9 cell)) infected with a ceDNA-baculovirus.

As used herein, the term “ceDNA” refers to capsid-free closed-ended linear double stranded (ds) duplex DNA for non-viral gene transfer, synthetic or otherwise. Detailed description of ceDNA is described in International Patent Application No. PCT/US2017/020828, filed Mar. 3, 2017, the entire contents of which are expressly incorporated herein by reference. Certain methods for the production of ceDNA comprising various inverted terminal repeat (ITR) sequences and configurations using cell-based methods are described in Example 1 of International Patent Application Nos. PCT/US18/49996, filed Sep. 7, 2018, and PCT/US2018/064242, filed Dec. 6, 2018 each of which is incorporated herein in its entirety by reference. Certain methods for the production of synthetic ceDNA vectors comprising various ITR sequences and configurations are described, e.g., in International Patent Application No. PCT/US2019/14122, filed Jan. 18, 2019, the entire content of which is incorporated herein by reference.

As used herein, the term “closed-ended DNA vector” refers to a capsid-free DNA vector with at least one covalently closed end and where at least part of the vector has an intramolecular duplex structure.

As used herein, the terms “ceDNA vector” and “ceDNA” are used interchangeably and refer to a closed-ended DNA vector comprising at least one terminal palindrome. In some embodiments, the ceDNA comprises two covalently-closed ends.

As used herein, the term “neDNA” or “nicked ceDNA” refers to a closed-ended DNA having a nick or a gap of 1-100 base pairs in a stem region or spacer region 5′ upstream of an open reading frame (e.g., a promoter and transgene to be expressed).

As used herein, the terms “gap” and “nick” are used interchangeably and refer to a discontinued portion of synthetic DNA vector of the present disclosure, creating a stretch of single stranded DNA portion in otherwise double stranded ceDNA. The gap can be 1 base-pair to 100 base-pair long in length in one strand of a duplex DNA. Typical gaps, designed and created by the methods described herein and synthetic vectors generated by the methods can be, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 bp long in length. Exemplified gaps in the present disclosure can be 1 bp to 10 bp long, 1 to 20 bp long, 1 to 30 bp long in length.

As defined herein, “reporters” refer to proteins that can be used to provide detectable read-outs. Reporters generally produce a measurable signal such as fluorescence, color, or luminescence. Reporter protein coding sequences encode proteins whose presence in the cell or organism is readily observed. For example, fluorescent proteins cause a cell to fluoresce when excited with light of a particular wavelength, luciferases cause a cell to catalyze a reaction that produces light, and enzymes such as β-galactosidase convert a substrate to a colored product. Exemplary reporter polypeptides useful for experimental or diagnostic purposes include, but are not limited to β-lactamase, β-galactosidase (LacZ), alkaline phosphatase (AP), thymidine kinase (TK), green fluorescent protein (GFP) and other fluorescent proteins, chloramphenicol acetyltransferase (CAT), luciferase, and others well known in the art.

As used herein, the terms “sense” and “antisense” refer to the orientation of the structural element on the polynucleotide. The sense and antisense versions of an element are the reverse complement of each other.

As used herein, the term “synthetic AAV vector” and “synthetic production of AAV vector” refers to an AAV vector and synthetic production methods thereof in an entirely cell-free environment.

As used herein, “reporters” refer to proteins that can be used to provide detectable read-outs. Reporters generally produce a measurable signal such as fluorescence, color, or luminescence. Reporter protein coding sequences encode proteins whose presence in the cell or organism is readily observed. For example, fluorescent proteins cause a cell to fluoresce when excited with light of a particular wavelength, luciferases cause a cell to catalyze a reaction that produces light, and enzymes such as β-galactosidase convert a substrate to a colored product. Exemplary reporter polypeptides useful for experimental or diagnostic purposes include, but are not limited to β-lactamase, β-galactosidase (LacZ), alkaline phosphatase (AP), thymidine kinase (TK), green fluorescent protein (GFP) and other fluorescent proteins, chloramphenicol acetyltransferase (CAT), luciferase, and others well known in the art.

As used herein, the term “effector protein” refers to a polypeptide that provides a detectable read-out, either as, for example, a reporter polypeptide, or more appropriately, as a polypeptide that kills a cell, e.g., a toxin, or an agent that renders a cell susceptible to killing with a chosen agent or lack thereof. Effector proteins include any protein or peptide that directly targets or damages the host cell's DNA and/or RNA. For example, effector proteins can include, but are not limited to, a restriction endonuclease that targets a host cell DNA sequence (whether genomic or on an extrachromosomal element), a protease that degrades a polypeptide target necessary for cell survival, a DNA gyrase inhibitor, and a ribonuclease-type toxin. In some embodiments, the expression of an effector protein controlled by a synthetic biological circuit as described herein can participate as a factor in another synthetic biological circuit to thereby expand the range and complexity of a biological circuit system's responsiveness.

Transcriptional regulators refer to transcriptional activators and repressors that either activate or repress transcription of a gene of interest, such as PAH. Promoters are regions of nucleic acid that initiate transcription of a particular gene Transcriptional activators typically bind nearby to transcriptional promoters and recruit RNA polymerase to directly initiate transcription. Repressors bind to transcriptional promoters and sterically hinder transcriptional initiation by RNA polymerase. Other transcriptional regulators may serve as either an activator or a repressor depending on where they bind and cellular and environmental conditions. Non-limiting examples of transcriptional regulator classes include, but are not limited to homeodomain proteins, zinc-finger proteins, winged-helix (forkhead) proteins, and leucine-zipper proteins.

As used herein, a “repressor protein” or “inducer protein” is a protein that binds to a regulatory sequence element and represses or activates, respectively, the transcription of sequences operatively linked to the regulatory sequence element. Preferred repressor and inducer proteins as described herein are sensitive to the presence or absence of at least one input agent or environmental input. Preferred proteins as described herein are modular in form, comprising, for example, separable DNA-binding and input agent-binding or responsive elements or domains.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Supplementary active ingredients can also be incorporated into the compositions. The phrase “pharmaceutically-acceptable” refers to molecular entities and compositions that do not produce a toxic, an allergic, or similar untoward reaction when administered to a host.

As used herein, an “input agent responsive domain” is a domain of a transcription factor that binds to or otherwise responds to a condition or input agent in a manner that renders a linked DNA binding fusion domain responsive to the presence of that condition or input. In one embodiment, the presence of the condition or input results in a conformational change in the input agent responsive domain, or in a protein to which it is fused, that modifies the transcription-modulating activity of the transcription factor.

The term “in vivo” refers to assays or processes that occur in or within an organism, such as a multicellular animal. In some of the aspects described herein, a method or use can be said to occur “in vivo” when a unicellular organism, such as a bacterium, is used. The term “ex vivo” refers to methods and uses that are performed using a living cell with an intact membrane that is outside of the body of a multicellular animal or plant, e.g., explants, cultured cells, including primary cells and cell lines, transformed cell lines, and extracted tissue or cells, including blood cells, among others. The term “in vitro” refers to assays and methods that do not require the presence of a cell with an intact membrane, such as cellular extracts, and can refer to the introducing of a programmable synthetic biological circuit in a non-cellular system, such as a medium not comprising cells or cellular systems, such as cellular extracts.

The term “promoter,” as used herein, refers to any nucleic acid sequence that regulates the expression of another nucleic acid sequence by driving transcription of the nucleic acid sequence, which can be a target gene, e.g., a heterologous target gene, encoding a protein or an RNA. Promoters can be constitutive, inducible, repressible, tissue-specific, or any combination thereof. A promoter is a control region of a nucleic acid sequence at which initiation and rate of transcription of the remainder of a nucleic acid sequence are controlled. A promoter can also contain genetic elements at which regulatory proteins and molecules can bind, such as RNA polymerase and other transcription factors. In some embodiments of the aspects described herein, a promoter can drive the expression of a transcription factor that regulates the expression of the promoter itself. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the expression of transgenes in the ceDNA vectors disclosed herein. A promoter sequence may be bounded at its 3′ terminus by the transcription initiation site and extends upstream (5′ direction) to include the minimum number of bases or elements necessary to initiate transcription at levels detectable above background. According to some embodiments, the promoter is selected from the group consisting of the VD promoter, human alpha 1-antitrypsin (hAAT) promoter (including the hAAT(979) promoter and the CpGmin_hAAT promoters such as hAAT_core_C06; hAAT_core_C07; hAAT_core_C08; hAAT_core_C09; hAAT_core_C10; or hAAT_core truncated and the transthyretin (TTR) liver specific promoter, including the minimal TTR (TTRm).

The term “enhancer” as used herein refers to a cis-acting regulatory sequence (e.g., 50-1,500 base pairs) that binds one or more proteins (e.g., activator proteins, or transcription factor) to increase transcriptional activation of a nucleic acid sequence. Enhancers can be positioned up to 1,000,000 base pars upstream of the gene start site or downstream of the gene start site that they regulate. An enhancer can be positioned within an intronic region, or in the exonic region of an unrelated gene. According to some embodiments, the enhancer is selected from the group consisting of a serpin enhancer (SerpEnh) of human origin or other mammalian origin such as bushbaby or Chinese tree shrew, TTR enhancer (TTRe), Hepatic Nuclear Factor 1 binding site (HNF1), Hepatic Nuclear Factor 4 binding site (HNF4), human apolipoprotein E/C-I Liver specific enhancer (ApoE Enh), enhancer regions from Pro-albumin gene (ProEnh), CpG minimized ApoE enhancers (e.g., ApoE enhancer C03, ApoE enhancer C04, ApoE enhancer C09, or ApoE enhancer C10 as described herein), HCR1 footprint123 (embedded HCR1 footprint123), Hepatic Nuclear Factor enhancer array (Embedded enhancer HNF array), and derivative of human apolipoprotine E/C-I liver specific enhancer (e.g., ApoE Enh v2). According to some embodiments, the enhancer can be a multitude (e.g., tandem repeat) of a single enhancer element, or different type of enhancers like 3×HNF1-4_Pro-Albumin Enhancer as in ceDNA1471 in which the enhancers are linked to a TTR promoter) or 5×HNF1_Pro-Albumin Enhancer (as in ceDNA1473 in which the enhancers are linked to a TTR promoter). According to some embodiments, the multitude of enhancers, such as HNF1 and/or HNF4 may contain spacer regions between every two enhancer elements, e.g., 1-20 nucleotides, or about 1, 2, 3, 4, 5, 6, 7, 8, 9, 10, 11, 12, 13, 14, 15, 16, 17, 18, 19, or 20 nucleotides.

A promoter can be said to drive expression or drive transcription of the nucleic acid sequence that it regulates. The phrases “operably linked,” “operatively positioned,” “operatively linked,” “under control,” and “under transcriptional control” indicate that a promoter is in a correct functional location and/or orientation in relation to a nucleic acid sequence it regulates to control transcriptional initiation and/or expression of that sequence. An “inverted promoter,” as used herein, refers to a promoter in which the nucleic acid sequence is in the reverse orientation, such that what was the coding strand is now the non-coding strand, and vice versa. Inverted promoter sequences can be used in various embodiments to regulate the state of a switch. In addition, in various embodiments, a promoter can be used in conjunction with an enhancer.

A promoter can be one naturally associated with a gene or sequence, as can be obtained by isolating the 5′ non-coding sequences located upstream of the coding segment and/or exon of a given gene or sequence. Such a promoter can be referred to as “endogenous.” Similarly, in some embodiments, an enhancer can be one naturally associated with a nucleic acid sequence, located either downstream or upstream of that sequence.

In some embodiments, a coding nucleic acid segment is positioned under the control of a “recombinant promoter” or “heterologous promoter,” both of which refer to a promoter that is not normally associated with the encoded nucleic acid sequence it is operably linked to in its natural environment. A recombinant or heterologous enhancer refers to an enhancer not normally associated with a given nucleic acid sequence in its natural environment. Such promoters or enhancers can include promoters or enhancers of other genes; promoters or enhancers isolated from any other prokaryotic, viral, or eukaryotic cell; and synthetic promoters or enhancers that are not “naturally occurring,” i.e., comprise different elements of different transcriptional regulatory regions, and/or mutations that alter expression through methods of genetic engineering that are known in the art. In addition to producing nucleic acid sequences of promoters and enhancers synthetically, promoter sequences can be produced using recombinant cloning and/or nucleic acid amplification technology, including PCR, in connection with the synthetic biological circuits and modules disclosed herein (see, e.g., U.S. Pat. Nos. 4,683,202, 5,928,906, each incorporated herein by reference). Furthermore, it is contemplated that control sequences that direct transcription and/or expression of sequences within non-nuclear organelles such as mitochondria, chloroplasts, and the like, can be employed as well. Non-limiting examples of such a recombinant or heterologous promoter/enhancer include a serpin enhancer with a TTR promoter (referred to as “Vandendriessche promoter” (VD) or Vandendriesche (VD) promoter set (see, e.g., U.S. Pat. No. 10,149,914, incorporated herein by reference) or 3× serpin enhancers with a TTR promoter (referred to as “3× VD” promoter set; see, e.g., U.S. Patent Application Publication No. US 2018/0071406A1, incorporated herein by reference).

In some embodiments, a promoter can be a promoter set. The term “promoter set,” as used herein, refers to a system comprising one or more promoters (or promoter sequences) as defined herein and one or more enhancers (or enhancer sequences) as defined herein. The term “promoter set” as used herein encompasses sequences whereby the promoter and enhancer elements or sequences are separated by spacer regions or sequences that are about 1-50 nucleotides in length, e.g., about 2, 5, 7, 8, 10, 11, 12, 13, 15, 17, 18, 20, 22, 23, 25, 27, 28, 30, 32, 33, 35, 37, 38, 40, 42, 43, 45, 47, 48, or 50 nucleotides.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., DNA-targeting RNA) or a coding sequence (e.g., site-directed modifying polypeptide, or Cas9/Csn1 polypeptide) and/or regulate translation of an encoded polypeptide.

“Operably linked” refers to a juxtaposition wherein the components so described are in a relationship permitting them to function in their intended manner. For instance, a promoter is operably linked to a coding sequence if the promoter affects its transcription or expression. An “expression cassette” includes a DNA sequence, e.g., a heterologous DNA sequence, that is operably linked to a promoter or other regulatory sequence sufficient to direct transcription of the transgene in the ceDNA vector. Suitable promoters include, for example, tissue specific promoters. Promoters can also be of AAV origin.

The term “subject” as used herein refers to a human or animal, to whom treatment, including prophylactic treatment, with the ceDNA vector according to the present disclosure, is provided. Usually, the animal is a vertebrate such as, but not limited to a primate, rodent, domestic animal or game animal. Primates include but are not limited to, chimpanzees, cynomologous monkeys, spider monkeys, and macaques, e.g., Rhesus. Rodents include mice, rats, woodchucks, ferrets, rabbits and hamsters. Domestic and game animals include, but are not limited to, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments of the aspects described herein, the subject is a mammal, e.g., a primate or a human. A subject can be male or female. Additionally, a subject can be an infant or a child. In some embodiments, the subject can be a neonate or an unborn subject, e.g., the subject is in utero. Preferably, the subject is a mammal. The mammal can be a human, non-human primate, mouse, rat, dog, cat, horse, or cow, but is not limited to these examples. Mammals other than humans can be advantageously used as subjects that represent animal models of diseases and disorders. In addition, the methods and compositions described herein can be used for domesticated animals and/or pets. A human subject can be of any age, gender, race or ethnic group, e.g., Caucasian (white), Asian, African, black, African American, African European, Hispanic, Mideastern, etc. In some embodiments, the subject can be a patient or other subject in a clinical setting. In some embodiments, the subject is already undergoing treatment. In some embodiments, the subject is an embryo, a fetus, neonate, infant, child, adolescent, or adult. In some embodiments, the subject is a human fetus, human neonate, human infant, human child, human adolescent, or human adult. In some embodiments, the subject is an animal embryo, or non-human embryo or non-human primate embryo. In some embodiments, the subject is a human embryo.

As used herein, the term “host cell”, includes any cell type that is susceptible to transformation, transfection, transduction, and the like with a nucleic acid construct or ceDNA expression vector of the present disclosure. As non-limiting examples, a host cell can be an isolated primary cell, pluripotent stem cells, CD34⁺ cells), induced pluripotent stem cells, or any of a number of immortalized cell lines (e.g., HepG2 cells). Alternatively, a host cell can be an in situ or in vivo cell in a tissue, organ or organism.

The term “exogenous” refers to a substance present in a cell other than its native source. The term “exogenous” when used herein can refer to a nucleic acid (e.g., a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell.

The term “sequence identity” refers to the relatedness between two nucleic acid sequences. For purposes of the present disclosure, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows: (Identical Deoxyribonucleotides.times.100)/(Length of Alignment−Total Number of Gaps in Alignment). The length of the alignment is preferably at least 10 nucleotides, preferably at least 25 nucleotides more preferred at least 50 nucleotides and most preferred at least 100 nucleotides.

The term “homology” or “homologous” as used herein is defined as the percentage of nucleotide residues that are identical to the nucleotide residues in the corresponding sequence on the target chromosome, after aligning the sequences and introducing gaps, if necessary, to achieve the maximum percent sequence identity. Alignment for purposes of determining percent nucleotide sequence homology can be achieved in various ways that are within the skill in the art, for instance, using publicly available computer software such as BLAST, BLAST-2, ALIGN, ClustalW2 or Megalign (DNASTAR) software. Those skilled in the art can determine appropriate parameters for aligning sequences, including any algorithms needed to achieve maximal alignment over the full length of the sequences being compared. In some embodiments, a nucleic acid sequence (e.g., DNA sequence), for example of a homology arm, is considered “homologous” when the sequence is at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to the corresponding native or unedited nucleic acid sequence (e.g., genomic sequence) of the host cell.

The term “heterologous,” as used herein, means a nucleic acid or polypeptide sequence that is not found in the native nucleic acid or protein, respectively. A heterologous nucleic acid sequence may be linked to a naturally-occurring nucleic acid sequence (or a variant thereof) (e.g., by genetic engineering) to generate a chimeric nucleic acid sequence encoding a chimeric polypeptide. A heterologous nucleic acid sequence may be linked to a variant polypeptide (e.g., by genetic engineering) to generate a nucleic acid sequence encoding a fusion variant polypeptide.

A “vector” or “expression vector” is a replicon, such as plasmid, bacmid, phage, virus, virion, or cosmid, to which another DNA segment, i.e., an “insert”, may be attached so as to bring about the replication of the attached segment in a cell. A vector can be a nucleic acid construct designed for delivery to a host cell or for transfer between different host cells. As used herein, a vector can be viral or non-viral in origin and/or in final form, however for the purpose of the present disclosure, a “vector” generally refers to a ceDNA vector, as that term is used herein. The term “vector” encompasses any genetic element that is capable of replication when associated with the proper control elements and that can transfer gene sequences to cells. In some embodiments, a vector can be an expression vector or recombinant vector.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

By “recombinant vector” is meant a vector that includes a nucleic acid sequence, e.g., a heterologous nucleic acid or “transgene”, that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

The phrase “genetic disease” as used herein refers to a disease, partially or completely, directly or indirectly, caused by one or more abnormalities in the genome, especially a condition that is present from birth. The abnormality may be a mutation, an insertion or a deletion. The abnormality may affect the coding sequence of the gene or its regulatory sequence. The genetic disease may be, but not limited to PKU, DMD, hemophilia, cystic fibrosis, Huntington's chorea, familial hypercholesterolemia (LDL receptor defect), hepatoblastoma, Wilson's disease, congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, and Tay-Sachs disease.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the disclosure.

As used in this specification and the appended claims, the singular forms “a,” “an,” and “the” include plural references unless the context clearly dictates otherwise. Thus, for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the disclosure disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the disclosure.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. It should be understood that this disclosure is not limited to the particular methodology, protocols, and reagents, etc., described herein and as such can vary. The terminology used herein is for the purpose of describing particular embodiments only, and is not intended to limit the scope of the present disclosure, which is defined solely by the claims.

### II. Expression of an PAH Protein from a Closed Ended DNA (ceDNA) Vector

The technology described herein is directed in general to the expression and/or production of PAH protein in a cell from a non-viral DNA vector, e.g., a ceDNA vector as described herein. ceDNA vectors for expression of PAH protein are described herein in the section entitled “ceDNA vectors in general”. In particular, ceDNA vectors for expression of PAH protein comprise a pair of ITRs (e.g., symmetric or asymmetric as described herein) and between the ITR pair, a nucleic acid encoding an PAH protein, wherein the nucleic acid is codon optimized, as described herein, operatively linked to a promoter or regulatory sequence. A distinct advantage of ceDNA vectors for expression of PAH protein over traditional AAV vectors, and even lentiviral vectors, is that there is no size constraint for the nucleic acid sequences encoding a desired protein. Thus, even a full length 6.8 kb PAH protein can be expressed from a single ceDNA vector. Thus, the ceDNA vectors described herein can be used to express a therapeutic PAH protein in a subject in need thereof, e.g., a subject with PKU.

As one will appreciate, the ceDNA vector technologies described herein can be adapted to any level of complexity or can be used in a modular fashion, where expression of different components of a PAH protein can be controlled in an independent manner. For example, it is specifically contemplated that the ceDNA vector technologies designed herein can be as simple as using a single ceDNA vector to express a single gene sequence (e.g., a PAH protein) or can be as complex as using multiple ceDNA vectors, where each vector expresses multiple PAH proteins or associated co-factors or accessory proteins that are each independently controlled by different promoters. The following embodiments are specifically contemplated herein and can adapted by one of skill in the art as desired.

In one embodiment, a single ceDNA vector can be used to express a single component of a PAH protein. Alternatively, a single ceDNA vector can be used to express multiple components (e.g., at least 2) of a PAH protein under the control of a single promoter (e.g., a strong promoter), optionally using an IRES sequence(s) to ensure appropriate expression of each of the components, e.g., co-factors or accessory proteins.

According to the present disclosure, the nucleic acids encoding the human PAH protein are codon optimized.

Additional variations of ceDNA vector technologies can be envisioned by one of skill in the art or can be adapted from protein production methods using conventional vectors.

### A. Nucleic Acids

The characterization and development of nucleic acid molecules for potential therapeutic use are provided herein. As described herein, the nucleic acids for therapeutic use encode a PAH protein, wherein the nucleic acids are codon optimized. In some embodiments, chemical modification of oligonucleotides for the purpose of altered and improved in vivo properties (delivery, stability, life-time, folding, target specificity), as well as their biological function and mechanism that directly correlate with therapeutic application, are described where appropriate.

Illustrative therapeutic nucleic acids of the present disclosure that can be immunostimulatory and require use of immunosuppressants disclosed herein can include, but are not limited to, minigenes, plasmids, minicircles, small interfering RNA (siRNA), microRNA (miRNA), antisense oligonucleotides (ASO), ribozymes, closed ended double stranded DNA (e.g., ceDNA, CELiD, linear covalently closed DNA (“ministring”), doggybone (dbDNA™), protelomere closed ended DNA, or dumbbell linear DNA), dicer-substrate dsRNA, small hairpin RNA (shRNA), asymmetrical interfering RNA (aiRNA), guide RNA (gRNA), microRNA (miRNA), mRNA, tRNA, rRNA, DNA viral vectors, viral RNA vector, and any combination thereof.

According to some embodiments, the therapeutic nucleic acid is a closed ended double stranded DNA, e.g., a ceDNA. According to some embodiments, the expression and/or production of a therapeutic protein in a cell is from a non-viral DNA vector, e.g., a ceDNA vector. A distinct advantage of ceDNA vectors for expression of a therapeutic protein over traditional AAV vectors, and even lentiviral vectors, is that there is no size constraint for the nucleic acid sequences, e.g., heterologous nucleic acid sequences, encoding a desired protein. Thus, even a large therapeutic protein can be expressed from a single ceDNA vector. Thus, ceDNA vectors can be used to express a therapeutic protein in a subject in need thereof.

In general, a ceDNA vector for expression of a therapeutic protein as disclosed herein, comprises in the 5′ to 3′ direction: a first adeno-associated virus (AAV) inverted terminal repeat (ITR), a nucleic acid sequence of interest (for example an expression cassette as described herein) and a second AAV ITR. The ITR sequences selected from any of: (i) at least one WT ITR and at least one modified AAV inverted terminal repeat (mod-ITR) (e.g., asymmetric modified ITRs); (ii) two modified ITRs where the mod-ITR pair have a different three-dimensional spatial organization with respect to each other (e.g., asymmetric modified ITRs), or (iii) symmetrical or substantially symmetrical WT-WT ITR pair, where each WT-ITR has the same three-dimensional spatial organization, or (iv) symmetrical or substantially symmetrical modified ITR pair, where each mod-ITR has the same three-dimensional spatial organization.

In some embodiments, a transgene encoding the PAH protein can also encode a secretory sequence so that the PAH protein is directed to the Golgi Apparatus and Endoplasmic Reticulum where a PAH protein is folded into the correct conformation by chaperone molecules as it passes through the ER and out of the cell. Exemplary secretory sequences include, but are not limited to VH-02 (SEQ ID NO: 88) and VK-A26 (SEQ ID NO: 89) and Igx signal sequence (SEQ ID NO: 126), as well as a Glue secretory signal that allows the tagged protein to be secreted out of the cytosol (SEQ ID NO: 188), TMD-ST secretory sequence, that directs the tagged protein to the golgi (SEQ ID NO: 189).

Regulatory switches can also be used to fine tune the expression of the PAH protein so that the PAH protein is expressed as desired, including but not limited to expression of the PAH protein at a desired expression level or amount, or alternatively, when there is the presence or absence of particular signal, including a cellular signaling event. For instance, as described herein, expression of the PAH protein from the ceDNA vector can be turned on or turned off when a particular condition occurs, as described herein in the section entitled Regulatory Switches.

For example, and for illustration purposes only, PAH proteins can be used to turn off undesired reaction, such as too high a level of production of the PAH protein. The PAH gene can contain a signal peptide marker to bring the PAH protein to the desired cell. However, in either situation it can be desirable to regulate the expression of the PAH protein. ceDNA vectors readily accommodate the use of regulatory switches.

A distinct advantage of ceDNA vectors over traditional AAV vectors, and even lentiviral vectors, is that there is no size constraint for the nucleic acid sequences, e.g., heterologous nucleic acid sequences, encoding the PAH protein. Thus, even a full-length PAH, as well as optionally any co-factors or assessor proteins can be expressed from a single ceDNA vector. In addition, depending on the necessary stiochemistry one can express multiple segments of the same PAH protein, and can use same or different promoters, and can also use regulatory switches to fine tune expression of each region. For example, as shown in the Examples, a ceDNA vector that comprises a dual promoter system can be used, so that a different promoter is used for each domain of the PAH protein. Use of a ceDNA plasmid to produce the PAH protein can include a unique combination of promoters for expression of the domains of the PAH protein that results in the proper ratios of each domain for the formation of functional PAH protein. Accordingly, in some embodiments, a ceDNA vector can be used to express different regions of PAH protein separately (e.g., under control of a different promoter).

In another embodiment, the PAH protein expressed from the ceDNA vectors further comprises an additional functionality, such as fluorescence, enzyme activity, secretion signal or immune cell activator.

In some embodiments, the ceDNA encoding the PAH protein can further comprise a linker domain, for example. As used herein “linker domain” refers to an oligo- or polypeptide region from about 2 to 100 amino acids in length, which links together any of the domains/regions of the PAH protein as described herein. In some embodiment, linkers can include or be composed of flexible residues such as glycine and serine so that the adjacent protein domains are free to move relative to one another. Longer linkers may be used when it is desirable to ensure that two adjacent domains do not sterically interfere with one another. Linkers may be cleavable or non-cleavable. Examples of cleavable linkers include 2A linkers (for example T2A), 2A-like linkers or functional equivalents thereof and combinations thereof. The linker can be a linker region is T2A derived from Thosea asigna virus.

It is well within the abilities of one of skill in the art to take a known and/or publically available protein sequence of e.g., the PAH etc., and reverse engineer a cDNA sequence to encode such a protein. The cDNA can then be codon optimized to match the intended host cell and inserted into a ceDNA vector as described herein.

B. ceDNA Vectors Expressing PAH Protein

A ceDNA vector for expression of PAH protein having one or more sequences encoding a desired PAH can comprise regulatory sequences such as promoters, secretion signals, polyA regions, and enhancers. At a minimum, a ceDNA vector comprises one or more nucleic acid sequences, e.g., heterologous nucleic acid sequences, encoding a PAH protein, wherein the nucleic acid sequence are codon optimized. According to some embodiments, the codon optimized nucleic acids are combined with particular cis-acting elements (e.g., specific promoters and/or specific enhancers) to achieve optimal transgene expression and duration.

According to some embodiments, the PAH protein comprise an endoplasmic reticulum ER leader sequence to direct it to the ER, where protein folding occurs. For example, a sequence that directs the expressed protein(s) to the ER for folding.

In some embodiments, a cellular or extracellular localization signal (e.g., secretory signal, nuclear localization signal, mitochondrial localization signal etc.) is comprised in the ceDNA vector to direct the secretion or desired subcellular localization of PAH such that the PAH protein can bind to intracellular target(s) (e.g., an intrabody) or extracellular target(s).

In some embodiments, a ceDNA vector for expression of PAH protein as described herein permits the assembly and expression of any desired PAH protein in a modular fashion. As used herein, the term “modular” refers to elements in a ceDNA expressing plasmid that can be readily removed from the construct.

In some embodiments, a ceDNA vector for expression of PAH can have a sequence encoding a full-length PAH protein. In some other embodiments, a ceDNA vector expression of PAH can have a sequence encoding a truncated PAH protein. For example, the truncated PAH may have a deletion at the N-terminal end to remove an autoregulatory region of PAH (e.g., amino acids 1-19 of the full-length PAH). In one embodiment, a ceDNA vector for expression of PAH has a N-terminal truncation of amino acids 1-19.

In some embodiments, a ceDNA vector can have a PAH sequence with an intron inside of the open reading frame for a functional PAH. In some other embodiments, a ceDNA can have a PAH sequence with heterologous signal sequence (SS). In yet some other embodiments, a ceDNA can have a PAH sequence with DNA nuclear targeting sequence. In yet some other embodiments, a ceDNA vector of a codon optimized PAH can have a 5′ UTR sequence. In yet some other embodiments, a ceDNA vector of a codon optimized PAH can have an intron sequence. In yet some other embodiments, a ceDNA vector of a codon optimized PAH can have a 3′ UTR sequence. In yet some other embodiments, a ceDNA vector of a codon optimized PAH can have one or more enhancer sequences. In yet some other embodiments, a ceDNA vector of a codon optimized PAH can have a promoter sequence. In yet some other embodiments, a ceDNA vector of a codon optimized PAH can have a Kozak sequence. In yet some other embodiments, a ceDNA vector of a codon optimized PAH can have a linker/spacer sequence between two cis-acting elements (e.g., enhancer elements) or between a cis-acting element and an open reading frame (ORF).

C. Exemplary PAH Proteins Expressed by ceDNA Vectors

In particular, a ceDNA vector for expression of PAH protein as disclosed herein can encode, for example, PAH proteins, as well as variants, and/or active fragments thereof, for use in the treatment, prophylaxis, and/or amelioration of one or more symptoms of Phenylketonuria (PKU). In one aspect, the Phenylketonuria (PKU) is a human Phenylketonuria (PKU).
(i) PAH Therapeutic Proteins and Fragments Thereof

The present disclosure provides PAH therapeutic proteins or fragments thereof (e.g., functional fragments) that are encoded by codon optimized nucleic acids and expressed in and from a ceDNA vector as described herein. One of skill in the art will understand that PAH therapeutic protein includes all splice variants and orthologs of the PAH protein. PAH therapeutic protein includes intact molecules as well as truncated fragments (e.g., functional) thereof.

A distinct advantage of ceDNA vectors over traditional AAV vectors, and even lentiviral vectors, is that there is no size constraint for the nucleic acid sequences, e.g., heterologous nucleic acid sequences, encoding a desired protein. Thus, multiple full-length PAH therapeutic proteins can be expressed from a single ceDNA vector.

PAH protein and gene: The PAH gene is located on chromosome 12 in the bands 12q22-q24.2. As of 2000, around 400 disease-causing mutations had been found in the PAH gene. Phenylalanine Hydroxylase (PAH) can also be referred to as Phenylalanine 4-Monooxygenase, Phenylalanine-4-Hydroxylase, Phe-4-Monooxygenase, EC 1.14.16.1, EC 1.14.16, PKU1, PKU, or PH.

The protein sequence for PAH is as follows: Homo sapiens PAH enzyme translation (450 amino acids), accession number NM_000277.3.

(SEQ ID NO: 1025)
MSTAVLENPGLGRKLSDFGQETSYIEDNCNQNGAISLIFSL
KEEVGALAKVLRLFEENDVNLTHIESRPSRLKKDEYEFFT
HLDKRSLPALTNIIKILRHDIGATVHELSRDKKKDTVPWF
PRTIQELDRFANQILSYGAELDADHPGFKDPVYRARRKQF
ADIAYNYRHGQPIPRVEYMEEEKKTWGTVFKTLKSLYKTH
ACYEYNHIFPLLEKYCGFHEDNIPQLEDVSQFLQTCTGFR
LRPVAGLLSSRDFLGGLAFRVFHCTQYIRHGSKPMYTPEP
DICHELLGHVPLFSDRSFAQFSQEIGLASLGAPDEYIEKL
ATIYWFTVEFGLCKQGDSIKAYGAGLLSSFGELQYCLSEK
PKLLPLELEKTAIQNYTVTEFQPLYYVAESFNDAKEKVRN
FAATIPRPFSVRYDPYTQRIEVLDNTQQLKILADSINSEI
GILCSALQK

PAH is predominantly expressed in the liver, with moderate expression in the kidneys and gallbladder. Low levels of PAH expression can also be detected in the prostate, adrenal gland. During fetal development, PAH can be expressed in the adrenal gland, heart, intestine, lung, and stomach. Accordingly, one can administer a ceDNA vector expressing PAH to any one or more tissues selected from: liver, kidneys, gallbladder, prostate, adrenal. In some embodiments, when a ceDNA vector expressing PAH is administered to an infant, or administered to a subject in utero, one can administer a ceDNA vector expressing PAH to any one or more tissues selected from: liver, adrenal gland, heart, intestine, lung, and stomach.

Expression of PAH therapeutic protein or fragment thereof from a ceDNA vector can be achieved both spatially and temporally using one or more of the promoters as described herein. In some embodiments, the promoter is selected from the group consisting of: the VD promoter, human alpha 1-antitrypsin (hAAT) promoter (including the hAAT(979) promoter and the CpGmin_hAAT promoter) and the transthyretin (TTR) liver specific promoter.

According to some embodiments, the nucleic acid encoding the PAH protein is codon optimized, and inserted into a ceDNA vector as described herein.

As used herein, the term “codon optimized” or “codon optimization” refers to the process of modifying a nucleic acid sequence for enhanced expression in the cells of the vertebrate of interest, e.g., mouse or human, by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid. Typically, codon optimization does not alter the amino acid sequence of the original translated protein. Optimized codons can be determined using e.g., Aptagen's GENE FORGE® codon optimization and custom gene synthesis platform (Aptagen, Inc., 2190 Fox Mill Rd. Suite 300, Herndon, Va. 20171) or another publicly available database. In some embodiments, the nucleic acid encoding the PAH protein is optimized for human expression, and/or is a human PAH, or functional fragment thereof. Exemplary PAH sequences altered for ceDNA expression in conjunction with various cis-acting elements are disclosed herein.

(ii) PAH Therapeutic Protein Expressing ceDNA Vectors

A ceDNA vector as described herein comprises one or more codon optimized nucleic acid sequences, e.g., heterologous nucleic acid sequences, encoding a PAH therapeutic protein or functional fragment thereof. In one embodiment, the ceDNA vector comprises a codon optimized nucleic acid sequence encoding a PAH sequence selected from those listed in Table 1A herein.

TABLE 1A
Exemplary human PAH sequences for treatment of PKU

| | SEQ ID |
|---|---|
| Description | NO: |
| Human Phenylalanine Hydroxylase (PAH) Codon Optimized | 382 |
| (hPAH_codop_ORF_v2) | |
| Human Phenylalanine Hydroxylase (PAH) cDNA | 383 |
| (hPAH_cDNA_ORF_v3). | |
| Human Phenylalanine Hydroxylase (PAH) Codon Optimized | 384 |
| (hPAH_codop_ORF_v2_delta2KbIntron_33bpFlanks) | |
| Human Phenylalanine Hydroxylase (PAH) Codon Optimized | 385 |
| (hPAH_codop_ORF_v2_hIVS-intron1_33bpFlanks) | |
| Human Phenylalanine Hydroxylase (PAH) Codon Optimized | 386 |
| (hPAH_codop_ORF_v2_mIVS-intron1B_1bpFlank) | |
| Human Phenylalanine Hydroxylase (PAH) Codon Optimized | 387 |
| (hPAH_codop_ORF_v2_mIVS-intron1B_33bpFlanks) | |
| Human Phenylalanine Hydroxylase (PAH) Codon Optimized | 388 |
| (hPAH_Genscript_codop_ORF) | |
| Human Phenylalanine Hydroxylase (PAH) Codon Optimized | 389 |
| (hPAH_codop_ORF_v2_hIVS-intron1_1bpFlank) | |
| Human Phenylalanine Hydroxylase (PAH) Codon Optimized | 390 |
| (hPAH_codop_ORF_v2_33bpFlanks) | |
| Human Phenylalanine Hydroxylase (PAH) Codon Optimized | 391 |
| (hPAH_codop_ORF_v2_5pSpliceFix) | |
| Human Phenylalanine Hydroxylase (PAH) Codon Optimized | 392 |
| (hPAH_codop_ORF_v2_modified_Intron1_33bpFlanks) | |
| Human Phenylalanine Hydroxylase (PAH) Codon Optimized | 393 |
| (hPAH_codop_ORF_v2_C237D) | |
| Human Phenylalanine Hydroxylase (PAH) Codon Optimized | 394 |
| (hPAH_codop_ORF_v2_delta1-29aa) | |
| Human Phenylalanine Hydroxylase (PAH) Codon Optimized | 1011 |
| (hPAH_codop_ORF_v2_R68A) | |
| Human Phenylalanine Hydroxylase (PAH) Codon Optimized | 395 |
| hPAH-ORF_v2B) | |
| Human Phenylalanine Hydroxylase (PAH) Codon Optimized | 396 |
| (hPAH-r3-s7) | |
| Human Phenylalanine Hydroxylase (PAH) Codon Optimized | 397 |
| (hPAH-r3-s32) | |
| Human Phenylalanine Hydroxylase (PAH) Codon Optimized | 398 |
| (hPAH-r3-s34) | |
| Human Phenylalanine Hydroxylase (PAH) Codon Optimized | 399 |
| (hPAH-r3-s41) | |
| Human Phenylalanine Hydroxylase (PAH) Codon Optimized | 400 |
| (hPAH-r4-s21) | |
| Human Phenylalanine Hydroxylase (PAH) Codon Optimized | 401 |
| (hPAH-r4-s27) | |
| Human Phenylalanine Hydroxylase (PAH) Codon Optimized | 1012 |
| (hPAH-r5-s3) | |
| Human Phenylalanine Hydroxylase (PAH) Codon Optimized | 402 |
| (hPAH-r5-s8) | |
| Human Phenylalanine Hydroxylase (PAH) Codon Optimized | 403 |
| (hPAH-r5-s29) | |
| Human Phenylalanine Hydroxylase (PAH) Codon Optimized | 404 |
| (hPAH-r6-28) | |
| Human Phenylalanine Hydroxylase (PAH) Codon Optimized | 405 |
| (hPAH-r6-s20) | |
| Human Phenylalanine Hydroxylase (PAH) Codon Optimized | 406 |
| (hPAH-r7-s1) | |
| Human Phenylalanine Hydroxylase (PAH) Codon Optimized | 407 |
| (hPAH-r7-s23) | |
| Human Phenylalanine Hydroxylase (PAH) Codon Optimized | 408 |
| (hPAH-r8-s1) | |
| Human Phenylalanine Hydroxylase (PAH) Codon Optimized | 1013 |
| (hPAH-r8-s29) | |
| Human Phenylalanine Hydroxylase (PAH) Codon Optimized | 409 |
| (hPAH-r9-s20) | |
| Human Phenylalanine Hydroxylase (PAH) Codon Optimized | 410 |
| (hPAH-r9-s37) | |
| Human Phenylalanine Hydroxylase (PAH) Codon Optimized | 411 |
| (hPAH-r9-s52) | |
| Human Phenylalanine Hydroxylase (PAH) Codon Optimized | 412 |
| (hPAH-r10-s18) | |
| Human Phenylalanine Hydroxylase (PAH) Codon Optimized | 413 |
| (hPAH-r10-s21) | |
| Human Phenylalanine Hydroxylase (PAH) Codon Optimized | 414 |
| (hPAH-r10-s55) | |
| Human Phenylalanine Hydroxylase (PAH) Codon Optimized | 415 |
| (hPAH-r11-s6) | |
| Human Phenylalanine Hydroxylase (PAH) Codon Optimized | 1014 |
| (hPAH-r11-s26) | |
| Human Phenylalanine Hydroxylase (PAH) Codon Optimized | 416 |
| (hPAH-r11-s27) | |
| Human Phenylalanine Hydroxylase (PAH) Codon Optimized | 417 |
| (hPAH-r3-s34::hIVS1B) | |
| Human Phenylalanine Hydroxylase (PAH) Codon Optimized | 418 |
| (hPAH-r3-s34::hIVS1B_33bpFlanks) | |
| Human Phenylalanine Hydroxylase (PAH) Codon Optimized | 419 |
| (hPAH-r3-s34::mIVS) | |
| Human Phenylalanine Hydroxylase (PAH) Codon Optimized | 420 |
| (hPAH-r3-s34::mIVS_33bpFlanks) | |
| Human Phenylalanine Hydroxylase (PAH) Codon Optimized | 421 |
| (hPAH-r3-s34::mod-Intron) | |
| Human Phenylalanine Hydroxylase (PAH) Codon Optimized | 422 |
| (hPAH-r3-s34::mod-Intron_33bpFlanks) | |
| Human Phenylalanine Hydroxylase (PAH) Codon Optimized | 1015 |
| (hPAH-r3-s34::mod-Intron_oIVS-v2) | |
| Human Phenylalanine Hydroxylase (PAH) Codon Optimized | 423 |
| (hPAH-r3-s34::mod-Intron_oIVS-v2_33bpFlanks) | |
| Human Phenylalanine Hydroxylase (PAH) Codon Optimized | 424 |
| (hPAH-r5-s29::hIVS1B) | |
| Human Phenylalanine Hydroxylase (PAH) Codon Optimized | 425 |
| (hPAH-r5-s29::hIVS1B_33bpFlanks) | |
| Human Phenylalanine Hydroxylase (PAH) Codon Optimized | 426 |
| (hPAH-r5-s29::mIVS) | |
| Human Phenylalanine Hydroxylase (PAH) Codon Optimized | 427 |
| (hPAH-r5-s29::mIVS_33bpFlanks) | |
| Human Phenylalanine Hydroxylase (PAH) Codon Optimized | 428 |
| (hPAH-r5-s29::mod-Intron) | |
| Human Phenylalanine Hydroxylase (PAH) Codon Optimized | 429 |
| (hPAH-r5-s29::mod-Intron_33bpFlanks) | |
| Human Phenylalanine Hydroxylase (PAH) Codon Optimized | 430 |
| (hPAH-r5-s29::mod-Intron_oIVS-v2) | |
| Human Phenylalanine Hydroxylase (PAH) Codon Optimized | 431 |
| (hPAH-r5-s29::mod-Intron_oIVS-v2_33bpFlanks) | |
| Human Phenylalanine Hydroxylase (PAH) Codon Optimized | 432 |
| (hPAH_codop_ORF_v2-mod-Intron-oIVS-v2) | |
| Human Phenylalanine Hydroxylase (PAH) Codon Optimized | 433 |
| (hPAH_codop_ORF_v2-mIVS-CpGfree_33bpFlanks) | |
| Human Phenylalanine Hydroxylase (PAH) Codon Optimized | 434 |
| (hPAH_codop_ORF_v2::hIVS-1B-Wt_33bpFlanks) | |
| Human Phenylalanine Hydroxylase (PAH) Codon Optimized | 435 |
| (hPAH-cDNA_0CpG1_ORF) | |
| Human Phenylalanine Hydroxylase (PAH) Codon Optimized | 436 |
| (hPAH-cDNA_0CpG2_ORF) | |
| Human Phenylalanine Hydroxylase (PAH) Codon Optimized | 437 |
| (hPAH-cDNA_0CpG3_ORF) | |
| Human Phenylalanine Hydroxylase (PAH) Codon Optimized | 438 |
| (hPAH-cDNA_0CpG4_ORF) | |
| Human Phenylalanine Hydroxylase (PAH) | 439 |
| (hPAH-cDNA_1_ORF) | |
| Human Phenylalanine Hydroxylase (PAH) | 440 |
| (hPAH-cDNA_2_ORF) | |

TABLE 1B
Exemplary mouse PAH sequence

| Description | SEQ ID NO: |
|---|---|
| Mouse Phenyalanine Hydroxylase PAH | 585 |
| (mousePAH_codop_ORF_v2 | |

In one embodiment, the ceDNA vector comprises a codon optimized human PAH sequence listed in Table 1A herein. In one embodiment, the ceDNA vector comprises a codon optimized PAH sequence having at least 90% identity to a sequence listed in Table 1A. In one embodiment, the ceDNA vector comprises a codon optimized PAH sequence having at least 91% identity to a sequence listed in Table 1A. In one embodiment, the ceDNA vector comprises a codon optimized PAH sequence having at least 92% identity to a PAH sequence listed in Table 1A. In one embodiment, the ceDNA vector comprises a codon optimized PAH sequence having at least 93% identity to a PAH sequence listed in Table 1A. In one embodiment, the ceDNA vector comprises a codon optimized PAH sequence having at least 94% identity to a PAH sequence listed in Table 1A. In one embodiment, the ceDNA vector comprises a codon optimized PAH sequence having at least 95% identity to a PAH sequence listed in Table 1A. In one embodiment, the ceDNA vector comprises a codon optimized PAH sequence having at least 96% identity to a PAH sequence listed in Table 1A. In one embodiment, the ceDNA vector comprises a codon optimized PAH sequence having at least 97% identity to a PAH sequence listed in Table 1A. In one embodiment, the ceDNA vector comprises a codon optimized PAH sequence having at least 98% identity to a PAH sequence listed in Table 1A. In one embodiment, the ceDNA vector comprises a codon optimized PAH sequence having at least 99% identity to a PAH sequence listed in Table 1A.

In one embodiment, the PAH sequence has at least 90% identity to SEQ ID NO:382, SEQ ID NO:383, SEQ ID NO:384, SEQ ID NO:385, SEQ ID NO:386, SEQ ID NO:387, NO:389, SEQ ID NO:390, SEQ ID NO:391, SEQ ID NO:392, SEQ ID NO:393, SEQ ID NO:394, SEQ ID NO:395, SEQ ID NO:396, SEQ ID NO:397, SEQ ID NO:398, SEQ ID NO:399, SEQ ID NO:400, SEQ ID NO:401, SEQ ID NO:402, SEQ ID NO:403, SEQ ID NO:404, SEQ ID NO:405, SEQ ID NO:406, SEQ ID NO:407, SEQ ID NO:408, SEQ ID NO:409, SEQ ID NO:410, SEQ ID NO:411, SEQ ID NO:412, SEQ ID NO:413, SEQ ID NO:414, SEQ ID NO:415, SEQ ID NO:416, SEQ ID NO:417, SEQ ID NO:418, SEQ ID NO:419, SEQ ID NO:420, SEQ ID NO:421, SEQ ID NO:422, SEQ ID NO:423, SEQ ID NO:424, SEQ ID NO:425, SEQ ID NO:426, SEQ ID NO:427, SEQ ID NO:428, SEQ ID NO:429, SEQ ID NO:430, SEQ ID NO:431, SEQ ID NO:432, SEQ ID NO:433, SEQ ID NO:434, SEQ ID NO:435, SEQ ID NO:436, SEQ ID NO:437, SEQ ID NO:438, SEQ ID NO:439, SEQ ID NO:440, SEQ ID NO:1011, SEQ ID NO:1012, SEQ ID NO:1013, SEQ ID NO:1014 or SEQ ID NO: 1015. In one embodiment, the PAH sequence has at least 91% identity to any one of SEQ ID NOs:382-440 or SEQ ID NOs: 1011-1015. In one embodiment, the PAH sequence has at least 92% identity to any one of SEQ ID NOs:382-440 or SEQ ID NOs: 1011-1015. In one embodiment, the PAH sequence has at least 93% identity to any one of SEQ ID NOs:382-440 or SEQ ID NOs: 1011-1015. In one embodiment, the PAH sequence has at least 94% identity to any one of SEQ ID NOs:382-440 or SEQ ID NOs: 1011-1015. In one embodiment, the PAH sequence has at least 95% identity to any one of SEQ ID NOs:382-440 or SEQ ID NOs: 1011-1015. In one embodiment, the PAH sequence has at least 96% identity to any one of SEQ ID NOs:382-440 or SEQ ID NOs: 1011-1015. In one embodiment, the PAH sequence has at least 97% identity to any one of SEQ ID NOs:382-440 or SEQ ID NOs: 1011-1015. In one embodiment, the PAH sequence has at least 98% identity to any one of SEQ ID NOs:382-440 or SEQ ID NOs: 1011-1015. In one embodiment, the PAH sequence has at least 99% identity to any one of SEQ ID NOs:382-440 or SEQ ID NOs: 1011-1015. In one embodiment, the PAH sequence comprises a sequence selected from the group consisting of SEQ ID NO:382, SEQ ID NO:383, SEQ ID NO:384, SEQ ID NO:385, SEQ ID NO:386, SEQ ID NO:387, NO:389, SEQ ID NO:390, SEQ ID NO:391, SEQ ID NO:392, SEQ ID NO:393, SEQ ID NO:394, SEQ ID NO:395, SEQ ID NO:396, SEQ ID NO:397, SEQ ID NO:398, SEQ ID NO:399, SEQ ID NO:400, SEQ ID NO:401, SEQ ID NO:402, SEQ ID NO:403, SEQ ID NO:404, SEQ ID NO:405, SEQ ID NO:406, SEQ ID NO:407, SEQ ID NO:408, SEQ ID NO:409, SEQ ID NO:410, SEQ ID NO:411, SEQ ID NO:412, SEQ ID NO:413, SEQ ID NO:414, SEQ ID NO:415, SEQ ID NO:416, SEQ ID NO:417, SEQ ID NO:418, SEQ ID NO:419, SEQ ID NO:420, SEQ ID NO:421, SEQ ID NO:422, SEQ ID NO:423, SEQ ID NO:424, SEQ ID NO:425, SEQ ID NO:426, SEQ ID NO:427, SEQ ID NO:428, SEQ ID NO:429, SEQ ID NO:430, SEQ ID NO:431, SEQ ID NO:432, SEQ ID NO:433, SEQ ID NO:434, SEQ ID NO:435, SEQ ID NO:436, SEQ ID NO:437, SEQ ID NO:438, SEQ ID NO:439, SEQ ID NO:440, SEQ ID NO: 1011, SEQ ID NO: 1012, SEQ ID NO: 1013, SEQ ID NO: 1014, and SEQ ID NO: 1015. In one embodiment, the PAH sequence consists of SEQ ID NO:382, SEQ ID NO:383, SEQ ID NO:384, SEQ ID NO:385, SEQ ID NO:386, SEQ ID NO:387, NO:389, SEQ ID NO:390, SEQ ID NO:391, SEQ ID NO:392, SEQ ID NO:393, SEQ ID NO:394, SEQ ID NO:395, SEQ ID NO:396, SEQ ID NO:397, SEQ ID NO:398, SEQ ID NO:399, SEQ ID NO:400, SEQ ID NO:401, SEQ ID NO:402, SEQ ID NO:403, SEQ ID NO:404, SEQ ID NO:405, SEQ ID NO:406, SEQ ID NO:407, SEQ ID NO:408, SEQ ID NO:409, SEQ ID NO:410, SEQ ID NO:411, SEQ ID NO:412, SEQ ID NO:413, SEQ ID NO:414, SEQ ID NO:415, SEQ ID NO:416, SEQ ID NO:417, SEQ ID NO:418, SEQ ID NO:419, SEQ ID NO:420, SEQ ID NO:421, SEQ ID NO:422, SEQ ID NO:423, SEQ ID NO:424, SEQ ID NO:425, SEQ ID NO:426, SEQ ID NO:427, SEQ ID NO:428, SEQ ID NO:429, SEQ ID NO:430, SEQ ID NO:431, SEQ ID NO:432, SEQ ID NO:433, SEQ ID NO:434, SEQ ID NO:435, SEQ ID NO:436, SEQ ID NO:437, SEQ ID NO:438, SEQ ID NO:439, or SEQ ID NO:440, SEQ ID NO: 1011, SEQ ID NO: 1012, SEQ ID NO: 1013, SEQ ID NO: 1014, or SEQ ID NO: 1015.

(iii) PAH Therapeutic Proteins and Uses Thereof for the Treatment of PKU

The ceDNA vectors described herein can be used to deliver therapeutic PAH proteins for treatment of PKU associated with inappropriate expression of the PAH protein and/or mutations within the PAH proteins.

ceDNA vectors as described herein can be used to express any desired PAH therapeutic protein. Exemplary therapeutic PAH therapeutic proteins include, but are not limited to any PAH protein expressed by the sequences as set forth in Table 1A herein.

In one embodiment, the expressed PAH therapeutic protein is functional for the treatment of a Phenylketonuria (PKU). In some embodiments, PAH therapeutic protein does not cause an immune system reaction.

In some embodiments, the ceDNA vector comprises a codon optimized sequence encoding a truncated (fragment) of PAH. In one embodiment, the ceDNA vector comprises a codon optimized sequence encoding a truncated PAH having a deletion of an N-terminal autoregulatory region, e.g., from amino acid 1 to 29 of the full-length PAH protein. In one embodiment, the ceDNA vector comprises SEQ ID NO:394.

In another embodiment, the ceDNA vectors encoding PAH therapeutic protein or fragment thereof (e.g., functional fragment) can be used to generate a chimeric protein. Thus, it is specifically contemplated herein that a ceDNA vector expressing a chimeric protein can be administered to e.g., to any one or more tissues selected from: liver, kidneys, gallbladder, prostate, adrenal gland. In some embodiments, when a ceDNA vector expressing PAH is administered to an infant, or administered to a subject in utero, one can administer a ceDNA vector expressing PAH to any one or more tissues selected from: liver, adrenal gland, heart, intestine, lung, and stomach, or to a liver stem cell precursor thereof for the in vivo or ex vivo treatment of Phenylketonuria (PKU).

PKU: PKU is a rare, inherited inborn error of metabolism caused by a mutation in the PAH gene. PAH is an enzyme that is normally expressed in the liver and is necessary to metabolize dietary phenylalanine into tyrosine, an amino acid responsible for the production of neurotransmitters. PKU results from mutations in PAH that render its enzymatic activity deficient. Accordingly, ceDNA vectors expressing an PAH protein can be express PAH in liver. In some embodiments, ceDNA vectors express at least one PAH protein in hepatocytes.

PAH is normally endogenously expressed in both PR and RPE cell types. It is also reported that low level of PAH expression in RPE may also be required for normal retinal function. Accordingly, low-level or high-level of expression of the PAH protein by the ceDNA vector in PRs and also, optionally RPE cells, may sometimes be needed to prevent retinal degeneration. This level of expression can be fine-tuned by promoters and/or regulatory switches as described herein.

Accordingly, in some embodiments, the ceDNA vector is used for expression of PAH protein, which is a 6.8 kb protein, from the endogenous promoter (˜1 kb) to restore normal retinoid processing in both photoreceptors and RPE. In some embodiments, a ceDNA vector expressing a PAH protein is via a suprachoroidal or intravitreal route of administration to treat larger area of retina. In some embodiments, the ceDNA vector is administered by any one or more of: subretinal injection, suprachoroidal injection or intravitreal injection.

The methods comprise administering to the subject an effective amount of a composition comprising a ceDNA vector encoding the PAH therapeutic protein or fragment thereof (e.g., functional fragment) as described herein. As will be appreciated by a skilled practitioner, the term “effective amount” refers to the amount of the ceDNA composition administered that results in expression of the protein in a “therapeutically effective amount” for the treatment of a disease or disorder.

The dosage ranges for the composition comprising a ceDNA vector encoding the PAH therapeutic protein or fragment thereof (e.g., functional fragment) depends upon the potency (e.g., efficiency of the promoter), and includes amounts large enough to produce the desired effect, e.g., expression of the desired PAH therapeutic protein, for the treatment of Phenylketonuria (PKU). The dosage should not be so large as to cause unacceptable adverse side effects. Generally, the dosage will vary with the particular characteristics of the ceDNA vector, expression efficiency and with the age, condition, and sex of the patient. The dosage can be determined by one of skill in the art and, unlike traditional AAV vectors, can also be adjusted by the individual physician in the event of any complication because ceDNA vectors do not comprise immune activating capsid proteins that prevent repeat dosing.

Administration of the ceDNA compositions described herein can be repeated for a limited period of time. In some embodiments, the doses are given periodically or by pulsed administration. In a preferred embodiment, the doses recited above are administered over several months. The duration of treatment depends upon the subject's clinical progress and responsiveness to therapy. Booster treatments over time are contemplated. Further, the level of expression can be titrated as the subject grows.

An PAH therapeutic protein can be expressed in a subject for at least 1 week, at least 2 weeks, at least 1 month, at least 2 months, at least 6 months, at least 12 months/one year, at least 2 years, at least 5 years, at least 10 years, at least 15 years, at least 20 years, at least 30 years, at least 40 years, at least 50 years or more. Long-term expression can be achieved by repeated administration of the ceDNA vectors described herein at predetermined or desired intervals.

As used herein, the term “therapeutically effective amount” is an amount of an expressed PAH therapeutic protein, or functional fragment thereof that is sufficient to produce a statistically significant, measurable change in expression of a disease biomarker or reduction in a given disease symptom (see “Efficacy Measurement” below). Such effective amounts can be gauged in clinical trials as well as animal studies for a given ceDNA composition.

Precise amounts of the ceDNA vector required to be administered depend on the judgment of the practitioner and are particular to each individual. Suitable regimes for administration are also variable, but are typified by an initial administration followed by repeated doses at one or more intervals by a subsequent injection or other administration. Alternatively, continuous intravenous infusion sufficient to maintain concentrations in the blood in the ranges specified for in vivo therapies are contemplated, particularly for the treatment of acute diseases/disorders.

Agents useful in the methods and compositions described herein can be administered topically, intravenously (by bolus or continuous infusion), intracellular injection, intratissue injection, orally, by inhalation, intraperitoneally, intramuscularly, subcutaneously, intracavity, and can be delivered by peristaltic means, if desired, or by other means known by those skilled in the art. The agent can be administered systemically, if so desired. It can also be administered in utero.

The efficacy of a given treatment for Phenylketonuria (PKU), can be determined by the skilled clinician. However, a treatment is considered “effective treatment,” as the term is used herein, if any one or all of the signs or symptoms of the disease or disorder is/are altered in a beneficial manner, or other clinically accepted symptoms or markers of disease are improved, or ameliorated, e.g., by at least 10% following treatment with a ceDNA vector encoding PAH, or a functional fragment thereof. Efficacy can also be measured by failure of an individual to worsen as assessed by stabilization of the disease, or the need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing progression of the disease or disorder; or (2) relieving the disease, e.g., causing regression of symptoms; and (3) preventing or reducing the likelihood of the development of the disease, or preventing secondary diseases/disorders associated with the disease, such as liver or kidney failure. An effective amount for the treatment of a disease means that amount which, when administered to a mammal in need thereof, is sufficient to result in effective treatment as that term is defined herein, for that disease.

Efficacy of an agent can be determined by assessing physical indicators that are particular to Phenylketonuria (PKU). Standard methods of analysis of PKU indicators are known in the art.

In some embodiments, a ceDNA vector for expression of PAH protein as disclosed herein can also encode co-factors or other polypeptides, sense or antisense oligonucleotides, or RNAs (coding or non-coding; e.g., siRNAs, shRNAs, gRNA, micro-RNAs, and their antisense counterparts (e.g., antagoMiR)) that can be used in conjunction with the PAH protein expressed from the ceDNA. Additionally, expression cassettes comprising sequence encoding an PAH protein can also include an exogenous sequence that encodes a reporter protein to be used for experimental or diagnostic purposes, such as β-lactamase, β-galactosidase (LacZ), alkaline phosphatase, thymidine kinase, green fluorescent protein (GFP), chloramphenicol acetyltransferase (CAT), luciferase, and others well known in the art.

In one embodiment, the ceDNA vector comprises a nucleic acid sequence to express the PAH protein that is functional for the treatment of PKU. In a preferred embodiment, the therapeutic PAH protein does not cause an immune system reaction, unless so desired.

### III. ceDNA Vector in General for Use in Production of PAH Therapeutic Proteins

Embodiments of the disclosure are based on methods and compositions comprising close ended linear duplexed (ceDNA) vectors that can express the PAH transgene. In some embodiments, the transgene is a codon optimized sequence encoding a PAH protein (see, e.g., SEQ ID NOs:382-440 or SEQ ID NOs: 1011-1015). The ceDNA vectors for expression of PAH protein as described herein are not limited by size, thereby permitting, for example, expression of all of the components necessary for expression of a transgene from a single vector. The ceDNA vector for expression of PAH protein is preferably duplex, e.g., self-complementary, over at least a portion of the molecule, such as the expression cassette (e.g., ceDNA is not a double stranded circular molecule). The ceDNA vector has covalently closed ends, and thus is resistant to exonuclease digestion (e.g., exonuclease I or exonuclease III), e.g., for over an hour at 37° C.

In general, a ceDNA vector for expression of PAH protein as disclosed herein, comprises in the 5′ to 3′ direction: a first adeno-associated virus (AAV) inverted terminal repeat (ITR), a nucleic acid sequence of interest (for example an expression cassette as described herein) and a second AAV ITR. The ITR sequences selected from any of: (i) at least one WT ITR and at least one modified AAV inverted terminal repeat (mod-ITR) (e.g., asymmetric modified ITRs); (ii) two modified ITRs where the mod-ITR pair have a different three-dimensional spatial organization with respect to each other (e.g., asymmetric modified ITRs), or (iii) symmetrical or substantially symmetrical WT-WT ITR pair, where each WT-ITR has the same three-dimensional spatial organization, or (iv) symmetrical or substantially symmetrical modified ITR pair, where each mod-ITR has the same three-dimensional spatial organization. A ceDNA vector for expression of PAH protein can be made from cell-based (e.g., Sf9 or HEK293) production or synthetically using a plurality of oligonucleotides in a cell-free environment.

Encompassed herein are methods and compositions comprising the ceDNA vector for PAH protein production, which may further include a delivery system, such as a liposome nanoparticle delivery system. Non-limiting exemplary liposome nanoparticle systems are disclosed herein. In some aspects, the disclosure provides for a lipid nanoparticle comprising ceDNA and an ionizable lipid. For example, a lipid nanoparticle formulation that is made and loaded with a ceDNA vector obtained by the process is disclosed in International Application PCT/US2018/050042, filed on Sep. 7, 2018, and International Application PCT/US2020/049266, filed on Sep. 3, 2020 each of which is incorporated herein by reference in its entirety.

The ceDNA vectors for expression of PAH protein as disclosed herein have no packaging constraints imposed by the limiting space within the viral capsid. ceDNA vectors represent a viable eukaryotically-produced alternative to prokaryote-produced plasmid DNA vectors, as opposed to encapsulated AAV genomes. This permits the insertion of control elements, e.g., regulatory switches as disclosed herein, large transgenes, multiple transgenes etc.

ceDNA vectors for expression of PAH protein are capsid-free and can be obtained from a plasmid encoding in this order: a first ITR, an expression cassette comprising a transgene and a second ITR. The expression cassette may include one or more regulatory sequences that allows and/or controls the expression of the transgene, e.g., where the expression cassette can comprise one or more of, in this order: an enhancer/promoter, an ORF (transgene encoding PAH), a post-transcription regulatory element (e.g., WPRE), and a polyadenylation and termination signal (e.g., BGH polyA).

The expression cassette can also comprise an internal ribosome entry site (IRES) and/or a 2A element. The cis-regulatory elements include, but are not limited to, a promoter, a riboswitch, an insulator, a mir-regulatable element, a post-transcriptional regulatory element, a tissue- and cell type-specific promoter and an enhancer. In some embodiments the ITR can act as the promoter for the transgene, e.g., PAH protein. In some embodiments, the ceDNA vector comprises additional components to regulate expression of the transgene, for example, a regulatory switch, which are described herein in the section entitled “Regulatory Switches” for controlling and regulating the expression of the PAH protein, and can include if desired, a regulatory switch which is a kill switch to enable controlled cell death of a cell comprising a ceDNA vector.

The expression cassette can comprise more than 4000 nucleotides, 5000 nucleotides, 10,000 nucleotides or 20,000 nucleotides, or 30,000 nucleotides, or 40,000 nucleotides or 50,000 nucleotides, or any range between about 4000-10,000 nucleotides or 10,000-50,000 nucleotides, or more than 50,000 nucleotides. In some embodiments, the expression cassette can comprise a transgene in the range of 500 to 50,000 nucleotides in length. In some embodiments, the expression cassette can comprise a transgene in the range of 500 to 75,000 nucleotides in length. In some embodiments, the expression cassette can comprise a transgene which is in the range of 500 to 10,000 nucleotides in length. In some embodiments, the expression cassette can comprise a transgene which is in the range of 1000 to 10,000 nucleotides in length. In some embodiments, the expression cassette can comprise a transgene which is in the range of 500 to 5,000 nucleotides in length. The ceDNA vectors do not have the size limitations of encapsidated AAV vectors, thus enable delivery of a large-size expression cassette to provide efficient transgene expression. In some embodiments, the ceDNA vector is devoid of prokaryote-specific methylation.

ceDNA expression cassette can include, for example, an expressible exogenous sequence (e.g., open reading frame) or transgene that encodes a protein that is either absent, inactive, or insufficient activity in the recipient subject or a gene that encodes a protein having a desired biological or a therapeutic effect. The transgene can encode a gene product that can function to correct the expression of a defective gene or transcript. In principle, the expression cassette can include any gene that encodes a protein, polypeptide or RNA that is either reduced or absent due to a mutation or which conveys a therapeutic benefit when overexpressed is considered to be within the scope of the disclosure.

The expression cassette can comprise any transgene (e.g., encoding PAH protein), for example, PAH protein useful for treating PKU in a subject, i.e., a therapeutic PAH protein. According to aspects of the disclosure as described herein, the expression cassette comprises a codon optimized transgene. According to further embodiments, the codon optimized transgene is selected from a nucleic acid sequence set forth in Table 1A.

A ceDNA vector can be used to deliver and express any PAH protein of interest in the subject, alone or in combination with nucleic acids encoding polypeptides, or non-coding nucleic acids (e.g., RNAi, miRs etc.), as well as exogenous genes and nucleic acid sequences, including virus sequences in a subjects' genome, e.g., HIV virus sequences and the like. Preferably a ceDNA vector disclosed herein is used for therapeutic purposes (e.g., for medical, diagnostic, or veterinary uses) or immunogenic polypeptides. In certain embodiments, a ceDNA vector is useful to express any gene of interest in the subject, which includes one or more polypeptides, peptides, ribozymes, peptide nucleic acids, siRNAs, RNAis, antisense oligonucleotides, antisense polynucleotides, or RNAs (coding or non-coding; e.g., siRNAs, shRNAs, gRNA, micro-RNAs, and their antisense counterparts (e.g., antagoMiR)), antibodies, fusion proteins, or any combination thereof.

The expression cassette can also encode polypeptides, sense or antisense oligonucleotides, or RNAs (coding or non-coding; e.g., siRNAs, shRNAs, gRNA, micro-RNAs, and their antisense counterparts (e.g., antagoMiR)). Expression cassettes can include an exogenous sequence that encodes a reporter protein to be used for experimental or diagnostic purposes, such as β-lactamase, β-galactosidase (LacZ), alkaline phosphatase, thymidine kinase, green fluorescent protein (GFP), chloramphenicol acetyltransferase (CAT), luciferase, and others well known in the art.

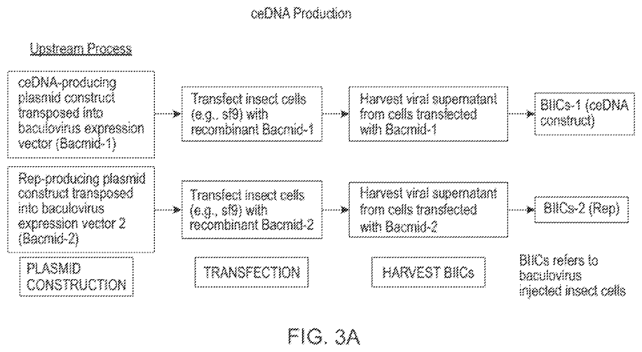

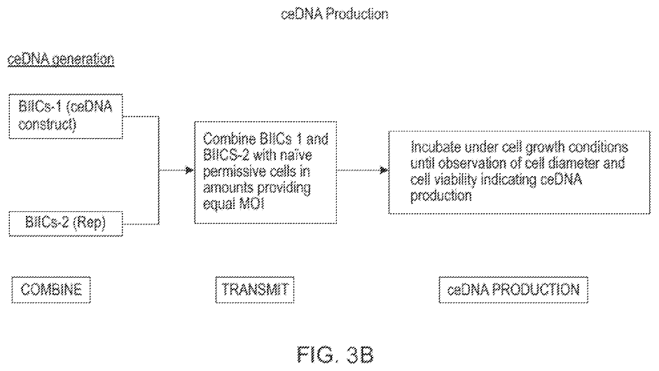

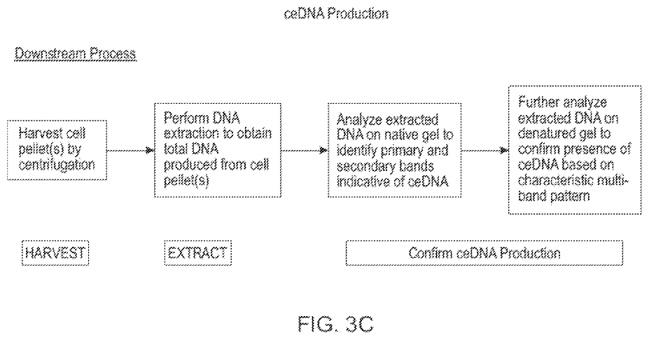

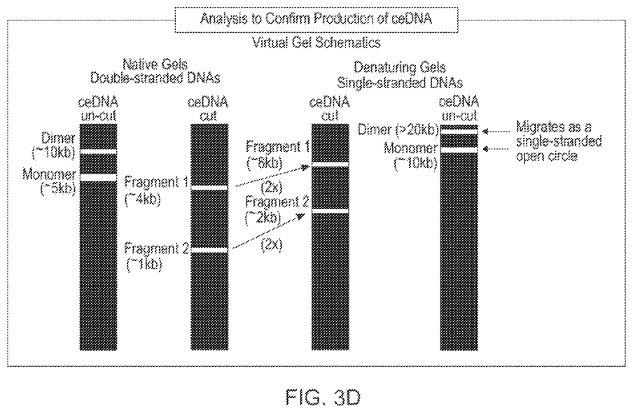

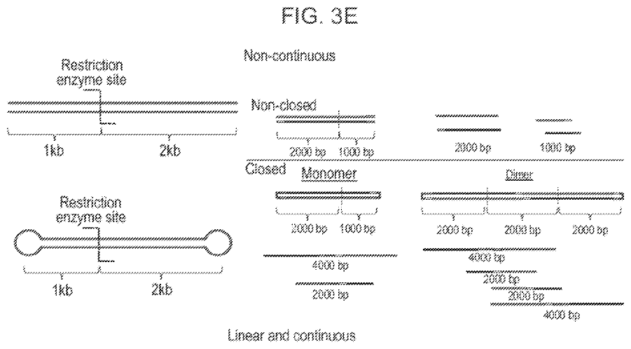

ceDNA vectors for expression of PAH protein produced by the methods provided herein preferably have a linear and continuous structure rather than a non-continuous structure, as determined by restriction enzyme digestion assay (FIG. 3D). The linear and continuous structure is believed to be more stable from attack by cellular endonucleases, as well as less likely to be recombined and cause mutagenesis. Thus, a ceDNA vector in the linear and continuous structure is a preferred embodiment. The continuous, linear, single strand intramolecular duplex ceDNA vector can have covalently bound terminal ends, without sequences encoding AAV capsid proteins. These ceDNA vectors are structurally distinct from plasmids (including ceDNA plasmids described herein), which are circular duplex nucleic acid molecules of bacterial origin. The complimentary strands of plasmids may be separated following denaturation to produce two nucleic acid molecules, whereas in contrast, ceDNA vectors, while having complimentary strands, are a single DNA molecule and therefore even if denatured, remain a single molecule. In some embodiments, ceDNA vectors as described herein can be produced without DNA base methylation of prokaryotic type, unlike plasmids. Therefore, the ceDNA vectors and ceDNA-plasmids are different both in term of structure (in particular, linear versus circular) and also in view of the methods used for producing and purifying these different objects (see below), and also in view of their DNA methylation which is of prokaryotic type for ceDNA-plasmids and of eukaryotic type for the ceDNA vector.

There are several advantages of using a ceDNA vector for expression of PAH protein as described herein over plasmid-based expression vectors, such advantages include, but are not limited to: 1) plasmids contain bacterial DNA sequences and are subjected to prokaryotic-specific methylation, e.g., 6-methyl adenosine and 5-methyl cytosine methylation, whereas capsid-free AAV vector sequences are of eukaryotic origin and do not undergo prokaryotic-specific methylation; as a result, capsid-free AAV vectors are less likely to induce inflammatory and immune responses compared to plasmids; 2) while plasmids require the presence of a resistance gene during the production process, ceDNA vectors do not; 3) while a circular plasmid is not delivered to the nucleus upon introduction into a cell and requires overloading to bypass degradation by cellular nucleases, ceDNA vectors contain viral cis-elements, i.e., ITRs, that confer resistance to nucleases and can be designed to be targeted and delivered to the nucleus. It is hypothesized that the minimal defining elements indispensable for ITR function are a Rep-binding site (RBS; 5′-GCGCGCTCGCTCGCTC-3′ (SEQ ID NO: 60) for AAV2) and a terminal resolution site (TRS; 5′-AGTTGG-3′ (SEQ ID NO: 64) for AAV2) plus a variable palindromic sequence allowing for hairpin formation; and 4) ceDNA vectors do not have the over-representation of CpG dinucleotides often found in prokaryote-derived plasmids that reportedly binds a member of the Toll-like family of receptors, eliciting a T cell-mediated immune response. In contrast, transductions with capsid-free AAV vectors disclosed herein can efficiently target cell and tissue-types that are difficult to transduce with conventional AAV virions using various delivery reagent.

### IV. Inverted Terminal Repeats (ITRs)

As disclosed herein, ceDNA vectors for expression of PAH protein contain nucleic acid, e.g., a transgene or heterologous nucleic acid sequence (e.g., a codon optimized heterologous nucleic acid sequence), positioned between two inverted terminal repeat (ITR) sequences, where the ITR sequences can be an asymmetrical ITR pair or a symmetrical- or substantially symmetrical ITR pair, as these terms are defined herein. A ceDNA vector as disclosed herein can comprise ITR sequences that are selected from any of: (i) at least one WT ITR and at least one modified AAV inverted terminal repeat (mod-ITR) (e.g., asymmetric modified ITRs); (ii) two modified ITRs where the mod-ITR pair have a different three-dimensional spatial organization with respect to each other (e.g., asymmetric modified ITRs), or (iii) symmetrical or substantially symmetrical WT-WT ITR pair, where each WT-ITR has the same three-dimensional spatial organization, or (iv) symmetrical or substantially symmetrical modified ITR pair, where each mod-ITR has the same three-dimensional spatial organization, where the methods of the present disclosure may further include a delivery system, such as but not limited to a liposome nanoparticle delivery system.

In some embodiments, the ITR sequence can be from viruses of the Parvoviridae family, which includes two subfamilies: Parvovirinae, which infect vertebrates, and Densovirinae, which infect insects. The subfamily Parvovirinae (referred to as the parvoviruses) includes the genus Dependovirus, the members of which, under most conditions, require coinfection with a helper virus such as adenovirus or herpes virus for productive infection. The genus Dependovirus includes adeno-associated virus (AAV), which normally infects humans (e.g., serotypes 2, 3A, 3B, 5, and 6) or primates (e.g., serotypes 1 and 4), and related viruses that infect other warm-blooded animals (e.g., bovine, canine, equine, and ovine adeno-associated viruses). The parvoviruses and other members of the Parvoviridae family are generally described in Kenneth I. Berns, “Parvoviridae: The Viruses and Their Replication,” Chapter 69 in FIELDS VIROLOGY (3d Ed. 1996).

While ITRs exemplified in the specification and Examples herein are AAV2 WT-ITRs, one of ordinary skill in the art is aware that one can as stated above use ITRs from any known parvovirus, for example a Dependovirus such as AAV (e.g., AAV1, AAV2, AAV3, AAV4, AAV5, AAV 5, AAV7, AAV8, AAV9, AAV10, AAV 11, AAV12, AAVrh8, AAVrh10, AAV-DJ, and AAV-DJ8 genome. E.g., NCBI: NC 002077; NC 001401; NC001729; NC001829; NC006152; NC 006260; NC 006261), chimeric ITRs, or ITRs from any synthetic AAV. In some embodiments, the AAV can infect warm-blooded animals, e.g., avian (AAAV), bovine (BAAV), canine, equine, and ovine adeno-associated viruses. In some embodiments the ITR is from B19 parvovirus (GenBank Accession No: NC 000883), Minute Virus from Mouse (MVM) (GenBank Accession No. NC 001510); goose parvovirus (GenBank Accession No. NC 001701); snake parvovirus 1 (GenBank Accession No. NC 006148). In some embodiments, the 5′ WT-ITR can be from one serotype and the 3′ WT-ITR from a different serotype, as discussed herein.

An ordinarily skilled artisan is aware that ITR sequences have a common structure of a double-stranded Holliday junction, which typically is a T-shaped or Y-shaped hairpin structure, where each WT-ITR is formed by two palindromic arms or loops (B-B′ and C-C′) embedded in a larger palindromic arm (A-A′), and a single stranded D sequence, (where the order of these palindromic sequences defines the flip or flop orientation of the ITR). See, for example, structural analysis and sequence comparison of ITRs from different AAV serotypes (AAV1-AAV6) and described in Grimm et al., J. Virology, 2006; 80(1); 426-439; Yan et al., J. Virology, 2005; 364-379; Duan et al., Virology 1999; 261; 8-14. One of ordinary skill in the art can readily determine WT-ITR sequences from any AAV serotype for use in a ceDNA vector or ceDNA-plasmid based on the exemplary AAV2 ITR sequences provided herein. See, for example, the sequence comparison of ITRs from different AAV serotypes (AAV1-AAV6, and avian AAV (AAAV) and bovine AAV (BAAV)) described in Grimm et al., J. Virology, 2006; 80(1); 426-439; that show the % identity of the left ITR of AAV2 to the left ITR from other serotypes: AAV-1 (84%), AAV-3 (86%), AAV-4 (79%), AAV-5 (58%), AAV-6 (left ITR) (100%) and AAV-6 (right ITR) (82%).

### A. Symmetrical ITR Pairs

In some embodiments, a ceDNA vector for expression of PAH protein as described herein comprises, in the 5′ to 3′ direction: a first adeno-associated virus (AAV) inverted terminal repeat (ITR), a nucleic acid sequence of interest (for example an expression cassette as described herein) and a second AAV ITR, where the first ITR (5′ ITR) and the second ITR (3′ ITR) are symmetric, or substantially symmetrical with respect to each other—that is, a ceDNA vector can comprise ITR sequences that have a symmetrical three-dimensional spatial organization such that their structure is the same shape in geometrical space, or have the same A, C-C′ and B-B′ loops in 3D space. In such an embodiment, a symmetrical ITR pair, or substantially symmetrical ITR pair can be modified ITRs (e.g., mod-ITRs) that are not wild-type ITRs. A mod-ITR pair can have the same sequence which has one or more modifications from wild-type ITR and are reverse complements (inverted) of each other. In alternative embodiments, a modified ITR pair are substantially symmetrical as defined herein, that is, the modified ITR pair can have a different sequence but have corresponding or the same symmetrical three-dimensional shape.

(i) Wildtype ITRs

In some embodiments, the symmetrical ITRs, or substantially symmetrical ITRs are wild type (WT-ITRs) as described herein. That is, both ITRs have a wild-type sequence, but do not necessarily have to be WT-ITRs from the same AAV serotype. That is, in some embodiments, one WT-ITR can be from one AAV serotype, and the other WT-ITR can be from a different AAV serotype. In such an embodiment, a WT-ITR pair are substantially symmetrical as defined herein, that is, they can have one or more conservative nucleotide modification while still retaining the symmetrical three-dimensional spatial organization.

Accordingly, as disclosed herein, ceDNA vectors contain a nucleic acid sequence, e.g., transgene or heterologous nucleic acid sequence, positioned between two flanking wild-type inverted terminal repeat (WT-ITR) sequences, that are either the reverse complement (inverted) of each other, or alternatively, are substantially symmetrical relative to each other—that is a WT-ITR pair have symmetrical three-dimensional spatial organization. In some embodiments, a wild-type ITR sequence (e.g., AAV WT-ITR) comprises a functional Rep binding site (RBS; e.g., 5′-GCGCGCTCGCTCGCTC-3′ for AAV2, SEQ ID NO: 60) and a functional terminal resolution site (TRS; e.g., 5′-AGTT-3′, SEQ ID NO: 62).

In one aspect, ceDNA vectors for expression of PAH protein are obtainable from a vector polynucleotide that encodes a nucleic acid, e.g., heterologous nucleic acid, operatively positioned between two WT inverted terminal repeat sequences (WT-ITRs) (e.g., AAV WT-ITRs). That is, both ITRs have a wild-type sequence, but do not necessarily have to be WT-ITRs from the same AAV serotype. That is, in some embodiments, one WT-ITR can be from one AAV serotype, and the other WT-ITR can be from a different AAV serotype. In such an embodiment, the WT-ITR pair are substantially symmetrical as defined herein, that is, they can have one or more conservative nucleotide modification while still retaining the symmetrical three-dimensional spatial organization. In some embodiments, the 5′ WT-ITR is from one AAV serotype, and the 3′ WT-ITR is from the same or a different AAV serotype. In some embodiments, the 5′ WT-ITR and the 3′WT-ITR are mirror images of each other, that is they are symmetrical. In some embodiments, the 5′ WT-ITR and the 3′ WT-ITR are from the same AAV serotype.

WT ITRs are well known. In one embodiment the two ITRs are from the same AAV2 serotype. In certain embodiments one can use WT from other serotypes. There are a number of serotypes that are homologous, e.g., AAV2, AAV4, AAV6, AAV8. In one embodiment, closely homologous ITRs (e.g., ITRs with a similar loop structure) can be used. In another embodiment, one can use AAV WT ITRs that are more diverse, e.g., AAV2 and AAV5, and still another embodiment, one can use an ITR that is substantially WT—that is, it has the basic loop structure of the WT but some conservative nucleotide changes that do not alter or affect the properties. When using WT-ITRs from the same viral serotype, one or more regulatory sequences may further be used. In certain embodiments, the regulatory sequence is a regulatory switch that permits modulation of the activity of the ceDNA, e.g., the expression of the encoded PAH protein.

In some embodiments, one aspect of the technology described herein relates to a ceDNA vector for expression of PAH protein, wherein the ceDNA vector comprises at least one nucleotide sequence, e.g., heterologous nucleic acid sequence, encoding the PAH protein, operably positioned between two wild-type inverted terminal repeat sequences (WT-ITRs), wherein the WT-ITRs can be from the same serotype, different serotypes or substantially symmetrical with respect to each other (i.e., have the symmetrical three-dimensional spatial organization such that their structure is the same shape in geometrical space, or have the same A, C-C′ and B-B′ loops in 3D space). In some embodiments, the symmetric WT-ITRs comprises a functional terminal resolution site and a Rep binding site. In some embodiments, the nucleic acid, e.g., heterologous nucleic acid, sequence encodes a transgene, and wherein the vector is not in a viral capsid.

In some embodiments, the WT-ITRs are the same but the reverse complement of each other. For example, the sequence AACG in the 5′ ITR may be CGTT (i.e., the reverse complement) in the 3′ ITR at the corresponding site. In one example, the 5′ WT-ITR sense strand comprises the sequence of ATCGATCG (SEQ ID NO: 605) and the corresponding 3′ WT-ITR sense strand comprises CGATCGAT (SEQ ID NO: 606) (i.e., the reverse complement of ATCGATCG (SEQ ID NO: 607)). In some embodiments, the WT-ITRs ceDNA further comprises a terminal resolution site and a replication protein binding site (RPS) (sometimes referred to as a replicative protein binding site), e.g., a Rep binding site.

Exemplary WT-ITR sequences for use in the ceDNA vectors for expression of PAH protein comprising WT-ITRs are shown in Table 3 herein, which shows pairs of WT-ITRs (5′ WT-ITR and the 3′ WT-ITR).

In some embodiments, the flanking WT-ITRs are identical and symmetrical with respect to each other. In some other embodiments, the flanking WT-ITRs are substantially symmetrical with respect to each other. In this embodiment, the 5′ WT-ITR can be from one serotype of AAV, and the 3′ WT-ITR from a different serotype of AAV, such that the WT-ITRs are not identical reverse complements. For example, the 5′ WT-ITR can be from AAV2, and the 3′ WT-ITR from a different serotype (e.g. AAV1, 3, 4, 5, 6, 7, 8, 9, 10, 11, and 12. In some embodiments, WT-ITRs can be selected from two different parvoviruses selected from any to of: AAV1, AAV2, AAV3, AAV4, AAV5, AAV6, AAV7, AAV8, AAV9, AAV10, AAV11, AAV12, AAV13, snake parvovirus (e.g., royal python parvovirus), bovine parvovirus, goat parvovirus, avian parvovirus, canine parvovirus, equine parvovirus, shrimp parvovirus, porcine parvovirus, or insect AAV. In some embodiments, such a combination of WT ITRs is the combination of WT-ITRs from AAV2 and AAV6. In one embodiment, the substantially symmetrical WT-ITRs are when one is inverted relative to the other ITR at least 90% identical, at least 95% identical, at least 96% . . . 97% . . . 98% . . . 99% . . . 99.5% and all points in between, and has the same symmetrical three-dimensional spatial organization. In some embodiments, a WT-ITR pair are substantially symmetrical as they have symmetrical three-dimensional spatial organization, e.g., have the same 3D organization of the A, C-C′. B-B′ and D arms. In one embodiment, a substantially symmetrical WT-ITR pair are inverted relative to the other, and are at least 95% identical, at least 96% . . . 97% . . . 98% . . . 99% . . . 99.5% and all points in between, to each other, and one WT-ITR retains the Rep-binding site (RBS) of 5′-GCGCGCTCGCTCGCTC-3′ (SEQ ID NO: 60) and a terminal resolution site (trs). In some embodiments, a substantially symmetrical WT-ITR pair are inverted relative to each other, and are at least 95% identical, at least 96% . . . 97% . . . 98% . . . 99% . . . 99.5% and all points in between, to each other, and one WT-ITR retains the Rep-binding site (RBS) of 5′-GCGCGCTCGCTCGCTC-3′ (SEQ ID NO: 60) and a terminal resolution site (trs) and in addition to a variable palindromic sequence allowing for hairpin secondary structure formation. Homology can be determined by standard means well known in the art such as BLAST (Basic Local Alignment Search Tool), BLASTN at default setting.

In some embodiments, the structural element of the ITR can be any structural element that is involved in the functional interaction of the ITR with a large Rep protein (e.g., Rep 78 or Rep 68). In certain embodiments, the structural element provides selectivity to the interaction of an ITR with a large Rep protein, i.e., determines at least in part which Rep protein functionally interacts with the ITR. In other embodiments, the structural element physically interacts with a large Rep protein when the Rep protein is bound to the ITR. Each structural element can be, e.g., a secondary structure of the ITR, a nucleic acid sequence of the ITR, a spacing between two or more elements, or a combination of any of the above. In one embodiment, the structural elements are selected from the group consisting of an A and an A′ arm, a B and a B′ arm, a C and a C′ arm, a D arm, a Rep binding site (RBE) and an RBE′ (i.e., complementary RBE sequence), and a terminal resolution sire (trs).

By way of example only, Table 2 indicates exemplary combinations of WT-ITRs.

Table 2: Exemplary combinations of WT-ITRs from the same serotype or different serotypes, or different parvoviruses. The order shown is not indicative of the ITR position, for example, “AAV1, AAV2” demonstrates that the ceDNA can comprise a WT-AAV1 ITR in the 5′ position, and a WT-AAV2 ITR in the 3′ position, or vice versa, a WT-AAV2 ITR the 5′ position, and a WT-AAV1 ITR in the 3′ position. Abbreviations: AAV serotype 1 (AAV1), AAV serotype 2 (AAV2), AAV serotype 3 (AAV3), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), or AAV serotype 12 (AAV12); AAVrh8, AAVrh10, AAV-DJ, and AAV-DJ8 genome (E.g., NCBI: NC 002077; NC 001401; NC001729; NC001829; NC006152; NC 006260; NC 006261), ITRs from warm-blooded animals (avian AAV (AAAV), bovine AAV (BAAV), canine, equine, and ovine AAV), ITRs from B19 parvovirus (GenBank Accession No: NC 000883), Minute Virus from Mouse (MVM) (GenBank Accession No. NC 001510); Goose: goose parvovirus (GenBank Accession No. NC 001701); snake: snake parvovirus 1 (GenBank Accession No. NC 006148).

TABLE 2

| | | | | |
|---|---|---|---|---|
| AAV1, AAV1 | AAV2, AAV2 | AAV3, AAV3 | AAV4, AAV4 | AAV5, AAV5 |
| AAV1, AAV2 | AAV2, AAV3 | AAV3, AAV4 | AAV4, AAV5 | AAV5, AAV6 |
| AAV1, AAV3 | AAV2, AAV4 | AAV3, AAV5 | AAV4, AAV6 | AAV5, AAV7 |
| AAV1, AAV4 | AAV2, AAV5 | AAV3, AAV6 | AAV4, AAV7 | AAV5, AAV8 |
| AAV1, AAV5 | AAV2, AAV6 | AAV3, AAV7 | AAV4, AAV8 | AAV5, AAV9 |
| AAV1, AAV6 | AAV2, AAV7 | AAV3, AAV8 | AAV4, AAV9 | AAV5, AAV10 |
| AAV1, AAV7 | AAV2, AAV8 | AAV3, AAV9 | AAV4, AAV10 | AAV5, AAV11 |
| AAV1, AAV8 | AAV2, AAV9 | AAV3, AAV10 | AAV4, AAV11 | AAV5, AAV12 |
| AAV1, AAV9 | AAV2, AAV10 | AAV3, AAV11 | AAV4, AAV12 | AAV5, AAVRH8 |
| AAV1, AAV10 | AAV2, AAV11 | AAV3, AAV12 | AAV4, AAVRH8 | AAV5, AAVRH10 |
| AAV1, AAV11 | AAV2, AAV12 | AAV3, AAVRH8 | AAV4, AAVRH10 | AAV5, AAV13 |
| AAV1, AAV12 | AAV2, AAVRH8 | AAV3, AAVRH10 | AAV4, AAV13 | AAV5, AAVDJ |
| AAV1, AAVRH8 | AAV2, AAVRH10 | AAV3, AAV13 | AAV4, AAVDJ | AAV5, AAVDJ8 |
| AAV1, AAVRH10 | AAV2, AAV13 | AAV3, AAVDJ | AAV4, AAVDJ8 | AAV5, AVIAN |
| AAV1, AAV13 | AAV2, AAVDJ | AAV3, AAVDJ8 | AAV4, AVIAN | AAV5, BOVINE |
| AAV1, AAVDJ | AAV2, AAVDJ8 | AAV3, AVIAN | AAV4, BOVINE | AAV5, CANINE |
| AAV1, AAVDJ8 | AAV2, AVIAN | AAV3, BOVINE | AAV4, CANINE | AAV5, EQUINE |
| AAV1, AVIAN | AAV2, BOVINE | AAV3, CANINE | AAV4, EQUINE | AAV5, GOAT |
| AAV1, BOVINE | AAV2, CANINE | AAV3, EQUINE | AAV4, GOAT | AAV5, SHRIMP |
| AAV1, CANINE | AAV2, EQUINE | AAV3, GOAT | AAV4, SHRIMP | AAV5, PORCINE |
| AAV1, EQUINE | AAV2, GOAT | AAV3, SHRIMP | AAV4, PORCINE | AAV5, INSECT |
| AAV1, GOAT | AAV2, SHRIMP | AAV3, PORCINE | AAV4, INSECT | AAV5, OVINE |
| AAV1, SHRIMP | AAV2, PORCINE | AAV3, INSECT | AAV4, OVINE | AAV5, B19 |
| AAV1, PORCINE | AAV2, INSECT | AAV3, OVINE | AAV4, B19 | AAV5, MVM |
| AAV1, INSECT | AAV2, BOVINE | AAV3, B19 | AAV4, MVM | AAV5, GOOSE |
| AAV1, OVINE | AAV2, B19 | AAV3, MVM | AAV4, GOOSE | AAV5, SNAKE |
| AAV1, B19 | AAV2, MVM | AAV3, GOOSE | AAV4, SNAKE | |
| AAV1, MVM | AAV2, GOOSE | AAV3, SNAKE | | |
| AAV1, GOOSE | AAV2, SNAKE | | | |
| AAV1, SNAKE | | | | |
| AAV6, AAV6 | AAV7, AAV7 | AAV8, AAV8 | AAV9, AAV9 | AAV10, AAV10 |
| AAV6, AAV7 | AAV7, AAV8 | AAV8, AAV9 | AAV9, AAV10 | AAV10, AAV11 |
| AAV6, AAV8 | AAV7, AAV9 | AAV8, AAV10 | AAV9, AAV11 | AAV10, AAV12 |
| AAV6, AAV9 | AAV7, AAV10 | AAV8, AAV11 | AAV9, AAV12 | AAV10, AAVRH8 |
| AAV6, AAV10 | AAV7, AAV11 | AAV8, AAV12 | AAV9, AAVRH8 | AAV10, AAVRH10 |
| AAV6, AAV11 | AAV7, AAV12 | AAV8, AAVRH8 | AAV9, AAVRH10 | AAV10, AAV13 |
| AAV6, AAV12 | AAV7, AAVRH8 | AAV8, AAVRH10 | AAV9, AAV13 | AAV10, AAVDJ |
| AAV6, AAVRH8 | AAV7, AAVRH10 | AAV8, AAV13 | AAV9, AAVDJ | AAV10, AAVDJ8 |
| AAV6, AAVRH10 | AAV7, AAV13 | AAV8, AAVDJ | AAV9, AAVDJ8 | AAV10, AVIAN |
| AAV6, AAV13 | AAV7, AAVDJ | AAV8, AAVDJ8 | AAV9, AVIAN | AAV10, BOVINE |
| AAV6, AAVDJ | AAV7, AAVDJ8 | AAV8, AVIAN | AAV9, BOVINE | AAV10, CANINE |
| AAV6, AAVDJ8 | AAV7, AVIAN | AAV8, BOVINE | AAV9, CANINE | AAV10, EQUINE |
| AAV6, AVIAN | AAV7, BOVINE | AAV8, CANINE | AAV9, EQUINE | AAV10, GOAT |
| AAV6, BOVINE | AAV7, CANINE | AAV8, EQUINE | AAV9, GOAT | AAV10, SHRIMP |
| AAV6, CANINE | AAV7, EQUINE | AAV8, GOAT | AAV9, SHRIMP | AAV10, PORCINE |
| AAV6, EQUINE | AAV7, GOAT | AAV8, SHRIMP | AAV9, PORCINE | AAV10, INSECT |
| AAV6, GOAT | AAV7, SHRIMP | AAV8, PORCINE | AAV9, INSECT | AAV10, OVINE |
| AAV6, SHRIMP | AAV7, PORCINE | AAV8, INSECT | AAV9, OVINE | AAV10, B19 |
| AAV6, PORCINE | AAV7, INSECT | AAV8, OVINE | AAV9, B19 | AAV10, MVM |
| AAV6, INSECT | AAV7, OVINE | AAV8, B19 | AAV9, MVM | AAV10, GOOSE |
| AAV6, OVINE | AAV7, B19 | AAV8, MVM | AAV9, GOOSE | AAV10, SNAKE |
| AAV6, B19 | AAV7, MVM | AAV8, GOOSE | AAV9, SNAKE | |
| AAV6, MVM | AAV7, GOOSE | AAV8, SNAKE | | |
| AAV6, GOOSE | AAV7, SNAKE | | | |
| AAV6, SNAKE | | | | |
| AAV11, AAV11 | AAV12, AAV12 | AAVRH8, AAVRH8 | AAVRH10, AAVRH10 | AAV13, AAV13 |
| AAV11, AAV12 | AAV12, AAVRH8 | AAVRH8, AAVRH10 | AAVRH10, AAV13 | AAV13, AAVDJ |
| AAV11, AAVRH8 | AAV12, AAVRH10 | AAVRH8, AAV13 | AAVRH10, AAVDJ | AAV13, AAVDJ8 |
| AAV11, AAVRH10 | AAV12, AAV13 | AAVRH8, AAVDJ | AAVRH10, AAVDJ8 | AAV13, AVIAN |
| AAV11, AAV13 | AAV12, AAVDJ | AAVRH8, AAVDJ8 | AAVRH10, AVIAN | AAV13, BOVINE |
| AAV11, AAVDJ | AAV12, AAVDJ8 | AAVRH8, AVIAN | AAVRH10, BOVINE | AAV13, CANINE |
| AAV11, AAVDJ8 | AAV12, AVIAN | AAVRH8, BOVINE | AAVRH10, CANINE | AAV13, EQUINE |
| AAV11, AVIAN | AAV12, BOVINE | AAVRH8, CANINE | AAVRH10, EQUINE | AAV13, GOAT |
| AAV11, BOVINE | AAV12, CANINE | AAVRH8, EQUINE | AAVRH10, GOAT | AAV13, SHRIMP |
| AAV11, CANINE | AAV12, EQUINE | AAVRH8, GOAT | AAVRH10, SHRIMP | AAV13, PORCINE |
| AAV11, EQUINE | AAV12, GOAT | AAVRH8, SHRIMP | AAVRH10, PORCINE | AAV13, INSECT |
| AAV11, GOAT | AAV12, SHRIMP | AAVRH8, PORCINE | AAVRH10, INSECT | AAV13, OVINE |
| AAV11, SHRIMP | AAV12, PORCINE | AAVRH8, INSECT | AAVRH10, OVINE | AAV13, B19 |
| AAV11, PORCINE | AAV12, INSECT | AAVRH8, OVINE | AAVRH10, B19 | AAV13, MVM |
| AAV11, INSECT | AAV12, OVINE | AAVRH8, B19 | AAVRH10, MVM | AAV13, GOOSE |
| AAV11, OVINE | AAV12, B19 | AAVRH8, MVM | AAVRH10, GOOSE | AAV13, SNAKE |
| AAV11, B19 | AAV12, MVM | AAVRH8, GOOSE | AAVRH10, SNAKE | |
| AAV11, MVM | AAV12, GOOSE | AAVRH8, SNAKE | | |
| AAV11, GOOSE | AAV12, SNAKE | | | |
| AAV11, SNAKE | | | | |
| AAVDJ, AAVDJ | AAVDJ8, AVVDJ8 | AVIAN, AVIAN | BOVINE, BOVINE | CANINE, CANINE |
| AAVDJ, AAVDJ8 | AAVDJ8, AVIAN | AVIAN, BOVINE | BOVINE, CANINE | CANINE, EQUINE |
| AAVDJ, AVIAN | AAVDJ8, BOVINE | AVIAN, CANINE | BOVINE, EQUINE | CANINE, GOAT |
| AAVDJ, BOVINE | AAVDJ8, CANINE | AVIAN, EQUINE | BOVINE, GOAT | CANINE, SHRIMP |
| AAVDJ, CANINE | AAVDJ8, EQUINE | AVIAN, GOAT | BOVINE, SHRIMP | CANINE, PORCINE |
| AAVDJ, EQUINE | AAVDJ8, GOAT | AVIAN, SHRIMP | BOVINE, PORCINE | CANINE, INSECT |
| AAVDJ, GOAT | AAVDJ8, SHRIMP | AVIAN, PORCINE | BOVINE, INSECT | CANINE, OVINE |
| AAVDJ, SHRIMP | AAVDJ8, PORCINE | AVIAN, INSECT | BOVINE, OVINE | CANINE, B19 |
| AAVDJ, PORCINE | AAVDJ8, INSECT | AVIAN, OVINE | BOVINE, B19 | CANINE, MVM |
| AAVDJ, INSECT | AAVDJ8, OVINE | AVIAN, B19 | BOVINE, MVM | CANINE, GOOSE |
| AAVDJ, OVINE | AAVDJ8, B19 | AVIAN, MVM | BOVINE, GOOSE | CANINE, SNAKE |
| AAVDJ, B19 | AAVDJ8, MVM | AVIAN, GOOSE | BOVINE, SNAKE | |
| AAVDJ, MVM | AAVDJ8, GOOSE | AVIAN, SNAKE | | |
| AAVDJ, GOOSE | AAVDJ8, SNAKE | | | |
| AAVDJ, SNAKE | | | | |
| EQUINE, EQUINE | GOAT, GOAT | SHRIMP, SHRIMP | PORCINE, PORCINE | INSECT, INSECT |
| EQUINE, GOAT | GOAT, SHRIMP | SHRIMP, PORCINE | PORCINE, INSECT | INSECT, OVINE |
| EQUINE, SHRIMP | GOAT, PORCINE | SHRIMP, INSECT | PORCINE, OVINE | INSECT, B19 |
| EQUINE, PORCINE | GOAT, INSECT | SHRIMP, OVINE | PORCINE, B19 | INSECT, MVM |
| EQUINE, INSECT | GOAT, OVINE | SHRIMP, B19 | PORCINE, MVM | INSECT, GOOSE |
| EQUINE, OVINE | GOAT, B19 | SHRIMP, MVM | PORCINE, GOOSE | INSECT, SNAKE |
| EQUINE, B19 | GOAT, MVM | SHRIMP, GOOSE | PORCINE, SNAKE | |
| EQUINE, MVM | GOAT, GOOSE | SHRIMP, SNAKE | | |
| EQUINE, GOOSE | GOAT, SNAKE | | | |
| EQUINE, SNAKE | | | | |
| OVINE, OVINE | B19, B19 | MVM, MVM | GOOSE, GOOSE | SNAKE, SNAKE |
| OVINE, B19 | B19, MVM | MVM, GOOSE | GOOSE, SNAKE | |
| OVINE, MVM | B19, GOOSE | MVM, SNAKE | | |
| OVINE, GOOSE | B19, SNAKE | | | |
| OVINE, SNAKE | | | | |

By way of example only, Table 3 shows the sequences of exemplary WT-ITRs from some different AAV serotypes.

TABLE 3

| AAV | 5′ WT-ITR | 3′ WT-ITR |
|---|---|---|
| serotype | (LEFT) | (RIGHT) |
| AAV1 | 5′- | 5′- |
| | TTGCCCACTCCCTCT | TTACCCTAGTGATGG |
| | CTGCGCGCTCGCTCG | AGTTGCCCACTCCCT |
| | CTCGGTGGGGCCTGC | CTCTGCGCGCGTCGC |
| | GGACCAAAGGTCCGC | TCGCTCGGTGGGGCC |
| | AGACGGCAGAGGTCT | GGCAGAGGAGACCTC |
| | CCTCTGCCGGCCCCA | TGCCGTCTGCGGACC |
| | CCGAGCGAGCGACGC | TTTGGTCCGCAGGCC |
| | GCGCAGAGAGGGAGT | CCACCGAGCGAGCGA |
| | GGGCAACTCCATCAC | GCGCGCAGAGAGGGA |
| | TAGGGTAA-3′ | GTGGGCAA-3′ |
| | (SEQ ID NO: 5) | (SEQ ID NO: 10) |
| AAV2 | CCTGCAGGCAGCTGC | AGGAACCCCTAGTGA |
| | GCGCTCGCTCGCTCA | TGGAGTTGGCCACTC |
| | CTGAGGCCGCCCGGG | CCTCTCTGCGCGCTC |
| | CAAAGCCCGGGCGTC | GCTCGCTCACTGAGG |
| | GGGCGACCTTTGGTC | CCGGGCGACCAAAGG |
| | GCCCGGCCTCAGTGA | TCGCCCGACGCCCGG |
| | GCGAGCGAGCGCGCA | GCTTTGCCCGGGCGG |
| | GAGAGGGAGTGGCCA | CCTCAGTGAGCGAGC |
| | ACTCCATCACTAGGG | GAGCGCGCAGCTGCC |
| | GTTCCT | TGCAGG |
| | (SEQ ID NO: 2) | (SEQ ID NO: 1) |
| AAV3 | 5′- | 5′- |
| | TTGGCCACTCCCTCT | ATACCTCTAGTGATG |
| | ATGCGCACTCGCTCG | GAGTTGGCCACTCCC |
| | CTCGGTGGGGCCTGG | TCTATGCGCACTCGC |
| | CGACCAAAGGTCGCC | TCGCTCGGTGGGGCC |
| | AGACGGACGTGGGTT | GGACGTGGAAACCCA |
| | TCCACGTCCGGCCCC | CGTCCGTCTGGCGAC |
| | ACCGAGCGAGCGAGT | CTTTGGTCGCCAGGC |
| | GCGCATAGAGGGAGT | CCCACCGAGCGAGCG |
| | GGCCAACTCCATCAC | AGTGCGCATAGAGGG |
| | TAGAGGTAT-3′ | AGTGGCCAA-3′ |
| | (SEQ ID NO: 6) | (SEQ ID NO: 11) |
| AAV4 | 5′- | 5′- |
| | TTGGCCACTCCCTCT | AGTTGGCCACATTAG |
| | ATGCGCGCTCGCTCA | CTATGCGCGCTCGCT |
| | CTCACTCGGCCCTGG | CACTCACTCGGCCCT |
| | AGACCAAAGGTCTCC | GGAGACCAAAGGTCT |
| | AGACTGCCGGCCTCT | CCAGACTGCCGGCCT |
| | GGCCGGCAGGGCCGA | CTGGCCGGCAGGGCC |
| | GTGAGTGAGCGAGCG | GAGTGAGTGAGCGAG |
| | CGCATAGAGGGAGTG | CGCGCATAGAGGGAG |
| | GCCAACT-3′ | TGGCCAA-3′ |
| | (SEQ ID NO: 7) | (SEQ ID NO: 12) |
| AAV5 | 5′- | 5′- |
| | TCCCCCCTGTCGCGT | CTTACAAAACCCCCT |
| | TCGCTCGCTCGCTGG | TGCTTGAGAGTGTGG |
| | CTCGTTTGGGGGGGC | CACTCTCCCCCCTGT |
| | GACGGCCAGAGGGCC | CGCGTTCGCTCGCTC |
| | GTCGTCTGGCAGCTC | GCTGGCTCGTTTGGG |
| | TTTGAGCTGCCACCC | GGGGTGGCAGCTCAA |
| | CCCCAAACGAGCCAG | AGAGCTGCCAGACGA |
| | CGAGCGAGCGAACGC | CGGCCCTCTGGCCGT |
| | GACAGGGGGGAGAGT | CGCCCCCCCAAACGA |
| | GCCACACTCTCAAGC | GCCAGCGAGCGAGCG |
| | AAGGGGGTTTTGTAA | AACGCGACAGGGGGG |
| | G-3′ | A-3′ |
| | (SEQ ID NO: 8) | (SEQ ID NO: 13) |
| AAV6 | 5′- | 5′- |
| | TTGCCCACTCCCTCT | ATACCCCTAGTGATG |
| | AATGCGCGCTCGCTC | GAGTTGCCCACTCCC |
| | GCTCGGTGGGGCCTG | TCTATGCGCGCTCGC |
| | CGGACCAAAGGTCCG | TCGCTCGGTGGGGCC |
| | CAGACGGCAGAGGTC | GGCAGAGGAGACCTC |
| | TCCTCTGCCGGCCCC | TGCCGTCTGCGGACC |
| | ACCGAGCGAGCGAGC | TTTGGTCCGCAGGCC |
| | GCGCATAGAGGGAGT | CCACCGAGCGAGCGA |
| | GGGCAACTCCATCAC | GCGCGCATTAGAGGG |
| | TAGGGGTAT-3′ | AGTGGGCAA |
| | (SEQ ID NO: 9) | (SEQ ID NO: 14) |

In some embodiments, the nucleic acid sequence of the WT-ITR sequence can be modified (e.g., by modifying 1, 2, 3, 4 or 5, or more nucleotides or any range therein), whereby the modification is a substitution for a complementary nucleotide, e.g., G for a C, and vice versa, and T for an A, and vice versa.

In certain embodiments of the present disclosure, the ceDNA vector for expression of PAH protein does not have a WT-ITR consisting of the nucleic acid sequence selected from any of: SEQ ID NOs: 1, 2, 5-14. In alternative embodiments of the present disclosure, if a ceDNA vector has a WT-ITR comprising the nucleic acid sequence selected from any of: SEQ ID NOs: 1, 2, 5-14, then the flanking ITR is also WT and the ceDNA vector comprises a regulatory switch, e.g., as disclosed herein and in International Patent Application No. PCT/US18/49996 (e.g., see Table 11 of PCT/US 18/49996, incorporated by reference in its entirety herein). In some embodiments, the ceDNA vector for expression of PAH protein comprises a regulatory switch as disclosed herein and a WT-ITR selected having the nucleic acid sequence selected from any of the group consisting of: SEQ ID NO: 1, 2, 5-14.

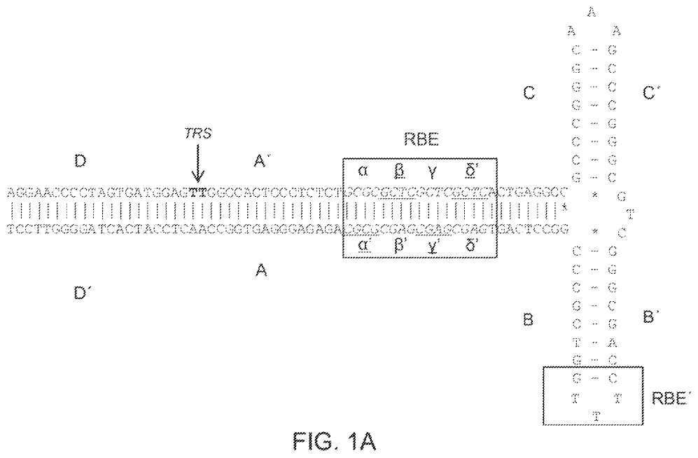

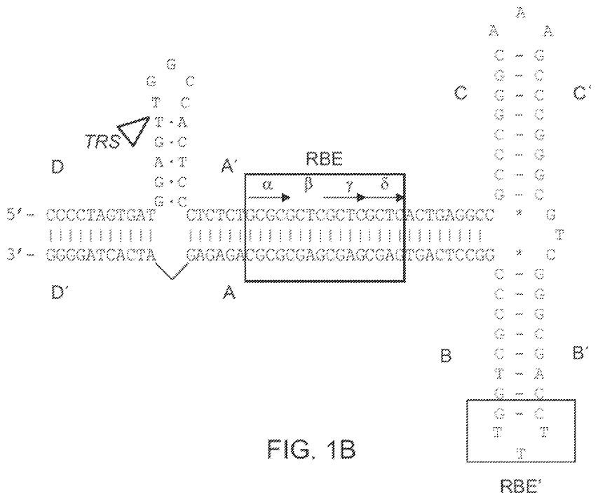

The ceDNA vector for expression of PAH protein as described herein can include WT-ITR structures that retains an operable RBE, trs and RBE′ portion. FIG. 1A and FIG. 1B, using wild-type ITRs for exemplary purposes, show one possible mechanism for the operation of a trs site within a wild type ITR structure portion of a ceDNA vector. In some embodiments, the ceDNA vector for expression of PAH protein contains one or more functional WT-ITR polynucleotide sequences that comprise a Rep-binding site (RBS; 5′-GCGCGCTCGCTCGCTC-3′ (SEQ ID NO: 60) for AAV2) and a terminal resolution site (TRS; 5′-AGTT (SEQ ID NO: 62)). In some embodiments, at least one WT-ITR is functional. In alternative embodiments, where a ceDNA vector for expression of PAH protein comprises two WT-ITRs that are substantially symmetrical to each other, at least one WT-ITR is functional and at least one WT-ITR is non-functional.

B. Modified ITRs (Mod-ITRs) in General for ceDNA Vectors Comprising Asymmetric ITR Pairs or Symmetric ITR Pairs

As discussed herein, a ceDNA vector for expression of PAH protein can comprise a symmetrical ITR pair or an asymmetrical ITR pair. In both instances, one or both of the ITRs can be modified ITRs—the difference being that in the first instance (i.e., symmetric mod-ITRs), the mod-ITRs have the same three-dimensional spatial organization (i.e., have the same A-A′, C-C′ and B-B′ arm configurations), whereas in the second instance (i.e., asymmetric mod-ITRs), the mod-ITRs have a different three-dimensional spatial organization (i.e., have a different configuration of A-A′, C-C′ and B-B′ arms).

In some embodiments, a modified ITR is an ITRs that is modified by deletion, insertion, and/or substitution as compared to a wild-type ITR sequence (e.g., AAV ITR). In some embodiments, at least one of the ITRs in the ceDNA vector comprises a functional Rep binding site (RBS; e.g., 5′-GCGCGCTCGCTCGCTC-3′ for AAV2, SEQ ID NO: 60) and a functional terminal resolution site (TRS; e.g., 5′-AGTT-3′, SEQ ID NO: 62.) In one embodiment, at least one of the ITRs is a non-functional ITR. In one embodiment, the different or modified ITRs are not each wild type ITRs from different serotypes.

Specific alterations and mutations in the ITRs are described in detail herein, but in the context of ITRs, “altered” or “mutated” or “modified”, it indicates that nucleotides have been inserted, deleted, and/or substituted relative to the wild-type, reference, or original ITR sequence. The altered or mutated ITR can be an engineered ITR. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature.

In some embodiments, a mod-ITR may be synthetic. In one embodiment, a synthetic ITR is based on ITR sequences from more than one AAV serotype. In another embodiment, a synthetic ITR includes no AAV-based sequence. In yet another embodiment, a synthetic ITR preserves the ITR structure described above although having only some or no AAV-sourced sequence. In some aspects, a synthetic ITR may interact preferentially with a wild type Rep or a Rep of a specific serotype, or in some instances will not be recognized by a wild-type Rep and be recognized only by a mutated Rep.

The skilled artisan can determine the corresponding sequence in other serotypes by known means. For example, determining if the change is in the A, A′, B, B′, C, C′ or D region and determine the corresponding region in another serotype. One can use BLAST® (Basic Local Alignment Search Tool) or other homology alignment programs at default status to determine the corresponding sequence. The disclosure further provides populations and pluralities of ceDNA vectors comprising mod-ITRs from a combination of different AAV serotypes—that is, one mod-ITR can be from one AAV serotype and the other mod-ITR can be from a different serotype. Without wishing to be bound by theory, in one embodiment one ITR can be from or based on an AAV2 ITR sequence and the other ITR of the ceDNA vector can be from or be based on any one or more ITR sequence of AAV serotype 1 (AAV1), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), or AAV serotype 12 (AAV12).

Any parvovirus ITR can be used as an ITR or as a base ITR for modification. Preferably, the parvovirus is a dependovirus. More preferably AAV. The serotype chosen can be based upon the tissue tropism of the serotype. AAV2 has a broad tissue tropism, AAV1 preferentially targets to neuronal and skeletal muscle, and AAV5 preferentially targets neuronal, retinal pigmented epithelia, and photoreceptors. AAV6 preferentially targets skeletal muscle and lung. AAV8 preferentially targets liver, skeletal muscle, heart, and pancreatic tissues. AAV9 preferentially targets liver, skeletal and lung tissue. In one embodiment, the modified ITR is based on an AAV2 ITR.

More specifically, the ability of a structural element to functionally interact with a particular large Rep protein can be altered by modifying the structural element. For example, the nucleic acid sequence of the structural element can be modified as compared to the wild-type sequence of the ITR. In one embodiment, the structural element (e.g., A arm, A′ arm, B arm, B′ arm, C arm, C′ arm, D arm, RBE, RBE′, and trs) of an ITR can be removed and replaced with a wild-type structural element from a different parvovirus. For example, the replacement structure can be from AAV1, AAV2, AAV3, AAV4, AAV5, AAV6, AAV7, AAV8, AAV9, AAV10, AAV11, AAV12, AAV13, snake parvovirus (e.g., royal python parvovirus), bovine parvovirus, goat parvovirus, avian parvovirus, canine parvovirus, equine parvovirus, shrimp parvovirus, porcine parvovirus, or insect AAV. For example, the ITR can be an AAV2 ITR and the A or A′ arm or RBE can be replaced with a structural element from AAV5. In another example, the ITR can be an AAV5 ITR and the C or C′ arms, the RBE, and the trs can be replaced with a structural element from AAV2. In another example, the AAV ITR can be an AAV5 ITR with the B and B′ arms replaced with the AAV2 ITR B and B′ arms.

By way of example only, Table 4 indicates exemplary modifications of at least one nucleotide (e.g., a deletion, insertion and/or substitution) in regions of a modified ITR, where X is indicative of a modification of at least one nucleic acid (e.g., a deletion, insertion and/or substitution) in that section relative to the corresponding wild-type ITR. In some embodiments, any modification of at least one nucleotide (e.g., a deletion, insertion and/or substitution) in any of the regions of C and/or C′ and/or B and/or B′ retains three sequential T nucleotides (i.e., TTT) in at least one terminal loop. For example, if the modification results in any of: a single arm ITR (e.g., single C-C′ arm, or a single B-B′ arm), or a modified C-B′ arm or C′-B arm, or a two arm ITR with at least one truncated arm (e.g., a truncated C-C′ arm and/or truncated B-B′ arm), at least the single arm, or at least one of the arms of a two arm ITR (where one arm can be truncated) retains three sequential T nucleotides (i.e., TTT) in at least one terminal loop. In some embodiments, a truncated C-C′ arm and/or a truncated B-B′ arm has three sequential T nucleotides (i.e., TTT) in the terminal loop.

TABLE 4
Exemplary combinations of modifications of at least one nucleotide (e.g.,
a deletion, insertion and/or substitution) to different B-B' and C-C'
regions or arms of ITRs (X indicates a nucleotide modification, e.g.,
addition, deletion or substitution of at least one nucleotide in the region).

| B region | B' region | C region | C' region |
|---|---|---|---|
| X | | | |
| | X | | |
| X | X | | |
| | | X | |
| | | | X |
| | | X | X |
| X | | X | |
| X | | | X |
| | X | X | |
| | X | | X |
| X | X | X | |
| X | X | | X |
| X | | X | X |
| | X | X | X |
| X | X | X | X |

In some embodiments, mod-ITR for use in a ceDNA vector for expression of PAH protein comprises an asymmetric ITR pair, or a symmetric mod-ITR pair as disclosed herein, can comprise any one of the combinations of modifications shown in Table 4, and also a modification of at least one nucleotide in any one or more of the regions selected from: between A′ and C, between C and C′, between C′ and B, between B and B′ and between B′ and A. In some embodiments, any modification of at least one nucleotide (e.g., a deletion, insertion and/or substitution) in the C or C′ or B or B′ regions, still preserves the terminal loop of the stem-loop. In some embodiments, any modification of at least one nucleotide (e.g., a deletion, insertion and/or substitution) between C and C′ and/or B and B′ retains three sequential T nucleotides (i.e., TTT) in at least one terminal loop. In alternative embodiments, any modification of at least one nucleotide (e.g., a deletion, insertion and/or substitution) between C and C′ and/or B and B′ retains three sequential A nucleotides (i.e., AAA) in at least one terminal loop. In some embodiments, a modified ITR for use herein can comprise any one of the combinations of modifications shown in Table 4, and also a modification of at least one nucleotide (e.g., a deletion, insertion and/or substitution) in any one or more of the regions selected from: A′, A and/or D. For example, in some embodiments, a modified ITR for use herein can comprise any one of the combinations of modifications shown in Table 4, and also a modification of at least one nucleotide (e.g., a deletion, insertion and/or substitution) in the A region. In some embodiments, a modified ITR for use herein can comprise any one of the combinations of modifications shown in Table 4, and also a modification of at least one nucleotide (e.g., a deletion, insertion and/or substitution) in the A′ region. In some embodiments, a modified ITR for use herein can comprise any one of the combinations of modifications shown in Table 4, and also a modification of at least one nucleotide (e.g., a deletion, insertion and/or substitution) in the A and/or A′ region. In some embodiments, a modified ITR for use herein can comprise any one of the combinations of modifications shown in Table 4, and also a modification of at least one nucleotide (e.g., a deletion, insertion and/or substitution) in the D region.

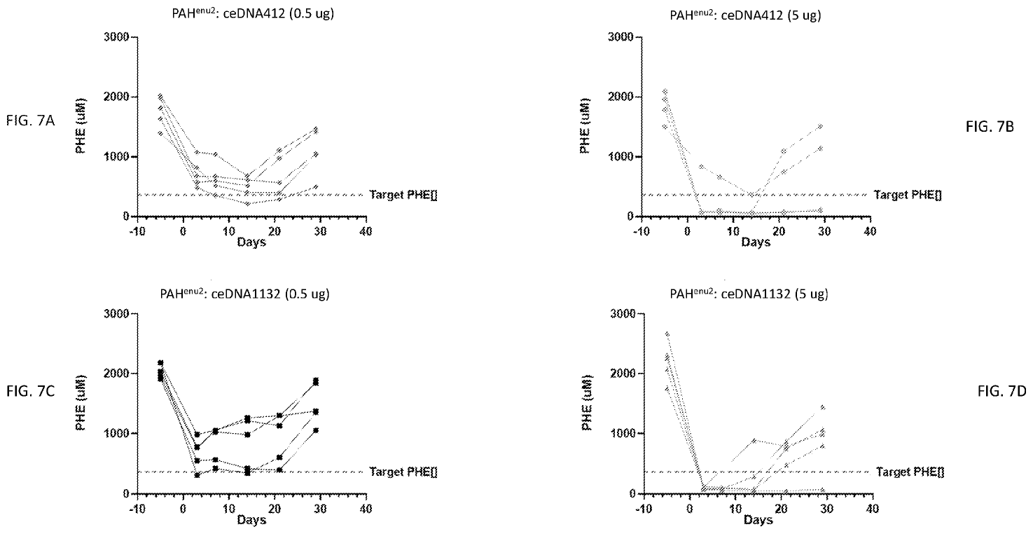

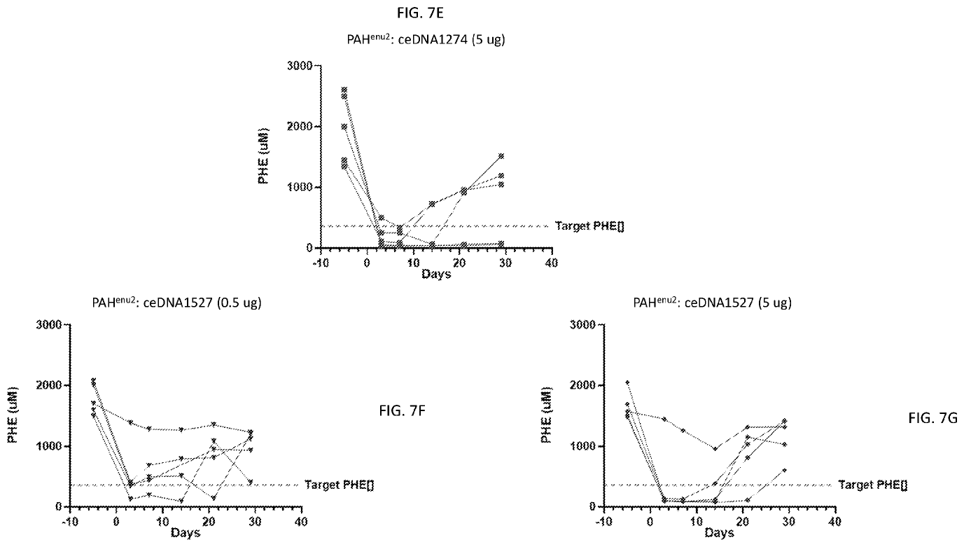

In one embodiment, the nucleotide sequence of the structural element can be modified (e.g., by modifying 1, 2, 3, 4, 5, 6, 7, 8, 9, 10, 11, 12, 13, 14, 15, 16, 17, 18, 19, or 20 or more nucleotides or any range therein) to produce a modified structural element. In one embodiment, the specific modifications to the ITRs are exemplified herein (e.g., SEQ ID NOS: 3, 4, 15-47, 101-116 or 165-187, or shown in FIG. 7A-7B of PCT/US2018/064242, filed on Dec. 6, 2018 (e.g., SEQ ID NOS: 97-98, 101-103, 105-108, 111-112, 117-134, 545-54 in PCT/US2018/064242). In some embodiments, an ITR can be modified (e.g., by modifying 1, 2, 3, 4, 5, 6, 7, 8, 9, 10, 11, 12, 13, 14, 15, 16, 17, 18, 19, or 20 or more nucleotides or any range therein). In other embodiments, the ITR can have at least 80%, at least 85%, at least 90%, at least 95%, at least 96%, at least 97%, at least 98%, at least 99%, or more sequence identity with one of the modified ITRs of SEQ ID NOS: 3, 4, 15-47, 101-116 or 165-187, or the RBE-containing section of the A-A′ arm and C-C′ and B-B′ arms of SEQ ID NO: 3, 4, 15-47, 101-116 or 165-187, or shown in Tables 2-9 (i.e., SEQ ID NO: 110-112, 115-190, 200-468) of International Patent Application No. PCT/US18/49996, which is incorporated herein in its entirety by reference.

In some embodiments, a modified ITR can for example, comprise removal or deletion of all of a particular arm, e.g., all or part of the A-A′ arm, or all or part of the B-B′ arm or all or part of the C-C′ arm, or alternatively, the removal of 1, 2, 3, 4, 5, 6, 7, 8, 9 or more base pairs forming the stem of the loop so long as the final loop capping the stem (e.g., single arm) is still present (e.g., see ITR-21 in FIG. 7A of International Patent Application No. PCT/US2018/064242, filed Dec. 6, 2018, incorporated by reference in its entirety herein). In some embodiments, a modified ITR can comprise the removal of 1, 2, 3, 4, 5, 6, 7, 8, 9 or more base pairs from the B-B′ arm. In some embodiments, a modified ITR can comprise the removal of 1, 2, 3, 4, 5, 6, 7, 8, 9 or more base pairs from the C-C′ arm (see, e.g., ITR-1 in FIG. 3B, or ITR-45 in FIG. 7A of International Patent Application No. PCT/US2018/064242, filed Dec. 6, 2018, incorporated by reference in its entirety herein). In some embodiments, a modified ITR can comprise the removal of 1, 2, 3, 4, 5, 6, 7, 8, 9 or more base pairs from the C-C′ arm and the removal of 1, 2, 3, 4, 5, 6, 7, 8, 9 or more base pairs from the B-B′ arm. Any combination of removal of base pairs is envisioned, for example, 6 base pairs can be removed in the C-C′ arm and 2 base pairs in the B-B′ arm. As an illustrative example, FIG. 3B shows an exemplary modified ITR with at least 7 base pairs deleted from each of the C portion and the C′ portion, a substitution of a nucleotide in the loop between C and C′ region, and at least one base pair deletion from each of the B region and B′ regions such that the modified ITR comprises two arms where at least one arm (e.g., C-C′) is truncated. In some embodiments, the modified ITR also comprises at least one base pair deletion from each of the B region and B′ regions, such that the B-B′ arm is also truncated relative to WT ITR.

In some embodiments, a modified ITR can have between 1 and 50 (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) nucleotide deletions relative to a full-length wild-type ITR sequence. In some embodiments, a modified ITR can have between 1 and 30 nucleotide deletions relative to a full-length WT ITR sequence. In some embodiments, a modified ITR has between 2 and 20 nucleotide deletions relative to a full-length wild-type ITR sequence.

In some embodiments, a modified ITR does not contain any nucleotide deletions in the RBE-containing portion of the A or A′ regions, so as not to interfere with DNA replication (e.g. binding to an RBE by Rep protein, or nicking at a terminal resolution site). In some embodiments, a modified ITR encompassed for use herein has one or more deletions in the B, B′, C, and/or C region as described herein.

In some embodiments, a ceDNA vector for expression of PAH protein comprising a symmetric ITR pair or asymmetric ITR pair comprises a regulatory switch as disclosed herein and at least one modified ITR selected having the nucleotide sequence selected from any of the group consisting of: SEQ ID NO: 3, 4, 15-47, 101-116 or 165-187.

In another embodiment, the structure of the structural element can be modified. For example, the structural element a change in the height of the stem and/or the number of nucleotides in the loop. For example, the height of the stem can be about 2, 3, 4, 5, 6, 7, 8, or 9 nucleotides or more or any range therein. In one embodiment, the stem height can be about 5 nucleotides to about 9 nucleotides and functionally interacts with Rep. In another embodiment, the stem height can be about 7 nucleotides and functionally interacts with Rep. In another example, the loop can have 3, 4, 5, 6, 7, 8, 9, or 10 nucleotides or more or any range therein.

In another embodiment, the number of GAGY binding sites or GAGY-related binding sites within the RBE or extended RBE can be increased or decreased. In one example, the RBE or extended RBE, can comprise 1, 2, 3, 4, 5, or 6 or more GAGY binding sites or any range therein. Each GAGY binding site can independently be an exact GAGY sequence or a sequence similar to GAGY as long as the sequence is sufficient to bind a Rep protein.

In another embodiment, the spacing between two elements (such as but not limited to the RBE and a hairpin) can be altered (e.g., increased or decreased) to alter functional interaction with a large Rep protein. For example, the spacing can be about 1, 2, 3, 4, 5, 6, 7, 8, 9, 10, 11, 12, 13, 14, 15, 16, 17, 18, 19, 20, or 21 nucleotides or more or any range therein.

The ceDNA vector for expression of PAH protein as described herein can include an ITR structure that is modified with respect to the wild type AAV2 ITR structure disclosed herein, but still retains an operable RBE, trs and RBE′ portion. In some embodiments, the ceDNA vector for expression of PAH protein contains one or more functional ITR polynucleotide sequences that comprise a Rep-binding site (RBS; 5′-GCGCGCTCGCTCGCTC-3′ (SEQ ID NO: 60) for AAV2) and a terminal resolution site (TRS; 5′-AGTT (SEQ ID NO: 62)). In some embodiments, at least one ITR (wt or modified ITR) is functional. In alternative embodiments, where a ceDNA vector for expression of PAH protein comprises two modified ITRs that are different or asymmetrical to each other, at least one modified ITR is functional and at least one modified ITR is non-functional.

In some embodiments, the modified ITR (e.g., the left or right ITR) of a ceDNA vector for expression of PAH protein as described herein has modifications within the loop arm, the truncated arm, or the spacer. Exemplary sequences of ITRs having modifications within the loop arm, the truncated arm, or the spacer are listed in Table 2 (i.e., SEQ ID NOs: 135-190, 200-233); Table 3 (e.g., SEQ ID NOs: 234-263); Table 4 (e.g., SEQ ID NOs: 264-293); Table 5 (e.g., SEQ ID NOs: 294-318 herein); Table 6 (e.g., SEQ ID NOs: 319-468; and Tables 7-9 (e.g., SEQ ID NOs: 101-110, 111-112, 115-134) or Table 10A or 10B (e.g., SEQ ID NOs: 9, 100, 469-483, 484-499) of International application PCT/US18/49996, which is incorporated herein in its entirety by reference.

In some embodiments, the modified ITR for use in a ceDNA vector for expression of PAH protein comprising an asymmetric ITR pair, or symmetric mod-ITR pair is selected from any or a combination of those shown in Tables 2, 3, 4, 5, 6, 7, 8, 9 and 10A-10B of International application PCT/US18/49996 which is incorporated herein in its entirety by reference.

Additional exemplary modified ITRs for use in a ceDNA vector for expression of PAH protein comprising an asymmetric ITR pair, or symmetric mod-ITR pair in each of the above classes are provided in Tables 5A and 5B. The predicted secondary structure of the Right modified ITRs in Table 5A are shown in FIG. 7A of International Application PCT/US2018/064242, filed Dec. 6, 2018, and the predicted secondary structure of the Left modified ITRs in Table 5B are shown in FIG. 7B of International Application PCT/US2018/064242, filed Dec. 6, 2018, which is incorporated herein in its entirety by reference.

Table 5A and Table 5B show exemplary right and left modified ITRs.

TABLE 5A
Exemplary modified right ITRs. These exemplary modified right ITRs can
comprise the RBE of GCGCGCTCGCTCGCTC-3′ (SEQ ID NO: 60),
spacer of ACTGAGGC (SEQ ID NO: 69), the spacer complement GCCTCAGT
(SEQ ID NO: 70) and RBE′ (i.e., complement to RBE) of
GAGCGAGCGAGCGCGC (SEQ ID NO: 71).
Exemplary Right modified ITRs

| ITR | | SEQ ID |
|---|---|---|
| Construct | Sequence | NO: |
| ITR-18 | AGGAACCCCTAGTGATGGAGTTGGCCACTCCCTCTCTGCGCGCTCG | 15 |
| Right | CTCGCTCACTGAGGCGCACGCCCGGGTTTCCCGGGCGGCCTCAGTG | |
| | AGCGAGCGAGCGCGCAGCTGCCTGCAGG | |
| ITR-19 | AGGAACCCCTAGTGATGGAGTTGGCCACTCCCTCTCTGCGCGCTCG | 16 |
| Right | CTCGCTCACTGAGGCCGACGCCCGGGCTTTGCCCGGGCGGCCTCA | |
| | GTGAGCGAGCGAGCGCGCAGCTGCCTGCAGG | |
| ITR-20 | AGGAACCCCTAGTGATGGAGTTGGCCACTCCCTCTCTGCGCGCTCG | 17 |
| Right | CTCGCTCACTGAGGCCGGGCGACCAAAGGTCGCCCGACGCCCGGG | |
| | CGCCTCAGTGAGCGAGCGAGCGCGCAGCTGCCTGCAGG | |
| ITR-21 | AGGAACCCCTAGTGATGGAGTTGGCCACTCCCTCTCTGCGCGCTCG | 18 |
| Right | CTCGCTCACTGAGGCTTTGCCTCAGTGAGCGAGCGAGCGCGCAGC | |
| | TGCCTGCAGG | |
| ITR-22 | AGGAACCCCTAGTGATGGAGTTGGCCACTCCCTCTCTGCGCGCTCG | 19 |
| Right | CTCGCTCACTGAGGCCGGGCGACAAAGTCGCCCGACGCCCGGGCT | |
| | TTGCCCGGGCGGCCTCAGTGAGCGAGCGAGCGCGCAGCTGCCTGC | |
| | AGG | |
| ITR-23 | AGGAACCCCTAGTGATGGAGTTGGCCACTCCCTCTCTGCGCGCTCG | 20 |
| Right | CTCGCTCACTGAGGCCGGGCGAAAATCGCCCGACGCCCGGGCTTT | |
| | GCCCGGGCGGCCTCAGTGAGCGAGCGAGCGCGCAGCTGCCTGCAG | |
| | G | |
| ITR-24 | AGGAACCCCTAGTGATGGAGTTGGCCACTCCCTCTCTGCGCGCTCG | 21 |
| Right | CTCGCTCACTGAGGCCGGGCGAAACGCCCGACGCCCGGGCTTTGC | |
| | CCGGGCGGCCTCAGTGAGCGAGCGAGCGCGCAGCTGCCTGCAGG | |
| ITR-25 | AGGAACCCCTAGTGATGGAGTTGGCCACTCCCTCTCTGCGCGCTCG | 22 |
| Right | CTCGCTCACTGAGGCCGGGCAAAGCCCGACGCCCGGGCTTTGCCC | |
| | GGGCGGCCTCAGTGAGCGAGCGAGCGCGCAGCTGCCTGCAGG | |
| ITR-26 | AGGAACCCCTAGTGATGGAGTTGGCCACTCCCTCTCTGCGCGCTCG | 23 |
| Right | CTCGCTCACTGAGGCCGGGCGACCAAAGGTCGCCCGACGCCCGGG | |
| | TTTCCCGGGCGGCCTCAGTGAGCGAGCGAGCGCGCAGCTGCCTGC | |
| | AGG | |
| ITR-27 | AGGAACCCCTAGTGATGGAGTTGGCCACTCCCTCTCTGCGCGCTCG | 24 |
| Right | CTCGCTCACTGAGGCCGGGCGACCAAAGGTCGCCCGACGCCCGGT | |
| | TTCCGGGCGGCCTCAGTGAGCGAGCGAGCGCGCAGCTGCCTGCAG | |
| | G | |
| ITR-28 | AGGAACCCCTAGTGATGGAGTTGGCCACTCCCTCTCTGCGCGCTCG | 25 |
| Right | CTCGCTCACTGAGGCCGGGCGACCAAAGGTCGCCCGACGCCCGTT | |
| | TCGGGCGGCCTCAGTGAGCGAGCGAGCGCGCAGCTGCCTGCAGG | |
| ITR-29 | AGGAACCCCTAGTGATGGAGTTGGCCACTCCCTCTCTGCGCGCTCG | 26 |
| Right | CTCGCTCACTGAGGCCGGGCGACCAAAGGTCGCCCGACGCCCTTT | |
| | GGGCGGCCTCAGTGAGCGAGCGAGCGCGCAGCTGCCTGCAGG | |
| ITR-30 | AGGAACCCCTAGTGATGGAGTTGGCCACTCCCTCTCTGCGCGCTCG | 27 |
| Right | CTCGCTCACTGAGGCCGGGCGACCAAAGGTCGCCCGACGCCTTTG | |
| | GCGGCCTCAGTGAGCGAGCGAGCGCGCAGCTGCCTGCAGG | |
| ITR-31 | AGGAACCCCTAGTGATGGAGTTGGCCACTCCCTCTCTGCGCGCTCG | 28 |
| Right | CTCGCTCACTGAGGCCGGGCGACCAAAGGTCGCCCGACGCTTTGC | |
| | GGCCTCAGTGAGCGAGCGAGCGCGCAGCTGCCTGCAGG | |
| ITR-32 | AGGAACCCCTAGTGATGGAGTTGGCCACTCCCTCTCTGCGCGCTCG | 29 |
| Right | CTCGCTCACTGAGGCCGGGCGACCAAAGGTCGCCCGACGTTTCGG | |
| | CCTCAGTGAGCGAGCGAGCGCGCAGCTGCCTGCAGG | |
| ITR-49 | AGGAACCCCTAGTGATGGAGTTGGCCACTCCCTCTCTGCGCGCTCG | 30 |
| Right | CTCGCTCACTGAGGCCGGGCGACCAAAGGTCGCCCGACGGCCTCA | |
| | GTGAGCGAGCGAGCGCGCAGCTGCCTGCAGG | |
| ITR-50 | AGGAACCCCTAGTGATGGAGTTGGCCACTCCCTCTCTGCGCGCTCG | 31 |
| right | CTCGCTCACTGAGGCCGGGCGACCAAAGGTCGCCCGACGCCCGGG | |
| | CGGCCTCAGTGAGCGAGCGAGCGCGCAGCTGCCTGCAGG | |

TABLE 5B
Exemplary modified left ITRs. These exemplary modified left ITRs
can comprise the RBE of GCGCGCTCGCTCGCTC-3′ (SEQ ID NO: 60),
spacer of ACTGAGGC (SEQ ID NO: 69), the spacer complement
GCCTCAGT (SEQ ID NO: 70) and RBE complement (RBE′) of
GAGCGAGCGAGCGCGC (SEQ ID NO: 71).
Exemplary modified left ITRs

| | | |
|---|---|---|
| ITR-33 | CCTGCAGGCAGCTGCGCGCTCGCTCGCTCACTGAGGCCGCCCGGG | 32 |
| Left | AAACCCGGGCGTGCGCCTCAGTGAGCGAGCGAGCGCGCAGAGAG | |
| | GGAGTGGCCAACTCCATCACTAGGGGTTCCT | |
| ITR-34 | CCTGCAGGCAGCTGCGCGCTCGCTCGCTCACTGAGGCCGTCGGGC | 33 |
| Left | GACCTTTGGTCGCCCGGCCTCAGTGAGCGAGCGAGCGCGCAGAGA | |
| | GGGAGTGGCCAACTCCATCACTAGGGGTTCCT | |
| ITR-35 | CCTGCAGGCAGCTGCGCGCTCGCTCGCTCACTGAGGCCGCCCGGG | 34 |
| Left | CAAAGCCCGGGCGTCGGCCTCAGTGAGCGAGCGAGCGCGCAGAG | |
| | AGGGAGTGGCCAACTCCATCACTAGGGGTTCCT | |
| ITR-36 | CCTGCAGGCAGCTGCGCGCTCGCTCGCTCACTGAGGCGCCCGGGC | 35 |
| Left | GTCGGGCGACCTTTGGTCGCCCGGCCTCAGTGAGCGAGCGAGCGC | |
| | GCAGAGAGGGAGTGGCCAACTCCATCACTAGGGGTTCCT | |
| ITR-37 | CCTGCAGGCAGCTGCGCGCTCGCTCGCTCACTGAGGCAAAGCCTC | 36 |
| Left | AGTGAGCGAGCGAGCGCGCAGAGAGGGAGTGGCCAACTCCATCA | |
| | CTAGGGGTTCCT | |
| ITR-38 | CCTGCAGGCAGCTGCGCGCTCGCTCGCTCACTGAGGCCGCCCGGG | 37 |
| Left | CAAAGCCCGGGCGTCGGGCGACTTTGTCGCCCGGCCTCAGTGAGC | |
| | GAGCGAGCGCGCAGAGAGGGAGTGGCCAACTCCATCACTAGGGGT | |
| | TCCT | |
| ITR-39 | CCTGCAGGCAGCTGCGCGCTCGCTCGCTCACTGAGGCCGCCCGGG | 38 |
| Left | CAAAGCCCGGGCGTCGGGCGATTTTCGCCCGGCCTCAGTGAGCGA | |
| | GCGAGCGCGCAGAGAGGGAGTGGCCAACTCCATCACTAGGGGTTC | |
| | CT | |
| ITR-40 | CCTGCAGGCAGCTGCGCGCTCGCTCGCTCACTGAGGCCGCCCGGG | 39 |
| Left | CAAAGCCCGGGCGTCGGGCGTTTCGCCCGGCCTCAGTGAGCGAGC | |
| | GAGCGCGCAGAGAGGGAGTGGCCAACTCCATCACTAGGGGTTCCT | |
| ITR-41 | CAAAGCCCGGGCGTCGGGCTTTGCCCGGCCTCAGTGAGCGAGCGA | 40 |
| Left | CCTGCAGGCAGCTGCGCGCTCGCTCGCTCACTGAGGCCGCCCGGG | |
| | CAAAGCCCGGGCGTCGGGCTTTGCCCGGCCTCAGTGAGCGAGCGA | |
| | GCGCGCAGAGAGGGAGTGGCCAACTCCATCACTAGGGGTTCCT | |
| ITR-42 | CCTGCAGGCAGCTGCGCGCTCGCTCGCTCACTGAGGCCGCCCGGG | 41 |
| Left | AAACCCGGGCGTCGGGCGACCTTTGGTCGCCCGGCCTCAGTGAGC | |
| | GAGCGAGCGCGCAGAGAGGGAGTGGCCAACTCCATCACTAGGGGT | |
| | TCCT | |
| ITR-43 | CCTGCAGGCAGCTGCGCGCTCGCTCGCTCACTGAGGCCGCCCGGA | 42 |
| Left | AACCGGGCGTCGGGCGACCTTTGGTCGCCCGGCCTCAGTGAGCGA | |
| | GCGAGCGCGCAGAGAGGGAGTGGCCAACTCCATCACTAGGGGTTC | |
| | CT | |
| ITR-44 | CCTGCAGGCAGCTGCGCGCTCGCTCGCTCACTGAGGCCGCCCGAA | 43 |
| Left | ACGGGCGTCGGGCGACCTTTGGTCGCCCGGCCTCAGTGAGCGAGC | |
| | GAGCGCGCAGAGAGGGAGTGGCCAACTCCATCACTAGGGGTTCCT | |
| ITR-45 | CCTGCAGGCAGCTGCGCGCTCGCTCGCTCACTGAGGCCGCCCAAA | 44 |
| Left | GGGCGTCGGGCGACCTTTGGTCGCCCGGCCTCAGTGAGCGAGCGA | |
| | GCGCGCAGAGAGGGAGTGGCCAACTCCATCACTAGGGGTTCCT | |
| ITR-46 | CCTGCAGGCAGCTGCGCGCTCGCTCGCTCACTGAGGCCGCCAAAG | 45 |
| Left | GCGTCGGGCGACCTTTGGTCGCCCGGCCTCAGTGAGCGAGCGAGC | |
| | GCGCAGAGAGGGAGTGGCCAACTCCATCACTAGGGGTTCCT | |
| ITR-47 | CCTGCAGGCAGCTGCGCGCTCGCTCGCTCACTGAGGCCGCAAAGC | 46 |
| Left | GTCGGGCGACCTTTGGTCGCCCGGCCTCAGTGAGCGAGCGAGCGC | |
| | GCAGAGAGGGAGTGGCCAACTCCATCACTAGGGGTTCCT | |
| ITR-48 | CCTGCAGGCAGCTGCGCGCTCGCTCGCTCACTGAGGCCGAAACGT | 47 |
| Left | CGGGCGACCTTTGGTCGCCCGGCCTCAGTGAGCGAGCGAGCGCGC | |
| | AGAGAGGGAGTGGCCAACTCCATCACTAGGGGTTCCT | |

In one embodiment, a ceDNA vector for expression of PAH protein comprises, in the 5′ to 3′ direction: a first adeno-associated virus (AAV) inverted terminal repeat (ITR), a nucleic acid sequence of interest (for example an expression cassette comprising a codon modified nucleic acid as described herein) and a second AAV ITR, where the first ITR (5′ ITR) and the second ITR (3′ ITR) are asymmetric with respect to each other—that is, they have a different 3D-spatial configuration from one another. As an exemplary embodiment, the first ITR can be a wild-type ITR and the second ITR can be a mutated or modified ITR, or vice versa, where the first ITR can be a mutated or modified ITR and the second ITR a wild-type ITR. In some embodiment, the first ITR and the second ITR are both mod-ITRs, but have different sequences, or have different modifications, and thus are not the same modified ITRs, and have different 3D spatial configurations. Stated differently, a ceDNA vector with asymmetric ITRs comprises ITRs where any changes in one ITR relative to the WT-ITR are not reflected in the other ITR; or alternatively, where the asymmetric ITRs have a modified asymmetric ITR pair can have a different sequence and different three-dimensional shape with respect to each other. Exemplary asymmetric ITRs in the ceDNA vector for expression of PAH protein and for use to generate a ceDNA-plasmid are shown in Table 5A and 5B.

In an alternative embodiment, a ceDNA vector for expression of PAH protein comprises two symmetrical mod-ITRs—that is, both ITRs have the same sequence, but are reverse complements (inverted) of each other. In some embodiments, a symmetrical mod-ITR pair comprises at least one or any combination of a deletion, insertion, or substitution relative to wild type ITR sequence from the same AAV serotype. The additions, deletions, or substitutions in the symmetrical ITR are the same but the reverse complement of each other. For example, an insertion of 3 nucleotides in the C region of the 5′ ITR would be reflected in the insertion of 3 reverse complement nucleotides in the corresponding section in the C′ region of the 3′ ITR. Solely for illustration purposes only, if the addition is AACG in the 5′ ITR, the addition is CGTT in the 3′ ITR at the corresponding site. For example, if the 5′ ITR sense strand is ATCGATCG (SEQ ID NO:608) with an addition of AACG between the G and A to result in the sequence ATCGAACGATCG (SEQ ID NO: 51). The corresponding 3′ ITR sense strand is CGATCGAT (SEQ ID NO:606) (the reverse complement of ATCGATCG (SEQ ID NO:607)) with an addition of CGTT (i.e., the reverse complement of AACG) between the T and C to result in the sequence CGATCGTTCGAT (SEQ ID NO: 49) (the reverse complement of ATCGAACGATCG) (SEQ ID NO: 51).

In alternative embodiments, the modified ITR pair are substantially symmetrical as defined herein—that is, the modified ITR pair can have a different sequence but have corresponding or the same symmetrical three-dimensional shape. For example, one modified ITR can be from one serotype and the other modified ITR be from a different serotype, but they have the same mutation (e.g., nucleotide insertion, deletion or substitution) in the same region. Stated differently, for illustrative purposes only, a 5′ mod-ITR can be from AAV2 and have a deletion in the C region, and the 3′ mod-ITR can be from AAV5 and have the corresponding deletion in the C′ region, and provided the 5′ mod-ITR and the 3′ mod-ITR have the same or symmetrical three-dimensional spatial organization, they are encompassed for use herein as a modified ITR pair.

In some embodiments, a substantially symmetrical mod-ITR pair has the same A, C-C′ and B-B′ loops in 3D space, e.g., if a modified ITR in a substantially symmetrical mod-ITR pair has a deletion of a C-C′ arm, then the cognate mod-ITR has the corresponding deletion of the C-C′ loop and also has a similar 3D structure of the remaining A and B-B′ loops in the same shape in geometric space of its cognate mod-ITR. By way of example only, substantially symmetrical ITRs can have a symmetrical spatial organization such that their structure is the same shape in geometrical space. This can occur, e.g., when a G-C pair is modified, for example, to a C-G pair or vice versa, or A-T pair is modified to a T-A pair, or vice versa. Therefore, using the exemplary example above of modified 5′ ITR as a ATCGAACGATCG (SEQ ID NO: 51), and modified 3′ ITR as CGATCGTTCGAT (SEQ ID NO: 49) (i.e., the reverse complement of ATCGAACGATCG (SEQ ID NO: 51)), these modified ITRs would still be symmetrical if, for example, the 5′ ITR had the sequence of ATCGAACCATCG (SEQ ID NO: 50), where G in the addition is modified to C, and the substantially symmetrical 3′ ITR has the sequence of CGATCGTTCGAT (SEQ ID NO: 49), without the corresponding modification of the T in the addition to a. In some embodiments, such a modified ITR pair are substantially symmetrical as the modified ITR pair has symmetrical stereochemistry.

Table 6 shows exemplary symmetric modified ITR pairs (i.e. a left modified ITRs and the symmetric right modified ITR) for use in a ceDNA vector for expression of PAH protein. The bold portion of the sequences identify partial ITR sequences (i.e., sequences of A-A′, C-C′ and B-B′ loops). These exemplary modified ITRs can comprise the RBE of GCGCGCTCGCTCGCTC-3′ (SEQ ID NO: 60), spacer of ACTGAGGC (SEQ ID NO: 69), the spacer complement GCCTCAGT (SEQ ID NO: 70) and RBE′ (i.e., complement to RBE) of GAGCGAGCGAGCGCGC (SEQ ID NO: 71).

TABLE 6
Exemplary symmetric modified ITR pairs in a ceDNA
vector for expression of PAH protein

| LEFT modified ITR | | Symmetric RIGHT modified ITR | |
|---|---|---|---|
| (modified 5′ ITR) | | (modified 3′ ITR) | |
| SEQ ID | CCTGCAGGCAGCTGCGCGCTC | SEQ ID NO: 15 | AGGAACCCCTAGTGATG |
| NO: 32 | GCTCGCTCACTGAGGCCGCC | (ITR-18, right) | GAGTTGGCCACTCCCTCT |
| (ITR-33 | CGGGAAACCCGGGCGTGCGC | | CTGCGCGCTCGCTCGC |
| left) | CTCAGTGAGCGAGCGAGCGC | | TCACTGAGGCGCACGC |
| | GCAGAGAGGGAGTGGCCAACT | | CCGGGTTTCCCGGGCG |
| | CCATCACTAGGGGTTCCT | | GCCTCAGTGAGCGAGC |
| | | | GAGCGCGCAGCTGCCT |
| | | | GCAGG |
| SEQ ID | CCTGCAGGCAGCTGCGCGCTC | SEQ ID NO: 48 | AGGAACCCCTAGTGATG |
| NO: 33 | GCTCGCTCACTGAGGCCGTC | (ITR-51, right) | GAGTTGGCCACTCCCTCT |
| (ITR-34 | GGGCGACCTTTGGTCGCCCG | | CTGCGCGCTCGCTCGC |
| left) | GCCTCAGTGAGCGAGCGAGC | | TCACTGAGGCCGGGCG |
| | GCGCAGAGAGGGAGTGGCCA | | ACCAAAGGTCGCCCGA |
| | ACTCCATCACTAGGGGTTCCT | | CGGCCTCAGTGAGCGA |
| | | | GCGAGCGCGCAGCTGC |
| | | | CTGCAGG |
| SEQ ID | CCTGCAGGCAGCTGCGCGCTC | SEQ ID NO: 16 | AGGAACCCCTAGTGATG |
| NO: 34 | GCTCGCTCACTGAGGCCGCC | (ITR-19, right) | GAGTTGGCCACTCCCTCT |
| (ITR-35 | CGGGCAAAGCCCGGGCGTCG | | CTGCGCGCTCGCTCGC |
| left) | GCCTCAGTGAGCGAGCGAGC | | TCACTGAGGCCGACGC |
| | GCGCAGAGAGGGAGTGGCCA | | CCGGGCTTTGCCCGGG |
| | ACTCCATCACTAGGGGTTCCT | | CGGCCTCAGTGAGCGA |
| | | | GCGAGCGCGCAGCTGC |
| | | | CTGCAGG |
| SEQ ID | CCTGCAGGCAGCTGCGCGCTC | SEQ ID NO: 17 | AGGAACCCCTAGTGATG |
| NO: 35 | GCTCGCTCACTGAGGCGCCC | (ITR-20, right) | GAGTTGGCCACTCCCTCT |
| (ITR-36 | GGGCGTCGGGCGACCTTTGG | | CTGCGCGCTCGCTCGC |
| left) | TCGCCCGGCCTCAGTGAGCG | | TCACTGAGGCCGGGCG |
| | AGCGAGCGCGCAGAGAGGGA | | ACCAAAGGTCGCCCGA |
| | GTGGCCAACTCCATCACTAGG | | CGCCCGGGCGCCTCAG |
| | GGTTCCT | | TGAGCGAGCGAGCGCG |
| | | | CAGCTGCCTGCAGG |
| SEQ ID | CCTGCAGGCAGCTGCGCGCTC | SEQ ID NO: 18 | AGGAACCCCTAGTGATG |
| NO: 36 | GCTCGCTCACTGAGGCAAAG | (ITR-21, right) | GAGTTGGCCACTCCCTCT |
| (ITR-37 | CCTCAGTGAGCGAGCGAGCG | | CTGCGCGCTCGCTCGC |
| left) | CGCAGAGAGGGAGTGGCCAAC | | TCACTGAGGCTTTGCC |
| | TCCATCACTAGGGGTTCCT | | TCAGTGAGCGAGCGAG |
| | | | CGCGCAGCTGCCTGCAG |
| | | | G |
| SEQ ID | CCTGCAGGCAGCTGCGCGCTC | SEQ ID NO: 19 | AGGAACCCCTAGTGATG |
| NO: 37 | GCTCGCTCACTGAGGCCGCC | (ITR-22 right) | GAGTTGGCCACTCCCTCT |
| (ITR-38 | CGGGCAAAGCCCGGGCGTCG | | CTGCGCGCTCGCTCGC |
| left) | GGCGACTTTGTCGCCCGGCC | | TCACTGAGGCCGGGCG |
| | TCAGTGAGCGAGCGAGCGCG | | ACAAAGTCGCCCGACG |
| | CAGAGAGGGAGTGGCCAACTC | | CCCGGGCTTTGCCCGG |
| | CATCACTAGGGGTTCCT | | GCGGCCTCAGTGAGCG |
| | | | AGCGAGCGCGCAGCTG |
| | | | CCTGCAGG |
| SEQ ID | CCTGCAGGCAGCTGCGCGCTC | SEQ ID NO: 20 | AGGAACCCCTAGTGATG |
| NO: 38 | GCTCGCTCACTGAGGCCGCC | (ITR-23, right) | GAGTTGGCCACTCCCTCT |
| (ITR-39 | CGGGCAAAGCCCGGGCGTCG | | CTGCGCGCTCGCTCGC |
| left) | GGCGATTTTCGCCCGGCCTC | | TCACTGAGGCCGGGCG |
| | AGTGAGCGAGCGAGCGCGCA | | AAAATCGCCCGACGCC |
| | GAGAGGGAGTGGCCAACTCCA | | CGGGCTTTGCCCGGGC |
| | TCACTAGGGGTTCCT | | GGCCTCAGTGAGCGAG |
| | | | CGAGCGCGCAGCTGCC |
| | | | TGCAGG |
| SEQ ID | CCTGCAGGCAGCTGCGCGCTC | SEQ ID NO: 21 | AGGAACCCCTAGTGATG |
| NO: 39 | GCTCGCTCACTGAGGCCGCC | (ITR-24, right) | GAGTTGGCCACTCCCTCT |
| (ITR-40 | CGGGCAAAGCCCGGGCGTCG | | CTGCGCGCTCGCTCGC |
| left | GGCGTTTCGCCCGGCCTCAG | | TCACTGAGGCCGGGCG |
| | TGAGCGAGCGAGCGCGCAGA | | AAACGCCCGACGCCCG |
| | GAGGGAGTGGCCAACTCCATC | | GGCTTTGCCCGGGCGG |
| | ACTAGGGGTTCCT | | CCTCAGTGAGCGAGCG |
| | | | AGCGCGCAGCTGCCTGC |
| | | | AGG |
| SEQ ID | CCTGCAGGCAGCTGCGCGCTC | SEQ ID NO: 22 | AGGAACCCCTAGTGATG |
| NO: 40 | GCTCGCTCACTGAGGCCGCC | (ITR-25 right) | GAGTTGGCCACTCCCTCT |
| (ITR-41 | CGGGCAAAGCCCGGGCGTCG | | CTGCGCGCTCGCTCGC |
| left) | GGCTTTGCCCGGCCTCAGTG | | TCACTGAGGCCGGGCA |
| | AGCGAGCGAGCGCGCAGAGA | | AAGCCCGACGCCCGGG |
| | GGGAGTGGCCAACTCCATCAC | | CTTTGCCCGGGCGGCC |
| | TAGGGGTTCCT | | TCAGTGAGCGAGCGAG |
| | | | CGCGCAGCTGCCTGCAG |
| | | | G |
| SEQ ID | CCTGCAGGCAGCTGCGCGCTC | SEQ ID NO: 23 | AGGAACCCCTAGTGATG |
| NO: 41 | GCTCGCTCACTGAGGCCGCC | (ITR-26 right) | GAGTTGGCCACTCCCTCT |
| (ITR-42 | CGGGAAACCCGGGCGTCGGG | | CTGCGCGCTCGCTCGC |
| left) | CGACCTTTGGTCGCCCGGCC | | TCACTGAGGCCGGGCG |
| | TCAGTGAGCGAGCGAGCGCG | | ACCAAAGGTCGCCCGA |
| | CAGAGAGGGAGTGGCCAACTC | | CGCCCGGGTTTCCCGG |
| | CATCACTAGGGGTTCCT | | GCGGCCTCAGTGAGCG |
| | | | AGCGAGCGCGCAGCTG |
| | | | CCTGCAGG |
| SEQ ID | CCTGCAGGCAGCTGCGCGCTC | SEQ ID NO: 24 | AGGAACCCCTAGTGATG |
| NO: | GCTCGCTCACTGAGGCCGCC | (ITR-27 right) | GAGTTGGCCACTCCCTCT |
| 42(ITR-43 | CGGAAACCGGGCGTCGGGCG | | CTGCGCGCTCGCTCGC |
| left) | ACCTTTGGTCGCCCGGCCTC | | TCACTGAGGCCGGGCG |
| | AGTGAGCGAGCGAGCGCGCA | | ACCAAAGGTCGCCCGA |
| | GAGAGGGAGTGGCCAACTCCA | | CGCCCGGTTTCCGGGC |
| | TCACTAGGGGTTCCT | | GGCCTCAGTGAGCGAG |
| | | | CGAGCGCGCAGCTGCC |
| | | | TGCAGG |
| SEQ ID | CCTGCAGGCAGCTGCGCGCTC | SEQ ID NO: 25 | AGGAACCCCTAGTGATG |
| NO: 43 | GCTCGCTCACTGAGGCCGCC | (ITR-28 right) | GAGTTGGCCACTCCCTCT |
| (ITR-44 | CGAAACGGGCGTCGGGCGAC | | CTGCGCGCTCGCTCGC |
| left) | CTTTGGTCGCCCGGCCTCAG | | TCACTGAGGCCGGGCG |
| | TGAGCGAGCGAGCGCGCAGA | | ACCAAAGGTCGCCCGA |
| | GAGGGAGTGGCCAACTCCATC | | CGCCCGTTTCGGGCGG |
| | ACTAGGGGTTCCT | | CCTCAGTGAGCGAGCG |
| | | | AGCGCGCAGCTGCCTGC |
| | | | AGG |
| SEQ ID | CCTGCAGGCAGCTGCGCGCTC | SEQ ID NO: 26 | AGGAACCCCTAGTGATG |
| NO: 44 | GCTCGCTCACTGAGGCCGCC | (ITR-29, right) | GAGTTGGCCACTCCCTCT |
| (ITR-45 | CAAAGGGCGTCGGGCGACCT | | CTGCGCGCTCGCTCGC |
| left) | TTGGTCGCCCGGCCTCAGTG | | TCACTGAGGCCGGGCG |
| | AGCGAGCGAGCGCGCAGAGA | | ACCAAAGGTCGCCCGA |
| | GGGAGTGGCCAACTCCATCAC | | CGCCCTTTGGGCGGCC |
| | TAGGGGTTCCT | | TCAGTGAGCGAGCGAG |
| | | | CGCGCAGCTGCCTGCAG |
| | | | G |
| SEQ ID | CCTGCAGGCAGCTGCGCGCTC | SEQ ID NO: | AGGAACCCCTAGTGATG |
| NO: 45 | GCTCGCTCACTGAGGCCGCC | 27(ITR-30, right) | GAGTTGGCCACTCCCTCT |
| (ITR-46 | AAAGGCGTCGGGCGACCTTT | | CTGCGCGCTCGCTCGC |
| left) | GGTCGCCCGGCCTCAGTGAG | | TCACTGAGGCCGGGCG |
| | CGAGCGAGCGCGCAGAGAGG | | ACCAAAGGTCGCCCGA |
| | GAGTGGCCAACTCCATCACTA | | CGCCTTTGGCGGCCTC |
| | GGGGTTCCT | | AGTGAGCGAGCGAGCG |
| | | | CGCAGCTGCCTGCAGG |
| SEQ ID | CCTGCAGGCAGCTGCGCGCTC | SEQ ID NO: 28 | AGGAACCCCTAGTGATG |
| NO: 46 | GCTCGCTCACTGAGGCCGCA | (ITR-31, right) | GAGTTGGCCACTCCCTCT |
| (ITR-47, | AAGCGTCGGGCGACCTTTGG | | CTGCGCGCTCGCTCGC |
| left) | TCGCCCGGCCTCAGTGAGCG | | TCACTGAGGCCGGGCG |
| | AGCGAGCGCGCAGAGAGGGA | | ACCAAAGGTCGCCCGA |
| | GTGGCCAACTCCATCACTAGG | | CGCTTTGCGGCCTCAG |
| | GGTTCCT | | TGAGCGAGCGAGCGCG |
| | | | CAGCTGCCTGCAGG |
| SEQ ID | CCTGCAGGCAGCTGCGCGCTC | SEQ ID NO: 29 | AGGAACCCCTAGTGATG |
| NO: 47 | GCTCGCTCACTGAGGCCGAA | (ITR-32 right) | GAGTTGGCCACTCCCTCT |
| (ITR-48, | ACGTCGGGCGACCTTTGGTC | | CTGCGCGCTCGCTCGC |
| left) | GCCCGGCCTCAGTGAGCGAG | | TCACTGAGGCCGGGCG |
| | CGAGCGCGCAGAGAGGGAGT | | ACCAAAGGTCGCCCGA |
| | GGCCAACTCCATCACTAGGGG | | CGTTTCGGCCTCAGTG |
| | TTCCT | | AGCGAGCGAGCGCGCA |
| | | | GCTGCCTGCAGG |

In some embodiments, a ceDNA vector for expression of PAH protein comprising an asymmetric ITR pair can comprise an ITR with a modification corresponding to any of the modifications in ITR sequences or ITR partial sequences shown in any one or more of Tables 5A-5B herein, or the sequences shown in FIG. 7A-7B3 of International Application PCT/US2018/064242, filed Dec. 6, 2018, which is incorporated herein in its entirety, or disclosed in Tables 2, 3, 4, 5, 6, 7, 8, 9 or 10A-10B3 of International application PCT/US18/49996 filed Sep. 7, 2018 which is incorporated herein in its entirety by reference.

### V. Exemplary CeDNA Vectors

As described above, the present disclosure relates to recombinant ceDNA expression vectors and ceDNA vectors comprising codon modified nucleic acids that encode PAH protein, comprising any one of: an asymmetrical ITR pair, a symmetrical ITR pair, or substantially symmetrical ITR pair as described above. In certain embodiments, the disclosure relates to recombinant ceDNA vectors for expression of PAH protein having flanking ITR sequences and a transgene, where the ITR sequences are asymmetrical, symmetrical or substantially symmetrical relative to each other as defined herein, and the ceDNA further comprises a nucleic acid sequence of interest (for example an expression cassette comprising the nucleic acid of a transgene) located between the flanking ITRs, wherein said nucleic acid molecule is devoid of viral capsid protein coding sequences.

The ceDNA expression vector for expression of PAH protein may be any ceDNA vector that can be conveniently subjected to recombinant DNA procedures including nucleic acid sequence(s) as described herein, provided at least one ITR is altered. The ceDNA vectors for expression of PAH protein of the present disclosure are compatible with the host cell into which the ceDNA vector is to be introduced. In certain embodiments, the ceDNA vectors may be linear. In certain embodiments, the ceDNA vectors may exist as an extrachromosomal entity. In certain embodiments, the ceDNA vectors of the present disclosure may contain an element(s) that permits integration of a donor sequence into the host cell's genome. As used herein “transgene” and “heterologous nucleic acid sequence” are synonymous, and may encode a PAH protein, as described herein.

### A. Regulatory Elements.

The ceDNA vectors for expression of PAH protein as described herein comprising an asymmetric ITR pair or symmetric ITR pair as defined herein, can further comprise a specific combination of cis-regulatory elements.

Described herein are ceDNA vectors that comprise a PAH nucleic acid sequence that has been codon optimized and combined with particular cis-acting elements (e.g., specific promoters, specific enhancers and specific promoter and enhancer combinations), that have been tested for optimal correction of phenylalanine level (e.g., expression and duration). According to some embodiments, particular codon optimized PAH nucleic acid sequences perform better when combined with a specific promoter sequence and/or a specific enhancer sequence, compared to the same codon optimized PAH nucleic acid sequence combined with, e.g., another promoter sequence and/or a specific enhancer sequence.

In certain embodiments, an enhancer sequence is provided upstream of the promoter to increase the efficacy of the promoter.

In certain embodiments, an intron sequence is provided upstream of the codon optimized nucleic acid sequence.

(i) Promoters:

It will be appreciated by one of ordinary skill in the art that promoters used in the ceDNA vectors for expression of PAH protein as disclosed herein should be tailored as appropriate for the specific sequences and types of tissue or cell in which they are promoting.

Expression cassettes of the ceDNA vector for expression of PAH protein can include a promoter, which can influence overall expression levels as well as cell-specificity. For transgene expression, e.g., expression of PAH protein, they can include a highly active virus-derived immediate early promoter. Expression cassettes can contain tissue-specific eukaryotic promoters to limit transgene expression to specific cell types and reduce toxic effects and immune responses resulting from unregulated, ectopic expression. Tables 7A and 7B list core promoter sequences that can be implemented in ceDNA PAH therapeutic.

TABLE 7A
Core Promoters

| | Name | SEQ |
|---|---|---|
| GE- | hAAT_core | GATCTTGCTACCAGTGGAACAGCCACTAAGGATTCTGCAGTGA |
| 015 | (SEQ ID NO: 441) | GAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAG |
| | | CTGTACACTGCCCAGGCAAAGCGTCCGGGCAGCGTAGGCGGGC |
| | | GACTCAGATCCCAGCCAGTGGACTTAGCCCCTGTTTGCTCCTCC |
| | | GATAACTGGGGTGACCTTGGTTAATATTCACCAGCAGCCTCCCC |
| | | CGTTGCCCCTCTGGATCCACTGCTTAAATACGGACGAGGACAG |
| | | G |
| GE- | TTRm | GTCTGTCTGCACATTTCGTAGAGCGAGTGTTCCGATACTCTAAT |
| 1121 | (SEQ ID NO: 442) | CTCCCTAGGCAAGGTTCATATTTGTGTAGGTTACTTATTCTCCTT |
| | | TTGTTGACTAAGTCAATAATCAGAATCAGCAGGTTTGGAGTCA |
| | | GCTTGGCAGGGATCAGCAGCCTGGGTTGGAAGGAGGGGGTATA |
| | | AAAGCCCCTTCACCAGGAGAAGCCGTC |
| GE- | hAAT_core_C06 | GATCTTGCTACCAGTGGAACAGCCACTAAGGATTCTGCAGTGA |
| 1133 | (SEQ ID NO: 443) | GAGCAGAGGGCCAGCTAAGTGGTACTCTCCCAGAGACTGTCTG |
| | | ACTCATGCCACCCCCTCCACCTTGGACACAGGACACTGTGGTTT |
| | | CTGAGCCAGGTACAATGACTCCTTTTGGTAAGTGCAGTGGAAG |
| | | CTGTACACTGCCCAGGCAAAGTGTCCGGGCAGCGTAGGCGGGC |
| | | GACTCAGATCCCAGCCAGTGGACTTAGCCCCTGTTTGCTCCTCC |
| | | GATAACTGGGGTGACCTTGGTTAATATTCACCAGCAGCCTCCCC |
| | | CGTTGCCCCTCTGGATCCACTGCTTAAATACGGACGAGGACAG |
| | | G |
| GE- | hAAT_core_C07 | GATCTTGCTACCAGTGGAACAGCCACTAAGGATTCTGCAGTGA |
| 1134 | (SEQ ID NO: 444) | GAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAG |
| | | CTGTACACTGCCCAGGCAAAGCGTCCGGGCAGCGTAGGCGGGC |
| | | GACTCAGATCCCAGCCAGTGGACTTAGCCCCTGTTTGCTCCTCT |
| | | GATAACTGGGGTGACCTTGGTTAATATTCACCAGCAGCCTCCCC |
| | | TGTTGCCCCTCTGGATCCACTGCTTAAATACGGACAAGGACAG |
| | | G |
| GE- | hAAT_core_C08 | GATCTTGCTACCAGTGGAACAGCCACTAAGGATTCTGCAGTGA |
| 1135 | (SEQ ID NO: 445) | GAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAG |
| | | CTGTACACTGCCCAGGCAAAGCGTCTGGGCAGCATAGGCAGGC |
| | | GACTCAGATCCCAGCCAGTGGACTTAGCCCCTGTTTGCTCCTCC |
| | | GATAACTGGGGTGACCTTGGTTAATATTCACCAGCAGCCTCCCC |
| | | CGTTGCCCCTCTGGATCCACTGCTTAAATACGGACGAGGACAG |
| | | G |
| GE- | hAAT_core_C09 | GATCTTGCTACCAGTGGAACAGCCACTAAGGATTCTGCAGTGA |
| 1136 | (SEQ ID NO: 446) | GAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAG |
| | | CTGTACACTGCCCAGGCAAAGCGTCCGGGCAGCGTAGGCGGGC |
| | | GACTCAGATCCCAGCCAGTGGACTTAGCCCCTGTTTGCTCCTCT |
| | | GATAACTGGGGTGACCTTGGTTAATATTCACCAGCAGCCTCCCC |
| | | CGTTGCCCCTCTGGATCCACTGCTTAAATACAGACGAGGACAG |
| | | G |
| GE- | hAAT_core_C10 | GATCTTGCTACCAGTGGAACAGCCACTAAGGATTCTGCAGTGA |
| 1137 | (SEQ ID NO: 447) | GAGCAGAGGGCCAGCTAAGTGGTACTCTCCCAGAGACTGTCTG |
| | | ACTCATGCCACCCCCTCCACCTTGGACACAGGACACTGTGGTTT |
| | | CTGAGCCAGGTACAATGACTCCTTTTGGTAAGTGCAGTGGAAG |
| | | CTGTACACTGCCCAGGCAAAGTGTCTGGGCAGCATAGGCAGGT |
| | | GACTCAGATCCCAGCCAGTGGACTTAGCCCCTGTTTGCTCCTCT |
| | | GATAACTGGGGTGACCTTGGTTAATATTCACCAGCAGCCTCCCC |
| | | TGTTGCCCCTCTGGATCCACTGCTTAAATACAGACAAGGACAG |
| | | G |
| GE- | hAAT_core_ | GATTCTGCAGTGAGAGCAGAGGGCCAGCTAAGTGGTACTCTCC |
| 1170 | truncated | CAGAGACTGTCTGACTCACGCCACCCCCTCCACCTTGGACACA |
| | (SEQ ID NO: 448) | GGACGCTGTGGTTTCTGAGCCAGGTACAATGACTCCTTTCGGTA |
| | | AGTGCAGTGGAAGCTGTACACTGCCCAGGCAAAGCGTCCGGGC |
| | | AGCGTAGGCGGGCGACTCAGATCCCAGCCAGTGGACTTAGCCC |
| | | CTGTTTGCTCCTCCGATAACTGGGGTGACCTTGGTTAATATTCA |
| | | CCAGCAGCCTCCCCCGTTGCCCCTCTGGATCCACTGCTTAAATA |
| | | CGGACGAGGACAGG |

TABLE 7B
Core Promoter Description

| SEQ ID | | |
|---|---|---|
| NO/GE # | Name | Description |
| GE-015 | hAAT_core | Core promoter sequence from human A1AT gene |
| GE-1121 | TTRm | Core promoter sequence from mouse Transthyretin gene |
| GE-1133 | hAAT_core_C06 | CpG minimized version of the hAAT core promoter |
| | | (A1AT gene promoter) |
| GE-1134 | hAAT_core_C07 | CpG minimized version of the hAAT core promoter |
| | | (A1AT gene promoter) |
| GE-1135 | hAAT_core_C08 | CpG minimized version of the hAAT core promoter |
| | | (A1AT gene promoter) |
| GE-1136 | hAAT_core_C09 | CpG minimized version of the hAAT core promoter |
| | | (A1AT gene promoter) |
| GE-1137 | hAAT_core_C10 | CpG minimized version of the hAAT core promoter |
| | | (A1AT gene promoter) |
| GE-1170 | hAAT_core_truncated | 5p truncated hAAT core promoter derived from GE-015 |

According to particular embodiments, the promoter is selected from the group consisting of: the VD (also referred to as “VanD”) promoter, human alpha 1-antitrypsin (hAAT) promoter (including the CpG minimized hAAT(979) promoter (CpGmin hAAT_core_C10) and other CpGmin_hAAT promoters like hAAT_core_C06; hAAT_core_C07; hAAT_core_C08; and hAAT_core_C09) and the transthyretin (TTR) liver specific promoter.

In some embodiments, the VD promoter comprises the minute virus mouse (MVM) intron, the minimal transthyretin promoter (TTRm), the serpin enhancer (72 bp) and TTRm 5′ UTR.

According to some embodiments, the TTRm comprises SEQ ID NO:442, shown below:

(SEQ ID NO: 442)
GTCTGTCTGCACATTTCGTAGAGCGAGTGTTCC
GATACTCTAATCTCCCTAGGCAAGGTTCATATT
TGTGTAGGTTACTTATTCTCCTTTTGTTGACTA
AGTCAATAATCAGAATCAGCAGGTTTGGAGTCA
GCTTGGCAGGGATCAGCAGCCTGGGTTGGAAGG
AGGGGGTATAAAAGCCCCTTCACCAGGAGAAGC
CGTC.

According to some embodiments, the serpin enhancer comprises SEQ ID NO:449, shown below:

(SEQ ID NO: 449)
GGGGGAGGCTGCTGGTGAATATTAACCAAGGTCACC
CCAGTTATCGGAGGAGCAAACAGGGGCTAAGTCCAC.

According to some embodiments, the TTRm 5′UTR comprises SEQ ID NO:498, shown below:

(SEQ ID NO: 498)
ACACAGATCCACAAGCTCCTG.

According to further embodiments, the VD promoter comprises SEQ ID NO:191 shown below:

(SEQ ID NO: 191)
CCGTCTGTCTGCACATTTCGTAGAGCGAGTGTTC
CGATACTCTAATCTCCCTAGGCAAGGTTCATATT
TGTGTAGGTTACTTATTCTCCTTTTGTTGACTAA
GTCAATAATCAGAATCAGCAGGTTTGGAGTCAGC
TTGGCAGGGATCAGCAGCCTGGGTTGGAAGGAGG
GGGTATAAAAGCCCCTTCACCAGGAGAAGCCGTC
ACACAGATCCACAAGCTCCTG

According to some embodiments, the hAAT(979) promoter comprises SEQ ID NO: 447. An hAAT (979) containing promoter set is exemplified in SEQ ID NO:479.

According to some embodiments, the CpGmin_hAAT promoter comprises a sequence selected from SEQ ID NOs:443-447. According to some embodiments, the CpGmin_hAAT promoter set is SEQ ID NO:475. According to some embodiments, the CpGmin_hAAT promoter set is SEQ ID NO:479. According to some embodiments, the transthyretin (TTR) liver specific promoter comprises a sequence set forth in Table 7A (SEQ ID NO:442).

According to some embodiments, the promoter comprises a nucleic acid sequence having at least 95% identity to SEQ ID NO: 191.

According to some embodiments, wherein the promoter comprises a nucleic acid sequence having at least 95% identity to SEQ ID NO:443. According to some embodiments, wherein the promoter comprises a nucleic acid sequence having at least 95% identity to SEQ ID NO:444. According to some embodiments, wherein the promoter comprises a nucleic acid sequence having at least 95% identity to SEQ ID NO:445. According to some embodiments, wherein the promoter comprises a nucleic acid sequence having at least 95% identity to SEQ ID NO:446. According to some embodiments, wherein the promoter comprises a nucleic acid sequence having at least 95% identity to SEQ ID NO:447.

According to some embodiments, wherein the promoter comprises a nucleic acid sequence having at least 96% identity to SEQ ID NO:443. According to some embodiments, wherein the promoter comprises a nucleic acid sequence having at least 96% identity to SEQ ID NO:444. According to some embodiments, wherein the promoter comprises a nucleic acid sequence having at least 96% identity to SEQ ID NO:445. According to some embodiments, wherein the promoter comprises a nucleic acid sequence having at least 96% identity to SEQ ID NO:446. According to some embodiments, wherein the promoter comprises a nucleic acid sequence having at least 96% identity to SEQ ID NO:447.

According to some embodiments, wherein the promoter comprises a nucleic acid sequence having at least 97% identity to SEQ ID NO:443. According to some embodiments, wherein the promoter comprises a nucleic acid sequence having at least 97% identity to SEQ ID NO:444. According to some embodiments, wherein the promoter comprises a nucleic acid sequence having at least 97% identity to SEQ ID NO:445. According to some embodiments, wherein the promoter comprises a nucleic acid sequence having at least 97% identity to SEQ ID NO:446. According to some embodiments, wherein the promoter comprises a nucleic acid sequence having at least 97% identity to SEQ ID NO:447.

According to some embodiments, wherein the promoter comprises a nucleic acid sequence having at least 98% identity to SEQ ID NO:443. According to some embodiments, wherein the promoter comprises a nucleic acid sequence having at least 98% identity to SEQ ID NO:444. According to some embodiments, wherein the promoter comprises a nucleic acid sequence having at least 98% identity to SEQ ID NO:445. According to some embodiments, wherein the promoter comprises a nucleic acid sequence having at least 98% identity to SEQ ID NO:446. According to some embodiments, wherein the promoter comprises a nucleic acid sequence having at least 98% identity to SEQ ID NO:447.

According to some embodiments, wherein the promoter comprises a nucleic acid sequence having at least 99% identity to SEQ ID NO:443. According to some embodiments, wherein the promoter comprises a nucleic acid sequence having at least 99% identity to SEQ ID NO:444. According to some embodiments, wherein the promoter comprises a nucleic acid sequence having at least 99% identity to SEQ ID NO:445. According to some embodiments, wherein the promoter comprises a nucleic acid sequence having at least 99% identity to SEQ ID NO:446. According to some embodiments, wherein the promoter comprises a nucleic acid sequence having at least 99% identity to SEQ ID NO:447.

In some embodiments, a promoter set comprising a promoter sequence and an enhancer sequence described herein. According to some embodiments, wherein the promoter set comprises a nucleic acid sequence having at least 95% identity to SEQ ID NO:475. According to some embodiments, wherein the promoter comprises a nucleic acid sequence having at least 96% identity to SEQ ID NO:475. According to some embodiments, wherein the promoter comprises a nucleic acid sequence having at least 97% identity to SEQ ID NO:475. According to some embodiments, wherein the promoter comprises a nucleic acid sequence having at least 98% identity to SEQ ID NO:475. According to some embodiments, wherein the promoter comprises a nucleic acid sequence having at least 99% identity to SEQ ID NO:475.

In some embodiments, a promoter set comprising a promoter sequence and an enhancer sequence described herein. According to some embodiments, wherein the promoter comprises a nucleic acid sequence having at least 95% identity to SEQ ID NO:479. According to some embodiments, wherein the promoter comprises a nucleic acid sequence having at least 96% identity to SEQ ID NO:479. According to some embodiments, wherein the promoter comprises a nucleic acid sequence having at least 97% identity to SEQ ID NO:479. According to some embodiments, wherein the promoter comprises a nucleic acid sequence having at least 98% identity to SEQ ID NO:479. According to some embodiments, wherein the promoter comprises a nucleic acid sequence having at least 99% identity to SEQ ID NO:479.

(ii) Enhancers

In some embodiments, a ceDNA expressing a codon optimized PAH comprises one or more enhancers. In some embodiments, an enhancer sequence is located 5′ of the promoter sequence. In some embodiments, the enhancer sequence is located 3′ of the promoter sequence. According to some embodiments, According to some embodiments, an enhancer is selected from the group consisting of a serpin enhancer, 3×HNF1-4_ProEnh_10mer, 5×HNF1_ProEnh_10mer.

TABLE 8A
Enhancers

| SEQ ID NO | |
|---|---|
| GE-## | Name |
| SEQ ID NO: 449 | Human Serpin Enhancer (hSerpEnh) |
| GE-1115 | |
| SEQ ID NO: 450 | TTRe |
| GE-1116 | |
| SEQ ID NO: 451 | HNF1 |
| GE-1117 | |
| SEQ ID NO: 452 | HNF4 |
| GE-1118 | |
| SEQ ID NO: 453 | ApoE_Enh |
| GE-1119 | |
| SEQ ID NO: 454 | ProEnh |
| GE-1120 | |
| SEQ ID NO: 455 | ApoE_Enh_C03 |
| GE-1129 | |
| SEQ ID NO: 456 | ApoE_Enh_C04 |
| GE-1130 | |
| SEQ ID NO: 457 | ApoE_Enh_C09 |
| GE-1131 | |
| SEQ ID NO: 458 | ApoE_Enh_C10 |
| GE-1132 | |
| SEQ ID NO: 459 | Embedded_HCR1_footprint123 |
| GE-1127 | |
| SEQ ID NO: 460 | Embedded_enhancer_HNF_array |
| GE-1128 | |
| SEQ ID NO: 461 | ApoE_Enh_v2 |
| GE-1237 | |
| SEQ ID NO: 586 | HS-CRM_FOXA_HNF4_consensus_v1 |
| SEQ ID NO: 587 | 3x_HNF4_FOXA_v1 |
| SEQ ID NO: 588 | 1x_Bushbaby SerpEnh |
| SEQ ID NO: 589 | 3x_Bushbaby_Aspacers |
| SEQ ID NO: 590 | 1x_Chinese Tree Shrew SerpEnh |
| SEQ ID NO: 591 | 3x_ChineseTreeShrew |
| | (“C” spancer inbetween the repeats) |
| SEQ ID NO: 592 | 3x_ChineseTreeShrew_CpGmin |
| SEQ ID NO: 593 | 3x_hSerpEnh_Aspacers |
| | (“A” spacer) |
| SEQ ID NO: 594 | 5x_Bushbaby_Aspacers |
| SEQ ID NO: 595 | 5x_ChineseTreeShrew |
| SEQ ID NO: 596 | 3x_hSerpEnh_11mer_spacers_v3 |
| SEQ ID NO: 597 | 3x_hSerpEnh_30mer_spacers_v3 |
| SEQ ID NO: 598 | 3x_hSerpEnh_30mer_spacers_ |
| | HNF4revmer_spacers_FOXArev |
| SEQ ID NO: 599 | 3x_hSerpEnh_2mer_spacers_v10 |
| SEQ ID NO: 600 | 3x_hSerpEnh_2mer_spacers_v12 |
| SEQ ID NO: 601 | 3x_hSerpEnh_2mer_spacers_v9 |

TABLE 8B
Descriptions for Enhancer Elements

| GE-## | Name | Description |
|---|---|---|
| GE-1115 | SerpEnh | Enhancer region for Serpin1 gene |
| | | as reported by Chuah, M., et al. |
| | | (2014). Liver-Specific |
| | | Transcriptional Modules |
| | | Identified by Genome-Wide In |
| | | Silico Analysis Enable Efficient |
| | | Gene Therapy in Mice and Non- |
| | | Human Primates Molecular Therapy |
| | | 22(9), 1605-1613. Available at |
| | | dx.doi.org/10.1038/mt.2014.114; |
| | | Disclosed herein also includes |
| | | modified SerpEnh sequences |
| | | originated from bushbaby and |
| | | Chinese tree shrew. |
| GE-1116 | TTRe | Enhancer region for Transthyretin |
| | | gene |
| GE-1117 | HNF1 | Hepatic Nuclear Factor 1 binding |
| | | site |
| GE-1118 | HNF4 | Hepatic Nuclear Factor 4 binding |
| | | site |
| GE-1119 | ApoE_Enh | Human apolipoprotein E/C-I liver |
| | | specific enhancer |
| GE-1120 | ProEnh | Enhancer region from Pro-albumin |
| | | gene |
| GE-1129 | ApoE_Enh_C03 | CpG minimized version of the |
| | | ApoE_Enh (Human apolipoprotein |
| | | E/C-I liver specific enhancer) |
| GE-1130 | ApoE_Enh_C04 | CpG minimized version of the |
| | | ApoE_Enh (Human apolipoprotein |
| | | E/C-I liver specific enhancer) |
| GE-1131 | ApoE_Enh_C09 | CpG minimized version of the |
| | | ApoE_Enh (Human apolipoprotein |
| | | E/C-I liver specific enhancer) |
| GE-1132 | ApoE_Enh_C10 | CpG minimized version of the |
| | | ApoE_Enh (Human apolipoprotein |
| | | E/C-I liver specific enhancer) |
| GE-1127 | Embedded_HCR1_ | HCR1 footprint123 embedded in |
| | footprint123 | GE-856 (aka between GE-859/ |
| | | GE-860) |
| GE-1128 | Embedded_enhancer_ | Hepatic nuclear factor enhancer |
| | HNF_array | array embedded in GE-856 (aka |
| | | between GE-859/GE-860) |
| GE-1237 | ApoE_Enh_v2 | Derivative of Human apolipoprotein |
| | | E/C-I liver specific enhancer |
| GE-1402 | HS-CRM_FOXA_ | Human SERPINA1 enhancer |
| | HNF4_consensus_v1 | variant: |
| | | FOXA_HNF4_consensus_v1 |
| GE-1502 | 3x_HNF4_FOXA_v1 | 3 repeats of HS- |
| | | CRM8_FOXA_HNF4_consensus_ |
| | | v1 separated by a cytosine |

| | |
|---|---|
| 1x_Bushbaby SerpEnh | 1x repeat of the Bushbaby |
| | SERPINA1 enhancer |
| 3x_Bushbaby_Aspacers | 3x repeat of the Bushbaby |
| | SERPINA1 enhancer with adenine |
| | nucleotide spacer (“A” spacer) |
| 1x_Chinese Tree Shrew SerpEnh | 1x repeat of the Chinese Tree |
| | Shrew SERPINA1 enhancer |
| 3x_ChineseTreeShrew | 3x repeat of the Chinese Tree |
| (“C” spacer inbetween | Shrew SERPINA1 enhancer |
| the repeats) | (“C” spaner inbetween the repeats) |
| 3x_ChineseTreeShrew_CpGmin | 3x repeat of the Chinese Tree |
| | Shrew SERPINA1 enhancer with |
| | CpG minimization) |
| 3x_hSerpEnh_Aspacers | 3x repeat of the human SERPINA1 |
| (“A” spacer) | enhancer with 1 adenine between |
| | the repeats (“A” spacer)) |
| 5x_Bushbaby_Aspacers | 5x repeat of the Bushbaby |
| | SERPINA1 enhancer with adenine |
| | nucleotide spacer (“A” spacer) |
| 5x_ChineseTreeShrew | 5x repeat of the Chinese Tree |
| | Shrew SERPINA1 enhancer |
| 3x_hSerpEnh_11mer_spacers_v3 | 3x repeat of hSerpEnh with 11mer |
| | spacers v3 |
| 3x_hSerpEnh_30mer_spacers_v3 | 3x repeat of hSerpEnh with 30mer |
| | spacers v3 |
| 3x_hSerpEnh_30mer_spacers_ | 3x repeat of hSerpEnh with 30mer |
| HNF4revmer_spacers_FOXArev | spacers with HNF4 binding site in |
| | orientation 2 & FOXA binding site |
| | in orientation 1 |
| 3x_hSerpEnh_2mer_spacers_v10 | 3x repeat of hSerpEnh with 2mer |
| | spacers version 10 |
| 3x_hSerpEnh_2mer_spacers_v12 | 3x repeat of hSerpEnh with 2mer |
| | spacers version 12 |
| 3x_hSerpEnh_2mer_spacers_v9 | 3x repeat of hSerpEnh with 2mer |
| | spacers version 9 |

According to some embodiments, the 3×HNFI-4_ProEnh (Pro-albumin enhancer) enhancer fused to TTR promoter comprises the sequence set forth in SEQ ID NO:462. According to some embodiments, the 3×HNFI-4_ProEnh (Pro-albumin enhancer) enhancer fused to 3× VanD-TTRe and TTR promoter comprises the sequence set forth in SEQ ID NO:463.

According to some embodiments, the 5×HNF1_ProEnh_enhancer fused to TTR promoter comprises the sequence set forth in SEQ ID NO:464. According to some embodiments, the 5×HNF1_ProEnh_enhancer fused to 3×VanD-TTRe and TTR promoter comprises the sequence set forth in SEQ ID NO:465.

According to some embodiments, the serpin enhancer (SerpEnh) comprises the sequence set forth as:

(SEQ ID NO: 449)
GGGGGAGGCTGCTGGTGAATATTAACCAAGGTCACCCCAGTT
ATCGGAGGAGCAAACAGGGGCTAAGTCCAC.

In some other embodiments, the enhancer can be used in multitudes of tandem or repeated sequences. Non-limiting examples include the enhancer sequences described in SEQ ID NOs: 587-601.

TABLE 9A
Promoter Sets (Core promoter and Enhancer(s) combined)

| GE | Name (SEQ ID NO:) |
|---|---|
| GE-1223 | PromoterSet for ceDNA1471 |
| | (SEQ ID NO: 462) |
| GE-1224 | PromoterSet for ceDNA1472 |
| | (SEQ ID NO: 463) |
| GE-1225 | PromoterSet for ceDNA1473 |
| | (SEQ ID NO: 464) |
| GE-1226 | PromoterSet for ceDNA1474 |
| | (SEQ ID NO: 465) |
| GE-1227 | PromoterSet for ceDNA1475 |
| | (SEQ ID NO: 466) |
| GE-1228 | PromoterSet for ceDNA1476 |
| | (SEQ ID NO: 467) |
| GE-1229 | PromoterSet for ceDNA1477 |
| | (SEQ ID NO: 468) |
| GE-1230 | PromoterSet for ceDNA1478 |
| | (SEQ ID NO: 469) |
| GE-1231 | PromoterSet for ceDNA1479 |
| | (SEQ ID NO: 470) |
| GE-1232 | PromoterSet for ceDNA1480 |
| | (SEQ ID NO: 471) |
| GE-1233 | PromoterSet for ceDNA1368 |
| | (SEQ ID NO: 472) |
| GE-1234 | PromoterSet for ceDNA1648 |
| | (SEQ ID NO: 473) |
| GE-1235 | PromoterSet for ceDNA1657 |
| | (SEQ ID NO: 474) |
| GE-1236 | PromoterSet for ceDNA1622 |
| | (SEQ ID NO: 475) |
| GE-1270 | PromoterSet for ceDNA1664 |
| | (SEQ ID NO: 478) |
| GE-1271 | PromoterSet for ceDNA979 |
| | (SEQ ID NO: 479) |
| GE-576 | hPAH-endoPromoter_v2 |
| | (SEQ ID NO: 480) |
| GE-586 | hPAH-endoPromoter_v2_deltaKozak |
| | (SEQ ID NO: 481) |

TABLE 9B
Promoter set (core promoter and enhancer(s) combined) Annotation

| GE-## | Name | Description |
|---|---|---|
| GE-1223 | Promoter Set for | Synthetic Liver specific PromoterSet including |
| | ceDNA1471 | enhancers and core promoter (without 5pUTR) |
| | (3xHNF1-4 \|\| Pro-Albumin | |
| | Enh \|\| TTR promoter) | |
| GE-1224 | Promoter Set for | Synthetic Liver specific PromoterSet including |
| | ceDNA1472 | enhancers and core promoter (without 5pUTR) |
| | (3xHNF1-4 \|\| Pro-Albumin | |
| | Enh \|\| 3xVanD-TTRe \|\| TTR | |
| | promoter) | |
| GE-1225 | PromoterSet for | Synthetic Liver specific PromoterSet including |
| | ceDNA1473 | enhancers and core promoter (without 5pUTR) |
| | (5xHNF1 \|\| Pro-Albumin | |
| | Enh \|\| TTR promoter) | |
| GE-1226 | Promoter Set for | Synthetic Liver specific PromoterSet including |
| | ceDNA1474 | enhancers and core promoter (without 5pUTR) |
| | (5xHNF1 \|\| Pro-Albumin | |
| | Enh \|\| 3xVanD-TTRe \|\| TTR | |
| | promoter) | |
| GE-1227 | Promoter Set for | Synthetic Liver specific PromoterSet including |
| | ceDNA1475 | enhancers and core promoter (without 5pUTR) |
| | (3xHNF1-4_Pro-Albumin | |
| | Enh \|\| hAAT promoter) | |
| GE-1228 | Promoter Set for | Synthetic Liver specific PromoterSet including |
| | ceDNA1476 | enhancers and core promoter (without 5pUTR) |
| | (3xHNF1-4_Pro-Albumin | |
| | Enh \|\| hAAT promoter) | |
| GE-1229 | Promoter Set for | Synthetic Liver specific PromoterSet including |
| | ceDNA1477 | enhancers and core promoter (without 5pUTR) |
| | (3xHNF1-4_Pro-hAAT- | |
| | 10mer-ApoE) | |
| GE-1230 | Promoter Set-1478 | Synthetic Liver specific PromoterSet including |
| | (5xHNF1-Pro-hAAT) | enhancers and core promoter (without 5pUTR) |
| GE-1231 | Promoter Set-1479 | Synthetic Liver specific PromoterSet including |
| | (5xHNF1-Pro-Albumin | enhancers and core promoter (without 5pUTR) |
| | Enh \|\| hAAT promoter) | |
| GE-1232 | Promoter Set-1480 | Synthetic Liver specific PromoterSet including |
| | (5xHNF1-Pro-hAAT- | enhancers and core promoter (without 5pUTR) |
| | 10mer-ApoE) | |
| GE-1233 | Promoter Set-1368 | Synthetic Liver specific PromoterSet including |
| | (VD Promoter set) | enhancers and core promoter (without 5pUTR) |
| GE-1234 | Promoter Set-1648 | Synthetic Liver specific PromoterSet including |
| | (3X serpin enhancer plus | enhancers and core promoter (without 5pUTR) |
| | TTRe; 3X VD Promoter set) | |
| GE-1235 | Promoter Set-1657 | Synthetic Liver specific PromoterSet including |
| | | enhancers and core promoter (without 5pUTR) |
| GE-1236 | Promoter Set-1622 | Synthetic Liver specific PromoterSet including |
| | (CpG minimized hAAT | enhancers and core promoter (without 5pUTR) |
| | promoter set) | |
| GE-1270 | Promoter Set-1664 | Synthetic Liver specific PromoterSet including |
| | | enhancers and core promoter (without 5pUTR) |
| GE-1271 | PromoterSet-979 | Synthetic Liver specific PromoterSet including |
| | (hAAT 979 promoter set) | enhancers and core promoter (without 5pUTR) |
| GE-576 | hPAH-endoPromoter_v2 | Human PAH endogenous promoter |
| GE-586 | hPAH-endoPromoter_ | Human PAH endogenous promoter |
| | v2_delta Kozak | |
| | 3x_Bushbaby SerpEnh- | |
| | TTRe-TTR promoter set | |
| | (SEQ ID NO: 602) | |
| | 3x_Chinese tree shrew | |
| | SerpEnh-TTRe-TTR | |
| | promoter set | |
| | (SEQ ID NO: 603) | |

TABLE 9C
Promoter sets: Combinations of the hAAT CpG minimized enhancer
and core promoters CpG minimized hAAT core_C10 (hAAT_979)
or hAAT_core_C06).

| | SEQ ID NO: |
|---|---|
| | CpG minimized hAAT |
| Name | Promoter Set Sequences |
| PromoterSet-970 | 604 |
| PromoterSet-971 | 1001 |
| PromoterSet-972 | 1002 |
| PromoterSet-973 | 1003 |
| PromoterSet-974 | 1004 |
| PromoterSet-975 | 1005 |
| PromoterSet-976 | 1006 |
| PromoterSet-977 | 1007 |
| PromoterSet-978 | 1008 |

(iii) 5′ UTR Sequences and Intron Sequences

In some embodiments, a ceDNA vector comprises a 5′ UTR sequence and/or an intron sequence that located 3′ of the 5′ ITR sequence. In some embodiments, the 5′ UTR is located 5′ of the transgene, e.g., sequence encoding the PAH protein. Exemplary 5′ UTR sequences listed in Table 10 below or in International Patent Application No. PCT/US2020/021328, for example in Table 9A, incorporated by reference in its entirety herein.

TABLE 10
5' UTR

| | | SEQ |
|---|---|---|
| GE# | Description | ID NO |
| GE-1124 | TTR-MVM-PmeI-Consensus-5pUTR | 482 |
| GE-1125 | TTR-MVM_v2-PmeI-Consensus-5pUTR | 483 |
| GE-1126 | TTR-MVM-PmeI*-Consensus-5pUTR | 484 |
| GE-1138 | hAAT-5pUTR_v2 | 485 |
| GE-1167 | TTR-MVMspliced-PmeI-Consensus-5pUTR | 486 |
| GE-772 | 5pUTR-325243 | 487 |
| GE-774 | 5pUTR-constant | 489 |
| GE-1208 | hAAT-SV40-PmeI-Mod-5pUTR | 490 |
| GE-1209 | hAAT-SV40-PmeI-Mod2-5pUTR | 491 |
| GE-1210 | hAAT-SV40-PmeI-Con-5pUTR | 492 |
| GE-1211 | hAAT-SV40-PmeI-325243-5pUTR | 493 |
| GE-1212 | hAAT-SV40-PmeI-536-5pUTR | 494 |
| GE-1219 | TTR-Xbal-MVM-PmeI-Consensus-5pUTR | 495 |
| GE-1220 | TTR-XbaI-MVM_v2-PmeI-Consensus-5pUTR | 496 |
| GE-1221 | TTR-XbaI-MVM-PmeI*-Consensus-5pUTR | 497 |
| GE-1122 | TTR-5pUTR | 498 |
| GE-1260 | hAAT-PmeI-Mod2-5pUTR | 400 |
| GE-1261 | TTR-MVM_v2-PmeI-Mod2-5pUTR | 500 |
| GE-1262 | TTR-MVM-PmeI-325243-5pUTR Copy | 502 |
| GE-1263 | TTR-MVM-PmeI*-Mod2-5pUTR | 502 |
| GE-1264 | TTR-MVM-PmeI-Mod2-5pUTR | 503 |
| GE-1265 | TTR-MVMspliced-PmeI-Mod2-5pUTR | 504 |
| GE-1266 | TTR-XbaI-MVM_v2-PmeI-Mod2-5pUTR | 505 |
| GE-1267 | TTR-XbaI-MVM-PmeI*-Mod2-5pUTR | 506 |
| GE-1268 | TTR-XbaI-MVM-PmeI-Mod2-5pUTR | 507 |
| GE-1269 | hAAT-PmeI-Con-5pUTR | 508 |

TABLE 10B
5' UTR Descriptions

| GE-## | Name | Description |
|---|---|---|
| GE-1124 | TTR-MVM-PmeI-Consensus- | 5pUTR formed form concatenation of 1) the |
| | 5pUTR | Transthyretin promoter 5pUTR, 2) Minute Virus of |
| | | Mouse Intron, 3) Pmel restriction site, and 4) |
| | | consensus kozak sequence |
| GE-1125 | TTR-MVM_v2-PmeI- | 5pUTR formed form concatenation of 1) the |
| | Consensus-5pUTR | Transthyretin promoter 5pUTR, 2) Minute Virus of |
| | | Mouse Intron_v2, 3) Pmel restriction site, and 4) |
| | | consensus kozak sequence |
| GE-1126 | TTR-MVM-PmeI*- | 5pUTR formed form concatenation of 1) the |
| | Consensus-5pUTR | Transthyretin promoter 5pUTR, 2) Minute Virus of |
| | | Mouse Intron, 3) Mutated Pmel restriction site, and |
| | | 4) consensus kozak sequence |
| GE-1138 | hAAT-5pUTR_v2 | 5pUTR region derived from SERPINA1 (A1AT) |
| | | gene |
| GE-1167 | TTR-MVMspliced-PmeI- | 5pUTR formed form concatenation of 1) the |
| | Consensus-5pUTR | Transthyretin promoter 5pUTR, 2) Spliced form of |
| | | Minute Virus of Mouse Intron, 3) Pmel restriction |
| | | site, and 4) consensus kozak sequence |
| GE-772 | 5pUTR-325243 | 5pUTR variable region #325243 |
| GE-774 | 5pUTR-constant | 5pUTR constant region |
| GE-1208 | hAAT-SV40-PmeI-Mod- | 5pUTR formed form concatenation of 1) the hAAT |
| | 5pUTR | promoter 5pUTR, 2) SV40 intron, 3) PmeI restriction |
| | | site, and 4) modified kozak sequence |
| GE-1209 | hAAT-SV40-PmeI-Mod2- | 5pUTR formed form concatenation of 1) the hAAT |
| | 5pUTR | promoter 5pUTR, 2) SV40 intron, 3) Pmel restriction |
| | | site, and 4) modified kozak sequence v2 |
| GE-1210 | hAAT-SV40-PmeI-Con- | 5pUTR formed form concatenation of 1) the hAAT |
| | 5pUTR | promoter 5pUTR, 2) SV40 intron, 3) PmeI restriction |
| | | site, and 4) consensus kozak sequence |
| GE-1211 | hAAT-SV40-PmeI-325243- | 5pUTR formed form concatenation of 1) the hAAT |
| | 5pUTR | promoter 5pUTR, 2) SV40 intron, 3) PmeI restriction |
| | | site, and 4) 325243-5pUTR |
| GE-1212 | hAAT-SV40-PmeI-536- | 5pUTR formed form concatenation of 1) the hAAT |
| | 5pUTR | promoter 5pUTR, 2) SV40 intron, 3) PmeI restriction |
| | | site, and 4) 536-kozak |
| GE-1219 | TTR-Xbal-MVM-PmeI- | 5pUTR formed form concatenation of 1) the |
| | Consensus-5pUTR | Transthyretin promoter 5pUTR, 2) Xbal restriction |
| | | site, 3)Minute Virus of Mouse Intron, 4) PmeI |
| | | restriction site, and 5) consensus kozak sequence |
| GE-1220 | TTR-XbaI-MVM_v2-PmeI- | 5pUTR formed form concatenation of 1) the |
| | Consensus-5pUTR | Transthyretin promoter 5pUTR, 2) Xbal restriction |
| | | site, 3) Minute Virus of Mouse Intron_v2, 4) Pmel |
| | | restriction site, and 5) consensus kozak seqeunce |
| GE-1221 | TTR-XbaI-MVM-PmeI*- | 5pUTR formed form concatenation of 1) the |
| | Consensus-5pUTR | Transthyretin promoter 5pUTR, 2) Xbal restriction |
| | | site, 3) Minute Virus of Mouse Intron, 4) Mutated |
| | | PmeI restriction site, and 5) consensus kozak |
| | | sequence |
| GE-1122 | TTR-5pUTR | 5pUTR from mouse Transthyretin gene |
| GE-1260 | hAAT-PmeI-Mod2-5pUTR | 5pUTR formed by concatenation of 1) the hAAT |
| | | promoter 5pUTR, 3) PmeI restriction site, and 4) |
| | | modified kozak sequence v2 |
| GE-1261 | TTR-MVM_v2-PmeI-Mod2- | 5pUTR formed by concatenation of 1) the |
| | 5pUTR | Transthyretin promoter 5pUTR, 2) Minute Virus of |
| | | Mouse Intron_v2, 3) Pmel restriction site, and 4) |
| | | Mod_Minimum_Consensus_Kozak_v2 |
| GE-1262 | TTR-MVM-PmeI-325243- | 5pUTR formed by concatenation of 1) the |
| | 5pUTR Copy | Transthyretin promoter 5pUTR, 2) Minute Virus of |
| | | Mouse Intron, 3) Pmel restriction site, and 4) |
| | | 325243-5pUTR |
| GE-1263 | TTR-MVM-PmeI*- Mod2- | 5pUTR formed by concatenation of 1) the |
| | 5pUTR | Transthyretin promoter 5pUTR, 2) Minute Virus of |
| | | Mouse Intron, 3) Mutated Pmel restriction site, and |
| | | 4) Mod_Minimum_Consensus_Kozak_v2 |
| GE-1264 | TTR-MVM-PmeI-Mod2- | 5pUTR formed by concatenation of 1) the |
| | 5pUTR | Transthyretin promoter 5pUTR, 2) Minute Virus of |
| | | Mouse Intron, 3) Pmel restriction site, and 4) |
| | | Mod_Minimum_Consensus_Kozak_v2 |
| GE-1265 | TTR-MVMspliced-PmeI- | 5pUTR formed by concatenation of 1) the |
| | Mod2-5pUTR | Transthyretin promoter 5pUTR, 2) Spliced form of |
| | | Minute Virus of Mouse Intron, 3) PmeI restriction |
| | | site, and 4) Mod_Minimum_Consensus_Kozak_v2 |
| GE-1266 | TTR-XbaI-MVM_v2-PmeI- | 5pUTR formed by concatenation of 1) the |
| | Mod2-5pUTR | Transthyretin promoter 5pUTR, 2) Xbal restriction |
| | | site, 3) Minute Virus of Mouse Intron_v2, 4) Pmel |
| | | restriction site, and 5) |
| | | Mod_Minimum_Consensus_Kozak_v2 |
| GE-1267 | TTR-XbaI-MVM-PmeI*- | 5pUTR formed by concatenation of 1) the |
| | Mod2-5pUTR | Transthyretin promoter 5pUTR, 2) Xbal restriction |
| | | site, 3) Minute Virus of Mouse Intron, 4) Mutated |
| | | Pmel restriction site, and 5) |
| | | Mod_Minimum_Consensus_Kozak_v2 |
| GE-1268 | TTR-XbaI-MVM-PmeI- | 5pUTR formed by concatenation of 1) the |
| | Mod2-5pUTR | Transthyretin promoter 5pUTR, 2) Xbal restriction |
| | | site, 3) Minute Virus of Mouse Intron, 4) PmeI |
| | | restriction site, and 5) |
| | | Mod_Minimum_Consensus_Kozak_v2 |
| GE-1269 | hAAT-PmeI-Con-5pUTR | 5pUTR formed by concatenation of 1) the hAAT |
| | | promoter 5pUTR, 3) PmeI restriction site, and 4) |
| | | Consensus Kozak Sequence |

In some embodiments, a ceDNA vector comprises an intron that is located 5′ of the ORF. In some other embodiments, a ceDNA vector comprises an intron that is within the ORF sequence. Suitable intron sequences that can be used are listed in Table 11A below.

TABLE 11A
Chimeric Intron Sequences

| | | Sequence |
|---|---|---|
| GE | Name | Identifier |
| GE-1252 | hIVS-1B intron | 509 |
| GE-1253 | hIVS-1B-Wt | 510 |
| GE-1254 | hPAH_Modified_Intron1_CpGfree_v1 | 511 |
| GE-1255 | hPAH-delta2KbIntron | 512 |
| GE-1256 | mIVS-1B intron | 513 |
| GE-1257 | mIVS-1B-CpGfree_v1 | 514 |
| GE-1258 | Modified_intron | 515 |
| GE-1259 | oIVS-v2 | 516 |
| GE-1260 | MVM_intron_v2 | 1000 |

TABLE 11B
Chimeric Intron Sequence Description

| GE # | Name | Description |
|---|---|---|
| GE-1252 | hIVS-1B intron | Chimeric intron derived from hPAH intron 1. |
| | | Engineered to Remove CpG motifs |
| GE-1253 | hIVS-1B-Wt | Chimeric intron derived from hPAH intron 1. |
| | | Wt sequence with CpG motifs |
| GE-1254 | hPAH_Modified_Intron1_CpGfree_v1 | chimeric intron derived from hPAH intron 1. |
| | | CpG's removed based on hIVS-1B sequence |
| GE-1255 | hPAH-delta2KbIntron | Chimeric intron derived from hPAH intron 1. |
| GE-1256 | mIVS-1B intron | Chimeric intron derived from mPAH intron 1 |
| GE-1257 | mIVS-1B-CpGfree_v1 | Chimeric intron derived from mPAH intron 1, |
| | | engineered to remove CpG motifs |
| GE-1258 | Modified_intron | Chimeric intron derived from hPAH intron 1. |
| GE-1259 | oIVS-v2 | Chimeric intron derived from orangutan PAH |
| | | intron 1. CpG free intron |
| GE-1260 | MVM_intron_v2 | Modified minute virus of mice (MVM) intron |

According to some embodiments, an MVM intron can be also implemented in 5′ of the PAH open reading frame (e.g., as part of 5′UTR). The MVM intron comprises SEQ ID NO:1026, shown below:

(SEQ ID NO: 1026)
AAGAGGTAAGGGTTTAAGGGATGGTTGGTTGGTGGGGTATT
AATGTTTAATTACCTGGAGCACCTGCCTGAAATCACTTTTT
TTCAGGTTG

(iv) 3′ UTR Sequences

In some embodiments, a ceDNA vector comprises a 3′ UTR sequence that located 5′ of the 3′ ITR sequence. In some embodiments, the 3′ UTR is located 3′ of the transgene, e.g., sequence encoding the PAH protein. Exemplary 3′ UTR sequences listed in Table 12 below or in International Application No. PCT/US2020/021328, for example in Table 9B3, incorporated by reference in its entirety herein.

TABLE 12
3' UTR Elements

| SEQ ID No. | Name | Description |
|---|---|---|
| SEQ ID NO:517 | bGH | Poly A signal derived from gene encoding bovine |
| (GE-001) | | growth hormone (bGH) |
| SEQ ID NO:518 | WPRE_3pUTR | Postranscriptional regulatory element derived from |
| (GE-007) | | Woodchuck Hepatitis Virus |
| SEQID NO:519 | SV40_polyA | PolyA region from SV40 virus |
| (GE-081) | | |
| SEQID NO:520 | HBB_3pUTR | Derived from Human hemoglobin beta (HBB) gene |
| (GE-080) | | 3pUTR |
| SEQID NO:521 | HBBv3_3pUTR | Derived from Human hemoglobin beta (HBB) gene |
| (GE-761) | | 3pUTR |
| SEQID NO:522 | HBBv2_3pUTR | Derived from Human hemoglobin beta (HBB) gene |
| (GE-720) | | 3pUTR |
| SEQID NO:523 | HBBv3_CpGmin | Derived from Human hemoglobin beta (HBB) gene |
| (GE-758) | | 3pUTR |
| SEQID NO:524 | HBBv2_CpGmin | Derived from Human hemoglobin beta (HBB) gene |
| (GE-760) | | 3pUTR |
| SEQID NO:525 | HBB-3pUTR- | Derived from Human hemoglobin beta (HBB) gene |
| (GE-582) | CpGmin_v1 | 3pUTR |

The ceDNA vectors for expression of PAH protein can further comprise a posttranscriptional regulatory element (WPRE) and BGH polyA.

(v) Polyadenylation Sequences

A sequence encoding a polyadenylation sequence can be included in the ceDNA vector for expression of PAH protein to stabilize an mRNA expressed from the ceDNA vector, and to aid in nuclear export and translation. In one embodiment, the ceDNA vector does not include a polyadenylation sequence. In other embodiments, the ceDNA vector for expression of PAH protein includes at least 1, at least 2, at least 3, at least 4, at least 5, at least 10, at least 15, at least 20, at least 25, at least 30, at least 40, least 45, at least 50 or more adenine dinucleotides. In some embodiments, the polyadenylation sequence comprises about 43 nucleotides, about 40-50 nucleotides, about 40-55 nucleotides, about 45-50 nucleotides, about 35-50 nucleotides, or any range there between.

The expression cassettes can include any poly-adenylation sequence known in the art or a variation thereof. In some embodiments, a poly-adenylation (polyA) sequence is selected from any of those listed in International Patent Application No. PCT/US2020/021328, for example in Table 10, incorporated by reference in its entirety herein. Other polyA sequences commonly known in the art can also be used, e.g., including but not limited to, naturally occurring sequence isolated from bovine BGHpA (e.g., SEQ ID NO: 68) or a virus SV40 pA (e.g., SEQ ID NO: 86), or a synthetic sequence (e.g., SEQ ID NO: 87). Some expression cassettes can also include SV40 late polyA signal upstream enhancer (USE) sequence. In some embodiments, a USE sequence can be used in combination with SV40 pA or heterologous poly-A signal. PolyA sequences are located 3′ of the transgene encoding the PAH protein.

The expression cassettes can also include a post-transcriptional element to increase the expression of a transgene. In some embodiments, Woodchuck Hepatitis Virus (WHP) posttranscriptional regulatory element (WPRE) (e.g., SEQ ID NO: 67) is used to increase the expression of a transgene. Other posttranscriptional processing elements such as the post-transcriptional element from the thymidine kinase gene of herpes simplex virus, or hepatitis B virus (HBV) can be used. Secretory sequences can be linked to the transgenes, e.g., VH-02 and VK-A26 sequences, e.g., SEQ ID NO: 88 and SEQ ID NO: 89.

(vi) Nuclear Localization Sequences and DNA Nuclear Targeting Sequences

In some embodiments, the ceDNA vector for expression of PAH protein comprises one or more nuclear localization sequences (NLSs), for example, 1, 2, 3, 4, 5, 6, 7, 8, 9, 10, or more NLSs. In some embodiments, the one or more NLSs are located at or near the amino-terminus, at or near the carboxy-terminus, or a combination of these (e.g., one or more NLS at the amino-terminus and/or one or more NLS at the carboxy terminus). When more than one NLS is present, each can be selected independently of the others, such that a single NLS is present in more than one copy and/or in combination with one or more other NLSs present in one or more copies. Non-limiting examples of NLSs are shown in Table 13A.

In some embodiments, the ceDNA vector for expression of PAH protein comprises one or more DNA nuclear targeting sequences (DTS) to promote ceDNA being taken into the nucleus of target cells. Table 13B listing non-limiting examples of DTS that can be implemented in a ceDNA vector expressing PAH protein.

TABLE 13A
Nuclear Localization Signals

| | SEQ |
|---|---|
| | ID |
| SOURCE | NO. |
| SV40 virus large T-antigen | 90 |
| nucleoplasmin | 92 |
| c-myc | 93 |
| | 94 |
| hRNPA1 M9 | 95 |
| IBB domain from importin-alpha | 96 |
| myoma T protein | 97 |
| | 98 |
| human p53 | 99 |
| mouse c-abl IV | 100 |
| influenza virus NS1 | 117 |
| | 118 |
| Hepatitis virus delta antigen | 119 |
| mouse Mx1 protein | 120 |
| human poly(ADP-ribose) polymerase | 121 |
| steroid hormone receptors (human) glucocorticoid | 122 |

TABLE 13B
DNA Nuclear Targeting Sequences (DTS)

| | | SEQ |
|---|---|---|
| | | ID |
| GE | Name | NO |
| GE-723 | 3NF_DTS | 526 |
| GE-767 | 3'DTS_primer_pad | 527 |
| GE-766 | 5'DTS_primer_pad | 528 |
| GE-763 | 5x_kB_mesika_DTS | 529 |
| GE-764 | 2x_GRE_dames_DTS | 530 |
| GE-765 | CREB_badding_DTS | 531 |
| GE-597 | SV40DNA_DTS_10mer Repeat | 532 |
| GE-784 | 2x_Cgt_GRE_meijsing_DTS | 533 |
| GE-596 | SV40DNA_DTS_72bpSingleRepeat | 534 |
| GE-595 | SV40DNA_DTS_72bpTandemRepeat | 535 |
| GE-1222 | 10xSV40-DTS-arrray | 536 |

TABLE 13C
DTS Descriptions

| GE-## | Name | Description |
|---|---|---|
| GE-723 | 3NF_DTS | nuclear factor kappa B (NFκB) |
| | | transcription factor binding site triplet, |
| | | comprising three 10-bp κB sites |
| | | (GGGACTTTCC (SEQ ID NO: 1018) |
| | | separated by a 5-bp optimized spacer |
| | | (AGCTG) |
| GE-767 | 3'DTS_primer_pad | CpG-minimized spacer optimized for |
| | | priming in PCR |
| GE-766 | 5'DTS_primer_pad | CpG-minimized spacer optimized for |
| | | priming in PCR |
| GE-763 | 5x_kB_mesika_DTS | 5X repeat of Igk kB motif 5'- |
| | | GGGGACTTTCC-3' (SEQ ID NO: 1019), |
| | | 3 bp spacer, as described by Mesika et al., |
| | | 2001 Mol Ther |
| GE-764 | 2x_GRE_dames_DTS | 2X repeat of glucocorticoid response |
| | | element (GRE; origin not described), SalI |
| | | restriction site as spacer, as described by |
| | | Dames et al., 2007 J Gene Med |
| GE-765 | CREB_badding_DTS | Single CREB binding site as described by |
| | | Badding et al., 2012 Gene Ther |
| GE-597 | SV40DNA_DTS_10merRepeat | 5x 72bp tandem repeat from SV40 genome |
| | | separated by random CpG free 20 mer |
| | | sequences. |
| GE-784 | 2x_Cgt_GRE_meijsing_DTS | High activity, high affinity GRE binding |
| | | site |
| GE-596 | SV40DNA_DTS_72bpSingleRepeat | 72 base pair single repeat region from |
| | | SV40 genome. |
| GE-595 | SV40DNA_DTS_72bpTandemRepeat | 72 base pair tandem repeat region from |
| | | SV40 genome. |
| GE-1222 | 10xSV40-DTS-arrray | 5x Dual SV40 Enhancer elements separated |
| | | by CpGfree spacer elements |

B. Additional Components of ceDNA Vectors

The ceDNA vectors for expression of PAH protein of the present disclosure may contain other components, such as, but not limited to, Kozak sequences (Table 14A), minute virus of mice (MVM) introns, spacers, CpG motifs. In some embodiments, the ceDNA vector for expression of PAH protein may comprise one or more micro RNA (MIR) sequences involved immune responses or hepato-homestasis (Table 14B3).

TABLE 14A
Kozak Sequences

| GE-# | | |
|---|---|---|
| (SEQ ID NO.) | Name | Description |
| GE-031 | Consensus_Kozak | Consensus Kozak sequence |
| (SEQ ID NO:537) | | |
| GE-366 | Mod_Minimum_Consensus_Kozak_v1 | Modified Consensus Kozak |
| (SEQ ID NO:538) | | sequence |
| GE-1206 | Mod_Minimum_Consensus_Kozak_v2 | Modified Consensus Kozak |
| (SEQ ID NO:539) | | sequence |
| GE-1207 | 536_Kozak | Minimal Kozak seqeuence |
| (SEQ ID NO:540) | | |

TABLE 14B
MIR Sequences

| SEQ ID NO: | | |
|---|---|---|
| GE# | Name | Description |
| SEQ ID NO: 1009 | mir122_4x | micro-RNA involved in regulation of immune reponses |
| GE-699 | | |
| SEQ ID NO: 1010 | mir-142_3pUTR | micro-RNA involved in liver homeostasis |
| GE-020 | | |

### C. Combination of Elements and ORFs

According to some embodiments, specific codon optimized nucleic acid sequences are paired with one or more of a combination of particular promoters, enhancers or other cis-elements.

According to some embodiments, the codon optimized sequence comprises hPAH_codop_ORF_v2, and is paired with a nucleic acid sequence encoding a VD_PromoterSet, as described herein. According to some embodiments, the codon optimized sequence comprises hPAH_codop_ORF_v2, and is paired with a nucleic acid sequence encoding a VD_PromoterSet, as described herein, wherein the VD Promoter (e.g., TTRm) further comprises a SERP enhancer. According to some embodiments, the codon optimized sequence comprises hPAH_codop_ORF_v2, and is paired with a nucleic acid sequence encoding a VD_PromoterSet, as described herein, wherein the VD Promoter (e.g., TTRm) comprises 3×SERP enhancer. According to some embodiments, the codon optimized sequence comprises hPAH_codop_ORF_v2, and is paired with a nucleic acid sequence encoding a VD_PromoterSet, as described herein, wherein the VD Promoter comprises 3×SERP enhancer and further comprises a MVM intron.

According to some embodiments, the codon optimized sequence comprises hPAH_codop_ORF_v2, and is paired with a nucleic acid sequence encoding a hAAT (979)_PromoterSet, as described herein. According to some embodiments, the codon optimized sequence comprises hPAH_codop_ORF_v2, a and is paired with a nucleic acid sequence encoding a TTR liver specific promoter, as described herein, and further comprises a ProEnh_10mer and a MVM intron.

According to some embodiments, the codon optimized sequence comprises hPAH_codop_ORF_v2, and is paired with a nucleic acid sequence encoding a transthyretin (TTR) liver specific promoter, as described herein. According to some embodiments, the codon optimized sequence comprises hPAH_codop_ORF_v2, and is paired with a nucleic acid sequence encoding a transthyretin (TTR) liver specific promoter, as described herein, and further comprises an MVM intron. According to some embodiments, the codon optimized sequence comprises hPAH_codop_ORF_v2, and is paired with a nucleic acid sequence encoding a minimal transthyretin (TTRm) liver specific promoter, as described herein.

According to some embodiments, the codon optimized sequence comprises hPAH_codop_ORF_v2 delta1-29aa, and is paired with a nucleic acid sequence encoding a VD_PromoterSet or CpG minimized hAAT as described herein.

According to some embodiments, the codon optimized sequence comprises hPAH-r5-s29, and is paired with a nucleic acid sequence encoding a VD_PromoterSet, as described herein, wherein the VD Promoter comprises 3×SERP enhancer.

According to some embodiments, the ceDNA construct comprises left-ITR_v1, spacer_left-ITR_v2.1, VD_PromoterSet, PmeI_site, Mod_Minimum_Consensus_Kozak, hPAH_codop_ORF_v2, PacI_site, WPRE_3pUTR, bGH, spacer_right-ITR_v1, right-ITR_v1. According to some embodiments, the ceDNA construct consists of left-ITR_v1, spacer_left-ITR_v2.1, VD_PromoterSet, PmeI_site, Mod_Minimum_Consensus_Kozak, hPAH_codop_ORF_v2, PacI_site, WPRE_3pUTR, bGH, spacer_right-ITR_v1, right-ITR_v1.

According to some embodiments, the ceDNA construct comprises Left-ITR_v1: spacer_left-ITR_v1: VD_Promoter Set: PmeI_site: Consensus_Kozak: hPAH_cDNA_ORF_v3: PacI_site: WPRE_3pUTR: bGH: spacer_right-ITR_v1: right-ITR_v1. According to some embodiments, the ceDNA construct consists of Left-ITR_v1: spacer_left-ITR_v1: VD_Promoter Set: PmeI_site: Consensus_Kozak: hPAH_cDNA_ORF_v3: PacI_site: WPRE_3pUTR: bGH: spacer_right-ITR_v1: right-ITR_v1.

According to some embodiments, the ceDNA construct comprises Left-ITR_v1, spacer_left-ITR_v2.1, 3×SerpEnh-TTRe-TTRm, MVM_intron, PmeI_site, Mod_Minimum_Consensus_Kozak, hPAH_codop_ORF_v2, PacI_site, WPRE_3pUTR, bGH, spacer_right-ITR_v1, right-ITR_v1. According to some embodiments, the ceDNA construct consists of Left-ITR_v1, spacer_left-ITR_v2.1, 3×SerpEnh-TTRe-TTRm, MVM_intron, PmeI_site, Mod_Minimum_Consensus_Kozak, hPAH_codop_ORF_v2, PacI_site, WPRE_3pUTR, bGH, spacer_right-ITR_v1, right-ITR_v1.

According to some embodiments, the ceDNA construct comprises left-ITR_v1, spacer_left-ITR_v2.1, VD_PromoterSet, PmeI_site, Mod_Minimum_Consensus_Kozak, hPAH_codop_ORF_v2_delta1-29aa, PacI_site, WPRE_3pUTR, bGH, spacer_right-ITR_v1, right-ITR_v1. According to some embodiments, the ceDNA construct consists of left-ITR_v1, spacer_left-ITR_v2.1, VD_PromoterSet, PmeI_site, Mod_Minimum_Consensus_Kozak, hPAH_codop_ORF_v2_delta1-29aa, PacI_site, WPRE_3pUTR, bGH, spacer_right-ITR_v1, right-ITR_v1.

According to some embodiments, the ceDNA construct comprises left-ITR_v1, spacer_left-ITR_v2.1, 3×VanD_TTRe_PromoterSet, PmeI_site, Mod_Minimum_Consensus_Kozak, hPAH-r5-s29::hIVS1B, PacI_site, WPRE_3pUTR, bGH, spacer_right-ITR_v1, right-ITR_v1. According to some embodiments, the ceDNA construct consists of left-ITR_v1, spacer_left-ITR_v2.1, 3×VanD_TTRe_PromoterSet, PmeI_site, Mod_Minimum_Consensus_Kozak, hPAH-r5-s29::hIVS1B, PacI_site, WPRE_3pUTR, bGH, spacer_right-ITR_v1, right-ITR_v1.

According to some embodiments, the ceDNA construct comprises left-ITR_v1, spacer_left-ITR_v2.1, 3×VanD_TTRe_PromoterSet, PmeI_site, Mod_Minimum_Consensus_Kozak, hPAH-r5-s29::hIVS1B_33bpFlanks, PacI_site, WPRE_3pUTR, bGH, spacer_right-ITR_v1, right-ITR_v1. According to some embodiments, the ceDNA construct consists of left-ITR_v1, spacer_left-ITR_v2.1, 3×VanD_TTRe_PromoterSet, PmeI_site, Mod_Minimum_Consensus_Kozak, hPAH-r5-s29::hIVS1B_33bpFlanks, PacI_site, WPRE_3pUTR, bGH, spacer_right-ITR_v1, right-ITR_v1.

According to some embodiments, the ceDNA construct comprises left-ITR_v1, spacer_left-ITR_v2.1, 3×VanD_TTRe_PromoterSet, PmeI_site, Mod_Minimum_Consensus_Kozak, hPAH-r5-s29::mod-Intron_oIVS-v2_33bpFlanks, PacI_site, WPRE_3pUTR, bGH, spacer_right-ITR_v1, right-ITR_v1. According to some embodiments, the ceDNA construct consists of left-ITR_v1, spacer_left-ITR_v2.1, 3×VanD_TTRe_PromoterSet, PmeI_site, Mod_Minimum_Consensus_Kozak, hPAH-r5-s29::mod-Intron_oIVS-v2_33bpFlanks, PacI_site, WPRE_3pUTR, bGH, spacer_right-ITR_v1, right-ITR_v1.

According to some embodiments, the ceDNA construct comprises left-ITR_v1, spacer_left-ITR_v2.1, 3×VanD_TTRe_PromoterSet, PmeI_site, Mod_Minimum_Consensus_Kozak, hPAH_codop_ORF_v2_mIVS-intron1B_33bpFlanks, PacI_site, WPRE_3pUTR, bGH, spacer_right-ITR_v1, right-ITR_v1. According to some embodiments, the ceDNA construct consists of left-ITR_v1, spacer_left-ITR_v2.1, 3×VanD_TTRe_PromoterSet, PmeI_site, Mod_Minimum_Consensus_Kozak, hPAH_codop_ORF_v2_mIVS-intron1B_33bpFlanks, PacI_site, WPRE_3pUTR, bGH, spacer_right-ITR_v1, right-ITR_v1.

According to some embodiments, the ceDNA construct comprises left-ITR_v1, spacer_left-ITR_v2.1, 3×VanD_TTRe_PromoterSet, PmeI_site, Mod_Minimum_Consensus_Kozak, hPAH_codop_ORF_v2_modified_Intron1_33bpFlanks, PacI_site, WPRE_3pUTR, bGH, spacer_right-ITR_v1, right-ITR_v1. According to some embodiments, the ceDNA construct consists of left-ITR_v1, spacer_left-ITR_v2.1, 3×VanD_TTRe_PromoterSet, PmeI_site, Mod_Minimum_Consensus_Kozak, hPAH_codop_ORF_v2_modified_Intron1_33bpFlanks, PacI_site, WPRE_3pUTR, bGH, spacer_right-ITR_v1, right-ITR_v1.

According to some embodiments, the ceDNA construct comprises left-ITR_v1, spacer_left-ITR_v2.1, hAAT(979)_PromoterSet, PmeI_site, Mod_Minimum_Consensus_Kozak, hPAH_codop_ORF_v2, PacI_site, WPRE_3pUTR, bGH, spacer_right-ITR_v1, right-ITR_v1. According to some embodiments, the ceDNA construct consists of left-ITR_v1, spacer_left-ITR_v2.1, hAAT(979)_PromoterSet, PmeI_site, Mod_Minimum_Consensus_Kozak, hPAH_codop_ORF_v2, PacI_site, WPRE_3pUTR, bGH, spacer_right-ITR_v1, right-ITR_v1

According to some embodiments, the ceDNA construct comprises left-ITR_v1, spacer_left-ITR_v2.1, VD_PromoterSet, PmeI_site, Mod_Minimum_Consensus_Kozak, hPAH_codop_ORF_v2, PacI_site, HBBv2_3pUTR, SV40_polyA, spacer_right-ITR_v1, right-ITR_v1. According to some embodiments, the ceDNA construct consists of left-ITR_v1, spacer_left-ITR_v2.1, VD_PromoterSet, PmeI_site, Mod_Minimum_Consensus_Kozak, hPAH_codop_ORF_v2, PacI_site, HBBv2_3pUTR, SV40_polyA, spacer_right-ITR_v1, right-ITR_v1.

According to some embodiments, the ceDNA construct comprises left-ITR_v1, spacer_left-ITR_v2.1, VD_PromoterSet, PmeI_site, Mod_Minimum_Consensus_Kozak, hPAH_codop_ORF_v2, PacI_site, HBBv3_3pUTR, SV40_polyA, spacer_right-ITR_v1, right-ITR_v1. According to some embodiments, the ceDNA construct consists of left-ITR_v1, spacer_left-ITR_v2.1, VD_PromoterSet, PmeI_site, Mod_Minimum_Consensus_Kozak, hPAH_codop_ORF_v2, PacI_site, HBBv3_3pUTR, SV40_polyA, spacer_right-ITR_v1, right-ITR_v1.

According to some embodiments, the ceDNA construct comprises left-ITR_v1, spacer_left-ITR_v2.1, VD_PromoterSet, PmeI_site, Mod_Minimum_Consensus_Kozak, hPAH_codop_ORF_v2, PacI_site, WPRE_3pUTR, SV40_polyA, spacer_right-ITR_v1, right-ITR_v1. According to some embodiments, the ceDNA construct consists of left-ITR_v1, spacer_left-ITR_v2.1, VD_PromoterSet, PmeI_site, Mod_Minimum_Consensus_Kozak, hPAH_codop_ORF_v2, PacI_site, WPRE_3pUTR, SV40_polyA, spacer_right-ITR_v1, right-ITR_v1.

According to some embodiments, the ceDNA construct comprises left-ITR_v1, spacer_left-ITR_v2.1, VD_PromoterSet, PmeI_site, Mod_Minimum_Consensus_Kozak, hPAH_codop_ORF_v2, PacI_site, WPRE_3pUTR, HBBv2_3pUTR, spacer_right-ITR_v1, right-ITR_v1. According to some embodiments, the ceDNA construct consists of left-ITR_v1, spacer_left-ITR_v2.1, VD_PromoterSet, PmeI_site, Mod_Minimum_Consensus_Kozak, hPAH_codop_ORF_v2, PacI_site, WPRE_3pUTR, HBBv2_3pUTR, spacer_right-ITR_v1, right-ITR_v1.

According to some embodiments, the ceDNA construct comprises left-ITR_v1, spacer_left-ITR_v2.1, 3×HNFI-4_ProEnh_10mer, BamHI_site, TTR_liver_specific_Promoter, MVM_intron, PmeI_site, Mod_Minimum_Consensus_Kozak, hPAH_codop_ORF_v2, PacI_site, WPRE_3pUTR, bGH, spacer_right-ITR_v1, right-ITR_v1. According to some embodiments, the ceDNA construct consists of left-ITR_v1, spacer_left-ITR_v2.1, 3×HNFI-4_ProEnh_10mer, BamHI_site, TTR_liver_specific_Promoter, MVM_intron, PmeI_site, Mod_Minimum_Consensus_Kozak, hPAH_codop_ORF_v2, PacI_site, WPRE_3pUTR, bGH, spacer_right-ITR_v1, right-ITR_v1.

According to some embodiments, the ceDNA construct comprises left-ITR_v1, spacer_left-ITR_v2.1, 3×HNF1-4_ProEnh_10mer, HS-CRM8_SERP_Enhancer_nospacer, HS-CRM8_SERP_Enhancer_nospacer, BamHI_site, TTR_liver_specific_Promoter, MVM_intron, PmeI_site, Mod_Minimum_Consensus_Kozak, hPAH_codop_ORF_v2, PacI_site, WPRE_3pUTR, bGH, spacer_right-ITR_v1, right-ITR_v1. According to some embodiments, the ceDNA construct consists of left-ITR_v1, spacer_left-ITR_v2.1, 3×HNFI-4_ProEnh_10mer, HS-CRM8_SERP_Enhancer_nospacer, HS-CRM8_SERP_Enhancer_nospacer, BamHI_site, TTR_liver_specific_Promoter, MVM_intron, PmeI_site, Mod_Minimum_Consensus_Kozak, hPAH_codop_ORF_v2, PacI_site, WPRE_3pUTR, bGH, spacer_right-ITR_v1, right-ITR_v1.

According to some embodiments, the ceDNA construct comprises left-ITR_v1, spacer_left-ITR_v2.1, CpGfree20mer_1, 5×HNF1_ProEnh_10mer, BamHI_site, TTR_liver_specific_Promoter, MVM_intron, PmeI_site, Mod_Minimum_Consensus_Kozak, hPAH_codop_ORF_v2, PacI_site, WPRE_3pUTR, bGH, spacer_right-ITR_v1, right-ITR_v1. According to some embodiments, the ceDNA construct consists of left-ITR_v1, spacer_left-ITR_v2.1, CpGfree20mer_1, 5×HNF1_ProEnh_10mer, BamHI_site, TTR_liver_specific_Promoter, MVM_intron, PmeI_site, Mod_Minimum_Consensus_Kozak, hPAH_codop_ORF_v2, PacI_site, WPRE_3pUTR, bGH, spacer_right-ITR_v1, right-ITR_v1.

According to some embodiments, the ceDNA construct comprises left-ITR_v1, spacer_left-ITR_v2.1, CpGfree20mer_1, 5×HNFI_ProEnh_10mer, 3×VanD_TTRe_PromoterSet_v2, PmeI_site, Mod_Minimum_Consensus_Kozak, hPAH_codop_ORF_v2, PacI_site, WPRE_3pUTR, bGH, spacer_right-ITR_v1, right-ITR_v1. According to some embodiments, the ceDNA construct consists of left-ITR_v1, spacer_left-ITR_v2.1, CpGfree20mer_1, 5×HNFI_ProEnh_10mer, 3×VanD_TTRe_PromoterSet_v2, PmeI_site, Mod_Minimum_Consensus_Kozak, hPAH_codop_ORF_v2, PacI_site, WPRE_3pUTR, bGH, spacer_right-ITR_v1, right-ITR_v1.

According to some embodiments, the ceDNA construct comprises left-ITR_v1, spacer_left-ITR_v2.1, 3×VanD_TTRe_PromoterSet_v2, PmeI_site, Mod_Minimum_Consensus_Kozak, hPAH_codop_ORF_v2, PacI_site, WPRE_3pUTR, bGH, spacer_right-ITR_v1, right-ITR_v1. According to some embodiments, the ceDNA construct consists of left-ITR_v1, spacer_left-ITR_v2.1, 3×VanD_TTRe_PromoterSet_v2, PmeI_site, Mod_Minimum_Consensus_Kozak, hPAH_codop_ORF_v2, PacI_site, WPRE_3pUTR, bGH, spacer_right-ITR_v1, right-ITR_v1.

According to some embodiments, the ceDNA construct comprises left-ITR_v1, spacer_left-ITR_v2.1, CpGmin_hAAT_Promoter_Set, PmeI_site, Mod_Minimum_Consensus_Kozak, hPAH_codop_ORF_v2, PacI_site, WPRE_3pUTR, bGH, spacer_right-ITR_v1, right-ITR_v1. According to some embodiments, the ceDNA construct consists of left-ITR_v1, spacer_left-ITR_v2.1, CpGmin_hAAT_Promoter_Set, PmeI_site, Mod_Minimum_Consensus_Kozak, hPAH_codop_ORF_v2, PacI_site, WPRE_3pUTR, bGH, spacer_right-ITR_v1, right-ITR_v1.

According to some embodiments, the ceDNA construct comprises left-ITR_v1, spacer_left-ITR_v2, HS-CRM8_SERP_Enhancer_nospacer, HS-CRM8_SERP_Enhancer_nospacer, HS-CRM8_SERP_Enhancer_nospacer, BamHI_site, TTR-promoter-d5pUTR, MVM_intron, PmeI_site, Mod_Minimum_Consensus_Kozak, hPAH-r3-s34, PacI_site, WPRE_3pUTR, bGH, spacer_right-ITR_v1, right-ITR_v1. According to some embodiments, the ceDNA construct consists of left-ITR_v1, spacer_left-ITR_v2, HS-CRM8_SERP_Enhancer_nospacer, HS-CRM8_SERP_Enhancer_nospacer, HS-CRM8_SERP_Enhancer_nospacer, BamHI_site, TTR-promoter-d5pUTR, MVM_intron, PmeI_site, Mod_Minimum_Consensus_Kozak, hPAH-r3-s34, PacI_site, WPRE_3pUTR, bGH, spacer_right-ITR_v1, right-ITR_v1.

According to some embodiments, the ceDNA construct comprises left-ITR_v1, spacer_left-ITR_v2, HS-CRM8_SERP_Enhancer_nospacer, HS-CRM8_SERP_Enhancer_nospacer, HS-CRM8_SERP_Enhancer_nospacer, BamHI_site, TTR-promoter-d5pUTR, MVM_intron, PmeI_site, Mod_Minimum_Consensus_Kozak, hPAH-r5-s29, PacI_site, WPRE_3pUTR, bGH, spacer_right-ITR_v1, right-ITR_v1. According to some embodiments, the ceDNA construct consists of left-ITR_v1, spacer_left-ITR_v2, HS-CRM8_SERP_Enhancer_nospacer, HS-CRM8_SERP_Enhancer_nospacer, HS-CRM8_SERP_Enhancer_nospacer, BamHI_site, TTR-promoter-d5pUTR, MVM_intron, PmeI_site, Mod_Minimum_Consensus_Kozak, hPAH-r5-s29, PacI_site, WPRE_3pUTR, bGH, spacer_right-ITR_v1, right-ITR_v1.

According to some embodiments, the ceDNA construct comprises a nucleic acid sequence that is at least 90% identical to a sequence selected from the group consisting of: SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, and SEQ ID NO: 213.

### D. Regulatory Switches

A molecular regulatory switch is one which generates a measurable change in state in response to a signal. Such regulatory switches can be usefully combined with the ceDNA vectors for expression of PAH protein as described herein to control the output of expression of PAH protein from the ceDNA vector. In some embodiments, the ceDNA vector for expression of PAH protein comprises a regulatory switch that serves to fine tune expression of the PAH protein. For example, it can serve as a biocontainment function of the ceDNA vector. In some embodiments, the switch is an “ON/OFF” switch that is designed to start or stop (i.e., shut down) expression of PAH protein in the ceDNA vector in a controllable and regulatable fashion. In some embodiments, the switch can include a “kill switch” that can instruct the cell comprising the ceDNA vector to undergo cell programmed death once the switch is activated. Exemplary regulatory switches encompassed for use in a ceDNA vector for expression of PAH protein can be used to regulate the expression of a transgene, and are more fully discussed in International application PCT/US18/49996, which is incorporated herein in its entirety by reference.

(i) Binary Regulatory Switches

In some embodiments, the ceDNA vector for expression of PAH protein comprises a regulatory switch that can serve to controllably modulate expression of PAH protein. For example, the expression cassette located between the ITRs of the ceDNA vector may additionally comprise a regulatory region, e.g., a promoter, cis-element, repressor, enhancer etc., that is operatively linked to the nucleic acid sequence encoding PAH protein, where the regulatory region is regulated by one or more cofactors or exogenous agents. By way of example only, regulatory regions can be modulated by small molecule switches or inducible or repressible promoters. Non-limiting examples of inducible promoters are hormone-inducible or metal-inducible promoters. Other exemplary inducible promoters/enhancer elements include, but are not limited to, an RU486-inducible promoter, an ecdysone-inducible promoter, a rapamycin-inducible promoter, and a metallothionein promoter.

(ii) Small molecule Regulatory Switches

A variety of art-known small-molecule based regulatory switches are known in the art and can be combined with the ceDNA vectors for expression of PAH protein as disclosed herein to form a regulatory-switch controlled ceDNA vector. In some embodiments, the regulatory switch can be selected from any one or a combination of: an orthogonal ligand/nuclear receptor pair, for example retinoid receptor variant/LG335 and GRQCIMFI, along with an artificial promoter controlling expression of the operatively linked transgene, such as that as disclosed in Taylor, et al. BMC Biotechnology 10 (2010): 15; engineered steroid receptors, e.g., modified progesterone receptor with a C-terminal truncation that cannot bind progesterone but binds RU486 (mifepristone) (U.S. Pat. No. 5,364,791); an ecdysone receptor from Drosophila and their ecdysteroid ligands (Saez, et al., PNAS, 97(26)(2000), 14512-14517; or a switch controlled by the antibiotic trimethoprim (TMP), as disclosed in Sando R 3^{rd}; Nat Methods. 2013, 10(11):1085-8. In some embodiments, the regulatory switch to control the transgene or expressed by the ceDNA vector is a pro-drug activation switch, such as that disclosed in U.S. Pat. Nos. 8,771,679, and 6,339,070, incorporated by reference in their entireties herein.

(iii) “Passcode” Regulatory Switches

In some embodiments the regulatory switch can be a “passcode switch” or “passcode circuit”. Passcode switches allow fine tuning of the control of the expression of the transgene from the ceDNA vector when specific conditions occur—that is, a combination of conditions need to be present for transgene expression and/or repression to occur. For example, for expression of a transgene to occur at least conditions A and B must occur. A passcode regulatory switch can be any number of conditions, e.g., at least 2, or at least 3, or at least 4, or at least 5, or at least 6 or at least 7 or more conditions to be present for transgene expression to occur. In some embodiments, at least 2 conditions (e.g., A, B conditions) need to occur, and in some embodiments, at least 3 conditions need to occur (e.g., A, B and C, or A, B and D). By way of an example only, for gene expression from a ceDNA to occur that has a passcode “ABC” regulatory switch, conditions A, B and C must be present. Conditions A, B and C could be as follows; condition A is the presence of a condition or disease, condition B is a hormonal response, and condition C is a response to the transgene expression. For example, if the transgene edits a defective EPO gene, Condition A is the presence of Chronic Kidney Disease (CKD), Condition B occurs if the subject has hypoxic conditions in the kidney, Condition C is that Erythropoietin-producing cells (EPC) recruitment in the kidney is impaired; or alternatively, HIF-2 activation is impaired. Once the oxygen levels increase or the desired level of EPO is reached, the transgene turns off again until 3 conditions occur, turning it back on.

In some embodiments, a passcode regulatory switch or “Passcode circuit” encompassed for use in the ceDNA vector comprises hybrid transcription factors (TFs) to expand the range and complexity of environmental signals used to define biocontainment conditions. As opposed to a deadman switch which triggers cell death in the presence of a predetermined condition, the “passcode circuit” allows cell survival or transgene expression in the presence of a particular “passcode”, and can be easily reprogrammed to allow transgene expression and/or cell survival only when the predetermined environmental condition or passcode is present.

Any and all combinations of regulatory switches disclosed herein, e.g., small molecule switches, nucleic acid-based switches, small molecule-nucleic acid hybrid switches, post-transcriptional transgene regulation switches, post-translational regulation, radiation-controlled switches, hypoxia-mediated switches and other regulatory switches known by persons of ordinary skill in the art as disclosed herein can be used in a passcode regulatory switch as disclosed herein. Regulatory switches encompassed for use are also discussed in the review article Kis et al., J R Soc Interface. 12: 20141000 (2015), and summarized in Table 1 of Kis. In some embodiments, a regulatory switch for use in a passcode system can be selected from any or a combination of the switches disclosed in Table 11 of International Patent Application No. PCT/US18/49996, which is incorporated herein in its entirety by reference.

(iv) Nucleic Acid-Based Regulatory Switches to Control Transgene Expression

In some embodiments, the regulatory switch to control the expression of PAH protein by the ceDNA is based on a nucleic-acid based control mechanism. Exemplary nucleic acid control mechanisms are known in the art and are envisioned for use. For example, such mechanisms include riboswitches, such as those disclosed in, e.g., US2009/0305253, US2008/0269258, US2017/0204477, WO2018026762A1, U.S. Pat. No. 9,222,093 and EP application EP288071, and also disclosed in the review by Villa J K et al., Microbiol Spectr. 2018 May; 6(3). Also included are metabolite-responsive transcription biosensors, such as those disclosed in WO2018/075486 and WO2017/147585. Other art-known mechanisms envisioned for use include silencing of the transgene with an siRNA or RNAi molecule (e.g., miR, shRNA). For example, the ceDNA vector can comprise a regulatory switch that encodes a RNAi molecule that is complementary to the part of the transgene expressed by the ceDNA vector. When such RNAi is expressed even if the transgene (e.g., PAH protein) is expressed by the ceDNA vector, it will be silenced by the complementary RNAi molecule, and when the RNAi is not expressed when the transgene is expressed by the ceDNA vector the transgene (e.g., PAH protein) is not silenced by the RNAi.

In some embodiments, the regulatory switch is a tissue-specific self-inactivating regulatory switch, for example as disclosed in US2002/0022018, whereby the regulatory switch deliberately switches transgene (e.g., PAH protein) off at a site where transgene expression might otherwise be disadvantageous. In some embodiments, the regulatory switch is a recombinase reversible gene expression system, for example as disclosed in US2014/0127162 and U.S. Pat. No. 8,324,436.

(v) Post-Transcriptional and Post-Translational Regulatory Switches.

In some embodiments, the regulatory switch to control the expression of PAH protein by the ceDNA vector is a post-transcriptional modification system. For example, such a regulatory switch can be an aptazyme riboswitch that is sensitive to tetracycline or theophylline, as disclosed in US2018/0119156, GB201107768, WO2001/064956A3, EP Patent 2707487 and Beilstein et al., ACS Synth. Biol., 2015, 4 (5), pp 526-534; Zhong et al., Elife. 2016 Nov. 2; 5. pii: e18858. In some embodiments, it is envisioned that a person of ordinary skill in the art could encode both the transgene and an inhibitory siRNA which contains a ligand sensitive (OFF-switch) aptamer, the net result being a ligand sensitive ON-switch.

(vi) Other Exemplary Regulatory Switches

Any known regulatory switch can be used in the ceDNA vector to control the expression of PAH protein by the ceDNA vector, including those triggered by environmental changes. Additional examples include, but are not limited to; the BOC method of Suzuki et al., Scientific Reports 8; 10051 (2018); genetic code expansion and a non-physiologic amino acid; radiation-controlled or ultra-sound controlled on/off switches (see, e.g., Scott S et al., Gene Ther. 2000 July; 7(13):1121-5; U.S. Pat. Nos. 5,612,318; 5,571,797; 5,770,581; 5,817,636; and WO1999/025385A1. In some embodiments, the regulatory switch is controlled by an implantable system, e.g., as disclosed in U.S. Pat. No. 7,840,263; US2007/0190028A1 where gene expression is controlled by one or more forms of energy, including electromagnetic energy, that activates promoters operatively linked to the transgene in the ceDNA vector.

In some embodiments, a regulatory switch envisioned for use in the ceDNA vector is a hypoxia-mediated or stress-activated switch, e.g., such as those disclosed in WO1999060142A2, U.S. Pat. Nos. 5,834,306; 6,218,179; 6,709,858; US2015/0322410; Greco et al., (2004) Targeted Cancer Therapies 9, S368, incorporated by reference in their entireties herein, as well as FROG, TOAD and NRSE elements and conditionally inducible silence elements, including hypoxia response elements (HREs), inflammatory response elements (IREs) and shear-stress activated elements (SSAEs), e.g., as disclosed in U.S. Pat. No. 9,394,526, incorporated by reference in its entirety herein. Such an embodiment is useful for turning on expression of the transgene from the ceDNA vector after ischemia or in ischemic tissues, and/or tumors.

(vii) Kill Switches

Other embodiments described herein relate to a ceDNA vector for expression of PAH protein as described herein comprising a kill switch. A kill switch as disclosed herein enables a cell comprising the ceDNA vector to be killed or undergo programmed cell death as a means to permanently remove an introduced ceDNA vector from the subject's system. It will be appreciated by one of ordinary skill in the art that use of kill switches in the ceDNA vectors for expression of PAH protein would be typically coupled with targeting of the ceDNA vector to a limited number of cells that the subject can acceptably lose or to a cell type where apoptosis is desirable (e.g., cancer cells). In all aspects, a “kill switch” as disclosed herein is designed to provide rapid and robust cell killing of the cell comprising the ceDNA vector in the absence of an input survival signal or other specified condition. Stated another way, a kill switch encoded by a ceDNA vector for expression of PAH protein as described herein can restrict cell survival of a cell comprising a ceDNA vector to an environment defined by specific input signals. Such kill switches serve as a biological biocontainment function should it be desirable to remove the ceDNA vector e expression of PAH protein in a subject or to ensure that it will not express the encoded PAH protein.

Other kill switches known to a person of ordinary skill in the art are encompassed for use in the ceDNA vector for expression of PAH protein as disclosed herein, e.g., as disclosed in US2010/0175141; US2013/0009799; US2011/0172826; US2013/0109568, as well as kill switches disclosed in Jusiak et al, Reviews in Cell Biology and molecular Medicine; 2014; 1-56; Kobayashi et al., PNAS, 2004; 101; 8419-9; Marchisio et al., Int. Journal of Biochem and Cell Biol., 2011; 43; 310-319; and in Reinshagen et al., Science Translational Medicine, 2018, 11, the contents of all of which are incorporated by reference in their entireties herein.

Accordingly, in some embodiments, the ceDNA vector for expression of PAH protein can comprise a kill switch nucleic acid construct, which comprises the nucleic acid encoding an effector toxin or reporter protein, where the expression of the effector toxin (e.g., a death protein) or reporter protein is controlled by a predetermined condition. For example, a predetermined condition can be the presence of an environmental agent, such as, e.g., an exogenous agent, without which the cell will default to expression of the effector toxin (e.g., a death protein) and be killed. In alternative embodiments, a predetermined condition is the presence of two or more environmental agents, e.g., the cell will only survive when two or more necessary exogenous agents are supplied, and without either of which, the cell comprising the ceDNA vector is killed.

In some embodiments, the ceDNA vector for expression of PAH protein is modified to incorporate a kill-switch to destroy the cells comprising the ceDNA vector to effectively terminate the in vivo expression of the transgene being expressed by the ceDNA vector (e.g., expression of PAH protein). Specifically, the ceDNA vector is further genetically engineered to express a switch-protein that is not functional in mammalian cells under normal physiological conditions. Only upon administration of a drug or environmental condition that specifically targets this switch-protein, the cells expressing the switch-protein will be destroyed thereby terminating the expression of the therapeutic protein or peptide. For instance, it was reported that cells expressing HSV-thymidine kinase can be killed upon administration of drugs, such as ganciclovir and cytosine deaminase. See, for example, Dey and Evans, Suicide Gene Therapy by Herpes Simplex Virus-1 Thymidine Kinase (HSV-TK), in Targets in Gene Therapy, edited by You (2011); and Beltinger et al., Proc. Natl. Acad. Sci. USA 96(15):8699-8704 (1999). In some embodiments the ceDNA vector can comprise a siRNA kill switch referred to as DISE (Death Induced by Survival gene Elimination) (Murmann et al., Oncotarget. 2017; 8:84643-84658. Induction of DISE in ovarian cancer cells in vivo).

### VI. Method of Production of a ceDNA Vector

### General Methods of Production

Certain methods for the production of a ceDNA vector for expression of PAH protein comprising an asymmetrical ITR pair or symmetrical ITR pair as defined herein is described in section IV of International application PCT/US18/49996 filed Sep. 7, 2018, which is incorporated herein in its entirety by reference. In some embodiments, a ceDNA vector for expression of PAH protein as disclosed herein can be produced using insect cells, as described herein. In alternative embodiments, a ceDNA vector for expression of PAH protein as disclosed herein can be produced synthetically and in some embodiments, in a cell-free method, as disclosed on International Application PCT/US19/14122, filed Jan. 18, 2019, which is incorporated herein in its entirety by reference.

As described herein, in one embodiment, a ceDNA vector for expression of PAH protein can be obtained, for example, by the process comprising the steps of: a) incubating a population of host cells (e.g. insect cells) harboring the polynucleotide expression construct template (e.g., a ceDNA-plasmid, a ceDNA-Bacmid, and/or a ceDNA-baculovirus), which is devoid of viral capsid coding sequences, in the presence of a Rep protein under conditions effective and for a time sufficient to induce production of the ceDNA vector within the host cells, and wherein the host cells do not comprise viral capsid coding sequences; and b) harvesting and isolating the ceDNA vector from the host cells. The presence of Rep protein induces replication of the vector polynucleotide with a modified ITR to produce the ceDNA vector in a host cell. However, no viral particles (e.g. AAV virions) are expressed. Thus, there is no size limitation such as that naturally imposed in AAV or other viral-based vectors.

The presence of the ceDNA vector isolated from the host cells can be confirmed by digesting DNA isolated from the host cell with a restriction enzyme having a single recognition site on the ceDNA vector and analyzing the digested DNA material on a non-denaturing gel to confirm the presence of characteristic bands of linear and continuous DNA as compared to linear and non-continuous DNA.

In yet another aspect, the disclosure provides for use of host cell lines that have stably integrated the DNA vector polynucleotide expression template (ceDNA template) into their own genome in production of the non-viral DNA vector, e.g., as described in Lee, L. et al. (2013) Plos One 8(8): e69879. Preferably, Rep is added to host cells at an MOI of about 3. When the host cell line is a mammalian cell line, e.g., HEK293 cells, the cell lines can have polynucleotide vector template stably integrated, and a second vector such as herpes virus can be used to introduce Rep protein into cells, allowing for the excision and amplification of ceDNA in the presence of Rep and helper virus.

In one embodiment, the host cells used to make the ceDNA vectors for expression of PAH protein as described herein are insect cells, and baculovirus is used to deliver both the polynucleotide that encodes Rep protein and the non-viral DNA vector polynucleotide expression construct template for ceDNA, e.g., as described in FIGS. 3A-3C and Example 1. In some embodiments, the host cell is engineered to express Rep protein.

The ceDNA vector is then harvested and isolated from the host cells. The time for harvesting and collecting ceDNA vectors described herein from the cells can be selected and optimized to achieve a high-yield production of the ceDNA vectors. For example, the harvest time can be selected in view of cell viability, cell morphology, cell growth, etc. In one embodiment, cells are grown under sufficient conditions and harvested a sufficient time after baculoviral infection to produce ceDNA vectors but before a majority of cells start to die because of the baculoviral toxicity. The DNA vectors can be isolated using plasmid purification kits such as Qiagen Endo-Free Plasmid kits. Other methods developed for plasmid isolation can be also adapted for DNA vectors. Generally, any nucleic acid purification methods can be adopted.

The DNA vectors can be purified by any means known to those of skill in the art for purification of DNA. In one embodiment, ceDNA vectors are purified as DNA molecules. In another embodiment, the ceDNA vectors are purified as exosomes or microparticles.

The presence of the ceDNA vector for expression of PAH protein can be confirmed by digesting the vector DNA isolated from the cells with a restriction enzyme having a single recognition site on the DNA vector and analyzing both digested and undigested DNA material using gel electrophoresis to confirm the presence of characteristic bands of linear and continuous DNA as compared to linear and non-continuous DNA. FIG. 3C and FIG. 3D illustrate one embodiment for identifying the presence of the closed ended ceDNA vectors produced by the processes herein.

### VII. Pharmaceutical Compositions

In another aspect, pharmaceutical compositions are provided. The pharmaceutical composition comprises a ceDNA vector for expression of PAH protein as described herein and a pharmaceutically acceptable carrier or diluent.

The ceDNA vectors for expression of PAH protein as disclosed herein can be incorporated into pharmaceutical compositions suitable for administration to a subject for in vivo delivery to cells, tissues, or organs of the subject. Typically, the pharmaceutical composition comprises a ceDNA-vector as disclosed herein and a pharmaceutically acceptable carrier. For example, the ceDNA vectors for expression of PAH protein as described herein can be incorporated into a pharmaceutical composition suitable for a desired route of therapeutic administration (e.g., parenteral administration). Passive tissue transduction via high pressure intravenous or intra-arterial infusion, as well as intracellular injection, such as intranuclear microinjection or intracytoplasmic injection, are also contemplated. Pharmaceutical compositions for therapeutic purposes can be formulated as a solution, microemulsion, dispersion, liposomes, or other ordered structure suitable to high ceDNA vector concentration. Sterile injectable solutions can be prepared by incorporating the ceDNA vector compound in the required amount in an appropriate buffer with one or a combination of ingredients enumerated above, as required, followed by filtered sterilization including a ceDNA vector can be formulated to deliver a transgene in the nucleic acid to the cells of a recipient, resulting in the therapeutic expression of the transgene or donor sequence therein. The composition can also include a pharmaceutically acceptable carrier.

Pharmaceutically active compositions comprising a ceDNA vector for expression of PAH protein can be formulated to deliver a transgene for various purposes to the cell, e.g., cells of a subject.

Pharmaceutical compositions for therapeutic purposes typically must be sterile and stable under the conditions of manufacture and storage. The composition can be formulated as a solution, microemulsion, dispersion, liposomes, or other ordered structure suitable to high ceDNA vector concentration. Sterile injectable solutions can be prepared by incorporating the ceDNA vector compound in the required amount in an appropriate buffer with one or a combination of ingredients enumerated above, as required, followed by filtered sterilization.

A ceDNA vector for expression of PAH protein as disclosed herein can be incorporated into a pharmaceutical composition suitable for topical, systemic, intra-amniotic, intrathecal, intracranial, intra-arterial, intravenous, intralymphatic, intraperitoneal, subcutaneous, tracheal, intra-tissue (e.g., intramuscular, intracardiac, intrahepatic, intrarenal, intracerebral), intrathecal, intravesical, conjunctival (e.g., extra-orbital, intraorbital, retroorbital, intraretinal, subretinal, choroidal, sub-choroidal, intrastromal, intracameral and intravitreal), intracochlear, and mucosal (e.g., oral, rectal, nasal) administration. Passive tissue transduction via high pressure intravenous or intraarterial infusion, as well as intracellular injection, such as intranuclear microinjection or intracytoplasmic injection, are also contemplated.

In some aspects, the methods provided herein comprise delivering one or more ceDNA vectors for expression of PAH protein as disclosed herein to a host cell. Also provided herein are cells produced by such methods, and organisms (such as animals, plants, or fungi) comprising or produced from such cells. Methods of delivery of nucleic acids can include lipofection, nucleofection, microinjection, biolistics, liposomes, immunoliposomes, polycation or lipid:nucleic acid conjugates, naked DNA, and agent-enhanced uptake of DNA. Lipofection is described in e.g., U.S. Pat. Nos. 5,049,386, 4,946,787; and 4,897,355, incorporated by reference in their entireties herein) and lipofection reagents are sold commercially (e.g., Transfectam™ and Lipofectin™). Delivery can be to cells (e.g., in vitro or ex vivo administration) or target tissues (e.g., in vivo administration).

Various techniques and methods are known in the art for delivering nucleic acids to cells. For example, nucleic acids, such as ceDNA for expression of PAH protein can be formulated into lipid nanoparticles (LNPs), lipidoids, liposomes, lipid nanoparticles, lipoplexes, or core-shell nanoparticles. Typically, LNPs are composed of nucleic acid (e.g., ceDNA) molecules, one or more ionizable or cationic lipids (or salts thereof), one or more non-ionic or neutral lipids (e.g., a phospholipid), a molecule that prevents aggregation (e.g., PEG or a PEG-lipid conjugate), and optionally a sterol (e.g., cholesterol).

Another method for delivering nucleic acids, such as ceDNA for expression of PAH protein to a cell is by conjugating the nucleic acid with a ligand that is internalized by the cell. For example, the ligand can bind a receptor on the cell surface and internalized via endocytosis. The ligand can be covalently linked to a nucleotide in the nucleic acid. Exemplary conjugates for delivering nucleic acids into a cell are described, example, in International Patent Application Publication Nos. WO2015/006740, WO2014/025805, WO2012/037254, WO2009/082606, WO2009/073809, WO2009/018332, WO2006/112872, WO2004/090108, WO2004/091515 and WO2017/177326, the contents of all of which are incorporated by reference in their entireties herein.

Nucleic acids, such as ceDNA vectors for expression of PAH protein can also be delivered to a cell by transfection. Useful transfection methods include, but are not limited to, lipid-mediated transfection, cationic polymer-mediated transfection, or calcium phosphate precipitation. Transfection reagents are well known in the art and include, but are not limited to, TurboFect Transfection Reagent (Thermo Fisher Scientific), Pro-Ject Reagent (Thermo Fisher Scientific), TRANSPASS™ P Protein Transfection Reagent (New England Biolabs), CHARIOT™ Protein Delivery Reagent (Active Motif), PROTEOJUICE™ Protein Transfection Reagent (EMD Millipore), 293fectin, LIPOFECTAMINE™ 2000, LIPOFECTAMINE™ 3000 (Thermo Fisher Scientific), LIPOFECTAMINE™ (Thermo Fisher Scientific), LIPOFECTIN™ (Thermo Fisher Scientific), DMRIE-C, CELLFECTIN™ (Thermo Fisher Scientific), OLIGOFECTAMINE™ (Thermo Fisher Scientific), LIPOFECTACE™, FUGENE™ (Roche, Basel, Switzerland), FUGENE™ HD (Roche), TRANSFECTAM™ (Transfectam, Promega, Madison, Wis.), TFX-10™ (Promega), TFX-20™ (Promega), TFX-50™ (Promega), TRANSFECTIN™ (BioRad, Hercules, Calif.), SILENTFECT™ (Bio-Rad), Effectene™ (Qiagen, Valencia, Calif.), DC-chol (Avanti Polar Lipids), GENEPORTER™ (Gene Therapy Systems, San Diego, Calif.), DHARMAFECT 1™ (Dharmacon, Lafayette, Colo.), DHARMAFECT 2™ (Dharmacon), DHARMAFECT 3™ (Dharmacon), DHARMAFECT 4™ (Dharmacon), ESCORT™ III (Sigma, St. Louis, Mo.), and ESCORT™ IV (Sigma Chemical Co.). Nucleic acids, such as ceDNA, can also be delivered to a cell via microfluidics methods known to those of skill in the art.

ceDNA vectors for expression of PAH protein as described herein can also be administered directly to an organism for transduction of cells in vivo. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Methods for introduction of a nucleic acid vector ceDNA vector for expression of PAH protein as disclosed herein can be delivered into hematopoietic stem cells, for example, by the methods as described, for example, in U.S. Pat. No. 5,928,638, incorporated by reference in its entirety herein.

The ceDNA vectors for expression of PAH protein in accordance with the present disclosure can be added to liposomes for delivery to a cell or target organ in a subject. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids. Exemplary liposomes and liposome formulations, including but not limited to polyethylene glycol (PEG)-functional group containing compounds are disclosed in International Application PCT/US2018/050042, filed on Sep. 7, 2018 and in International application PCT/US2018/064242, filed on Dec. 6, 2018, e.g., see the section entitled “Pharmaceutical Formulations”, the contents of each of which are incorporated by reference in their entireties herein.

Various delivery methods known in the art or modification thereof can be used to deliver ceDNA vectors in vitro or in vivo. For example, in some embodiments, ceDNA vectors for expression of PAH protein are delivered by making transient penetration in cell membrane by mechanical, electrical, ultrasonic, hydrodynamic, or laser-based energy so that DNA entrance into the targeted cells is facilitated. For example, a ceDNA vector can be delivered by transiently disrupting cell membrane by squeezing the cell through a size-restricted channel or by other means known in the art. In some cases, a ceDNA vector alone is directly injected as naked DNA into any one of: any one or more tissues selected from: liver, kidneys, gallbladder, prostate, adrenal gland, heart, intestine, lung, and stomach, skin, thymus, cardiac muscle or skeletal muscle. In some cases, a ceDNA vector is delivered by gene gun. Gold or tungsten spherical particles (1-3 m diameter) coated with capsid-free AAV vectors can be accelerated to high speed by pressurized gas to penetrate into target tissue cells.

Compositions comprising a ceDNA vector for expression of PAH protein and a pharmaceutically acceptable carrier are specifically contemplated herein. In some embodiments, the ceDNA vector is formulated with a lipid delivery system, for example, liposomes as described herein. In some embodiments, such compositions are administered by any route desired by a skilled practitioner. The compositions may be administered to a subject by different routes including orally, parenterally, sublingually, transdermally, rectally, transmucosally, topically, via inhalation, via buccal administration, intrapleurally, intravenous, intra-arterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian may readily determine the dosing regimen and route of administration that is most appropriate for a particular animal. The compositions may be administered by traditional syringes, needleless injection devices, “microprojectile bombardment gene guns”, or other physical methods such as electroporation (“EP”), hydrodynamic methods, or ultrasound.

In some cases, a ceDNA vector for expression of PAH protein is delivered by hydrodynamic injection, which is a simple and highly efficient method for direct intracellular delivery of any water-soluble compounds and particles into internal organs and skeletal muscle in an entire limb.

In some cases, ceDNA vectors for expression of PAH protein are delivered by ultrasound by making nanoscopic pores in membrane to facilitate intracellular delivery of DNA particles into cells of internal organs or tumors, so the size and concentration of plasmid DNA have great role in efficiency of the system. In some cases, ceDNA vectors are delivered by magnetofection by using magnetic fields to concentrate particles containing nucleic acid into the target cells.

In some cases, chemical delivery systems can be used, for example, by using nanomeric complexes, which include compaction of negatively charged nucleic acid by polycationic nanomeric particles, belonging to cationic liposome/micelle or cationic polymers. Cationic lipids used for the delivery method includes, but not limited to monovalent cationic lipids, polyvalent cationic lipids, guanidine containing compounds, cholesterol derivative compounds, cationic polymers, (e.g., poly(ethylenimine), poly-L-lysine, protamine, other cationic polymers), and lipid-polymer hybrid.

### A. Exosomes:

In some embodiments, a ceDNA vector for expression of PAH protein as disclosed herein is delivered by being packaged in an exosome. Exosomes are small membrane vesicles of endocytic origin that are released into the extracellular environment following fusion of multivesicular bodies with the plasma membrane. Their surface consists of a lipid bilayer from the donor cell's cell membrane, they contain cytosol from the cell that produced the exosome, and exhibit membrane proteins from the parental cell on the surface. Exosomes are produced by various cell types including epithelial cells, B and T lymphocytes, mast cells (MC) as well as dendritic cells (DC). Some embodiments, exosomes with a diameter between 10 nm and 1 m, between 20 nm and 500 nm, between 30 nm and 250 nm, between 50 nm and 100 nm are envisioned for use. Exosomes can be isolated for a delivery to target cells using either their donor cells or by introducing specific nucleic acids into them. Various approaches known in the art can be used to produce exosomes containing capsid-free AAV vectors of the present disclosure.

### A. Microparticle/Nanoparticles

In some embodiments, a ceDNA vector for expression of PAH protein as disclosed herein is delivered by a lipid nanoparticle. Generally, lipid nanoparticles comprise an ionizable amino lipid (e.g., heptatriaconta-6,9,28,31-tetraen-19-yl 4-(dimethylamino)butanoate, DLin-MC3-DMA, a phosphatidylcholine (1,2-distearoyl-sn-glycero-3-phosphocholine, DSPC), cholesterol and a coat lipid (polyethylene glycol-dimyristolglycerol, PEG-DMG), for example as disclosed by Tam et al. (2013). Advances in Lipid Nanoparticles for siRNA delivery. Pharmaceuticals 5(3): 498-507.

In some embodiments, a lipid nanoparticle has a mean diameter between about 10 and about 1000 nm. In some embodiments, a lipid nanoparticle has a diameter that is less than 300 nm. In some embodiments, a lipid nanoparticle has a diameter between about 10 and about 300 nm. In some embodiments, a lipid nanoparticle has a diameter that is less than 200 nm. In some embodiments, a lipid nanoparticle has a diameter between about 25 and about 200 nm. In some embodiments, a lipid nanoparticle preparation (e.g., composition comprising a plurality of lipid nanoparticles) has a size distribution in which the mean size (e.g., diameter) is about 70 nm to about 200 nm, and more typically the mean size is about 100 nm or less.

Various lipid nanoparticles known in the art can be used to deliver ceDNA vector for expression of PAH protein as disclosed herein. For example, various delivery methods using lipid nanoparticles are described in U.S. Pat. Nos. 9,404,127, 9,006,417 and 9,518,272.

In some embodiments, a ceDNA vector for expression of PAH protein as disclosed herein is delivered by a gold nanoparticle. Generally, a nucleic acid can be covalently bound to a gold nanoparticle or non-covalently bound to a gold nanoparticle (e.g., bound by a charge-charge interaction), for example as described by Ding et al. (2014). Gold Nanoparticles for Nucleic Acid Delivery. Mol. Ther. 22(6); 1075-1083. In some embodiments, gold nanoparticle-nucleic acid conjugates are produced using methods described, for example, in U.S. Pat. No. 6,812,334.

### B. Conjugates

In some embodiments, a ceDNA vector for expression of PAH protein as disclosed herein is conjugated (e.g., covalently bound to an agent that increases cellular uptake. An “agent that increases cellular uptake” is a molecule that facilitates transport of a nucleic acid across a lipid membrane. For example, a nucleic acid can be conjugated to a lipophilic compound (e.g., cholesterol, tocopherol, etc.), a cell penetrating peptide (CPP) (e.g., penetratin, TAT, Syn1B, etc.), and polyamines (e.g., spermine). Further examples of agents that increase cellular uptake are disclosed, for example, in Winkler (2013). Oligonucleotide conjugates for therapeutic applications. Ther. Deliv. 4(7); 791-809.

In some embodiments, a ceDNA vector for expression of PAH protein as disclosed herein is conjugated to a polymer (e.g., a polymeric molecule) or a folate molecule (e.g., folic acid molecule). Generally, delivery of nucleic acids conjugated to polymers is known in the art, for example as described in WO2000/34343 and WO2008/022309. In some embodiments, a ceDNA vector for expression of PAH protein as disclosed herein is conjugated to a poly(amide) polymer, for example as described by U.S. Pat. No. 8,987,377. In some embodiments, a nucleic acid described by the disclosure is conjugated to a folic acid molecule as described in U.S. Pat. No. 8,507,455.

In some embodiments, a ceDNA vector for expression of PAH protein as disclosed herein is conjugated to a carbohydrate, for example as described in U.S. Pat. No. 8,450,467.

### C. Nanocapsule

Alternatively, nanocapsule formulations of a ceDNA vector for expression of PAH protein as disclosed herein can be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

### D. Liposomes

The ceDNA vectors for expression of PAH protein in accordance with the present disclosure can be added to liposomes for delivery to a cell or target organ in a subject. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids.

The formation and use of liposomes is generally known to those of skill in the art. Liposomes have been developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587, incorporated by reference in their entireties herein).

### E. Exemplary Liposome and Lipid Nanoparticle (LNP) Compositions

The ceDNA vectors for expression of PAH protein in accordance with the present disclosure can be added to liposomes for delivery to a cell, e.g., a cell in need of expression of the transgene. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids.

Lipid nanoparticles (LNPs) comprising ceDNA vectors are disclosed in International Application PCT/US2018/050042, filed on Sep. 7, 2018, and International Application PCT/US2018/064242, filed on Dec. 6, 2018 which are incorporated herein in their entirety and envisioned for use in the methods and compositions for ceDNA vectors for expression of PAH protein as disclosed herein.

In some aspects, the disclosure provides for a liposome formulation that includes one or more compounds with a polyethylene glycol (PEG) functional group (so-called “PEG-ylated compounds”) which can reduce the immunogenicity/antigenicity of, provide hydrophilicity and hydrophobicity to the compound(s) and reduce dosage frequency. Or the liposome formulation simply includes polyethylene glycol (PEG) polymer as an additional component. In such aspects, the molecular weight of the PEG or PEG functional group can be from 62 Da to about 5,000 Da.

In some aspects, the disclosure provides for a liposome formulation that will deliver an API with extended release or controlled release profile over a period of hours to weeks. In some related aspects, the liposome formulation may comprise aqueous chambers that are bound by lipid bilayers. In other related aspects, the liposome formulation encapsulates an API with components that undergo a physical transition at elevated temperature which releases the API over a period of hours to weeks.

In some aspects, the liposome formulation comprises sphingomyelin and one or more lipids disclosed herein. In some aspects, the liposome formulation comprises optisomes.

In some aspects, the disclosure provides for a liposome formulation that includes one or more lipids selected from: N-(carbonyl-methoxypolyethylene glycol 2000)-1,2-distearoyl-sn-glycero-3-phosphoethanolamine sodium salt, (distearoyl-sn-glycero-phosphoethanolamine), MPEG (methoxy polyethylene glycol)-conjugated lipid, HSPC (hydrogenated soy phosphatidylcholine); PEG (polyethylene glycol); DSPE (distearoyl-sn-glycero-phosphoethanolamine); DSPC (distearoylphosphatidylcholine); DOPC (dioleoylphosphatidylcholine); DPPG (dipalmitoylphosphatidylglycerol); EPC (egg phosphatidylcholine); DOPS (dioleoylphosphatidylserine); POPC (palmitoyloleoylphosphatidylcholine); SM (sphingomyelin); MPEG (methoxy polyethylene glycol); DMPC (dimyristoyl phosphatidylcholine); DMPG (dimyristoyl phosphatidylglycerol); DSPG (distearoylphosphatidylglycerol); DEPC (dierucoylphosphatidylcholine); DOPE (dioleoly-sn-glycero-phophoethanolamine). cholesteryl sulphate (CS), dipalmitoylphosphatidylglycerol (DPPG), DOPC (dioleoly-sn-glycero-phosphatidylcholine) or any combination thereof.

In some aspects, the disclosure provides for a liposome formulation comprising phospholipid, cholesterol and a PEG-ylated lipid in a molar ratio of 56:38:5. In some aspects, the liposome formulation's overall lipid content is from 2-16 mg/mL. In some aspects, the disclosure provides for a liposome formulation comprising a lipid containing a phosphatidylcholine functional group, a lipid containing an ethanolamine functional group and a PEG-ylated lipid. In some aspects, the disclosure provides for a liposome formulation comprising a lipid containing a phosphatidylcholine functional group, a lipid containing an ethanolamine functional group and a PEG-ylated lipid in a molar ratio of 3:0.015:2 respectively. In some aspects, the disclosure provides for a liposome formulation comprising a lipid containing a phosphatidylcholine functional group, cholesterol and a PEG-ylated lipid. In some aspects, the disclosure provides for a liposome formulation comprising a lipid containing a phosphatidylcholine functional group and cholesterol. In some aspects, the PEG-ylated lipid is PEG-2000-DSPE. In some aspects, the disclosure provides for a liposome formulation comprising DPPG, soy PC, MPEG-DSPE lipid conjugate and cholesterol.

In some aspects, the disclosure provides for a liposome formulation comprising one or more lipids containing a phosphatidylcholine functional group and one or more lipids containing an ethanolamine functional group. In some aspects, the disclosure provides for a liposome formulation comprising one or more: lipids containing a phosphatidylcholine functional group, lipids containing an ethanolamine functional group, and sterols, e.g. cholesterol. In some aspects, the liposome formulation comprises DOPC/DEPC; and DOPE.

In some aspects, the disclosure provides for a liposome formulation further comprising one or more pharmaceutical excipients, e.g. sucrose and/or glycine.

In some aspects, the disclosure provides for a liposome formulation that is either unilamellar or multilamellar in structure. In some aspects, the disclosure provides for a liposome formulation that comprises multi-vesicular particles and/or foam-based particles. In some aspects, the disclosure provides for a liposome formulation that are larger in relative size to common nanoparticles and about 150 to 250 nm in size. In some aspects, the liposome formulation is a lyophilized powder.

In some aspects, the disclosure provides for a liposome formulation that is made and loaded with ceDNA vectors disclosed or described herein, by adding a weak base to a mixture having the isolated ceDNA outside the liposome. This addition increases the pH outside the liposomes to approximately 7.3 and drives the API into the liposome. In some aspects, the disclosure provides for a liposome formulation having a pH that is acidic on the inside of the liposome. In such cases the inside of the liposome can be at pH 4-6.9, and more preferably pH 6.5. In other aspects, the disclosure provides for a liposome formulation made by using intra-liposomal drug stabilization technology. In such cases, polymeric or non-polymeric highly charged anions and intra-liposomal trapping agents are utilized, e.g. polyphosphate or sucrose octasulfate.

In some aspects, the disclosure provides for a lipid nanoparticle comprising ceDNA and an ionizable lipid. For example, a lipid nanoparticle formulation that is made and loaded with ceDNA obtained by the process as disclosed in International Application PCT/US2018/050042, filed on Sep. 7, 2018, which is incorporated herein. This can be accomplished by high energy mixing of ethanolic lipids with aqueous ceDNA at low pH which protonates the ionizable lipid and provides favorable energetics for ceDNA/lipid association and nucleation of particles. The particles can be further stabilized through aqueous dilution and removal of the organic solvent. The particles can be concentrated to the desired level.

Generally, the lipid particles are prepared at a total lipid to ceDNA (mass or weight) ratio of from about 10:1 to 30:1. In some embodiments, the lipid to ceDNA ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and ceDNA can be adjusted to provide a desired N/P ratio, for example, N/P ratio of 3, 4, 5, 6, 7, 8, 9, 10 or higher. Generally, the lipid particle formulation's overall lipid content can range from about 5 mg/ml to about 30 mg/mL.

The ionizable lipid is typically employed to condense the nucleic acid cargo, e.g., ceDNA at low pH and to drive membrane association and fusogenicity. Generally, ionizable lipids are lipids comprising at least one amino group that is positively charged or becomes protonated under acidic conditions, for example at pH of 6.5 or lower. Ionizable lipids are also referred to as cationic lipids herein.

Exemplary ionizable lipids are described in International Patent Publication Nos. WO2015/095340, WO2015/199952, WO2018/011633, WO2017/049245, WO2015/061467, WO2012/040184, WO2012/000104, WO2015/074085, WO2016/081029, WO2017/004143, WO2017/075531, WO2017/117528, WO2011/022460, WO2013/148541, WO2013/116126, WO2011/153120, WO2012/044638, WO2012/054365, WO2011/090965, WO2013/016058, WO2012/162210, WO2008/042973, WO2010/129709, WO2010/144740, WO2012/099755, WO2013/049328, WO2013/086322, WO2013/086373, WO2011/071860, WO2009/132131, WO2010/048536, WO2010/088537, WO2010/054401, WO2010/054406, WO2010/054405, WO2010/054384, WO2012/016184, WO2009/086558, WO2010/042877, WO2011/000106, WO2011/000107, WO2005/120152, WO2011/141705, WO2013/126803, WO2006/007712, WO2011/038160, WO2005/121348, WO2011/066651, WO2009/127060, WO2011/141704, WO2006/069782, WO2012/031043, WO2013/006825, WO2013/033563, WO2013/089151, WO2017/099823, WO2015/095346, and WO2013/086354, and US Patent Publication Nos. US2016/0311759, US2015/0376115, US2016/0151284, US2017/0210697, US2015/0140070, US2013/0178541, US2013/0303587, US2015/0141678, US2015/0239926, US2016/0376224, US2017/0119904, US2012/0149894, US2015/0057373, US2013/0090372, US2013/0274523, US2013/0274504, US2013/0274504, US2009/0023673, US2012/0128760, US2010/0324120, US2014/0200257, US2015/0203446, US2018/0005363, US2014/0308304, US2013/0338210, US2012/0101148, US2012/0027796, US2012/0058144, US2013/0323269, US2011/0117125, US2011/0256175, US2012/0202871, US2011/0076335, US2006/0083780, US2013/0123338, US2015/0064242, US2006/0051405, US2013/0065939, US2006/0008910, US2003/0022649, US2010/0130588, US2013/0116307, US2010/0062967, US2013/0202684, US2014/0141070, US2014/0255472, US2014/0039032, US2018/0028664, US2016/0317458, and US2013/0195920, the contents of all of which are incorporated herein by reference in their entireties.

In some embodiments, the ionizable lipid is MC3 (6Z,9Z,28Z,31Z)-heptatriaconta-6,9,28,31-tetraen-19-yl-4-(dimethylamino) butanoate (DLin-MC3-DMA or MC3) having the following structure:

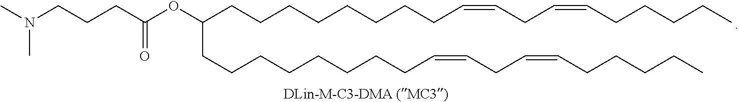 [SMILES: CCCCC/C=C\C/C=C\CCCCCCCCC(CCCCCCCC/C=C\C/C=C\CCCCC)OC(=O)CCCN(C)C]

The lipid DLin-MC3-DMA is described in Jayaraman et al., Angew. Chem. Int. Ed Engl. (2012), 51(34): 8529-8533, content of which is incorporated herein by reference in its entirety.

In some embodiments, the ionizable lipid is the lipid ATX-002 as described in WO2015/074085, content of which is incorporated herein by reference in its entirety.

In some embodiments, the ionizable lipid is (13Z,16Z)-N,N-dimethyl-3-nonyldocosa-13,16-dien-1-amine (Compound 32), as described in International Patent Application Publication No. WO2012/040184, content of which is incorporated herein by reference in its entirety.

In some embodiments, the ionizable lipid is Compound 6 or Compound 22 as described in International Patent Application Publication No. WO2015/199952, content of which is incorporated herein by reference in its entirety.

Without limitations, ionizable lipid can comprise 20-90% (mol) of the total lipid present in the lipid nanoparticle. For example, ionizable lipid molar content can be 20-70% (mol), 30-60% (mol) or 40-50% (mol) of the total lipid present in the lipid nanoparticle. In some embodiments, ionizable lipid comprises from about 50 mol % to about 90 mol % of the total lipid present in the lipid nanoparticle.

In some aspects, the lipid nanoparticle can further comprise a non-cationic lipid. Non-ionic lipids include amphipathic lipids, neutral lipids and anionic lipids. Accordingly, the non-cationic lipid can be a neutral uncharged, zwitterionic, or anionic lipid. Non-cationic lipids are typically employed to enhance fusogenicity.

Exemplary non-cationic lipids envisioned for use in the methods and compositions as disclosed herein are described in International Patent Application Nos. PCT/US2018/050042, filed on Sep. 7, 2018, and PCT/US2018/064242, filed on Dec. 6, 2018 which are each incorporated herein in its entirety. Exemplary non-cationic lipids are described in International Application Publication No. WO2017/099823 and US Patent Publication No. US2018/0028664, the contents of both of which are incorporated herein by reference in their entirety.

The non-cationic lipid can comprise 0-30% (mol) of the total lipid present in the lipid nanoparticle. For example, the non-cationic lipid content is 5-20% (mol) or 10-15% (mol) of the total lipid present in the lipid nanoparticle. In various embodiments, the molar ratio of ionizable lipid to the neutral lipid ranges from about 2:1 to about 8:1.

In some embodiments, the lipid nanoparticles do not comprise any phospholipids. In some aspects, the lipid nanoparticle can further comprise a component, such as a sterol, to provide membrane integrity.

One exemplary sterol that can be used in the lipid nanoparticle is cholesterol and derivatives thereof. Exemplary cholesterol derivatives are described in International Patent Application Publication No. WO2009/127060 and US Patent Application Publication No. US2010/0130588, contents of both of which are incorporated herein by reference in their entirety.

The component providing membrane integrity, such as a sterol, can comprise 0-50% (mol) of the total lipid present in the lipid nanoparticle. In some embodiments, such a component is 20-50% (mol) 30-40% (mol) of the total lipid content of the lipid nanoparticle.

In some aspects, the lipid nanoparticle can further comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid. Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di(tetradecanoyloxy)propyl-1-O-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in US Patent Nos. or Patent Application Publication Nos. U.S. Pat. Nos. 5,885,613, 6,287,591, US2003/0077829, US2003/0077829, US2005/0175682, US2008/0020058, US2011/0117125, US2010/0130588, US2016/0376224, and US2017/0119904, the contents of all of which are incorporated herein by reference in their entirety.

In some embodiments, a PEG-lipid is a compound as defined in Patent Application Publication No. US2018/0028664, the content of which is incorporated herein by reference in its entirety. In some embodiments, a PEG-lipid is disclosed in Patent Application Publication Nos. US2015/0376115 or US2016/0376224, the content of both of which is incorporated herein by reference in its entirety.

The PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some examples, the PEG-lipid can be selected from the group consisting of PEG-DMG, 1,2-dimyristoyl-sn-glycero-3-phosphoethanolamine-N-[methoxy(polyethylene glycol)-2000],

Lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (CPL) conjugates can be used in place of or in addition to the PEG-lipid. Exemplary conjugated lipids, i.e., PEG-lipids, (POZ)-lipid conjugates, ATTA-lipid conjugates and cationic polymer-lipids are described in the International Patent Application Publication Nos. WO1996/010392, WO1998/051278, WO2002/087541, WO2005/026372, WO2008/147438, WO2009/086558, WO2012/000104, WO2017/117528, WO2017/099823, WO2015/199952, WO2017/004143, WO2015/095346, WO2012/000104, WO2012/000104, and WO2010/006282, US Patent Application Publication Nos. US2003/0077829, US2005/0175682, US2008/0020058, US2011/0117125, US2013/0303587, US2018/0028664, US2015/0376115, US2016/0376224, US2016/0317458, US2013/0303587, US2013/0303587, and US20110123453, and U.S. Pat. Nos. 5,885,613, 6,287,591, 6,320,017, and 6,586,559, the contents of all of which are incorporated herein by reference in their entireties.

In some embodiments, the one or more additional compound can be a therapeutic agent. The therapeutic agent can be selected from any class suitable for the therapeutic objective. In other words, the therapeutic agent can be selected from any class suitable for the therapeutic objective. In other words, the therapeutic agent can be selected according to the treatment objective and biological action desired. For example, if the ceDNA within the LNP is useful for treating PKU, the additional compound can be an anti-PKU agent (e.g., a chemotherapeutic agent, or other PKU therapy (including, but not limited to, a small molecule or an antibody). In another example, if the LNP containing the ceDNA is useful for treating an infection, the additional compound can be an antimicrobial agent (e.g., an antibiotic or antiviral compound). In yet another example, if the LNP containing the ceDNA is useful for treating an immune disease or disorder, the additional compound can be a compound that modulates an immune response (e.g., an immunosuppressant, immunostimulatory compound, or compound modulating one or more specific immune pathways). In some embodiments, different cocktails of different lipid nanoparticles containing different compounds, such as a ceDNA encoding a different protein or a different compound, such as a therapeutic may be used in the compositions and methods of the disclosure.

In some embodiments, the additional compound is an immune modulating agent. For example, the additional compound is an immunosuppressant. In some embodiments, the additional compound is immune stimulatory agent. Also provided herein is a pharmaceutical composition comprising the lipid nanoparticle-encapsulated insect-cell produced, or a synthetically produced ceDNA vector for expression of PAH protein as described herein and a pharmaceutically acceptable carrier or excipient.

In some aspects, the disclosure provides for a lipid nanoparticle formulation further comprising one or more pharmaceutical excipients. In some embodiments, the lipid nanoparticle formulation further comprises sucrose, tris, trehalose and/or glycine.

The ceDNA vector can be complexed with the lipid portion of the particle or encapsulated in the lipid position of the lipid nanoparticle. In some embodiments, the ceDNA can be fully encapsulated in the lipid position of the lipid nanoparticle, thereby protecting it from degradation by a nuclease, e.g., in an aqueous solution. In some embodiments, the ceDNA in the lipid nanoparticle is not substantially degraded after exposure of the lipid nanoparticle to a nuclease at 37° C. for at least about 20, 30, 45, or 60 minutes. In some embodiments, the ceDNA in the lipid nanoparticle is not substantially degraded after incubation of the particle in serum at 37° C. for at least about 30, 45, or 60 minutes or at least about 2, 3, 4, 5, 6, 7, 8, 9, 10, 12, 14, 16, 18, 20, 22, 24, 26, 28, 30, 32, 34, or 36 hours.

In certain embodiments, the lipid nanoparticles are substantially non-toxic to a subject, e.g., to a mammal such as a human. In some aspects, the lipid nanoparticle formulation is a lyophilized powder.

In some embodiments, lipid nanoparticles are solid core particles that possess at least one lipid bilayer. In other embodiments, the lipid nanoparticles have a non-bilayer structure, i.e., a non-lamellar (i.e., non-bilayer) morphology. Without limitations, the non-bilayer morphology can include, for example, three dimensional tubes, rods, cubic symmetries, etc. For example, the morphology of the lipid nanoparticles (lamellar vs. non-lamellar) can readily be assessed and characterized using, e.g., Cryo-TEM analysis as described in US2010/0130588, the content of which is incorporated herein by reference in its entirety.

In some further embodiments, the lipid nanoparticles having a non-lamellar morphology are electron dense. In some aspects, the disclosure provides for a lipid nanoparticle that is either unilamellar or multilamellar in structure. In some aspects, the disclosure provides for a lipid nanoparticle formulation that comprises multi-vesicular particles and/or foam-based particles.

By controlling the composition and concentration of the lipid components, one can control the rate at which the lipid conjugate exchanges out of the lipid particle and, in turn, the rate at which the lipid nanoparticle becomes fusogenic. In addition, other variables including, e.g., pH, temperature, or ionic strength, can be used to vary and/or control the rate at which the lipid nanoparticle becomes fusogenic. Other methods which can be used to control the rate at which the lipid nanoparticle becomes fusogenic will be apparent to those of ordinary skill in the art based on this disclosure. It will also be apparent that by controlling the composition and concentration of the lipid conjugate, one can control the lipid particle size.

The pKa of formulated cationic lipids can be correlated with the effectiveness of the LNPs for delivery of nucleic acids (see Jayaraman et al, Angewandte Chemie, International Edition (2012), 51(34), 8529-8533; Semple et al, Nature Biotechnology 28, 172-176 (2010), both of which are incorporated by reference in their entirety). The preferred range of pKa is ˜5 to ˜7. The pKa of the cationic lipid can be determined in lipid nanoparticles using an assay based on fluorescence of 2-(p-toluidino)-6-napthalene sulfonic acid (TNS).

### VIII. Methods of Use

A ceDNA vector for expression of PAH protein as disclosed herein can also be used in a method for the delivery of a nucleic acid sequence of interest (e.g., encoding PAH protein) to a target cell (e.g., a host cell). The method may in particular be a method for delivering PAH protein to a cell of a subject in need thereof and treating PKU. The disclosure allows for the in vivo expression of PAH protein encoded in the ceDNA vector in a cell in a subject such that therapeutic effect of the expression of PAH protein occurs. These results are seen with both in vivo and in vitro modes of ceDNA vector delivery.

In addition, the disclosure provides a method for the delivery of PAH protein in a cell of a subject in need thereof, comprising multiple administrations of the ceDNA vector of the disclosure encoding said PAH protein. Since the ceDNA vector of the disclosure does not induce an immune response like that typically observed against encapsidated viral vectors, such a multiple administration strategy will likely have greater success in a ceDNA-based system. The ceDNA vector are administered in sufficient amounts to transfect the cells of a desired tissue and to provide sufficient levels of gene transfer and expression of the PAH protein without undue adverse effects. Conventional and pharmaceutically acceptable routes of administration include, but are not limited to, retinal administration (e.g., subretinal injection, suprachoroidal injection or intravitreal injection), intravenous (e.g., in a liposome formulation), direct delivery to the selected organ (e.g., any one or more tissues selected from: liver, kidneys, gallbladder, prostate, adrenal gland, heart, intestine, lung, and stomach), intramuscular, and other parental routes of administration. Routes of administration may be combined, if desired.

Delivery of a ceDNA vector for expression of PAH protein as described herein is not limited to delivery of the expressed PAH protein. For example, conventionally produced (e.g., using a cell-based production method (e.g., insect-cell production methods) or synthetically produced ceDNA vectors as described herein may be used with other delivery systems provided to provide a portion of the gene therapy. One non-limiting example of a system that may be combined with the ceDNA vectors in accordance with the present disclosure includes systems which separately deliver one or more co-factors or immune suppressors for effective gene expression of the ceDNA vector expressing the PAH protein.

The disclosure also provides for a method of treating PKU in a subject comprising introducing into a target cell in need thereof (in particular a muscle cell or tissue) of the subject a therapeutically effective amount of a ceDNA vector, optionally with a pharmaceutically acceptable carrier. While the ceDNA vector can be introduced in the presence of a carrier, such a carrier is not required. The ceDNA vector selected comprises a nucleic acid sequence encoding an PAH protein useful for treating PKU. In particular, the ceDNA vector may comprise a desired PAH protein sequence operably linked to control elements capable of directing transcription of the desired PAH protein encoded by the exogenous DNA sequence when introduced into the subject. The ceDNA vector can be administered via any suitable route as provided above, and elsewhere herein.

The compositions and vectors provided herein can be used to deliver an PAH protein for various purposes. In some embodiments, the transgene encodes an PAH protein that is intended to be used for research purposes, e.g., to create a somatic transgenic animal model harboring the transgene, e.g., to study the function of the PAH protein product. In another example, the transgene encodes an PAH protein that is intended to be used to create an animal model of PKU. In some embodiments, the encoded PAH protein is useful for the treatment or prevention of PKU states in a mammalian subject. The PAH protein can be transferred (e.g., expressed in) to a patient in a sufficient amount to treat PKU associated with reduced expression, lack of expression or dysfunction of the gene.

In principle, the expression cassette can include a nucleic acid or any transgene that encodes an PAH protein that is either reduced or absent due to a mutation or which conveys a therapeutic benefit when overexpressed is considered to be within the scope of the disclosure. Preferably, noninserted bacterial DNA is not present and preferably no bacterial DNA is present in the ceDNA compositions provided herein.

A ceDNA vector is not limited to one species of ceDNA vector. As such, in another aspect, multiple ceDNA vectors expressing different proteins or the same PAH protein but operatively linked to different promoters or cis-regulatory elements can be delivered simultaneously or sequentially to the target cell, tissue, organ, or subject. Therefore, this strategy can allow for the gene therapy or gene delivery of multiple proteins simultaneously. It is also possible to separate different portions of a PAH protein into separate ceDNA vectors (e.g., different domains and/or co-factors required for functionality of a PAH protein) which can be administered simultaneously or at different times, and can be separately regulatable, thereby adding an additional level of control of expression of a PAH protein. Delivery can also be performed multiple times and, importantly for gene therapy in the clinical setting, in subsequent increasing or decreasing doses, given the lack of an anti-capsid host immune response due to the absence of a viral capsid. It is anticipated that no anti-capsid response will occur as there is no capsid.

The disclosure also provides for a method of treating PKU in a subject comprising introducing into a target cell in need thereof (in particular a muscle cell or tissue) of the subject a therapeutically effective amount of a ceDNA vector as disclosed herein, optionally with a pharmaceutically acceptable carrier. While the ceDNA vector can be introduced in the presence of a carrier, such a carrier is not required. The ceDNA vector implemented comprises a nucleic acid sequence of interest useful for treating the PKU. In particular, the ceDNA vector may comprise a desired exogenous DNA sequence operably linked to control elements capable of directing transcription of the desired polypeptide, protein, or oligonucleotide encoded by the exogenous DNA sequence when introduced into the subject. The ceDNA vector can be administered via any suitable route as provided above, and elsewhere herein.

### IX. Methods of Delivering ceDNA Vectors for PAH Protein Production

In some embodiments, a ceDNA vector for expression of PAH protein can be delivered to a target cell in vitro or in vivo by various suitable methods. ceDNA vectors alone can be applied or injected. CeDNA vectors can be delivered to a cell without the help of a transfection reagent or other physical means. Alternatively, ceDNA vectors for expression of PAH protein can be delivered using any art-known transfection reagent or other art-known physical means that facilitates entry of DNA into a cell, e.g., liposomes, alcohols, polylysine-rich compounds, arginine-rich compounds, calcium phosphate, microvesicles, microinjection, electroporation and the like.

The ceDNA vectors for expression of PAH protein as disclosed herein can efficiently target cell and tissue-types that are normally difficult to transduce with conventional AAV virions using various delivery reagent.

One aspect of the technology described herein relates to a method of delivering an PAH protein to a cell. Typically, for in vivo and in vitro methods, a ceDNA vector for expression of PAH protein as disclosed herein may be introduced into the cell using the methods as disclosed herein, as well as other methods known in the art. A ceDNA vector for expression of PAH protein as disclosed herein are preferably administered to the cell in a biologically-effective amount. If the ceDNA vector is administered to a cell in vivo (e.g., to a subject), a biologically-effective amount of the ceDNA vector is an amount that is sufficient to result in transduction and expression of the PAH protein in a target cell.

Exemplary modes of administration of a ceDNA vector for expression of PAH protein as disclosed herein includes oral, rectal, transmucosal, intranasal, inhalation (e.g., via an aerosol), buccal (e.g., sublingual), vaginal, intrathecal, intraocular, transdermal, intraendothelial, in utero (or in ovo), parenteral (e.g., intravenous, subcutaneous, intradermal, intracranial, intramuscular [including administration to skeletal, diaphragm and/or cardiac muscle], intrapleural, intracerebral, and intraarticular). Administration can be systemically or direct delivery to the liver or elsewhere (e.g., any kidneys, gallbladder, prostate, adrenal gland, heart, intestine, lung, and stomach).

Administration can be topical (e.g., to both skin and mucosal surfaces, including airway surfaces, and transdermal administration), intralymphatic, and the like, as well as direct tissue or organ injection (e.g., but not limited to, liver, but also to eye, muscles, including skeletal muscle, cardiac muscle, diaphragm muscle, or brain).

Administration of the ceDNA vector can be to any site in a subject, including, without limitation, a site selected from the group consisting of the liver and/or also eyes, brain, a skeletal muscle, a smooth muscle, the heart, the diaphragm, the airway epithelium, the kidney, the spleen, the pancreas, the skin.

The most suitable route in any given case will depend on the nature and severity of the condition being treated, ameliorated, and/or prevented and on the nature of the particular ceDNA vector that is being used. Additionally, ceDNA permits one to administer more than one PAH protein in a single vector, or multiple ceDNA vectors (e.g. a ceDNA cocktail).

A. Intramuscular Administration of a ceDNA Vector

In some embodiments, a method of treating a disease in a subject comprises introducing into a target cell in need thereof (in particular a muscle cell or tissue) of the subject a therapeutically effective amount of a ceDNA vector encoding an PAH protein, optionally with a pharmaceutically acceptable carrier. In some embodiments, the ceDNA vector for expression of PAH protein is administered to a muscle tissue of a subject.

In some embodiments, administration of the ceDNA vector can be to any site in a subject, including, without limitation, a site selected from the group consisting of a skeletal muscle, a smooth muscle, the heart, the diaphragm, or muscles of the eye.

Administration of a ceDNA vector for expression of PAH protein as disclosed herein to a skeletal muscle according to the present disclosure includes but is not limited to administration to the skeletal muscle in the limbs (e.g., upper arm, lower arm, upper leg, and/or lower leg), back, neck, head (e.g., tongue), thorax, abdomen, pelvis/perineum, and/or digits. The ceDNA as disclosed herein vector can be delivered to skeletal muscle by intravenous administration, intra-arterial administration, intraperitoneal administration, limb perfusion, (optionally, isolated limb perfusion of a leg and/or arm; see, e.g. Arruda et al., (2005) Blood 105: 3458-3464), and/or direct intramuscular injection. In particular embodiments, the ceDNA vector as disclosed herein is administered to the liver, eye, a limb (arm and/or leg) of a subject (e.g., a subject with muscular dystrophy such as DMD) by limb perfusion, optionally isolated limb perfusion (e.g., by intravenous or intra-articular administration. In embodiments, the ceDNA vector as disclosed herein can be administered without employing “hydrodynamic” techniques.

For instance, tissue delivery (e.g., to retina) of conventional viral vectors is often enhanced by hydrodynamic techniques (e.g., intravenous/intravenous administration in a large volume), which increase pressure in the vasculature and facilitate the ability of the viral vector to cross the endothelial cell barrier. In particular embodiments, the ceDNA vectors described herein can be administered in the absence of hydrodynamic techniques such as high volume infusions and/or elevated intravascular pressure (e.g., greater than normal systolic pressure, for example, less than or equal to a 5%, 10%, 15%, 20%, 25% increase in intravascular pressure over normal systolic pressure). Such methods may reduce or avoid the side effects associated with hydrodynamic techniques such as edema, nerve damage and/or compartment syndrome.

Furthermore, a composition comprising a ceDNA vector for expression of PAH protein as disclosed herein that is administered to a skeletal muscle can be administered to a skeletal muscle in the limbs (e.g., upper arm, lower arm, upper leg, and/or lower leg), back, neck, head (e.g., tongue), thorax, abdomen, pelvis/perineum, and/or digits. Suitable skeletal muscles include but are not limited to abductor digiti minimi (in the hand), abductor digiti minimi (in the foot), abductor hallucis, abductor ossis metatarsi quinti, abductor pollicis brevis, abductor pollicis longus, adductor brevis, adductor hallucis, adductor longus, adductor magnus, adductor pollicis, anconeus, anterior scalene, articularis genus, biceps brachii, biceps femoris, brachialis, brachioradialis, buccinator, coracobrachialis, corrugator supercilii, deltoid, depressor anguli oris, depressor labii inferioris, digastric, dorsal interossei (in the hand), dorsal interossei (in the foot), extensor carpi radialis brevis, extensor carpi radialis longus, extensor carpi ulnaris, extensor digiti minimi, extensor digitorum, extensor digitorum brevis, extensor digitorum longus, extensor hallucis brevis, extensor hallucis longus, extensor indicis, extensor pollicis brevis, extensor pollicis longus, flexor carpi radialis, flexor carpi ulnaris, flexor digiti minimi brevis (in the hand), flexor digiti minimi brevis (in the foot), flexor digitorum brevis, flexor digitorum longus, flexor digitorum profundus, flexor digitorum superficialis, flexor hallucis brevis, flexor hallucis longus, flexor pollicis brevis, flexor pollicis longus, frontalis, gastrocnemius, geniohyoid, gluteus maximus, gluteus medius, gluteus minimus, gracilis, iliocostalis cervicis, iliocostalis lumborum, iliocostalis thoracis, illiacus, inferior gemellus, inferior oblique, inferior rectus, infraspinatus, interspinalis, intertransversi, lateral pterygoid, lateral rectus, latissimus dorsi, levator anguli oris, levator labii superioris, levator labii superioris alaeque nasi, levator palpebrae superioris, levator scapulae, long rotators, longissimus capitis, longissimus cervicis, longissimus thoracis, longus capitis, longus colli, lumbricals (in the hand), lumbricals (in the foot), masseter, medial pterygoid, medial rectus, middle scalene, multifidus, mylohyoid, obliquus capitis inferior, obliquus capitis superior, obturator externus, obturator internus, occipitalis, omohyoid, opponens digiti minimi, opponens pollicis, orbicularis oculi, orbicularis oris, palmar interossei, palmaris brevis, palmaris longus, pectineus, pectoralis major, pectoralis minor, peroneus brevis, peroneus longus, peroneus tertius, piriformis, plantar interossei, plantaris, platysma, popliteus, posterior scalene, pronator quadratus, pronator teres, psoas major, quadratus femoris, quadratus plantae, rectus capitis anterior, rectus capitis lateralis, rectus capitis posterior major, rectus capitis posterior minor, rectus femoris, rhomboid major, rhomboid minor, risorius, sartorius, scalenus minimus, semimembranosus, semispinalis capitis, semispinalis cervicis, semispinalis thoracis, semitendinosus, serratus anterior, short rotators, soleus, spinalis capitis, spinalis cervicis, spinalis thoracis, splenius capitis, splenius cervicis, sternocleidomastoid, sternohyoid, sternothyroid, stylohyoid, subclavius, subscapularis, superior gemellus, superior oblique, superior rectus, supinator, supraspinatus, temporalis, tensor fascia lata, teres major, teres minor, thoracis, thyrohyoid, tibialis anterior, tibialis posterior, trapezius, triceps brachii, vastus intermedius, vastus lateralis, vastus medialis, zygomaticus major, and zygomaticus minor, and any other suitable skeletal muscle as known in the art.

Administration of a ceDNA vector for expression of PAH protein as disclosed herein to diaphragm muscle can be by any suitable method including intravenous administration, intra-arterial administration, and/or intra-peritoneal administration. In some embodiments, delivery of an expressed transgene from the ceDNA vector to a target tissue can also be achieved by delivering a synthetic depot comprising the ceDNA vector, where a depot comprising the ceDNA vector is implanted into skeletal, smooth, cardiac and/or diaphragm muscle tissue or the muscle tissue can be contacted with a film or other matrix comprising the ceDNA vector as described herein. Such implantable matrices or substrates are described in U.S. Pat. No. 7,201,898, incorporated by reference in its entirety herein.

Administration of a ceDNA vector for expression of PAH protein as disclosed herein to cardiac muscle includes administration to the left atrium, right atrium, left ventricle, right ventricle and/or septum. The ceDNA vector as described herein can be delivered to cardiac muscle by intravenous administration, intra-arterial administration such as intra-aortic administration, direct cardiac injection (e.g., into left atrium, right atrium, left ventricle, right ventricle), and/or coronary artery perfusion.

Administration of a ceDNA vector for expression of PAH protein as disclosed herein to smooth muscle can be by any suitable method including intravenous administration, intra-arterial administration, and/or intra-peritoneal administration. In one embodiment, administration can be to endothelial cells present in, near, and/or on smooth muscle. Non-limiting examples of smooth muscles include the iris of the eye, bronchioles of the lung, laryngeal muscles (vocal cords), muscular layers of the stomach, esophagus, small and large intestine of the gastrointestinal tract, ureter, detrusor muscle of the urinary bladder, uterine myometrium, penis, or prostate gland.

In some embodiments, of a ceDNA vector for expression of PAH protein as disclosed herein is administered to skeletal muscle, diaphragm muscle and/or cardiac muscle. In representative embodiments, a ceDNA vector according to the present disclosure is used to treat and/or prevent disorders of skeletal, cardiac and/or diaphragm muscle.

Specifically, it is contemplated that a composition comprising a ceDNA vector for expression of PAH protein as disclosed herein can be delivered to one or more muscles of the eye (e.g., Lateral rectus, Medial rectus, Superior rectus, Inferior rectus, Superior oblique, Inferior oblique), facial muscles (e.g., Occipitofrontalis muscle, Temporoparietalis muscle, Procerus muscle, Nasalis muscle, Depressor septi nasi muscle, Orbicularis oculi muscle, Corrugator supercilii muscle, Depressor supercilii muscle, Auricular muscles, Orbicularis oris muscle, Depressor anguli oris muscle, Risorius, Zygomaticus major muscle, Zygomaticus minor muscle, Levator labii superioris, Levator labii superioris alaeque nasi muscle, Depressor labii inferioris muscle, Levator anguli oris, Buccinator muscle, Mentalis) or tongue muscles (e.g., genioglossus, hyoglossus, chondroglossus, styloglossus, palatoglossus, superior longitudinal muscle, inferior longitudinal muscle, the vertical muscle, and the transverse muscle).

(i) Intramuscular Injection:

In some embodiments, a composition comprising a ceDNA vector for expression of PAH protein as disclosed herein can be injected into one or more sites of a given muscle, for example, skeletal muscle (e.g., deltoid, vastus lateralis, ventrogluteal muscle of dorsogluteal muscle, or anterolateral thigh for infants) in a subject using a needle. The composition comprising ceDNA can be introduced to other subtypes of muscle cells. Non-limiting examples of muscle cell subtypes include skeletal muscle cells, cardiac muscle cells, smooth muscle cells and/or diaphragm muscle cells.

Methods for intramuscular injection are known to those of skill in the art and as such are not described in detail herein.

In some embodiments, intramuscular injection can be combined with electroporation, delivery pressure or the use of transfection reagents to enhance cellular uptake of the ceDNA vector.

(ii) Transfection Reagents

In some embodiments, a ceDNA vector for expression of PAH protein as disclosed herein is formulated in compositions comprising one or more transfection reagents to facilitate uptake of the vectors into myotubes or muscle tissue. Thus, in one embodiment, the nucleic acids described herein are administered to a muscle cell, myotube or muscle tissue by transfection using methods described elsewhere herein.
(iii) Electroporation

In certain embodiments, a ceDNA vector for expression of PAH protein as disclosed herein is administered in the absence of a carrier to facilitate entry of ceDNA into the cells, or in a physiologically inert pharmaceutically acceptable carrier (i.e., any carrier that does not improve or enhance uptake of the capsid free, non-viral vectors into the myotubes). In such embodiments, the uptake of the capsid free, non-viral vector can be facilitated by electroporation of the cell or tissue.

Cell membranes naturally resist the passage of extracellular into the cell cytoplasm. One method for temporarily reducing this resistance is “electroporation”, where electrical fields are used to create pores in cells without causing permanent damage to the cells. These pores are large enough to allow DNA vectors, pharmaceutical drugs, DNA, and other polar compounds to gain access to the interior of the cell. With time, the pores in the cell membrane close and the cell once again becomes impermeable.

Electroporation can be used in both in vitro and in vivo applications to introduce e.g., exogenous DNA into living cells. In vitro applications typically mix a sample of live cells with the composition comprising e.g., DNA. The cells are then placed between electrodes such as parallel plates and an electrical field is applied to the cell/composition mixture.

There are a number of methods for in vivo electroporation; electrodes can be provided in various configurations such as, for example, a caliper that grips the epidermis overlying a region of cells to be treated. Alternatively, needle-shaped electrodes may be inserted into the tissue, to access more deeply located cells. In either case, after the composition comprising e.g., nucleic acids are injected into the treatment region, the electrodes apply an electrical field to the region. In some electroporation applications, this electric field comprises a single square wave pulse on the order of 100 to 500 V/cm. of about 10 to 60 ms duration. Such a pulse may be generated, for example, in known applications of the Electro Square Porator T820, made by the BTX Division of Genetronics, Inc.

Typically, successful uptake of e.g., nucleic acids occurs only if the muscle is electrically stimulated immediately, or shortly after administration of the composition, for example, by injection into the muscle.

In certain embodiments, electroporation is achieved using pulses of electric fields or using low voltage/long pulse treatment regimens (e.g., using a square wave pulse electroporation system). Exemplary pulse generators capable of generating a pulsed electric field include, for example, the ECM600, which can generate an exponential wave form, and the ElectroSquarePorator (T820), which can generate a square wave form, both of which are available from BTX, a division of Genetronics, Inc. (San Diego, Calif.). Square wave electroporation systems deliver controlled electric pulses that rise quickly to a set voltage, stay at that level for a set length of time (pulse length), and then quickly drop to zero.

In some embodiments, a local anesthetic is administered, for example, by injection at the site of treatment to reduce pain that may be associated with electroporation of the tissue in the presence of a composition comprising a capsid free, non-viral vector as described herein. In addition, one of skill in the art will appreciate that a dose of the composition should be chosen that minimizes and/or prevents excessive tissue damage resulting in fibrosis, necrosis or inflammation of the muscle.

(iv) Delivery Pressure

In some embodiments, delivery of a ceDNA vector for expression of PAH protein as disclosed herein to muscle tissue is facilitated by delivery pressure, which uses a combination of large volumes and rapid injection into an artery supplying a limb (e.g., iliac artery). This mode of administration can be achieved through a variety of methods that involve infusing limb vasculature with a composition comprising a ceDNA vector, typically while the muscle is isolated from the systemic circulation using a tourniquet of vessel clamps. In one method, the composition is circulated through the limb vasculature to permit extravasation into the cells. In another method, the intravascular hydrodynamic pressure is increased to expand vascular beds and increase uptake of the ceDNA vector into the muscle cells or tissue. In one embodiment, the ceDNA composition is administered into an artery.

(v) Lipid Nanoparticle Compositions

In some embodiments, a ceDNA vector for expression of PAH protein as disclosed herein for intramuscular delivery are formulated in a composition comprising a liposome as described elsewhere herein.

(vi) Systemic Administration of a ceDNA Vector Targeted to Muscle Tissue

In some embodiments, a ceDNA vector for expression of PAH protein as disclosed herein is formulated to be targeted to the muscle via indirect delivery administration, where the ceDNA is transported to the muscle as opposed to the liver. Accordingly, the technology described herein encompasses indirect administration of compositions comprising a ceDNA vector for expression of PAH protein as disclosed herein to muscle tissue, for example, by systemic administration. Such compositions can be administered topically, intravenously (by bolus or continuous infusion), intracellular injection, intratissue injection, orally, by inhalation, intraperitoneally, subcutaneously, intracavity, and can be delivered by peristaltic means, if desired, or by other means known by those skilled in the art. The agent can be administered systemically, for example, by intravenous infusion, if so desired.

In some embodiments, uptake of a ceDNA vector for expression of PAH protein as disclosed herein into muscle cells/tissue is increased by using a targeting agent or moiety that preferentially directs the vector to muscle tissue. Thus, in some embodiments, a capsid free, ceDNA vector can be concentrated in muscle tissue as compared to the amount of capsid free ceDNA vectors present in other cells or tissues of the body.

In some embodiments, the composition comprising a ceDNA vector for expression of PAH protein as disclosed herein further comprises a targeting moiety to muscle cells. In other embodiments, the expressed gene product comprises a targeting moiety specific to the tissue in which it is desired to act. The targeting moiety can include any molecule, or complex of molecules, which is/are capable of targeting, interacting with, coupling with, and/or binding to an intracellular, cell surface, or extracellular biomarker of a cell or tissue. The biomarker can include, for example, a cellular protease, a kinase, a protein, a cell surface receptor, a lipid, and/or fatty acid. Other examples of biomarkers that the targeting moieties can target, interact with, couple with, and/or bind to include molecules associated with a particular disease. For example, the biomarkers can include cell surface receptors implicated in cancer development, such as epidermal growth factor receptor and transferrin receptor. The targeting moieties can include, but are not limited to, synthetic compounds, natural compounds or products, macromolecular entities, bioengineered molecules (e.g., polypeptides, lipids, polynucleotides, antibodies, antibody fragments), and small entities (e.g., small molecules, neurotransmitters, substrates, ligands, hormones and elemental compounds) that bind to molecules expressed in the target muscle tissue.

In certain embodiments, the targeting moiety may further comprise a receptor molecule, including, for example, receptors, which naturally recognize a specific desired molecule of a target cell. Such receptor molecules include receptors that have been modified to increase their specificity of interaction with a target molecule, receptors that have been modified to interact with a desired target molecule not naturally recognized by the receptor, and fragments of such receptors (see, e.g., Skerra, 2000, J. Molecular Recognition, 13:167-187). A preferred receptor is a chemokine receptor. Exemplary chemokine receptors have been described in, for example, Lapidot et al, 2002, Exp Hematol, 30:973-81 and Onuffer et al, 2002, Trends Pharmacol Sci, 23:459-67.

In other embodiments, the additional targeting moiety may comprise a ligand molecule, including, for example, ligands which naturally recognize a specific desired receptor of a target cell, such as a Transferrin (Tf) ligand. Such ligand molecules include ligands that have been modified to increase their specificity of interaction with a target receptor, ligands that have been modified to interact with a desired receptor not naturally recognized by the ligand, and fragments of such ligands.

In still other embodiments, the targeting moiety may comprise an aptamer. Aptamers are oligonucleotides that are selected to bind specifically to a desired molecular structure of the target cell. Aptamers typically are the products of an affinity selection process similar to the affinity selection of phage display (also known as in vitro molecular evolution). The process involves performing several tandem iterations of affinity separation, e.g., using a solid support to which the diseased immunogen is bound, followed by polymerase chain reaction (PCR) to amplify nucleic acids that bound to the immunogens. Each round of affinity separation thus enriches the nucleic acid population for molecules that successfully bind the desired immunogen. In this manner, a random pool of nucleic acids may be “educated” to yield aptamers that specifically bind target molecules. Aptamers typically are RNA, but may be DNA or analogs or derivatives thereof, such as, without limitation, peptide nucleic acids (PNAs) and phosphorothioate nucleic acids.

In some embodiments, the targeting moiety can comprise a photo-degradable ligand (i.e., a ‘caged’ ligand) that is released, for example, from a focused beam of light such that the capsid free, non-viral vectors or the gene product are targeted to a specific tissue.

It is also contemplated herein that the compositions be delivered to multiple sites in one or more muscles of the subject. That is, injections can be in at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100 injections sites. Such sites can be spread over the area of a single muscle or can be distributed among multiple muscles.

B. Administration of the ceDNA Vector for Expression of PAH Protein to Non-Muscle Locations

In another embodiment, a ceDNA vector for expression of PAH protein is administered to the liver. The ceDNA vector may also be administered to different regions of the eye such as the cornea and/or optic nerve The ceDNA vector may also be introduced into the spinal cord, brainstem (medulla oblongata, pons), midbrain (hypothalamus, thalamus, epithalamus, pituitary gland, substantia nigra, pineal gland), cerebellum, telencephalon (corpus striatum, cerebrum including the occipital, temporal, parietal and frontal lobes, cortex, basal ganglia, hippocampus and portaamygdala), limbic system, neocortex, corpus striatum, cerebrum, and inferior colliculus. The ceDNA vector may be delivered into the cerebrospinal fluid (e.g., by lumbar puncture). The ceDNA vector for expression of PAH protein may further be administered intravascularly to the CNS in situations in which the blood-brain barrier has been perturbed (e.g., brain tumor or cerebral infarct).

In some embodiments, the ceDNA vector for expression of PAH protein can be administered to the desired region(s) of the eye by any route known in the art, including but not limited to, intrathecal, intra-ocular, intracerebral, intraventricular, intravenous (e.g., in the presence of a sugar such as mannitol), intranasal, intra-aural, intra-ocular (e.g., intra-vitreous, sub-retinal, anterior chamber) and peri-ocular (e.g., sub-Tenon's region) delivery as well as intramuscular delivery with retrograde delivery to motor neurons.

In some embodiments, the ceDNA vector for expression of PAH protein is administered in a liquid formulation by direct injection (e.g., stereotactic injection) to the desired region or compartment in the CNS. In other embodiments, the ceDNA vector can be provided by topical application to the desired region or by intra-nasal administration of an aerosol formulation. Administration to the eye may be by topical application of liquid droplets. As a further alternative, the ceDNA vector can be administered as a solid, slow-release formulation (see, e.g., U.S. Pat. No. 7,201,898). In yet additional embodiments, the ceDNA vector can used for retrograde transport to treat, ameliorate, and/or prevent diseases and disorders involving motor neurons (e.g., amyotrophic lateral sclerosis (ALS); spinal muscular atrophy (SMA), etc.). For example, the ceDNA vector can be delivered to muscle tissue from which it can migrate into neurons.

### C. Ex Vivo Treatment

In some embodiments, cells are removed from a subject, a ceDNA vector for expression of PAH protein as disclosed herein is introduced therein, and the cells are then replaced back into the subject. Methods of removing cells from subject for treatment ex vivo, followed by introduction back into the subject are known in the art (see, e.g., U.S. Pat. No. 5,399,346; the disclosure of which is incorporated herein in its entirety). Alternatively, a ceDNA vector is introduced into cells from another subject, into cultured cells, or into cells from any other suitable source, and the cells are administered to a subject in need thereof.

Cells transduced with a ceDNA vector for expression of PAH protein as disclosed herein are preferably administered to the subject in a “therapeutically-effective amount” in combination with a pharmaceutical carrier. Those skilled in the art will appreciate that the therapeutic effects need not be complete or curative, as long as some benefit is provided to the subject.

In some embodiments, a ceDNA vector for expression of PAH protein as disclosed herein can encode an PAH protein as described herein (sometimes called a transgene or heterologous nucleotide sequence) that is to be produced in a cell in vitro, ex vivo, or in vivo. For example, in contrast to the use of the ceDNA vectors described herein in a method of treatment as discussed herein, in some embodiments a ceDNA vector for expression of PAH protein may be introduced into cultured cells and the expressed PAH protein isolated from the cells, e.g., for the production of antibodies and fusion proteins. In some embodiments, the cultured cells comprising a ceDNA vector for expression of PAH protein as disclosed herein can be used for commercial production of antibodies or fusion proteins, e.g., serving as a cell source for small or large scale biomanufacturing of antibodies or fusion proteins. In alternative embodiments, a ceDNA vector for expression of PAH protein as disclosed herein is introduced into cells in a host non-human subject, for in vivo production of antibodies or fusion proteins, including small scale production as well as for commercial large scale PAH protein production.

The ceDNA vectors for expression of PAH protein as disclosed herein can be used in both veterinary and medical applications. Suitable subjects for ex vivo gene delivery methods as described above include both avians (e.g., chickens, ducks, geese, quail, turkeys and pheasants) and mammals (e.g., humans, bovines, ovines, caprines, equines, felines, canines, and lagomorphs), with mammals being preferred. Human subjects are most preferred. Human subjects include neonates, infants, juveniles, and adults.

### D. Dose Ranges

Provided herein are methods of treatment comprising administering to the subject an effective amount of a composition comprising a ceDNA vector encoding an PAH protein as described herein. As will be appreciated by a skilled practitioner, the term “effective amount” refers to the amount of the ceDNA composition administered that results in expression of the PAH protein in a “therapeutically effective amount” for the treatment of PKU.

In vivo and/or in vitro assays can optionally be employed to help identify optimal dosage ranges for use. The precise dose to be employed in the formulation will also depend on the route of administration, and the seriousness of the condition, and should be decided according to the judgment of the person of ordinary skill in the art and each subject's circumstances. Effective doses can be extrapolated from dose-response curves derived from in vitro or animal model test systems, e.g.

A ceDNA vectors for expression of PAH protein as disclosed herein is administered in sufficient amounts to transfect the cells of a desired tissue and to provide sufficient levels of gene transfer and expression without undue adverse effects. Conventional and pharmaceutically acceptable routes of administration include, but are not limited to, those described above in the “Administration” section, such as direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration can be combined, if desired.

The dose of the amount of a ceDNA vectors for expression of PAH protein as disclosed herein required to achieve a particular “therapeutic effect,” will vary based on several factors including, but not limited to: the route of nucleic acid administration, the level of gene or RNA expression required to achieve a therapeutic effect, the specific disease or disorder being treated, and the stability of the gene(s), RNA product(s), or resulting expressed protein(s). One of skill in the art can readily determine a ceDNA vector dose range to treat a patient having a particular disease or disorder based on the aforementioned factors, as well as other factors that are well known in the art.

Dosage regime can be adjusted to provide the optimum therapeutic response. For example, the oligonucleotide can be repeatedly administered, e.g., several doses can be administered daily or the dose can be proportionally reduced as indicated by the exigencies of the therapeutic situation. One of ordinary skill in the art will readily be able to determine appropriate doses and schedules of administration of the subject oligonucleotides, whether the oligonucleotides are to be administered to cells or to subjects.

A “therapeutically effective dose” will fall in a relatively broad range that can be determined through clinical trials and will depend on the particular application (neural cells will require very small amounts, while systemic injection would require large amounts). For example, for direct in vivo injection into skeletal or cardiac muscle of a human subject, a therapeutically effective dose will be on the order of from about 1 μg to 100 g of the ceDNA vector. If exosomes or microparticles are used to deliver the ceDNA vector, then a therapeutically effective dose can be determined experimentally, but is expected to deliver from 1 μg to about 100 g of vector. Moreover, a therapeutically effective dose is an amount ceDNA vector that expresses a sufficient amount of the transgene to have an effect on the subject that results in a reduction in one or more symptoms of the disease, but does not result in significant off-target or significant adverse side effects. In one embodiment, a “therapeutically effective amount” is an amount of an expressed PAH protein that is sufficient to produce a statistically significant, measurable change in expression of PKU biomarker or reduction of a given disease symptom. Such effective amounts can be gauged in clinical trials as well as animal studies for a given ceDNA vector composition.

Formulation of pharmaceutically-acceptable excipients and carrier solutions is well-known to those of skill in the art, as is the development of suitable dosing and treatment regimens for using the particular compositions described herein in a variety of treatment regimens.

For in vitro transfection, an effective amount of a ceDNA vectors for expression of PAH protein as disclosed herein to be delivered to cells (1×10⁶ cells) will be on the order of 0.1 to 100 μg ceDNA vector, preferably 1 to 20 μg, and more preferably 1 to 15 μg or 8 to 10 μg. Larger ceDNA vectors will require higher doses. If exosomes or microparticles are used, an effective in vitro dose can be determined experimentally but would be intended to deliver generally the same amount of the ceDNA vector.

For the treatment of PKU, the appropriate dosage of a ceDNA vector that expresses an PAH protein as disclosed herein will depend on the specific type of disease to be treated, the type of a PAH protein, the severity and course of the PKU disease, previous therapy, the patient's clinical history and response to the antibody, and the discretion of the attending physician. The ceDNA vector encoding a PAH protein is suitably administered to the patient at one time or over a series of treatments. Various dosing schedules including, but not limited to, single or multiple administrations over various time-points, bolus administration, and pulse infusion are contemplated herein.

Depending on the type and severity of the disease, a ceDNA vector is administered in an amount that the encoded PAH protein is expressed at about 0.3 mg/kg to 100 mg/kg (e.g. 15 mg/kg-100 mg/kg, or any dosage within that range), by one or more separate administrations, or by continuous infusion. One typical daily dosage of the ceDNA vector is sufficient to result in the expression of the encoded PAH protein at a range from about 15 mg/kg to 100 mg/kg or more, depending on the factors mentioned above. One exemplary dose of the ceDNA vector is an amount sufficient to result in the expression of the encoded PAH protein as disclosed herein in a range from about 10 mg/kg to about 50 mg/kg. Thus, one or more doses of a ceDNA vector in an amount sufficient to result in the expression of the encoded PAH protein at about 0.5 mg/kg, 1 mg/kg, 1.5 mg/kg, 2.0 mg/kg, 3 mg/kg, 4.0 mg/kg, 5 mg/kg, 10 mg/kg, 15 mg/kg, 20 mg/kg, 25 mg/kg, 30 mg/kg, 35 mg/kg, 40 mg/kg, 50 mg/kg, 60 mg/kg, 70 mg/kg, 80 mg/kg, 90 mg/kg, or 100 mg/kg (or any combination thereof) may be administered to the patient. In some embodiments, the ceDNA vector is an amount sufficient to result in the expression of the encoded PAH protein for a total dose in the range of 50 mg to 2500 mg. An exemplary dose of a ceDNA vector is an amount sufficient to result in the total expression of the encoded PAH protein at about 50 mg, about 100 mg, 200 mg, 300 mg, 400 mg, about 500 mg, about 600 mg, about 700 mg, about 720 mg, about 1000 mg, about 1050 mg, about 1100 mg, about 1200 mg, about 1300 mg, about 1400 mg, about 1500 mg, about 1600 mg, about 1700 mg, about 1800 mg, about 1900 mg, about 2000 mg, about 2050 mg, about 2100 mg, about 2200 mg, about 2300 mg, about 2400 mg, or about 2500 mg (or any combination thereof). As the expression of the PAH protein from ceDNA vector can be carefully controlled by regulatory switches herein, or alternatively multiple dose of the ceDNA vector administered to the subject, the expression of the PAH protein from the ceDNA vector can be controlled in such a way that the doses of the expressed PAH protein may be administered intermittently, e.g. every week, every two weeks, every three weeks, every four weeks, every month, every two months, every three months, or every six months from the ceDNA vector. The progress of this therapy can be monitored by conventional techniques and assays.

In certain embodiments, a ceDNA vector is administered an amount sufficient to result in the expression of the encoded PAH protein at a dose of 15 mg/kg, 30 mg/kg, 40 mg/kg, 45 mg/kg, 50 mg/kg, 60 mg/kg or a flat dose, e.g., 300 mg, 500 mg, 700 mg, 800 mg, or higher. In some embodiments, the expression of the PAH protein from the ceDNA vector is controlled such that the PAH protein is expressed every day, every other day, every week, every 2 weeks or every 4 weeks for a period of time. In some embodiments, the expression of the PAH protein from the ceDNA vector is controlled such that the PAH protein is expressed every 2 weeks or every 4 weeks for a period of time. In certain embodiments, the period of time is 6 months, one year, eighteen months, two years, five years, ten years, 15 years, 20 years, or the lifetime of the patient.

Treatment can involve administration of a single dose or multiple doses. In some embodiments, more than one dose can be administered to a subject; in fact, multiple doses can be administered as needed, because the ceDNA vector elicits does not elicit an anti-capsid host immune response due to the absence of a viral capsid. As such, one of skill in the art can readily determine an appropriate number of doses. The number of doses administered can, for example, be on the order of 1-100, preferably 2-20 doses.

Without wishing to be bound by any particular theory, the lack of typical anti-viral immune response elicited by administration of a ceDNA vector as described by the disclosure (i.e., the absence of capsid components) allows the ceDNA vector for expression of PAH protein to be administered to a host on multiple occasions. In some embodiments, the number of occasions in which a nucleic acid, e.g., heterologous nucleic acid, is delivered to a subject is in a range of 2 to 10 times (e.g., 2, 3, 4, 5, 6, 7, 8, 9, or 10 times). In some embodiments, a ceDNA vector is delivered to a subject more than 10 times.

In some embodiments, a dose of a ceDNA vector for expression of PAH protein as disclosed herein is administered to a subject no more than once per calendar day (e.g., a 24-hour period). In some embodiments, a dose of a ceDNA vector is administered to a subject no more than once per 2, 3, 4, 5, 6, or 7 calendar days. In some embodiments, a dose of a ceDNA vector for expression of PAH protein as disclosed herein is administered to a subject no more than once per calendar week (e.g., 7 calendar days). In some embodiments, a dose of a ceDNA vector is administered to a subject no more than bi-weekly (e.g., once in a two calendar week period). In some embodiments, a dose of a ceDNA vector is administered to a subject no more than once per calendar month (e.g., once in 30 calendar days). In some embodiments, a dose of a ceDNA vector is administered to a subject no more than once per six calendar months. In some embodiments, a dose of a ceDNA vector is administered to a subject no more than once per calendar year (e.g., 365 days or 366 days in a leap year).

In particular embodiments, more than one administration (e.g., two, three, four or more administrations) of a ceDNA vector for expression of PAH protein as disclosed herein may be employed to achieve the desired level of gene expression over a period of various intervals, e.g., daily, weekly, monthly, yearly, etc.

In some embodiments, a therapeutic a PAH protein encoded by a ceDNA vector as disclosed herein can be regulated by a regulatory switch, inducible or repressible promotor so that it is expressed in a subject for at least 1 hour, at least 2 hours, at least 5 hours, at least 10 hours, at least 12 hours, at least 18 hours, at least 24 hours, at least 36 hours, at least 48 hours, at least 72 hours, at least 1 week, at least 2 weeks, at least 1 month, at least 2 months, at least 6 months, at least 12 months/one year, at least 2 years, at least 5 years, at least 10 years, at least 15 years, at least 20 years, at least 30 years, at least 40 years, at least 50 years or more. In one embodiment, the expression can be achieved by repeated administration of the ceDNA vectors described herein at predetermined or desired intervals. Alternatively, a ceDNA vector for expression of PAH protein as disclosed herein can further comprise components of a gene editing system (e.g., CRISPR/Cas, TALENs, zinc finger endonucleases etc.) to permit insertion of the one or more nucleic acid sequences encoding the PAH protein for substantially permanent treatment or “curing” the disease. Such ceDNA vectors comprising gene editing components are disclosed in International Application PCT/US18/64242, and can include the 5′ and 3′ homology arms (e.g., SEQ ID NO: 151-154, or sequences with at least 40%, 50%, 60%, 70% or 80% homology thereto) for insertion of the nucleic acid encoding the a PAH protein into safe harbor regions, such as, but not including albumin gene or CCR5 gene. By way of example, a ceDNA vector expressing a PAH protein can comprise at least one genomic safe harbor (GSH)-specific homology arms for insertion of the PAH transgene into a genomic safe harbor is disclosed in International Patent Application PCT/US2019/020225, filed on Mar. 1, 2019, which is incorporated herein in its entirety by reference.

The duration of treatment depends upon the subject's clinical progress and responsiveness to therapy. Continuous, relatively low maintenance doses are contemplated after an initial higher therapeutic dose.

### E. Unit Dosage Forms

In some embodiments, the pharmaceutical compositions comprising a ceDNA vector for expression of PAH protein as disclosed herein can conveniently be presented in unit dosage form. A unit dosage form will typically be adapted to one or more specific routes of administration of the pharmaceutical composition. In some embodiments, the unit dosage form is adapted for droplets to be administered directly to the eye. In some embodiments, the unit dosage form is adapted for administration by inhalation. In some embodiments, the unit dosage form is adapted for administration by a vaporizer. In some embodiments, the unit dosage form is adapted for administration by a nebulizer. In some embodiments, the unit dosage form is adapted for administration by an aerosolizer. In some embodiments, the unit dosage form is adapted for oral administration, for buccal administration, or for sublingual administration. In some embodiments, the unit dosage form is adapted for intravenous, intramuscular, or subcutaneous administration. In some embodiments, the unit dosage form is adapted for subretinal injection, suprachoroidal injection or intravitreal injection.

In some embodiments, the unit dosage form is adapted for intrathecal or intracerebroventricular administration. In some embodiments, the pharmaceutical composition is formulated for topical administration. The amount of active ingredient which can be combined with a carrier material to produce a single dosage form will generally be that amount of the compound which produces a therapeutic effect.

### X. Methods of Treatment

The technology described herein also demonstrates methods for making, as well as methods of using the disclosed ceDNA vectors for expression of PAH protein in a variety of ways, including, for example, ex vivo, ex situ, in vitro and in vivo applications, methodologies, diagnostic procedures, and/or gene therapy regimens.

In one embodiment, the expressed therapeutic PAH protein expressed from a ceDNA vector as disclosed herein is functional for the treatment of disease. In a preferred embodiment, the therapeutic PAH protein does not cause an immune system reaction, unless so desired.

Provided herein is a method of treating PKU in a subject comprising introducing into a target cell in need thereof (for example, a muscle cell or tissue, or other affected cell type) of the subject a therapeutically effective amount of a ceDNA vector for expression of PAH protein as disclosed herein, optionally with a pharmaceutically acceptable carrier. While the ceDNA vector can be introduced in the presence of a carrier, such a carrier is not required. The ceDNA vector implemented comprises a nucleic acid sequence encoding an PAH protein as described herein useful for treating the disease. In particular, a ceDNA vector for expression of PAH protein as disclosed herein may comprise a desired PAH protein DNA sequence operably linked to control elements capable of directing transcription of the desired PAH protein encoded by the exogenous DNA sequence when introduced into the subject. The ceDNA vector for expression of PAH protein as disclosed herein can be administered via any suitable route as provided above, and elsewhere herein.

Disclosed herein are ceDNA vector compositions and formulations for expression of PAH protein as disclosed herein that include one or more of the ceDNA vectors of the present disclosure together with one or more pharmaceutically-acceptable buffers, diluents, or excipients. Such compositions may be included in one or more diagnostic or therapeutic kits, for diagnosing, preventing, treating or ameliorating one or more symptoms of PKU. In one aspect the disease, injury, disorder, trauma or dysfunction is a human disease, injury, disorder, trauma or dysfunction.

Another aspect of the technology described herein provides a method for providing a subject in need thereof with a diagnostically- or therapeutically-effective amount of a ceDNA vector for expression of PAH protein as disclosed herein, the method comprising providing to a cell, tissue or organ of a subject in need thereof, an amount of the ceDNA vector as disclosed herein; and for a time effective to enable expression of the PAH protein from the ceDNA vector thereby providing the subject with a diagnostically- or a therapeutically-effective amount of the PAH protein expressed by the ceDNA vector. In a further aspect, the subject is human.

Another aspect of the technology described herein provides a method for diagnosing, preventing, treating, or ameliorating at least one or more symptoms of PKU, a disorder, a dysfunction, an injury, an abnormal condition, or trauma in a subject. In an overall and general sense, the method includes at least the step of administering to a subject in need thereof one or more of the disclosed ceDNA vector for PAH protein production, in an amount and for a time sufficient to diagnose, prevent, treat or ameliorate the one or more symptoms of the disease, disorder, dysfunction, injury, abnormal condition, or trauma in the subject. In such an embodiment, the subject can be evaluated for efficacy of the PAH protein, or alternatively, detection of the PAH protein or tissue location (including cellular and subcellular location) of the PAH protein in the subject. As such, the ceDNA vector for expression of PAH protein as disclosed herein can be used as an in vivo diagnostic tool, e.g., for the detection of cancer or other indications. In a further aspect, the subject is human.

Another aspect is use of a ceDNA vector for expression of PAH protein as disclosed herein as a tool for treating or reducing one or more symptoms of PKU or disease states. There are a number of inherited diseases in which defective genes are known, and typically fall into two classes: deficiency states, usually of enzymes, which are generally inherited in a recessive manner, and unbalanced states, which may involve regulatory or structural proteins, and which are typically but not always inherited in a dominant manner. For unbalanced disease states, a ceDNA vector for expression of PAH protein as disclosed herein can be used to create PKU state in a model system, which could then be used in efforts to counteract the disease state. Thus the ceDNA vector for expression of PAH protein as disclosed herein permit the treatment of genetic diseases. As used herein, PKU state is treated by partially or wholly remedying the deficiency or imbalance that causes the disease or makes it more severe.

### A. Host Cells

In some embodiments, a ceDNA vector for expression of PAH protein as disclosed herein delivers the PAH protein transgene into a subject host cell. In some embodiments, the cells are photoreceptor cells. In some embodiments, the cells are RPE cells. In some embodiments, the subject host cell is a human host cell, including, for example blood cells, stem cells, hematopoietic cells, CD34⁺ cells, liver cells, cancer cells, vascular cells, muscle cells, pancreatic cells, neural cells, ocular or retinal cells, epithelial or endothelial cells, dendritic cells, fibroblasts, or any other cell of mammalian origin, including, without limitation, hepatic (i.e., liver) cells, lung cells, cardiac cells, pancreatic cells, intestinal cells, diaphragmatic cells, renal (i.e., kidney) cells, neural cells, blood cells, bone marrow cells, or any one or more selected tissues of a subject for which gene therapy is contemplated. In one aspect, the subject host cell is a human host cell.

The present disclosure also relates to recombinant host cells as mentioned above, including a ceDNA vector for expression of PAH protein as disclosed herein. Thus, one can use multiple host cells depending on the purpose as is obvious to the skilled artisan. A construct or a ceDNA vector for expression of PAH protein as disclosed herein including donor sequence is introduced into a host cell so that the donor sequence is maintained as a chromosomal integrant as described earlier. The term host cell encompasses any progeny of a parent cell that is not identical to the parent cell due to mutations that occur during replication. The choice of a host cell will to a large extent depend upon the donor sequence and its source.

The host cell may also be a eukaryote, such as a mammalian, insect, plant, or fungal cell. In one embodiment, the host cell is a human cell (e.g., a primary cell, a stem cell, or an immortalized cell line). In some embodiments, the host cell can be administered a ceDNA vector for expression of PAH protein as disclosed herein ex vivo and then delivered to the subject after the gene therapy event. A host cell can be any cell type, e.g., a somatic cell or a stem cell, an induced pluripotent stem cell, or a blood cell, e.g., T-cell or B-cell, or bone marrow cell. In certain embodiments, the host cell is an allogenic cell. For example, T-cell genome engineering is useful for cancer immunotherapies, disease modulation such as HIV therapy (e.g., receptor knock out, such as CXCR4 and CCR5) and immunodeficiency therapies. MHC receptors on B-cells can be targeted for immunotherapy. In some embodiments, gene modified host cells, e.g., bone marrow stem cells, e.g., CD34⁺ cells, or induced pluripotent stem cells can be transplanted back into a patient for expression of a therapeutic protein.

### B. Additional Diseases for Gene Therapy

In general, a ceDNA vector for expression of PAH protein as disclosed herein can be used to deliver any PAH protein in accordance with the description above to treat, prevent, or ameliorate the symptoms associated with PKU related to an aberrant protein expression or gene expression in a subject.

In some embodiments, a ceDNA vector for expression of PAH protein as disclosed herein can be used to deliver an PAH protein to skeletal, cardiac or diaphragm muscle, for production of an PAH protein for secretion and circulation in the blood or for systemic delivery to other tissues to treat, ameliorate, and/or prevent PKU.

The a ceDNA vector for expression of PAH protein as disclosed herein can be administered to the lungs of a subject by any suitable means, optionally by administering an aerosol suspension of respirable particles comprising the ceDNA vectors, which the subject inhales. The respirable particles can be liquid or solid. Aerosols of liquid particles comprising the ceDNA vectors may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the ceDNA vectors may likewise be produced with any solid particulate medicament aerosol generator, by techniques known in the pharmaceutical art.

In some embodiments, a ceDNA vector for expression of PAH protein as disclosed herein can be administered to tissues of the CNS (e.g., brain, eye).

Ocular disorders that may be treated, ameliorated, or prevented with a ceDNA vector for expression of PAH protein as disclosed herein include ophthalmic disorders involving the retina, posterior tract, and optic nerve (e.g., retinitis pigmentosa, diabetic retinopathy and other retinal degenerative diseases, uveitis, age-related macular degeneration, glaucoma). Many ophthalmic diseases and disorders are associated with one or more of three types of indications: (1) angiogenesis, (2) inflammation, and (3) degeneration. In some embodiments, the ceDNA vector as disclosed herein can be employed to deliver anti-angiogenic factors; anti-inflammatory factors; factors that retard cell degeneration, promote cell sparing, or promote cell growth and combinations of the foregoing. Diabetic retinopathy, for example, is characterized by angiogenesis. Diabetic retinopathy can be treated by delivering one or more anti-angiogenic antibodies or fusion proteins either intraocularly (e.g., in the vitreous) or periocularly (e.g., in the sub-Tenon's region). Additional ocular diseases that may be treated, ameliorated, or prevented with the ceDNA vectors of the disclosure include geographic atrophy, vascular or “wet” macular degeneration, PKU, Leber Congenital Amaurosis (LCA), Usher syndrome, pseudoxanthoma elasticum (PXE), x-linked retinitis pigmentosa (XLRP), x-linked retinoschisis (XLRS), Choroideremia, Leber hereditary optic neuropathy (LHON), Archomatopsia, cone-rod dystrophy, Fuchs endothelial corneal dystrophy, diabetic macular edema and ocular cancer and tumors.

In some embodiments, inflammatory ocular diseases or disorders (e.g., uveitis) can be treated, ameliorated, or prevented by a ceDNA vector for expression of PAH protein as disclosed herein. One or more anti-inflammatory antibodies or fusion proteins can be expressed by intraocular (e.g., vitreous or anterior chamber) administration of the ceDNA vector as disclosed herein.

In some embodiments, a ceDNA vector for expression of PAH protein as disclosed herein can encode an PAH protein that is associated with transgene encoding a reporter polypeptide (e.g., an enzyme such as Green Fluorescent Protein, or alkaline phosphatase). In some embodiments, a transgene that encodes a reporter protein useful for experimental or diagnostic purposes, is selected from any of: β-lactamase, β-galactosidase (LacZ), alkaline phosphatase, thymidine kinase, green fluorescent protein (GFP), chloramphenicol acetyltransferase (CAT), luciferase, and others well known in the art. In some aspects, ceDNA vectors expressing an PAH protein linked to a reporter polypeptide may be used for diagnostic purposes, as well as to determine efficacy or as markers of the ceDNA vector's activity in the subject to which they are administered.

C. Testing for Successful Gene Expression Using a ceDNA Vector

Assays well known in the art can be used to test the efficiency of gene delivery of an PAH protein by a ceDNA vector can be performed in both in vitro and in vivo models. Levels of the expression of the PAH protein by ceDNA can be assessed by one skilled in the art by measuring mRNA and protein levels of the PAH protein (e.g., reverse transcription PCR, western blot analysis, and enzyme-linked immunosorbent assay (ELISA)). In one embodiment, ceDNA comprises a reporter protein that can be used to assess the expression of the PAH protein, for example by examining the expression of the reporter protein by fluorescence microscopy or a luminescence plate reader. For in vivo applications, protein function assays can be used to test the functionality of a given PAH protein to determine if gene expression has successfully occurred. One skilled will be able to determine the best test for measuring functionality of an PAH protein expressed by the ceDNA vector in vitro or in vivo.

It is contemplated herein that the effects of gene expression of an PAH protein from the ceDNA vector in a cell or subject can last for at least 1 month, at least 2 months, at least 3 months, at least four months, at least 5 months, at least six months, at least 10 months, at least 12 months, at least 18 months, at least 2 years, at least 5 years, at least 10 years, at least 20 years, or can be permanent.

In some embodiments, an PAH protein in the expression cassette, expression construct, or ceDNA vector described herein can be codon optimized for the host cell. As used herein, the term “codon optimized” or “codon optimization” refers to the process of modifying a nucleic acid sequence for enhanced expression in the cells of the vertebrate of interest, e.g., mouse or human (e.g., humanized), by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid. Typically, codon optimization does not alter the amino acid sequence of the original translated protein. Optimized codons can be determined using e.g., Aptagen's Gene Forge® codon optimization and custom gene synthesis platform (Aptagen, Inc.) or another publicly available database.

D. Determining Efficacy by Assessing PAH Protein Expression from the ceDNA Vector

Essentially any method known in the art for determining protein expression can be used to analyze expression of a PAH protein from a ceDNA vector. Non-limiting examples of such methods/assays include enzyme-linked immunoassay (ELISA), affinity ELISA, ELISPOT, serial dilution, flow cytometry, surface plasmon resonance analysis, kinetic exclusion assay, mass spectrometry, Western blot, immunoprecipitation, and PCR.

For assessing PAH protein expression in vivo, a biological sample can be obtained from a subject for analysis. Exemplary biological samples include a biofluid sample, a body fluid sample, blood (including whole blood), serum, plasma, urine, saliva, a biopsy and/or tissue sample etc. A biological sample or tissue sample can also refer to a sample of tissue or fluid isolated from an individual including, but not limited to, tumor biopsy, stool, spinal fluid, pleural fluid, nipple aspirates, lymph fluid, the external sections of the skin, respiratory, intestinal, and genitourinary tracts, tears, saliva, breast milk, cells (including, but not limited to, blood cells), tumors, organs, and also samples of in vitro cell culture constituent. The term also includes a mixture of the above-mentioned samples. The term “sample” also includes untreated or pretreated (or pre-processed) biological samples. In some embodiments, the sample used for the assays and methods described herein comprises a serum sample collected from a subject to be tested.

E. Determining Efficacy of the expressed PAH protein by Clinical Parameters

The efficacy of a given PAH protein expressed by a ceDNA vector for PKU (i.e., functional expression) can be determined by the skilled clinician. However, a treatment is considered “effective treatment,” as the term is used herein, if any one or all of the signs or symptoms of PKU is/are altered in a beneficial manner, or other clinically accepted symptoms or markers of disease are improved, or ameliorated, e.g., by at least 10% following treatment with a ceDNA vector encoding a therapeutic PAH protein as described herein. Efficacy can also be measured by failure of an individual to worsen as assessed by stabilization of PKU, or the need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting PKU, e.g., arresting, or slowing progression of PKU; or (2) relieving the PKU, e.g., causing regression of PKU symptoms; and (3) preventing or reducing the likelihood of the development of the PKU disease, or preventing secondary diseases/disorders associated with PKU. An effective amount for the treatment of a disease means that amount which, when administered to a mammal in need thereof, is sufficient to result in effective treatment as that term is defined herein, for that disease. Efficacy of an agent can be determined by assessing physical indicators that are particular to PKU disease. A physician can assess for any one or more of clinical symptoms of PKU which include: **(i) reduced serum phenylaline (Phe) levels on a regular diet. Reduction in Phe is a key biomarker in the development of treatments for PKU; (ii) restored Phe to tyrosine metabolic ratio on a normal diet. This pathway is responsible for the production of neurotransmitters; and/or (iii) assessment of reduced serum Phe levels.

### EXAMPLES

The following examples are provided by way of illustration not limitation. It will be appreciated by one of ordinary skill in the art that ceDNA vectors can be constructed from any of the wild-type or modified ITRs described herein, and that the following exemplary methods can be used to construct and assess the activity of such ceDNA vectors. While the methods are exemplified with certain ceDNA vectors, they are applicable to any ceDNA vector in keeping with the description.

### Example 1: Constructing ceDNA Vectors Using an Insect Cell-Based Method

Production of ceDNA vectors using a polynucleotide construct template is described in Example 1 of PCT/US18/49996, which is incorporated herein in its entirety by reference. For example, a polynucleotide construct template used for generating the ceDNA vectors of the present disclosure can be a ceDNA-plasmid, a ceDNA-Bacmid, and/or a ceDNA-baculovirus. Without being limited to theory, in a permissive host cell, in the presence of e.g., Rep, the polynucleotide construct template having two symmetric ITRs and an expression construct, where at least one of the ITRs is modified relative to a wild-type ITR sequence, replicates to produce ceDNA vectors. ceDNA vector production undergoes two steps: first, excision (“rescue”) of template from the template backbone (e.g., ceDNA-plasmid, ceDNA-bacmid, ceDNA-baculovirus genome etc.) via Rep proteins, and second, Rep mediated replication of the excised ceDNA vector.

Production of ceDNA-Bacmids:

DH10Bac competent cells (MAX EFFICIENCY® DH10Bac™ Competent Cells, Thermo Fisher) were transformed with either test or control plasmids following a protocol according to the manufacturer's instructions. Recombination between the plasmid and a baculovirus shuttle vector in the DH10Bac cells were induced to generate recombinant ceDNA-bacmids. The recombinant bacmids were selected by screening a positive selection based on blue-white screening in E. coli (Φ80dlacZΔM15 marker provides α-complementation of the β-galactosidase gene from the bacmid vector) on a bacterial agar plate containing X-gal and IPTG with antibiotics to select for transformants and maintenance of the bacmid and transposase plasmids. White colonies caused by transposition that disrupts the β-galactoside indicator gene were picked and cultured in 10 ml of media.

The recombinant ceDNA-bacmids were isolated from the E. coli and transfected into Sf9 or Sf21 insect cells using FugeneHD to produce infectious baculovirus. The adherent Sf9 or Sf21 insect cells were cultured in 50 ml of media in T25 flasks at 25° C. Four days later, culture medium (containing the P0 virus) was removed from the cells, filtered through a 0.45 μm filter, separating the infectious baculovirus particles from cells or cell debris.

Optionally, the first generation of the baculovirus (P0) was amplified by infecting naïve Sf9 or Sf21 insect cells in 50 to 500 ml of media. Cells were maintained in suspension cultures in an orbital shaker incubator at 130 rpm at 25° C., monitoring cell diameter and viability, until cells reach a diameter of 18-19 nm (from a naïve diameter of 14-15 nm), and a density of ˜4.0E+6 cells/mL. Between 3 and 8 days post-infection, the P1 baculovirus particles in the medium were collected following centrifugation to remove cells and debris then filtration through a 0.45 μm filter.

The ceDNA-baculovirus comprising the test constructs were collected and the infectious activity, or titer, of the baculovirus was determined. Specifically, four×20 ml Sf9 cell cultures at 2.5E+6 cells/ml were treated with P1 baculovirus at the following dilutions: 1/1000, 1/10,000, 1/50,000, 1/100,000, and incubated at 25-27° C. Infectivity was determined by the rate of cell diameter increase and cell cycle arrest, and change in cell viability every day for 4 to 5 days.

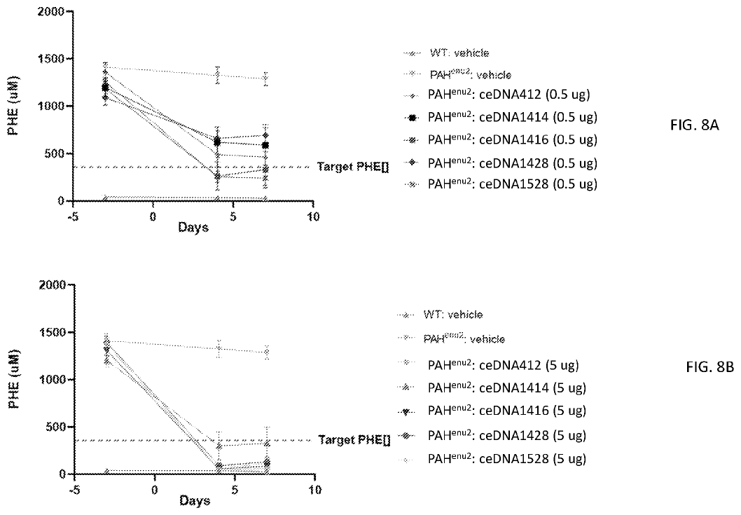

A “Rep-plasmid” as disclosed in FIG. 8A of PCT/US18/49996, which is incorporated herein in its entirety by reference, was produced in a pFASTBAC™-Dual expression vector (ThermoFisher) comprising both the Rep78 (SEQ ID NO: 131 or 133) and Rep52 (SEQ ID NO: 132) or Rep68 (SEQ ID NO: 130) and Rep40 (SEQ ID NO: 129). The Rep-plasmid was transformed into the DH10Bac competent cells (MAX EFFICIENCY@ DH10Bac™ Competent Cells (Thermo Fisher) following a protocol provided by the manufacturer. Recombination between the Rep-plasmid and a baculovirus shuttle vector in the DH10Bac cells were induced to generate recombinant bacmids (“Rep-bacmids”). The recombinant bacmids were selected by a positive selection that included-blue-white screening in E. coli ((D80dlacZΔM15 marker provides α-complementation of the 0-galactosidase gene from the bacmid vector) on a bacterial agar plate containing X-gal and IPTG. Isolated white colonies were picked and inoculated in 10 ml of selection media (kanamycin, gentamicin, tetracycline in LB broth). The recombinant bacmids (Rep-bacmids) were isolated from the E. coli and the Rep-bacmids were transfected into Sf9 or Sf21 insect cells to produce infectious baculovirus.

The Sf9 or Sf21 insect cells were cultured in 50 ml of media for 4 days, and infectious recombinant baculovirus (“Rep-baculovirus”) were isolated from the culture. Optionally, the first generation Rep-baculovirus (P0) were amplified by infecting naïve Sf9 or Sf21 insect cells and cultured in 50 to 500 ml of media. Between 3 and 8 days post-infection, the P1 baculovirus particles in the medium were collected either by separating cells by centrifugation or filtration or another fractionation process. The Rep-baculovirus were collected and the infectious activity of the baculovirus was determined. Specifically, four×20 mL Sf9 cell cultures at 2.5×10⁶ cells/mL were treated with P1 baculovirus at the following dilutions, 1/1000, 1/10,000, 1/50,000, 1/100,000, and incubated. Infectivity was determined by the rate of cell diameter increase and cell cycle arrest, and change in cell viability every day for 4 to 5 days.

ceDNA Vector Generation and Characterization

With reference to FIG. 3B, Sf9 insect cell culture media containing either (1) a sample-containing a ceDNA-bacmid or a ceDNA-baculovirus, and (2) Rep-baculovirus described above were then added to a fresh culture of Sf9 cells (2.5E+6 cells/ml, 20 ml) at a ratio of 1:1000 and 1:10,000, respectively. The cells were then cultured at 130 rpm at 25° C. 4-5 days after the co-infection, cell diameter and viability are detected. When cell diameters reached 18-20 nm with a viability of ˜70-80%, the cell cultures were centrifuged, the medium was removed, and the cell pellets were collected. The cell pellets are first resuspended in an adequate volume of aqueous medium, either water or buffer. The ceDNA vector was isolated and purified from the cells using Qiagen MIDI PLUS™ purification protocol (Qiagen, 0.2 mg of cell pellet mass processed per column).

Yields of ceDNA vectors produced and purified from the Sf9 insect cells were initially determined based on UV absorbance at 260 nm.

ceDNA vectors can be assessed by identified by agarose gel electrophoresis under native or denaturing conditions as illustrated in FIG. 3D, where (a) the presence of characteristic bands migrating at twice the size on denaturing gels versus native gels after restriction endonuclease cleavage and gel electrophoretic analysis and (b) the presence of monomer and dimer (2×) bands on denaturing gels for uncleaved material is characteristic of the presence of ceDNA vector.

Structures of the isolated ceDNA vectors were further analyzed by digesting the DNA obtained from co-infected Sf9 cells (as described herein) with restriction endonucleases selected for a) the presence of only a single cut site within the ceDNA vectors, and b) resulting fragments that were large enough to be seen clearly when fractionated on a 0.8% denaturing agarose gel (>800 bp). As illustrated in FIGS. 3D and 3E, linear DNA vectors with a non-continuous structure and ceDNA vector with the linear and continuous structure can be distinguished by sizes of their reaction products—for example, a DNA vector with a non-continuous structure is expected to produce 1 kb and 2 kb fragments, while a non-encapsidated vector with the continuous structure is expected to produce 2 kb and 4 kb fragments.

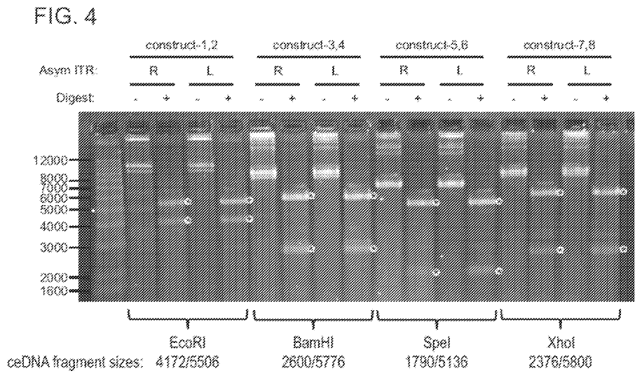

Therefore, to demonstrate in a qualitative fashion that isolated ceDNA vectors are covalently closed-ended as is required by definition, the samples were digested with a restriction endonuclease identified in the context of the specific DNA vector sequence as having a single restriction site, preferably resulting in two cleavage products of unequal size (e.g., 1000 bp and 2000 bp). Following digestion and electrophoresis on a denaturing gel (which separates the two complementary DNA strands), a linear, non-covalently closed DNA will resolve at sizes 1000 bp and 2000 bp, while a covalently closed DNA (i.e., a ceDNA vector) will resolve at 2× sizes (2000 bp and 4000 bp), as the two DNA strands are linked and are now unfolded and twice the length (though single stranded). Furthermore, digestion of monomeric, dimeric, and n-meric forms of the DNA vectors will all resolve as the same size fragments due to the end-to-end linking of the multimeric DNA vectors (see FIG. 3D). FIG. 4 is an exemplary picture of a denaturing gel running examples of ceDNA vectors with (+) or without (−) digestion with endonucleases (EcoRI for ceDNA construct 1 and 2; BamHI for ceDNA construct 3 and 4; SpeI for ceDNA construct 5 and 6; and XhoI for ceDNA construct 7 and 8) Constructs 1-8 are described in Example 1 of International Application PCT PCT/US18/49996, which is incorporated herein in its entirety by reference. Sizes of bands highlighted with an asterisk were determined and provided on the bottom of the picture.

As used herein, the phrase “assay for the Identification of DNA vectors by agarose gel electrophoresis under native gel and denaturing conditions” refers to an assay to assess the close-endedness of the ceDNA by performing restriction endonuclease digestion followed by electrophoretic assessment of the digest products. One such exemplary assay follows, though one of ordinary skill in the art will appreciate that many art-known variations on this example are possible. The restriction endonuclease is selected to be a single cut enzyme for the ceDNA vector of interest that will generate products of approximately ⅓× and ⅔× of the DNA vector length. This resolves the bands on both native and denaturing gels. Before denaturation, it is important to remove the buffer from the sample. The Qiagen PCR clean-up kit or desalting “spin columns,” e.g., GE HEALTHCARE ILUSTRA™ MICROSPIN™ G-25 columns are some art-known options for the endonuclease digestion. The assay includes for example, i) digest DNA with appropriate restriction endonuclease(s), 2) apply to e.g., a Qiagen PCR clean-up kit, elute with distilled water, iii) adding 10× denaturing solution (10×=0.5 M NaOH, 10 mM EDTA), add 10× dye, not buffered, and analyzing, together with DNA ladders prepared by adding 10× denaturing solution to 4×, on a 0.8-1.0% gel previously incubated with 1 mM EDTA and 200 mM NaOH to ensure that the NaOH concentration is uniform in the gel and gel box, and running the gel in the presence of 1× denaturing solution (50 mM NaOH, 1 mM EDTA). One of ordinary skill in the art will appreciate what voltage to use to run the electrophoresis based on size and desired timing of results. After electrophoresis, the gels are drained and neutralized in 1×TBE or TAE and transferred to distilled water or 1×TBE/TAE with 1×SYBR Gold. Bands can then be visualized with e.g. Thermo Fisher, SYBR® Gold Nucleic Acid Gel Stain (10,000× Concentrate in DMSO) and epifluorescent light (blue) or UV (312 nm).

The purity of the generated ceDNA vector can be assessed using any art-known method. As one exemplary and non-limiting method, contribution of ceDNA-plasmid to the overall UV absorbance of a sample can be estimated by comparing the fluorescent intensity of ceDNA vector to a standard. For example, if based on UV absorbance 4 μg of ceDNA vector was loaded on the gel, and the ceDNA vector fluorescent intensity is equivalent to a 2 kb band which is known to be 1 μg, then there is 1 μg of ceDNA vector, and the ceDNA vector is 25% of the total UV absorbing material. Band intensity on the gel is then plotted against the calculated input that band represents—for example, if the total ceDNA vector is 8 kb, and the excised comparative band is 2 kb, then the band intensity would be plotted as 25% of the total input, which in this case would be 0.25 μg for 1.0 μg input. Using the ceDNA vector plasmid titration to plot a standard curve, a regression line equation is then used to calculate the quantity of the ceDNA vector band, which can then be used to determine the percent of total input represented by the ceDNA vector, or percent purity.

For comparative purposes, Example 1 describes the production of ceDNA vectors using an insect cell-based method and a polynucleotide construct template, and is also described in Example 1 of International Patent Application No. PCT/US18/49996, which is incorporated herein in its entirety by reference. For example, a polynucleotide construct template used for generating the ceDNA vectors of the present disclosure according to Example 1 can be a ceDNA-plasmid, a ceDNA-Bacmid, and/or a ceDNA-baculovirus. Without being limited to theory, in a permissive host cell, in the presence of e.g., Rep, the polynucleotide construct template having two symmetric ITRs and an expression construct, where at least one of the ITRs is modified relative to a wild-type ITR sequence, replicates to produce ceDNA vectors. ceDNA vector production undergoes two steps: first, excision (“rescue”) of template from the template backbone (e.g., ceDNA-plasmid, ceDNA-bacmid, ceDNA-baculovirus genome etc.) via Rep proteins, and second, Rep mediated replication of the excised ceDNA vector.

### Example 2: Synthetic ceDNA Production Via Excision from a Double-Stranded DNA Molecule

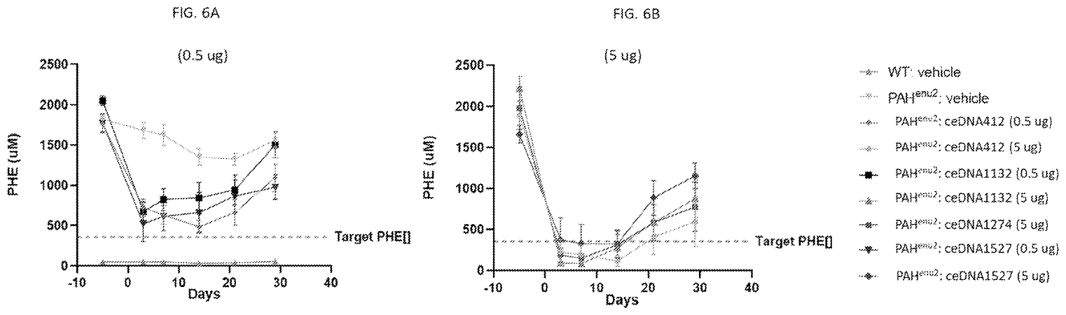

Synthetic production of the ceDNA vectors is described in Examples 2-6 of International Patent Application No. PCT/US19/14122, filed Jan. 18, 2019, which is incorporated herein in its entirety by reference. One exemplary method of producing a ceDNA vector using a synthetic method that involves the excision of a double-stranded DNA molecule. In brief, a ceDNA vector can be generated using a double stranded DNA construct, e.g., see FIGS. 7A-8E of PCT/US19/14122. In some embodiments, the double stranded DNA construct is a ceDNA plasmid, e.g., see, e.g., FIG. 6 in International Patent Application No. PCT/US2018/064242, filed Dec. 6, 2018).

In some embodiments, a construct to make a ceDNA vector comprises a regulatory switch as described herein.

For illustrative purposes, Example 2 describes producing ceDNA vectors as exemplary closed-ended DNA vectors generated using this method. However, while ceDNA vectors are exemplified in this Example to illustrate in vitro synthetic production methods to generate a closed-ended DNA vector by excision of a double-stranded polynucleotide comprising the ITRs and expression cassette (e.g., nucleic acid sequence, e.g., heterologous nucleic acid sequence) followed by ligation of the free 3′ and 5′ ends as described herein, one of ordinary skill in the art is aware that one can, as illustrated above, modify the double stranded DNA polynucleotide molecule such that any desired closed-ended DNA vector is generated, including but not limited to, doggybone DNA, dumbbell DNA and the like. Exemplary ceDNA vectors for production of antibodies or fusion proteins that can be produced by the synthetic production method described in Example 2 are discussed in the sections entitled “III ceDNA vectors in general”. Exemplary antibodies and fusion proteins expressed by the ceDNA vectors are described in the section entitled “IIC Exemplary antibodies and fusion proteins expressed by the ceDNA vectors”.

The method involves (i) excising a sequence encoding the expression cassette from a double-stranded DNA construct and (ii) forming hairpin structures at one or more of the ITRs and (iii) joining the free 5′ and 3′ ends by ligation, e.g., by T4 DNA ligase.

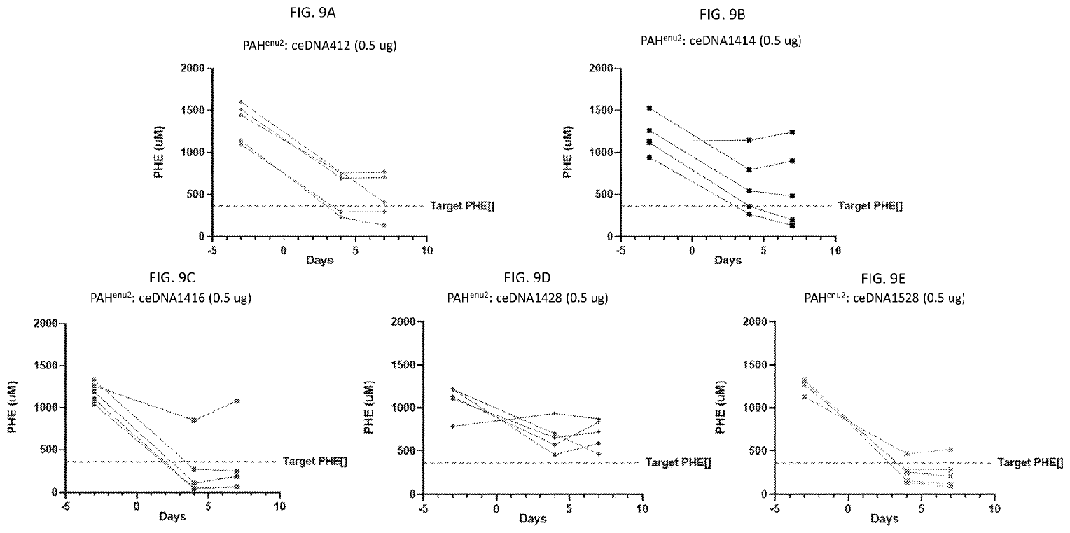

The double-stranded DNA construct comprises, in 5′ to 3′ order: a first restriction endonuclease site; an upstream ITR; an expression cassette; a downstream ITR; and a second restriction endonuclease site. The double-stranded DNA construct is then contacted with one or more restriction endonucleases to generate double-stranded breaks at both of the restriction endonuclease sites. One endonuclease can target both sites, or each site can be targeted by a different endonuclease as long as the restriction sites are not present in the ceDNA vector template. This excises the sequence between the restriction endonuclease sites from the rest of the double-stranded DNA construct (see FIG. 9 of PCT/US19/14122). Upon ligation a closed-ended DNA vector is formed.

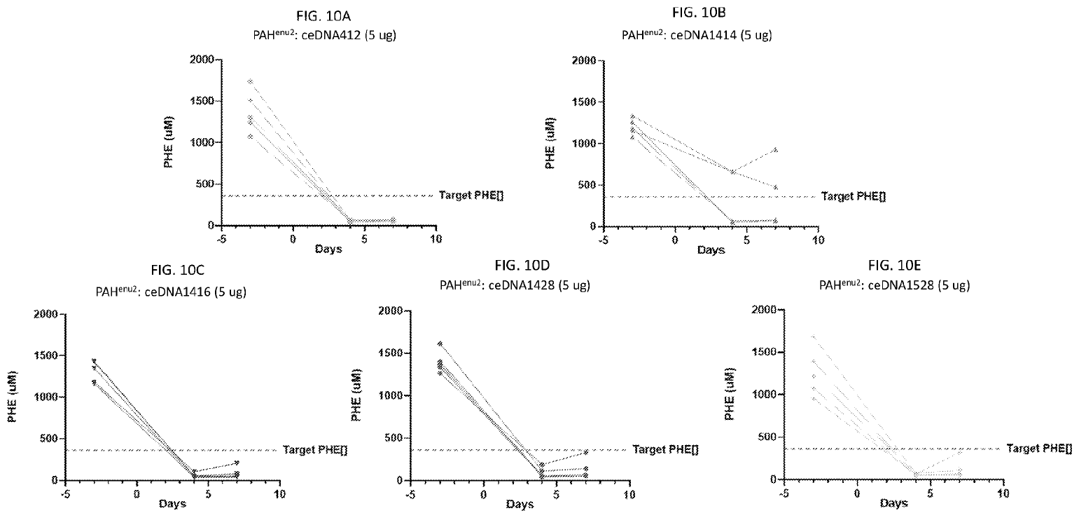

One or both of the ITRs used in the method may be wild-type ITRs. Modified ITRs may also be used, where the modification can include deletion, insertion, or substitution of one or more nucleotides from the wild-type ITR in the sequences forming B and B′ arm and/or C and C′ arm (see, e.g., FIGS. 6-8 and 10 FIG. 11B of PCT/US19/14122), and may have two or more hairpin loops (see, e.g., FIGS. 6-8 FIG. 11B of PCT/US19/14122) or a single hairpin loop (see, e.g., FIG. 10A-10B FIG. 11B of PCT/US19/14122). The hairpin loop modified ITR can be generated by genetic modification of an existing oligo or by de novo biological and/or chemical synthesis.

In a non-limiting example, ITR-6 Left and Right (SEQ ID NOS: 111 and 112), include 40 nucleotide deletions in the B-B′ and C-C′ arms from the wild-type ITR of AAV2. Nucleotides remaining in the modified ITR are predicted to form a single hairpin structure. Gibbs free energy of unfolding the structure is about −54.4 kcal/mol. Other modifications to the ITR may also be made, including optional deletion of a functional Rep binding site or a Trs site.

### Example 3: ceDNA Production Via Oligonucleotide Construction

Another exemplary method of producing a ceDNA vector using a synthetic method that involves assembly of various oligonucleotides, is provided in Example 3 of PCT/US19/14122, incorporated by reference in its entirety herein, where a ceDNA vector is produced by synthesizing a 5′ oligonucleotide and a 3′ ITR oligonucleotide and ligating the ITR oligonucleotides to a double-stranded polynucleotide comprising an expression cassette. FIG. 11B of PCT/US19/14122 shows an exemplary method of ligating a 5′ ITR oligonucleotide and a 3′ ITR oligonucleotide to a double stranded polynucleotide comprising an expression cassette.

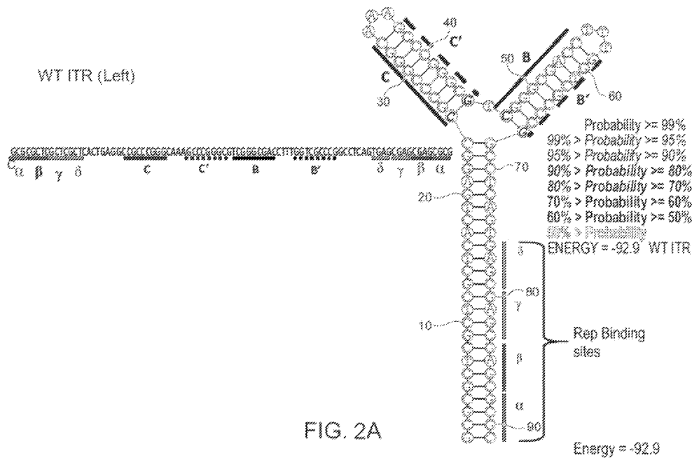

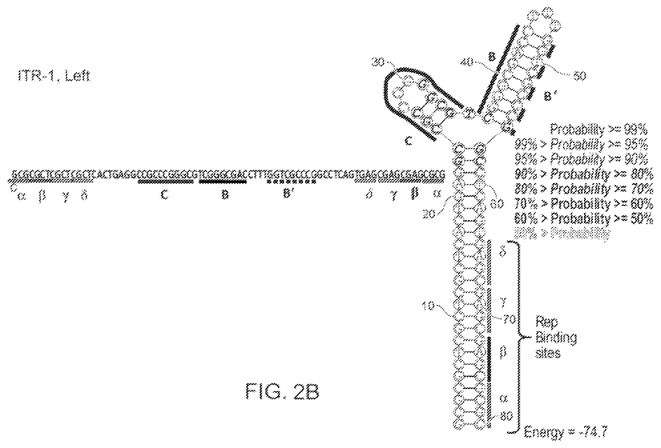

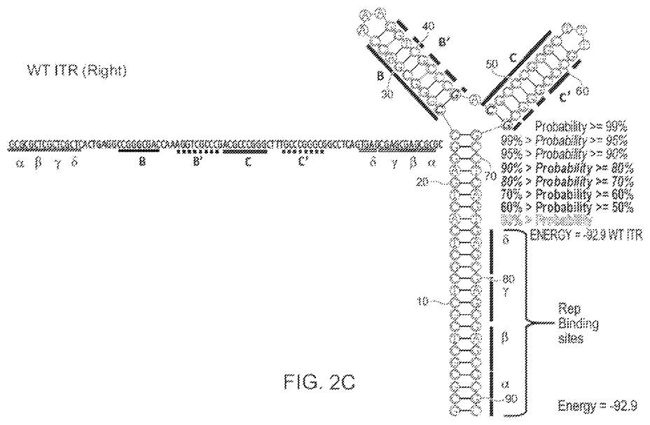

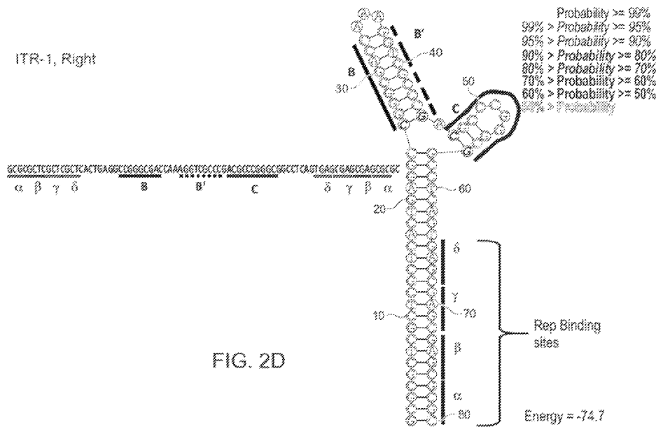

The ITR oligonucleotides can comprise WT-ITRs (e.g., see FIG. 2A, FIG. 2C), or modified ITRs (e.g., see, FIG. 2B and FIG. 2D). (See e.g., FIGS. 6A, 6B, 7A and 7B of PCT/US19/14122, which is incorporated herein in its entirety). Exemplary ITR oligonucleotides include, but are not limited to SEQ ID NOS: 134-145 (e.g., see Table 7 in of PCT/US19/14122). Modified ITRs can include deletion, insertion, or substitution of one or more nucleotides from the wild-type ITR in the sequences forming B and B′ arm and/or C and C′ arm. ITR oligonucleotides, comprising WT-ITRs or mod-ITRs as described herein, to be used in the cell-free synthesis, can be generated by genetic modification or biological and/or chemical synthesis. The ITR oligonucleotides can comprise WT-ITRs, or modified ITRs (mod-ITRs) in symmetrical or asymmetrical configurations, as discussed herein.

### Example 4: ceDNA Production Via a Single-Stranded DNA Molecule

Another exemplary method of producing a ceDNA vector using a synthetic method is provided in Example 4 of PCT/US19/14122, incorporated by reference in its entirety herein, and uses a single-stranded linear DNA comprising two sense ITRs which flank a sense expression cassette sequence and are attached covalently to two antisense ITRs which flank an antisense expression cassette, the ends of which single stranded linear DNA are then ligated to form a closed-ended single-stranded molecule. One non-limiting example comprises synthesizing and/or producing a single-stranded DNA molecule, annealing portions of the molecule to form a single linear DNA molecule which has one or more base-paired regions of secondary structure, and then ligating the free 5′ and 3′ ends to each other to form a closed single-stranded molecule.

An exemplary single-stranded DNA molecule for production of a ceDNA vector comprises, from 5′ to 3′: a sense first ITR; a sense expression cassette sequence; a sense second ITR; an antisense second ITR; an antisense expression cassette sequence; and an antisense first ITR.

A single-stranded DNA molecule for use in the exemplary method of Example 4 can be formed by any DNA synthesis methodology described herein, e.g., in vitro DNA synthesis, or provided by cleaving a DNA construct (e.g., a plasmid) with nucleases and melting the resulting dsDNA fragments to provide ssDNA fragments.

Annealing can be accomplished by lowering the temperature below the calculated melting temperatures of the sense and antisense sequence pairs. The melting temperature is dependent upon the specific nucleotide base content and the characteristics of the solution being used, e.g., the salt concentration. Melting temperatures for any given sequence and solution combination are readily calculated by one of ordinary skill in the art.

The free 5′ and 3′ ends of the annealed molecule can be ligated to each other, or ligated to a hairpin molecule to form the ceDNA vector. Suitable exemplary ligation methodologies and hairpin molecules are described in Examples 2 and 3.

### Example 5: Purifying and/or Confirming Production of ceDNA

Any of the DNA vector products produced by the methods described herein, e.g., including the insect cell based production methods described in Example 1, or synthetic production methods described in Examples 2-4 can be purified, e.g., to remove impurities, unused components, or byproducts using methods commonly known by a skilled artisan; and/or can be analyzed to confirm that DNA vector produced, (in this instance, a ceDNA vector) is the desired molecule. An exemplary method for purification of the DNA vector, e.g., ceDNA is using Qiagen Midi Plus purification protocol (Qiagen) and/or by gel purification,

The following is an exemplary method for confirming the identity of ceDNA vectors.

ceDNA vectors can be assessed by identified by agarose gel electrophoresis under native or denaturing conditions as illustrated in FIG. 3D, where (a) the presence of characteristic bands migrating at twice the size on denaturing gels versus native gels after restriction endonuclease cleavage and gel electrophoretic analysis and (b) the presence of monomer and dimer (2×) bands on denaturing gels for uncleaved material is characteristic of the presence of ceDNA vector.

Structures of the isolated ceDNA vectors were further analyzed by digesting the purified DNA with restriction endonucleases selected for a) the presence of only a single cut site within the ceDNA vectors, and b) resulting fragments that were large enough to be seen clearly when fractionated on a 0.8% denaturing agarose gel (>800 bp). As illustrated in FIGS. 3C and 3D, linear DNA vectors with a non-continuous structure and ceDNA vector with the linear and continuous structure can be distinguished by sizes of their reaction products—for example, a DNA vector with a non-continuous structure is expected to produce 1 kb and 2 kb fragments, while a ceDNA vector with the continuous structure is expected to produce 2 kb and 4 kb fragments.

Therefore, to demonstrate in a qualitative fashion that isolated ceDNA vectors are covalently closed-ended as is required by definition, the samples were digested with a restriction endonuclease identified in the context of the specific DNA vector sequence as having a single restriction site, preferably resulting in two cleavage products of unequal size (e.g., 1000 bp and 2000 bp). Following digestion and electrophoresis on a denaturing gel (which separates the two complementary DNA strands), a linear, non-covalently closed DNA will resolve at sizes 1000 bp and 2000 bp, while a covalently closed DNA (i.e., a ceDNA vector) will resolve at 2× sizes (2000 bp and 4000 bp), as the two DNA strands are linked and are now unfolded and twice the length (though single stranded). Furthermore, digestion of monomeric, dimeric, and n-meric forms of the DNA vectors will all resolve as the same size fragments due to the end-to-end linking of the multimeric DNA vectors (see FIG. 3E).

As used herein, the phrase “assay for the Identification of DNA vectors by agarose gel electrophoresis under native gel and denaturing conditions” refers to an assay to assess the close-endedness of the ceDNA by performing restriction endonuclease digestion followed by electrophoretic assessment of the digest products. One such exemplary assay follows, though one of ordinary skill in the art will appreciate that many art-known variations on this example are possible. The restriction endonuclease is selected to be a single cut enzyme for the ceDNA vector of interest that will generate products of approximately ⅓× and ⅔× of the DNA vector length. This resolves the bands on both native and denaturing gels. Before denaturation, it is important to remove the buffer from the sample. The Qiagen PCR clean-up kit or desalting “spin columns,” e.g. GE HEALTHCARE ILUSTRA™ MICROSPIN™ G-25 columns are some art-known options for the endonuclease digestion. The assay includes for example, i) digest DNA with appropriate restriction endonuclease(s), 2) apply to e.g., a Qiagen PCR clean-up kit, elute with distilled water, iii) adding 10× denaturing solution (10×=0.5 M NaOH, 10 mM EDTA), add 10× dye, not buffered, and analyzing, together with DNA ladders prepared by adding 10× denaturing solution to 4×, on a 0.8-1.0% gel previously incubated with 1 mM EDTA and 200 mM NaOH to ensure that the NaOH concentration is uniform in the gel and gel box, and running the gel in the presence of 1× denaturing solution (50 mM NaOH, 1 mM EDTA). One of ordinary skill in the art will appreciate what voltage to use to run the electrophoresis based on size and desired timing of results. After electrophoresis, the gels are drained and neutralized in 1×TBE or TAE and transferred to distilled water or 1×TBE/TAE with 1×SYBR Gold. Bands can then be visualized with e.g. Thermo Fisher, SYBR® Gold Nucleic Acid Gel Stain (10,000× Concentrate in DMSO) and epifluorescent light (blue) or UV (312 nm). The foregoing gel-based method can be adapted to purification purposes by isolating the ceDNA vector from the gel band and permitting it to renature.

The purity of the generated ceDNA vector can be assessed using any art-known method. As one exemplary and non-limiting method, contribution of ceDNA-plasmid to the overall UV absorbance of a sample can be estimated by comparing the fluorescent intensity of ceDNA vector to a standard. For example, if based on UV absorbance 4 μg of ceDNA vector was loaded on the gel, and the ceDNA vector fluorescent intensity is equivalent to a 2 kb band which is known to be 1 μg, then there is 1 μg of ceDNA vector, and the ceDNA vector is 25% of the total UV absorbing material. Band intensity on the gel is then plotted against the calculated input that band represents—for example, if the total ceDNA vector is 8 kb, and the excised comparative band is 2 kb, then the band intensity would be plotted as 25% of the total input, which in this case would be 0.25 μg for 1.0 μg input. Using the ceDNA vector plasmid titration to plot a standard curve, a regression line equation is then used to calculate the quantity of the ceDNA vector band, which can then be used to determine the percent of total input represented by the ceDNA vector, or percent purity.

### Example 6: Pharmacology Study to Evaluate Biochemical Correction of Phenylalanine Levels in PAHe^{nu2} Mice by IV and Hydrodynamic Administration of ceDNA

It has previously been shown in International Application No. PCT/US2020/022595, incorporated by reference in its entirety herein, that using a murine model of PAH deficiency, the PAH^{enu2} mouse, two different ceDNA vectors, each with a wild-type left ITR and a truncation mutant right ITR, and having a transgene region encoding human PAH, when administered by hydrodynamic injection, expressed active PAH, which was able to systemically reduce phenylalanine levels. Further, International Application No. PCT/US2020/022595 demonstrated that administration of ceDNA containing a VD promoter linked to human PAH codon optimized version 2 (“Codop2”) resulted in decreased serum PHE levels, indicating sufficient PAH activity to correct blood phenylalanine levels in murine PKU as early as day 3.

ceDNA vectors were prepared and purified as described above in Examples 1 and 5.

The nucleic acid sequence of ceDNA containing codon optimized human PAH version 2 (ceDNA “hPAH Codop2”) is shown herein as SEQ ID NO: 192, and includes the following elements: left-ITR_v1: spacer_left-ITR_v2.1: VD_Promoter Set (VD): PmeI_site: Modified_Minimum_Consensus_Kozak: hPAH_codop_ORF_v2: PacI_site: WPRE_3pUTR: bGH/spacer: spacer_right-ITR_v1: right-ITR_v1 (ceDNA412).

The nucleic acid sequence of ceDNA containing human PAH cDNA (ceDNA VD promoter linked to hPAH cDNA without codon optimization) is shown herein as SEQ ID NO: 193, and includes the following elements: Left-ITR_v1: spacer_left-ITR_v1: VD_Promoter Set (VD) PmeI_site: Consensus_Kozak: hPAH_cDNA_ORF_v3: PacI_site: WPRE_3pUTR: bGH: spacer_right-ITR_v1: right-ITR_v1 (ceDNA802).

The nucleic acid sequence of ceDNA containing codon optimized human PAH version 2 r5-s29 (ceDNA “hPAH-r5-s29”), with specific cis-regulatory elements is shown herein as SEQ ID NO: 213, and includes the following elements: left-ITR_v1, spacer_left-ITR_v2, HS-CRM8_SERP_Enhancer_nospacer, HS-CRM8_SERP_Enhancer_nospacer, HS-CRM8_SERP_Enhancer_nospacer, BamHI_site, TTR-promoter-d5pUTR, MVM_intron, PmeI_site, Mod_Minimum_Consensus_Kozak, hPAH-r5-s29, PacI_site, WPRE_3pUTR, bGH, spacer_right-ITR_v1, right-ITR_v1 (ceDNA1530).

Each of the ceDNA PAH vectors (alone, without any LNP encapsulation) and the control were administered to mixed gender, age-matched PAH^{enu2} mice approximately 4-6 weeks old. The naked ceDNA vectors were dosed at 0.5 μg or 5 μg per animal (5 animals per group) for ceDNA412 and ceDNA802 and 0.5 μg or 1 μg per animal (5 animals per group) for ceDNA1530 by hydrodynamic intravenous injection via lateral tail vein in a dose volume of 90-100 ml/kg on day 0. Day 21 was the terminal time point.

A well-known method of introducing nucleic acid to the liver in rodents is by hydrodynamic tail vein injection. In this system, the pressurized injection in a large volume of non-encapsulated nucleic acid results in a transient increase in cell permeability and delivery directly into tissues and cells. This provides an experimental mechanism to bypass many of the host immune systems, such as macrophage delivery, providing the opportunity to observe delivery and expression in the absence of such activity.

The study design is shown below in Table 15.

TABLE 15

| | Animals | | | | | | | Terminal |
|---|---|---|---|---|---|---|---|---|
| Group | per | | | Dose | | Dose | Treatment | Time |
| No. | Group | Genotype | Treatment | Level | | Volume | Regimen, IV | Point |
| 1 | 5 | WT | PBS | NA | | 90-100 ml/kg | Once on | Day 21 |
| | | littermates | | | | (set volume) | | |
| 2 | 5 | PAH^{enu2} | PBS | NA | | | Day 0 by IV | |
| 3 | 5 | MUT | ceDNA412 (TR) | 0.5 | μg | | Hydrodynamic | |
| 4 | 5 | | ceDNA412 (TR) | 5.0 | μg | | | |
| 5 | 5 | | ceDNA802 (BIICs) | 5.0 | μg | | | |
| 6 | 5 | | ceDNA1530 (TR) | 0.5 | μg | | | |
| 7 | 5 | | ceDNA1530 (TR) | 1.0 | μg | | | |
| 8 | 5 | | ceDNA1274 LNP | 5 | mg/kg | 5 ml/kg | Once on | |

| | | | |
|---|---|---|---|
| | | formulation 1 | Day 0 by IV |
| 9 | 5 | ceDNA274 LNP | |
| | | formulation 2 | |

No. = Number; IV = intravenous; WT = wild type; MUT = mutant.

Test articles were supplied in a concentrated stock and stored at −4° C. until use. Formulations were not vortexed or centrifuged. Groups were housed in clear polycarbonate cages with contact bedding on a ventilated rack in a procedure room. Food and filtered tap water acidified with 1N HCl to a targeted pH of 2.5-3.0 were be provided to the animals ad libitum.

Blood was collected at interim and terminal time points as follows in Tables 16A and 16B, respectively.

TABLE 16A
Blood Collection (Interim)

| | Whole Blood Sample Collection Times | |
|---|---|---|
| | Saphenous Only | |
| Group | (Orbital by permission of Vet Staff) | |
| Number | Serum^{a} | |
| 1-9 | | FASTED |
| | | Day -3, 4, 7, 14 |
| 1, 2, 8 & 9 | Day 0 | |
| | 6 hours post Test Material | |
| | dose (±5%) | |
| Volume / | ~150 μL whole blood | ~50 μL whole blood |
| Portion | | |
| Processing | One (1) aliquot | One (1) aliquot |
| | (Charles River) | (Pure Honey) |
| Storage | Frozen at nominally −70° C. | Frozen at nominally −70° C. |

^{a}Whole blood was collected into serum separator tubes, with clot activator; MOV = maximum obtainable volume

TABLE 16B
Terminal Blood and Tissue Collection

| Group | Sample Collection Times | |
|---|---|---|
| Number | Serum^{a} | Liver |
| 1-9 | FASTED: On Day 21 | |
| 1, 2, 8 & 9 | | FASTED: On Day 21 |
| Volume/ | MOV | Whole organ, divided |
| Portion | | |
| Processing | Two (2) aliquots | 2 x ~50 mg pieces, not weighed |
| | (Pure Honey) | and snap frozen individually |
| | | (Pure Honey) |
| Storage | Frozen at nominally −70° C. | Frozen at nominally −70° C. |

^{a}Whole blood collected into serum separator tubes, with clot activator; MOV = maximum obtainable volume

### Study Details are Provided as Follows:

Species (number, sex, age): 40+2 spare PAH^{enu2} Mutant (MUT) mice (mixed gender, ˜5-10 weeks old, age-matched at arrival); 5 Wild Type (WT); mixed gender, littermates; age-matched. Animals were ˜10-14 weeks of at dose initiation.
Cage Side Observations: Cage side observations were performed daily.
Class of Compound: Recombinant DNA vector, ceDNA
Body Weights: Body weights for all animals, as applicable) were recorded on Days −3, 0, 1, 2, 4, 7, 14 & 21 (prior to euthanasia). Additional body weights were recorded as requested.
Dose Formulation: Test articles supplied in a concentration stock. Stock diluted with PBS immediately prior to use. Prepared materials stored at ˜4° C. (or on wet ice) if dosing is not performed immediately.
Dose Administration: Test Materials for Groups 1-7 were dosed on Day 0 by hydrodynamic IV administration, at a set volume per animal, 90-100 ml/kg (dependent on the lightest animal in the group) via lateral tail vein. Groups 8 and 9 were dosed on Day 0 by hydrodynamic IV administration, at a set volume per animal, 5 ml/kg.
Fasting Prior to Blood Collection and necropsy (for serum and tissues): All animals (all groups) were fasted for a minimum of 4 hours prior to all blood collections and necropsy: Days −3, 4, 7, 14 & 21. Animals will not be fasted on Day 0.
Interim Blood Collection: All animals in Groups 1, 2, 8 & 9 only, will have interim blood collected on Day 0; 6 hours post Test Material dose (±5%).

All animals in Groups 1-9 will have blood collected on Days −3, −4, 0, 1, 2, 4 3, & 7, 14 & 21. Animals will have whole blood for fasted serum collection.
Euthanasia & Terminal Blood Collection: On Day 21, after a minimum 4 hour fast, will be euthanized by CO₂ asphyxiation followed by thoracotomy and exsanguination.
Phenylalanine (PHE) Levels: Serum samples were analyzed by the Pure Honey for PHE levels.
Activity Levels: Two (2) frozen liver samples were analyzed by Pure Honey for Activity levels.

### Results

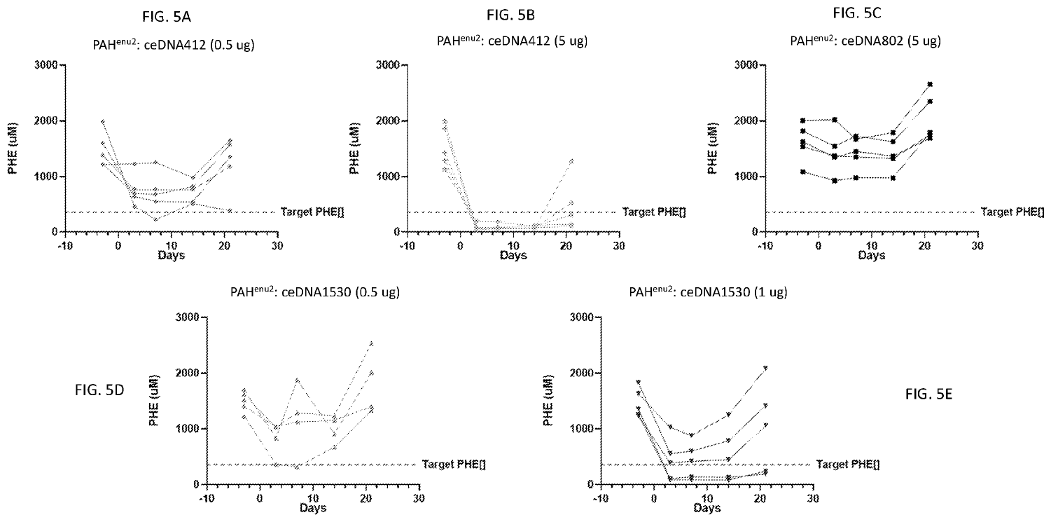

As shown in FIGS. 5A-5C, a ceDNA vector comprising a PAH nucleic acid sequence that has been codon optimized (ceDNA412; hPAH_codop_ORF_v2) corrected phenylalanine level (“PHE μM”) to below target concentration at a higher rate than the ceDNA802 (hPAH native cDNA sequence) at both a 0.5 μg and 5 μg hydrodynamic dose, while ceDNA802 did not correct PHE concentration. Results are shown for individual mice over 21 days. ceDNA1530 is a ceDNA vector comprising a PAH nucleic acid sequence that has been codon optimized (ceDNA1530; hPAH-r5-s29) with a 3×HS-CRM8_SERP_Enhancer, a TTR-promoter-d5pUTR and MVM_intron. As shown in FIGS. 5D and 5E, ceDNA1530 was not as effective at correcting PHE concentration at a 0.5 μg dose, but reached target PHE levels at a 1 μg dose.

### Example 7: Pharmacology Study to Evaluate Biochemical Correction of Phenylalanine Levels in PAH^{en2} Mice by Hydrodynamic Administration of ceDNA-Testing of an Autoregulatory Mutant ceDNA1274

ceDNA vectors were prepared and purified as described above in Examples 1 and 5.

The nucleic acid sequence of ceDNA containing codon optimized human PAH version 2 (ceDNA “hPAH Codop2”) is shown herein as SEQ ID NO: 192, and includes the following elements: left-ITR_v1: spacer_left-ITR_v2.1: VD_Promoter Set (VD): PmeI_site: Modified_Minimum_Consensus_Kozak: hPAH_codop_ORF_v2: PacI_site: WPRE_3pUTR: bGH/spacer: spacer_right-ITR_v1: right-ITR_v1 (ceDNA412)

The nucleic acid sequence of ceDNA containing codon optimized human PAH version 2 (ceDNA “hPAH Codop2”) with specific cis-regulatory elements is shown herein as SEQ ID NO: 194, and includes the following elements: left-ITR_v1, spacer_left-ITR_v2.1, 3×SerpEnh-TTRe-TTRm, MVM_intron, PmeI_site, Mod_Minimum_Consensus_Kozak, hPAH_codop_ORF_v2, PacI_site, WPRE_3pUTR, bGH, spacer_right-ITR_v1, right-ITR_v1 (ceDNA1132).

The nucleic acid sequence of ceDNA containing codon optimized human PAH version 2 (ceDNA “hPAH Codop2”) with a 29 amino acid deletion, with specific cis-regulatory elements is shown herein as SEQ ID NO: 195, and includes the following elements: left-ITR_v1, spacer_left-ITR_v2.1, VD_PromoterSet, PmeI_site, Mod_Minimum_Consensus_Kozak, hPAH_codop_ORF_v2_delta1-29aa, PacI_site, WPRE_3pUTR, bGH, spacer_right-ITR_v1, right-ITR_v1 (ceDNA1274).

The nucleic acid sequence of ceDNA codon optimized human PAH version 2 (ceDNA “hPAH_codop_ORF_v2”), with specific cis-regulatory elements is shown herein as SEQ ID NO: 210, and includes the following elements: left-ITR_v1, spacer_left-ITR_v2.1, 3×VanD_TTRe_PromoterSet_v2, PmeI_site, Mod_Minimum_Consensus_Kozak, hPAH_codop_ORF_v2, PacI_site, WPRE_3pUTR, bGH, spacer_right-ITR_v1, right-ITR_v1 (ceDNA1527).

Each of the ceDNA PAH vectors (alone, without any LNP encapsulation) and the control were administered to mixed gender, age-matched PAHenu2 mice approximately 4-6 weeks old. The naked ceDNA vectors were dosed at 0.5 μg or 5 μg per animal (5 animals per group) for ceDNA412, ceDNA1132 and ceDNA1527 and 5 μg per animal (5 animals per group) for ceDNA1527 by hydrodynamic intravenous injection via lateral tail vein in a dose volume of 90-100 ml/kg on day 0. Day 28 was the terminal time point. The study design is shown below in Table 17.

TABLE 17

| | | | | Dose | | Dosing | |
|---|---|---|---|---|---|---|---|
| Group | No. of | | | Levels | | Regimen | Terminal |
| No. | Animals | Genotype | Test Material | (μg) | Dose Volume | ROA | Time Point |
| 1 | 5 | WT | PBS | NA | 90-100 ml/kg | ONCE ON | Day 28 |
| 2 | 5 | PAH^{enu2} | PBS | NA | (set volume) | Day 0 by | |
| 3 | 5 | MUT | ceDNA412 | 0.5 | | Hydrodynamic | |
| 4 | 5 | | ceDNA412 | 5.0 | | IV | |
| 5 | 5 | | ceDNA1132 | 0.5 | | | |
| 6 | 5 | | ceDNA1132 | 5.0 | | | |
| 7 | 5 | | ceDNA1274 | 5.0 | | | |
| 8 | 5 | | ceDNA1527 | 0.5 | | | |
| 9 | 5 | | ceDNA1527 | 5.0 | | | |

No. = Number; IV = intravenous; ROA = route of administration; WT = wild type; MUT = homozygous mutant; min = minute; hr = hour

Test articles were supplied in a concentrated stock and stored at ˜4° C. until use. Formulations were not vortexed or centrifuged. Groups were housed in clear polycarbonate cages with contact bedding on a ventilated rack in a procedure room. Animals were provided ad libitum Mouse Diet 5058 and filtered tap water acidified with 1N HCl to a targeted pH of 2.5-3.0.

Blood was collected at interim and terminal time points as follows in Tables 18A and 18B, respectively.

TABLE 18A
Blood Collection (Interim)

| | Whole Blood Sample Collection Times |
|---|---|
| Group | Saphenous |
| Number | Serum^{a} |
| 1-9 | FASTED |
| | Day -5, 3, 7, 14, & 21 |
| Volume/Portion | ~50 μL whole blood |
| Processing | One (1) aliquot |
| | (Pure Honey) |
| Storage | Frozen at nominally −70° C. |

^{a}Whole blood will be collected into serum separator tubes, with clot activator

TABLE 18B
Terminal Blood and Tissue Collection

| | Sample Collection Times |
|---|---|
| | Terminal Whole Blood |
| Group | (cardiac) |
| Number | Serum^{a} |
| 1-9 | FASTED |
| | Day 29 |
| Portion | MOV |
| Processing/ | One (1) aliquot processed plasma |
| Storage | frozen at nominally −70° C. |

^{a}Whole blood will be collected into serum separator tubes, with clot activator
MOV = maximum obtainable volume

### Study Details are Provided as Follows:

Species (number, sex, age): 40+2 spare PAHenu2 Mutant (MUT) mice (mixed gender, ˜5-10 weeks old, age-matched at arrival); 5 Wild Type (WT); mixed gender, littermates; age-matched. Animals were ˜10-14 weeks of at dose initiation.
Cage Side Observations: Cage side observations were performed daily.
Clinical Observations: Clinical observations were performed ˜1, ˜5-6 and ˜24 hours post the Day 0 Test Material dose, as applicable for remaining groups.
Class of Compound: Recombinant DNA vector: ceDNA.
Body Weights: Body weights for all animals were recorded on Days −5, 0, 1, 2, 3, 7, 14, 21 & 28 (prior to euthanasia). Additional body weight may be recorded as requested.
Dose Formulation: Test articles were supplied in a concentration stock. Stock was warmed to room temperature and diluted with the provided PBS immediately prior to use. Prepared materials may be stored at ˜4° C. if dosing is not performed immediately.
Dose Administration: Test Materials for Groups 1-9 were dosed on Day 0 by hydrodynamic IV administration, at a set volume per animal, 90-100 ml/kg (dependent on the lightest animal in the group) via lateral tail vein.
Fasting Prior to Blood Collection: All animals (all groups) were fasted for a minimum of 4 hours prior to the all interim and terminal blood collections: Days −5, 3, 7, 14, 21 & 28.
Food was removed and bedding changed. Food was be returned at the conclusion of each interim blood collections for a fast of no more than 8 hours in duration.
Interim Blood Collection: All animals in Groups 1-9 had interim blood collected on Days −5, 3, 7, 14 & 21. Animals had whole blood for fasted serum collection. After collection animals received 0.5-1.0 mL lactated Ringer's, subcutaneously.
Blood Collection: Whole blood for serum was collected by saphenous vein. Whole blood was collected into a serum separator with clot activator tube and processed into one (1) aliquot of serum per facility SOPs. All samples were stored at nominally −70° C. until shipped to on dry ice.
Unscheduled Euthanasia: Terminal tissues were collected from moribund animals that are euthanized prior to their scheduled time point. If possible, tissues were collected and stored from animals that are found dead.
Euthanasia: On Day 28, after a minimum 4 hours fast, animals were euthanized by CO2 asphyxiation followed by thoracotomy and exsanguination.
Terminal Blood: Whole blood from exsanguination was collected into a serum separator with clot activator tube and processed into two (2) aliquot of serum per facility SOPs. All samples were stored at nominally −70° C. until shipped to on dry ice.
Phenylalanine (PHE) Levels: Serum samples were analyzed by the Pure Honey for PHE levels.
Activity Levels: Two (2) frozen liver samples were analyzed by Pure Honey for Activity levels.

### Results

As shown in the right panel of FIG. 6, when dosed hydrodynamically at 5 μg, all constructs tested in this study (ceDNA412, ceDNA1132, ceDNA1274 and ceDNA1527) corrected average PHE levels (PHE μM) to below target concentration (less than 350 μM) through day 7 of the study. PHE levels begin rising again around day 14 of the study. FIG. 6A shows the wild type mouse controls, which had normal PHE levels (below target concentration), as expected, and PAH^{enu2} mice, dosed with vehicle, had high levels of PHE, also as expected. While ceDNA vectors with codon optimized human PAH version 2 corrected PHE levels close to the target correction at a dose of 0.5 μg, the level of correction never reached below the target.

ceDNA1132 was previously examined in an in vivo study, where n=2/3 animals resulted in PHE correction (data not shown). Because ceDNA1132 expressed well in vitro, it was tested again in vivo to increase n. As shown in FIGS. 7D and 7E, at a 5 μg dose, all 5 animals in the ceDNA1132 and ceDNA1274 groups showed corrected PHE levels (PHE μM) to below target concentration (less than 350 μM) at day 7. There was only 1 non-responder in the ceDNA412 and ceDNA1527 groups. As also shown in FIGS. 7A, 7C, and 7F, at a dose of 0.5 μg, there was no distinguishable difference in PHE correction among the constructs tested; however it is considered that ceDNA1527 may be more potent if single non-responder is excluded. Taken together, the data show that all of the constructs tested in this study corrected average PHE levels below target concentration through at least day 7 when dosed at 5 μg, hydrodynamically. In some groups, correction below target levels was maintained for as long as 20 days or more in individual mice when dosed at 5 μg, hydrodynamically.

### Example 8: Pharmacology Study to Evaluate Biochemical Correction of Phenylalanine Levels in PAHe^{nu2} Mice by Hydrodynamic Administration of ceDNA-Effect of Different Promoters on PHE Correction

ceDNA vectors were prepared and purified as described above in Examples 1 and 5.

The nucleic acid sequence of ceDNA containing codon optimized human PAH version 2 (ceDNA “hPAH Codop2”) is shown herein as SEQ ID NO: 192, and includes the following elements: left-ITR_v1: spacer_left-ITR_v2.1: VD_Promoter Set (VD): PmeI_site: Modified_Minimum_Consensus_Kozak: hPAH_codop_ORF_v2: PacI_site: WPRE_3pUTR: bGH/spacer: spacer_right-ITR_v1: right-ITR_v1 (ceDNA412).

The nucleic acid sequence of ceDNA containing codon optimized human PAH version 2 r5-s29 (ceDNA “hPAH-r5-s29”) with specific cis-regulatory elements is shown herein as SEQ ID NO: 196, and includes the following elements: left-ITR_v1, spacer_left-ITR_v2.1, 3×VanD_TTRe_PromoterSet, PmeI_site, Mod_Minimum_Consensus_Kozak, hPAH-r5-s29::hIVS1B, PacI_site, WPRE_3pUTR, bGH, spacer_right-ITR_v1, right-ITR_v1 (ceDNA1414).

The nucleic acid sequence of ceDNA containing codon optimized human PAH version 2 r5-s29 (ceDNA “hPAH-r5-s29”), with specific cis-regulatory elements is shown herein as SEQ ID NO: 197, and includes the following elements: left-ITR_v1, spacer_left-ITR_v2.1, 3×VanD_TTRe_PromoterSet, PmeI_site, Mod_Minimum_Consensus_Kozak, hPAH-r5-s29::hIVS1B_33bpFlanks, PacI_site, WPRE_3pUTR, bGH, spacer_right-ITR_v1, right-ITR_v1 (ceDNA1416).

The nucleic acid sequence of ceDNA codon optimized human PAH version 2 r5-s29 (ceDNA “hPAH-r5-s29”) with specific cis-regulatory elements is shown herein as SEQ ID NO: 198, and includes the following elements: left-ITR_v1, spacer_left-ITR_v2.1, 3×VanD_TTRe_PromoterSet, PmeI_site, Mod_Minimum_Consensus_Kozak, hPAH-r5-s29::mod-Intron_oIVS-v2_33bpFlanks, PacI_site, WPRE_3pUTR, bGH, spacer_right-ITR_v1, right-ITR_v1 (ceDNA1428).

The nucleic acid sequence of ceDNA codon optimized human PAH version 2 (ceDNA “hPAH_codop_ORF_v2”), with specific cis-regulatory elements is shown herein as SEQ ID NO: 211, and includes the following elements: left-ITR_v1, spacer_left-ITR_v2.1, CpGmin_hAAT_Promoter_Set, PmeI_site, Mod_Minimum_Consensus_Kozak, hPAH_codop_ORF_v2, PacI_site, WPRE_3pUTR, bGH, spacer_right-ITR_v1, right-ITR_v1 1 (ceDNA1528).

Each of the ceDNA PAH vectors (alone, without any LNP encapsulation) and the control were administered to mixed gender, age-matched PAHenu2 mice approximately 4-6 weeks old. The naked ceDNA vectors were dosed at 0.5 μg or 5 μg per animal (5 animals per group) by hydrodynamic intravenous injection via lateral tail vein in a dose volume of 90-100 ml/kg on day 0. Day 28 was the terminal time point. The study design is shown below in Table 19.

TABLE 19

| | | | | Dose | | Dosing | |
|---|---|---|---|---|---|---|---|
| Group | No. of | | | Levels | | Regimen | Terminal |
| No. | Animals | Genotype | Test Material | (μg) | Dose Volume | ROA | Time Point |
| 1 | 5 | WT | PBS | NA | 90-100 ml/kg | ONCE ON | Day 28 |
| 2 | 5 | PAH^{enu2} | PBS | NA | (set volume) | Day 0 by | |
| 3 | 5 | MUT | ceDNA412 | 0.5 | | Hydrodynamic | |
| 4 | 5 | | ceDNA412 | 5.0 | | IV | |
| 5 | 5 | | ceDNA1414 | 0.5 | | | |
| 6 | 5 | | ceDNA1414 | 5.0 | | | |
| 7 | 5 | | ceDNA1416 | 0.5 | | | |
| 8 | 5 | | ceDNA1416 | 5.0 | | | |
| 9 | 5 | | ceDNA1428 | 0.5 | | | |
| 10 | 5 | | ceDNA1428 | 5.0 | | | |
| 11 | 5 | | ceDNA1528 | 0.5 | | | |
| 12 | 5 | | ceDNA1528 | 5.0 | | | |

No. = Number; IV = intravenous; ROA = route of administration; WT = wild type; MUT = homozygous mutant; min = minute; hr = hour

Blood was collected at interim and terminal time points as follows in Tables 20A and 20B, respectively.

TABLE 20A
Blood Collection (Interim)

| | Whole Blood Sample Collection Times |
|---|---|
| | Saphenous Only |
| Group | (Orbital by permission of Vet Staff) |
| Number | Serum^{a} |
| 1-12 | FASTED |
| | Day -3, 4, 7, 14, & 21 |
| Volume/ | ~50 μL whole blood |
| Portion | |
| Processing | One (1) aliquot |
| | (Pure Honey) |
| Storage | Frozen at nominally −70° C. |

^{a}Whole blood will be collected into serum separator tubes, with clot activator

TABLE 20B
Terminal Blood and Tissue Collection

| | Sample Collection Times |
|---|---|
| | Terminal Whole Blood |
| Group | (cardiac) |
| Number | Serum^{a} |
| 1-12 | FASTED |
| | Day 28 |
| Portion | MOV |
| Processing/ | One (1) aliquot processed plasma |
| Storage | frozen at nominally −70° C. |

^{a}Whole blood will be collected into serum separator tubes, with clot activator, MOV = maximum obtainable volume

### Study Details are Provided as Follows:

Species (number, sex, age): 55+2 spare PAH^{enu2} Mutant (MUT) mice (mixed gender, ˜5-10 weeks old, age-matched at arrival); 5 Wild Type (WT); mixed gender, littermates; age-matched. Animals were ˜10-14 weeks of at dose initiation.
Cage Side Observations: Cage side observations were performed daily.
Clinical Observations: Clinical observations were performed ˜1, ˜5-6 and ˜24 hours post the Day 0 Test Material dose, as applicable for remaining groups.
Class of Compound: Recombinant DNA vector, ceDNA.
Body Weights: Body weights for all animals were recorded on Days −5, 0, 1, 2, 3, 7, 14, 21 & 29 (prior to euthanasia). Additional body weight may be recorded as requested.
Dose Formulation: Test articles were supplied in a concentration stock. Stock was warmed to room temperature and diluted with the provided PBS immediately prior to use. Prepared materials may be stored at ˜4° C. if dosing is not performed immediately.
Dose Administration: Test Materials for Groups 1-12 were dosed on Day 0 by hydrodynamic IV administration, at a set volume per animal, 90-100 ml/kg (dependent on the lightest animal in the group) via lateral tail vein.
Fasting Prior to Blood Collection: All animals (all groups) were fasted for a minimum of 4 hours prior to the all interim and terminal blood collections: Days −5, 3, 7, 14, 21 & 29.

Food was removed and bedding changed. Food will be returned at the conclusion of each interim blood collections for a fast of no more than 8 hours in duration.
Interim Blood Collection: All animals in Groups 1-12 will have interim blood collected on Days −5, 3, 7, 14 & 21. Animals will have whole blood for fasted serum collection. After collection animals will receive 0.5-1.0 mL lactated Ringer's, subcutaneously
Blood Collection: Whole blood for serum was be collected by saphenous vein. Whole blood was collected into a serum separator with clot activator tube and processed into one (1) aliquot of serum per facility SOPs. All samples were be stored at nominally −70° C. until shipped to on dry ice.
Unscheduled Euthanasia: Terminal tissues were collected from moribund animals that are euthanized prior to their scheduled time point. If possible, tissues will be collected and stored from animals that are found dead.
Euthanasia: On Day 29, after a minimum 4 hours fast, animals were euthanized by CO2 asphyxiation followed by thoracotomy and exsanguination.
Terminal Blood: Whole blood from exsanguination was collected into a serum separator with clot activator tube and processed into two (2) aliquot of serum per facility SOPs. All samples were stored at nominally −70° C. until shipped to on dry ice.
Phenylalanine (PHE) Levels: Serum samples were analyzed by the Pure Honey for PHE levels.
Activity Levels: Two (2) frozen liver samples were analyzed by Pure Honey for Activity levels.

### Results

The present study tested two codon optimized human PAH sequences (codon optimized human PAH version 2 and codon optimized human PAH version 2 r5-s29 in combination with different promoters or combinations of cis-regulatory elements. Specifically, the following constructs were tested, each of which had a different combination of promoter, codon optimized sequence, CpG content (e.g., CpGmin), intron, etc.

TABLE 21

| ceDNA | Construct features |
|---|---|
| ceDNA412 | 1× hSerpEnh (VD)_PromoterSet ∥ hPAH_codop_ORF_v2 |
| ceDNA1414 | 3× hSerpEnh (VanD)_TTRe_PromoterSet ∥ |
| | hPAH-r5-s29::hIVS1B ∥ WPRE_3pUTR |
| ceDNA1416 | 3× hSerpEnh (VanD)_TTRe_PromoterSet ∥ hPAH-r5-s29:: |
| | hIVS1B_33bpFlanks ∥ WPRE_3pUTR |
| ceDNA1428 | 3× hSerpEnh (VanD)_TTRe_PromoterSet ∥ hPAH-r5-s29:: |
| | mod-Intron_oIVS-v2_33bpFlanks ∥ WPRE_3pUTR |
| ceDNA1528 | CpGmin_hAAT_Promoter_Set ∥ hPAH_codop_ORF_v2 |

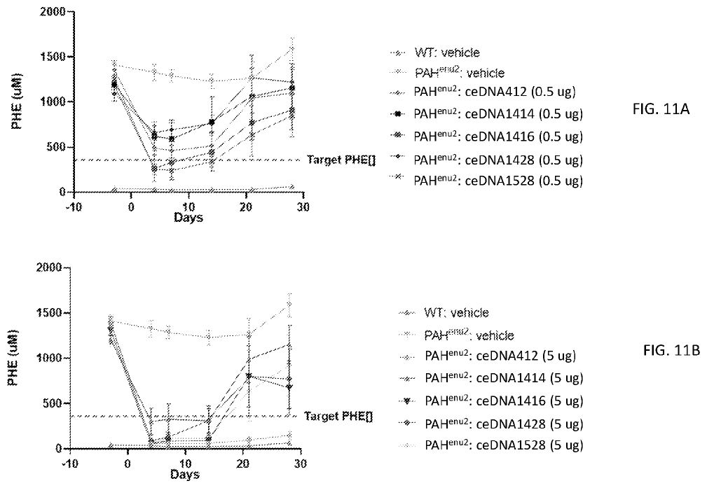

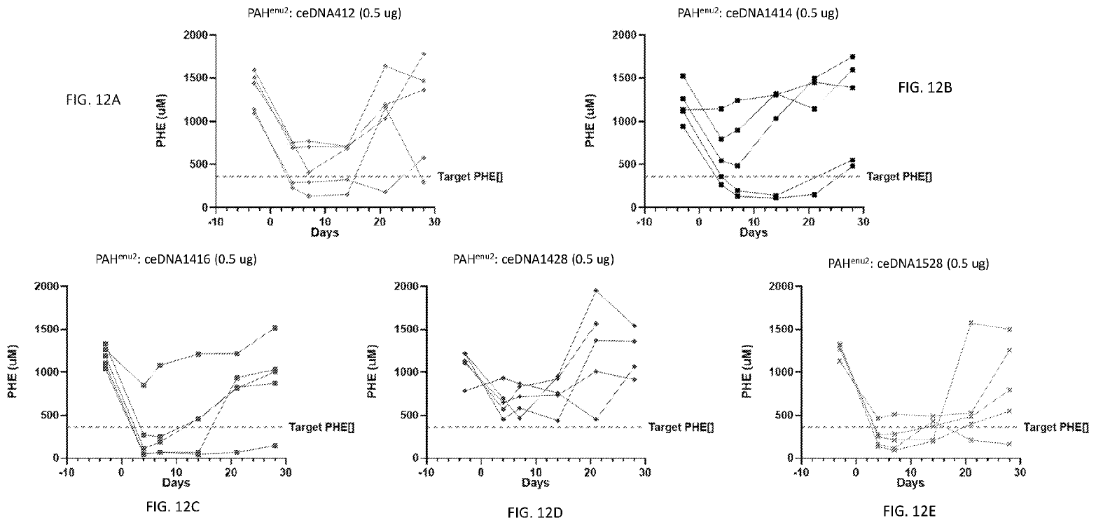

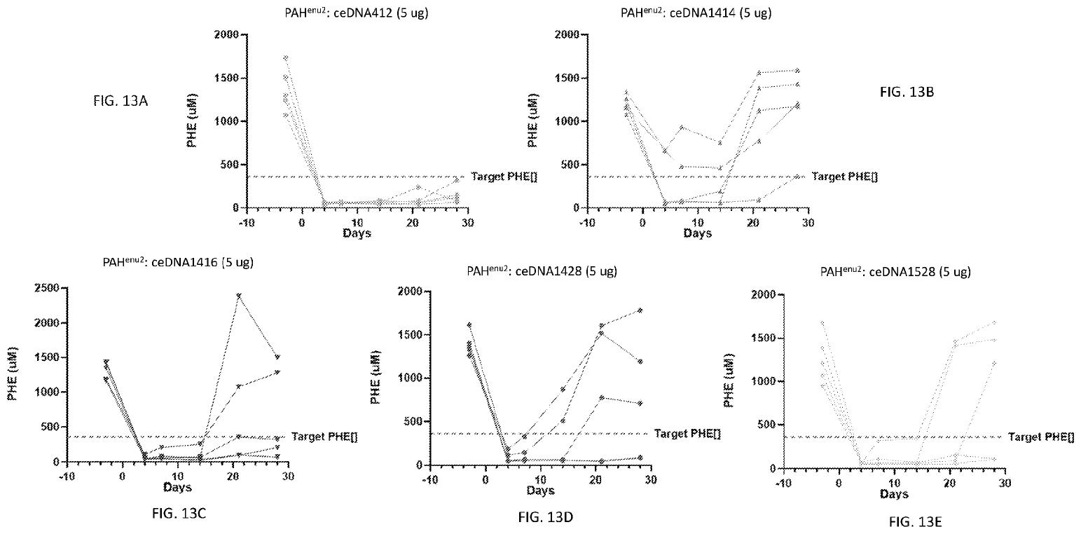

As shown in FIGS. 8A-8B, 9A-9E, and 10A-10E, 11A-11B, 12A-12E, 13A-13E, and 14A-14E, at a 5 μg dose, ceDNA1416, ceDNA1428, and ceDNA1528, but not ceDNA1414 groups showed acute correction of PHE levels (PHE μM) to below target concentration (less than 350 μM) in 5/5 mice within 7 days. As shown in FIGS. 8A-8r and 9A-9E, the data at the 0.5 μg dose suggests that ceDNA1416 and ceDNA1528 may be more potent than ceDNA412 (4/5 mice showed correction of PHE levels (PHE μM) to below target concentration (less than 350 μM) compared to 2/5 mice for ceDNA412 after 7 days). Based on this data, it is possible to conclude that the CpGmin_hAAT_Promoter_Set may show improvement over the standard VD Promoter. 3×VanD_TTRe is also a strong promoter, but further experimentation will be required for comparison to VanD (VD) Promoter in this data set.

As shown in FIGS. 11A-11B, 12A-12E, and 13A-13E, all of the mice (5/5) in the ceDNA412 group (5 μg dose) showed correction of PHE levels (PHE μM) to below target concentration (less than 350 μM) for the entire 28-day duration of the study. This result has not been seen before, and suggests a surprising durability of gene expression and efficacy. The other codon optimized constructs tested showed expected Phe increase after day 14 (at a 5 μg dose).

Finally, a further study was carried out testing codon optimized human PAH sequences in combination with different promoters or combinations of cis-regulatory elements. Specifically, the following constructs were tested, each of which had a different combination of promoter, codon optimized sequence, CpG content (e.g., CpGmin), intron, etc.

TABLE 22

| ceDNA | Construct features |
|---|---|
| ceDNA412 | 1× VD_PromoterSet ∥ hPAH_codop_ORF_v2 |
| ceDNA1430 | 3× hSerpEnh (VanD)_TTRe_PromoterSet ∥ |
| | hPAH_codop_ORF_v2_mIVS-intron1B_33bpFlanks ∥ WPRE_3pUTR |
| ceDNA1432 | 3× hSerpEnh (VanD)_TTRe_PromoterSet ∥ |
| | hPAH_codop_ORF_v2_modified_Intron1_33bpFlanks ∥ WPRE_3pUTR |
| ceDNA1473 | 5× HNF1 ∥ Pro-Albumin Enh ∥ TTR promoter ∥ hPAH_ORF_codop_v2 |
| ceDNA1474 | 5× HNF1 ∥ Pro-Albumin Enh ∥ 3× VanD-TTRe ∥ TTR promoter ∥ |
| | hPAH_ORF_codop_v2 |
| ceDNA1436 | hAAT(979)_PromoterSet ∥ hPAH_codop_ORF_v2 ∥ WPRE_3pUTR |
| ceDNA1471 | 3× HNF1-4 ∥ Pro-Albumin Enh ∥ TTR promoter ∥ hPAH_ORF_codop_v2 |
| ceDNA1472 | 3× HNF1-4 ∥ Pro-Albumin Enh ∥ 3× VanD-TTRe ∥ TTR promoter ∥ |
| | hPAH_ORF_codop_v2 |

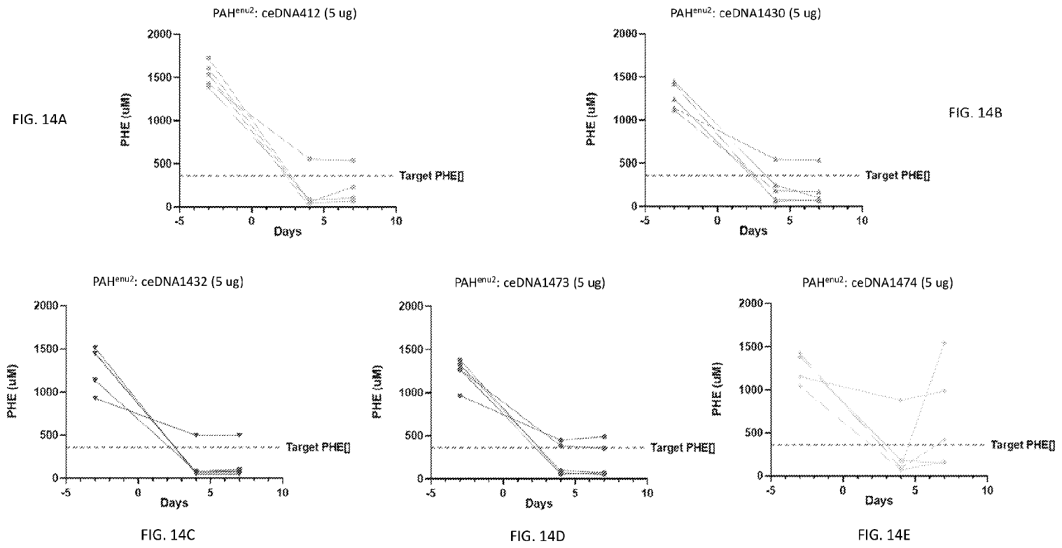

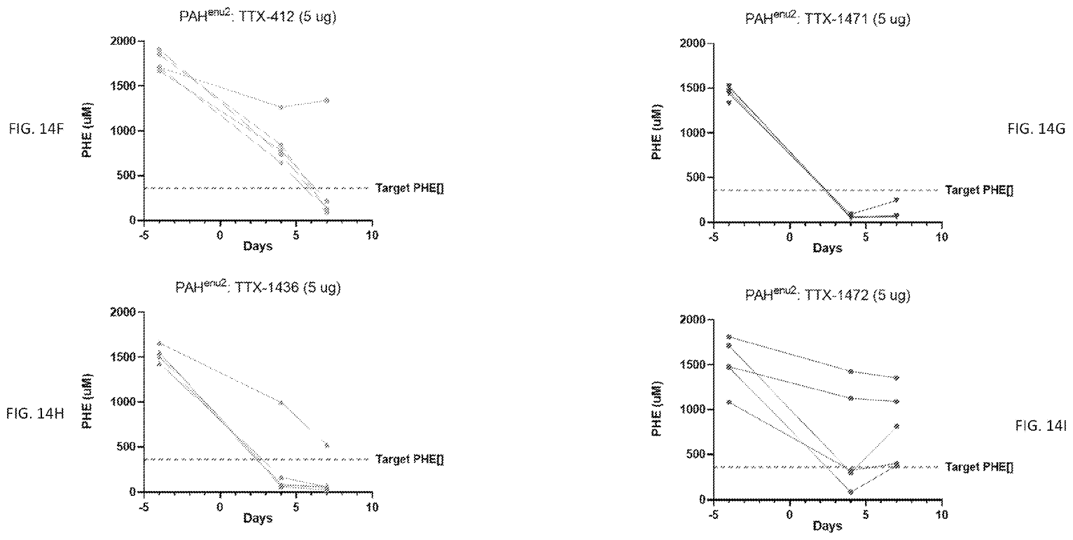

All mice were administered a 5 μg dose, hydrodynamically. Surprisingly, as shown in FIGS. 14A-14I, particular combinations of cis-regulatory elements with the codon optimized sequences showed correction of PHE levels (PHE μM) to below target concentration (less than 350 μM). In particular, ceDNA1471, which has the promoter combination 3×HNF1-4∥Pro-Albumin Enh∥TTR promoter lowers PHE concentration below target values at a 5 μg dose, showing greater potency than ceDNA412 (VD_PromoterSet∥hPAH_codop_ORF_v2) in this study.

### Example 9: Pharmacology Study to Evaluate Biochemical Correction of Phenylalanine Levels in PAHe^{nu2} Mice by Hydrodynamic Administration of ceDNA-Effect of Different Promoters or Promoter Sets and Different ORF's on PHE Correction

ceDNA Constructs

This study tested ceDNA vectors having different promoters or promoter sets (i.e., hAAT promoter and VD promoter set) and codon optimized PAH sequences or ORFs (i.e., human PAH cDNA with or without CpG minimization and human PAH version 2). ceDNA vectors were prepared and purified as described above in Examples 1 and 5.

The nucleic acid sequence of ceDNA containing codon optimized human PAH version 2 (ceDNA “hPAH Codop2”) is shown herein as SEQ ID NO: 192, and includes the following elements: left-ITR_v1: spacer_left-ITR_v2.1: VD_Promoter Set: PmeI_site: Modified_Minimum_Consensus_Kozak: hPAH_codop_ORF_v2: PacI_site: WPRE_3pUTR: bGH/spacer: spacer_right-ITR_v1: right-ITR_v1 (ceDNA412). ceDNA412 served as a control in this study described in Example 9.

The nucleic acid sequence of ceDNA containing codon optimized human PAH version 2 ORF (hPAH_codop_ORF_v2), with specific chimeric intron (mIVS-intron1B) and intron flanking region (33bpFlanks), is shown herein as SEQ ID NO: 546 and includes the hAAT promoter in combination with the Pro-Albumin enhancer and 6 copies of hepatic nuclear factors 1 and 4 binding sites (3×HNF1-4) (ceDNA1476).

The nucleic acid sequence of ceDNA containing codon optimized human PAH version 2 ORF (hPAH_codop_ORF_v2), with specific chimeric intron (mIVS-intron1B) and intron flanking region (33bpFlanks), is shown herein as SEQ ID NO: 549 and includes the hAAT promoter in combination with the Pro-Albumin enhancer and 5 copies of hepatic nuclear factors 1 binding sites HNF1 (5×HNF1) (ceDNA1479).

The nucleic acid sequence of ceDNA containing codon optimized human PAH CpG minimized cDNA version 1 ORF (hPAH-cDNA_0CpG1_ORF) is shown herein as SEQ ID NO: 562 and includes the VD_Promoter Set (ceDNA1939).

The nucleic acid sequence of ceDNA containing codon optimized human PAH CpG minimized cDNA version 2 ORF (hPAH-cDNA_0CpG2_ORF) is shown herein as SEQ ID NO: 563 and includes the VD_Promoter Set (ceDNA1940).

The nucleic acid sequence of ceDNA containing codon optimized human PAH CpG minimized cDNA version 3 ORF (hPAH-cDNA_0CpG3_ORF) is shown herein as SEQ ID NO: 564 and includes the VD_Promoter Set (ceDNA1941).

The nucleic acid sequence of ceDNA containing codon optimized human PAH CpG minimized cDNA version 4 ORF (hPAH-cDNA_0CpG4_ORF) is shown herein as SEQ ID NO: 565 and includes the VD_Promoter Set (ceDNA1942).

The nucleic acid sequence of ceDNA containing codon optimized human PAH cDNA version 1 ORF without CpG minimization (hPAH-cDNA_1_ORF) is shown herein as SEQ ID NO: 566 and includes the VD_Promoter Set (ceDNA1943).

The nucleic acid sequence of ceDNA containing codon optimized human PAH cDNA version 2 ORF without CpG minimization (CpG=99) (hPAH-cDNA_2_ORF) is shown herein as SEQ ID NO: 567 and includes the VD_Promoter Set (ceDNA1944).

The features of the ceDNA vectors used in this study and as described above are summarized below in Table 23.

TABLE 23
Construct features of ceDNA vectors in Example 9 study

| ceDNA | Construct features |
|---|---|
| ceDNA412 | 1× VD_PromoterSet ∥ hPAH_codop_ORF_v2 |
| ceDNA1476 | 3× HNF1-4 ∥ Pro-Albumin Enh ∥ hAAT promoter ∥ |
| | hPAH_codop_ORF_v2_mIVS-intron1B_33bpFlanks |
| ceDNA1479 | 5× HNF1 ∥ Pro-Albumin Enh ∥ hAAT promoter ∥ |
| | hPAH_codop_ORF_v2_mIVS-intron1B_33bpFlanks |
| ceDNA1939 | 1× VD_PromoterSet ∥ hPAH-cDNA_0CpG1_ORF |
| ceDNA1940 | 1× VD_PromoterSet ∥ hPAH-cDNA_0CpG2_ORF |
| ceDNA1941 | 1× VD_PromoterSet ∥ hPAH-cDNA_0CpG3_ORF |
| ceDNA1942 | 1× VD_PromoterSet ∥ hPAH-cDNA_0CpG4_ORF |
| ceDNA1943 | 1× VD_PromoterSet ∥ hPAH-cDNA_1_ORF |
| ceDNA1944 | 1× VD_PromoterSet ∥ hPAH-cDNA_2_ORF (CpG = 99) |

### Study Design

Each of the ceDNA PAH vectors (alone, without any LNP encapsulation) and the control were administered to mixed gender, age-matched PAHenu2 mice approximately 4-6 weeks old. The naked ceDNA vectors were dosed at 0.5 μg per animal (5 animals per group) by hydrodynamic intravenous injection via lateral tail vein in a dose volume of 90-100 ml/kg on day 0. Day 28 was the terminal time point. The study design is as shown below in Table 24.

TABLE 24
Test material administration in Example 9 study

| | | | | Dose | | Dosing | |
|---|---|---|---|---|---|---|---|
| Group | No. of | | | Levels | | Regimen | Terminal |
| No. | Animals | Genotype | Test Material | (μg) | Dose Volume | ROA | Time Point |
| 1 | 5 | WT | PBS | NA | 90-100 ml/kg | ONCE ON | Day 28 |
| 2 | 5 | PAH^{enu2} | PBS | NA | (set volume) | Day 0 by | |
| 3 | 5 | MUT | ceDNA412 | 0.5 | | Hydrodynamic | |
| 4 | 5 | | ceDNA1476 | 0.5 | | IV | |
| 5 | 5 | | ceDNA1479 | 0.5 | | | |
| 6 | 5 | | ceDNA1939 | 0.5 | | | |
| 7 | 5 | | ceDNA1940 | 0.5 | | | |
| 8 | 5 | | ceDNA1941 | 0.5 | | | |
| 9 | 5 | | ceDNA1942 | 0.5 | | | |
| 10 | 5 | | ceDNA1943 | 0.5 | | | |
| 11 | 5 | | ceDNA1944 | 0.5 | | | |

No. = Number; IV = intravenous; ROA = route of administration; WT = wild type; MUT = homozygous mutant; min = minute; hr = hour

Blood was collected at interim and terminal time points as follows in Tables 25A and 25B, respectively.

TABLE 25A
Blood Collection (Interim) Example 9 study

| | |
|---|---|
| Group | Whole Blood Sample Collection Times |
| Number | Saphenous Only |
| | (Orbital by permission of Vet Staff) |
| | Serum^{a} |
| 1-11 | FASTED |
| | Day −3, 4, 7, 14, & 21 |
| Volume/ | ~50 μL whole blood |
| Portion | |
| Processing | One (1) aliquot |
| | (Pure Honey) |
| Storage | Frozen at nominally −70° C. |

^{a}Whole blood was collected into serum separator tubes, with clot activator

TABLE 25B
Terminal Blood and Tissue Collection

| | |
|---|---|
| Group | Sample Collection Times |
| Number | Terminal Whole Blood |
| | (cardiac) |
| | Serum^{a} |
| 1-11 | FASTED |
| | Day 28 |
| Portion | MOV |
| Processing/ | One (1) aliquot processed plasma |
| Storage | frozen at nominally −70° C. |

^{a}Whole blood was be collected into serum separator tubes, with clot activator,
MOV = maximum obtainable volume

### Study Details

Species (number, sex, age): 55+4 spare PAH^{enu2} Mutant (MUT) mice (mixed gender, ˜6-8 weeks old, age-matched at arrival); 5 Wild Type (WT); mixed gender, littermates; age-matched. Animals were ˜7-9 weeks of at dose initiation.
Cage Side Observations: Cage side observations were performed daily.
Clinical Observations: Clinical observations were performed ˜1, ˜5-6 and ˜24 hours post the Day 0 Test Material dose, as applicable for remaining groups.
Class of Compound: Recombinant DNA vector, ceDNA.
Body Weights: Body weights for all animals were recorded on Days −4, 0, 1, 2, 3, 7, 14, 21 & 28 (prior to euthanasia).
Dose Formulation: Test articles were supplied in a concentration stock. Stock was warmed to room temperature and diluted with the provided PBS immediately prior to use. Prepared materials may be stored at ˜4° C. if dosing was not performed immediately.
Dose Administration: Test Materials for Groups 1-12 were dosed on Day 0 by hydrodynamic IV administration, at a set volume per animal, 90-100 ml/kg (dependent on the lightest animal in the group) via lateral tail vein.
Fasting Prior to Blood Collection: All animals (all groups) were fasted for a minimum of 4 hours prior to all interim and terminal blood collections: Days −4, 3, 7, 14, 21 & 28.

Food was removed and bedding changed. Food was returned at the conclusion of each interim blood collections for a fast of no more than 8 hours in duration.
Interim Blood Collection: All animals in Groups 1-11 had interim blood collected on Days −4, 3, 7, 14 & 21. Animals had whole blood for fasted serum collection. After collection animals would receive 0.5-1.0 mL lactated Ringer's, subcutaneously
Blood Collection: Whole blood for serum was collected by saphenous vein. Whole blood was collected into a serum separator with clot activator tube and processed into one (1) aliquot of serum per facility SOPs. All samples were stored at nominally −70° C. until shipped to on dry ice.
Unscheduled Euthanasia: Terminal tissues were collected from moribund animals that were euthanized prior to their scheduled time point. If possible, tissues were collected and stored from animals that were found dead.
Euthanasia: On Day 28, after a minimum 4 hours fast, animals were euthanized by CO₂ asphyxiation followed by thoracotomy and exsanguination.
Terminal Blood: Whole blood from exsanguination was collected into a serum separator with clot activator tube and processed into two (2) aliquot of serum per facility SOPs. All samples were stored at nominally −70° C. until shipped to on dry ice.
Phenylalanine (PHE) Levels: Serum samples were analyzed by the Pure Honey for PHE levels.
Activity Levels: Two (2) frozen liver samples were analyzed by Pure Honey for Activity levels.

### Results

The present study tested two codon optimized human PAH sequences (codon optimized human PAH version 2 and codon optimized human PAH cDNA (with or without CpG minimization) in combination with different promoters or combinations of cis-regulatory elements. The specific elements contained in each tested ceDNA construct are summarized in Table 23.

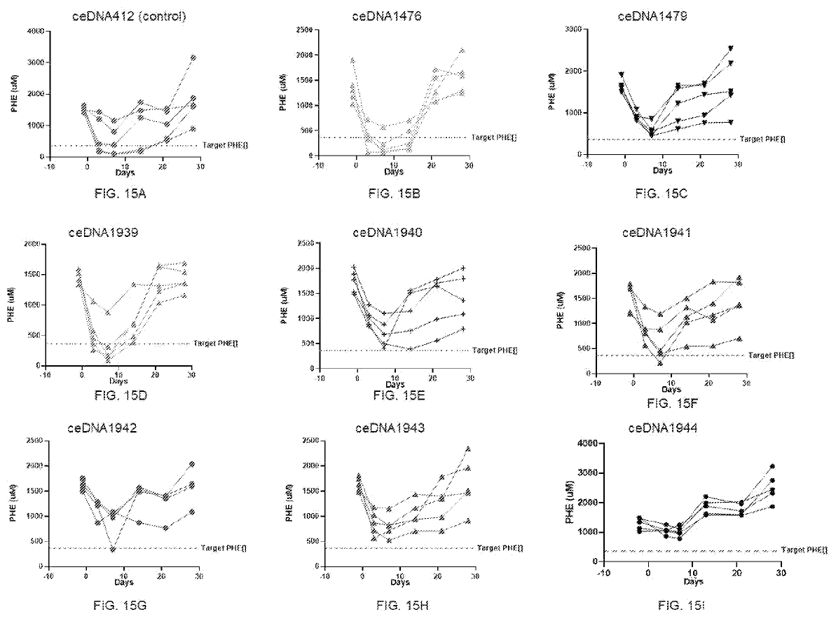

As shown in FIGS. 15A-15I, at a 0.5 μg dose, all ceDNA vectors tested in this study, namely ceDNA1476, ceDNA1939, and ceDNA1941, but not the rest, showed acute correction of PHE levels (PHE μM) to below target concentration (less than 350 μM) in at least one out of total 5 mice within 7 days. Of note, ceDNA1476 and ceDNA1939 each had 4 out of total 5 mice that exhibited acute correction of PHE levels below the target concentration. ceDNA1479, ceDNA1940, and ceDNA1942 each had a single timepoint associated with a single mouse that that was at the 350 μM threshold, but not below; while ceDNA1479, ceDNA1942, ceDNA1943, and ceDNA1944 had no timepoints that were close to the 350 μM threshold.

The only structural difference between ceDNA1476 and ceDNA1479 is that the latter contains 5 copies of HNF1 binding sites (with 10-mer spacers between every two copies of HNF1) while ceDNA1476 contains only 6 copies of the combination of HNF1 and HNF4, where HNF1 and HNF4 alternate one another. The inventors found that the combination of HNF1 and HNF4 resulted in improved PHE level correction (compare FIG. 15C with FIG. 15B).

The difference between ceDNA1943, ceDNA1944 and ceDNA1939, ceDNA1940, ceDNA1941, and ceDNA1942 are the open reading frames of ceDNA1943 and ceDNA1944 have not been subject to CpG minimization. The improved PHE level corrections in ceDNA1939, ceDNA1940, ceDNA1941, and ceDNA1942 (FIGS. 15D-15G) as compared to ceDNA1943 and ceDNA1944 (FIGS. 15H and 15I) illustrate the importance of CpG minimization towards PAH expression and consequently, PHE level correction.

Similar to the observation noted in Example 8 with ceDNA1471, here in Example 9 it was noted that ceDNA1476, which has the promoter combination 3×HNF1-4∥Pro-Albumin Enh∥TTR promoter lowers PHE concentration below target values at a 0.5 μg dose, showing greater potency than the control ceDNA412 (1× VD_PromoterSet∥hPAH_codop_ORF_v2) in this study.

### Example 10: Pharmacology Study to Evaluate Biochemical Correction of Phenylalanine Levels in PAHe^{nu2} Mice by Hydrodynamic Administration of ceDNA-Effect of Different Promoters or Promoter Sets and PAH from Mouse or Human on PHE Correction

ceDNA Constructs

This study tested ceDNA vectors having different promoters or promoter sets (i.e., hAAT promoter and VD promoter set) and codon optimized PAH sequences or ORFs from mouse or human (i.e., mousePAH_codop_ORF_v2 or hPAH_codop_ORF_v2). ceDNA vectors were prepared and purified as described above in Examples 1 and 5.

The nucleic acid sequence of ceDNA containing codon optimized human PAH version 2 (ceDNA “hPAH Codop2”) is shown herein as SEQ ID NO: 192, and includes the following elements: left-ITR_v1: spacer_left-ITR_v2.1: VD_Promoter Set: PmeI_site: Modified_Minimum_Consensus_Kozak: hPAH_codop_ORF_v2: PacI_site: WPRE_3pUTR: bGH/spacer: spacer_right-ITR_v1: right-ITR_v1 (ceDNA412). ceDNA412 served as a control in this study described in Example 10.

The nucleic acid sequence of ceDNA containing codon optimized human PAH CpG minimized cDNA version 1 ORF (hPAH-cDNA_0CpG1_ORF) is shown herein as SEQ ID NO: 562 and includes the VD_Promoter Set (ceDNA1939). ceDNA1939, which was previously studied in in Example 9, also served as a control in this study described in Example 10.

The nucleic acid sequence of ceDNA containing codon optimized mouse PAH version 2 ORF (mousePAH_codop_ORF_v2) is shown herein as SEQ ID NO: 568 and includes the VD_Promoter Set (ceDNA1955).

The nucleic acid sequence of ceDNA containing codon optimized human PAH version 2 ORF (hPAH_codop_ORF_v2) is shown herein as SEQ ID NO: 569 and includes the human AAT promoter with the enhancers that are present in ceDNA1476 and ceDNA1479 (ceDNA62).

The features of the ceDNA vectors used in this study and as described above are summarized below in Table 26.

TABLE 26
Construct features of ceDNA vectors in Example 10 study

| ceDNA | Construct features |
|---|---|
| ceDNA412 | VD_PromoterSet ∥ hPAH_codop_ORF_v2 |
| ceDNA1939 | VD_PromoterSet ∥ hPAH-cDNA_0CpG1_ORF |
| ceDNA1955 | VD_PromoterSet ∥ mousePAH_codop_ORF_v2 |
| ceDNA62 | hAAT ∥ mousePAH_codop_ORF_v2 |

### Study Design

Each of the ceDNA PAH vectors (alone, without any LNP encapsulation) and the control were administered to mixed gender, age-matched PAHenu2 mice approximately 7-9 weeks old. The naked ceDNA vectors were dosed at 0.5 μg per animal (5 animals per group) by hydrodynamic intravenous injection via lateral tail vein in a dose volume of 90-100 ml/kg on day 0. Day 28 was the terminal time point. The study design is as shown below in Table 27.

TABLE 27
Test material administration in Example 10 study

| | | | | Dose | | Dosing | |
|---|---|---|---|---|---|---|---|
| Group | No. of | | | Levels | | Regimen | Terminal |
| No. | Animals | Genotype | Test Material | (μg) | Dose Volume | ROA | Time Point |
| 1 | 5 | WT | PBS | NA | 90-100 ml/kg | ONCE ON | Day 28 |
| 2 | 5 | PAH^{enu2} | PBS | NA | (set volume) | Day 0 by | |
| 3 | 5 | MUT | ceDNA412 | 0.5 | | Hydrodynamic | |
| 4 | 5 | | ceDNA1939 | 0.5 | | IV | |
| 5 | 5 | | ceDNA1955 | 0.5 | | | |
| 6 | 5 | | ceDNA62 | 0.5 | | | |

No. = Number; IV = intravenous; ROA = route of administration; WT = wild type; MUT = homozygous mutant; min = minute; hr = hour

Blood was collected at interim and terminal time points as follows in Tables 28A and 28B, respectively.

TABLE 28A
Blood Collection (Interim) Example 10 study

| | |
|---|---|
| Group | Whole Blood Sample Collection Times |
| Number | Saphenous Only |
| | (Orbital by permission of Vet Staff) |
| | Serum^{a} |
| 1-6 | FASTED |
| | Day −1, 3, 7, 14, & 21 |
| Volume/ | ~50 μL whole blood |
| Portion | |
| Processing | One (1) aliquot |
| | (Pure Honey) |
| Storage | Frozen at nominally −70° C. |

^{a}Whole blood was collected into serum separator tubes, with clot activator

TABLE 28B
Terminal Blood and Tissue Collection

| | |
|---|---|
| Group | Sample Collection Times |
| Number | Terminal Whole Blood |
| | (cardiac) |
| | Serum^{a} |
| 1-6 | FASTED |
| | Day 28 |
| Portion | MOV |
| Processing/ | One (1) aliquot processed plasma |
| Storage | frozen at nominally −70° C. |

^{a}Whole blood was be collected into serum separator tubes, with clot activator,
MOV = maximum obtainable volume

### Study Details

Species (number, sex, age): 30+3 spare PAH^{enu2} Mutant (MUT) mice (mixed gender, ˜6-8 weeks old, age-matched at arrival); 5 Wild Type (WT); mixed gender, littermates; age-matched. Animals were ˜7-9 weeks of at dose initiation.
Cage Side Observations: Cage side observations were performed daily.
Clinical Observations: Clinical observations were performed ˜1, ˜5-6 and ˜24 hours post the Day 0 Test Material dose, as applicable for remaining groups.
Class of Compound: Recombinant DNA vector, ceDNA.
Body Weights: Body weights for all animals were recorded on Days −1, 0, 1, 2, 3, 7, 14, 21 & 28 (prior to euthanasia).
Dose Formulation: Test articles were supplied in a concentration stock. Stock was warmed to room temperature and diluted with the provided PBS immediately prior to use. Prepared materials may be stored at ˜4° C. if dosing is not performed immediately.
Dose Administration: Test Materials for Groups 1-5 were dosed on Day 0 by hydrodynamic IV administration, at a set volume per animal, 90-100 ml/kg (dependent on the lightest animal in the group) via lateral tail vein.
Fasting Prior to Blood Collection: All animals (all groups) were fasted for a minimum of 4 hours prior to all interim and terminal blood collections: Days −4, 3, 7, 14, 21 & 28.
Food was removed and bedding changed. Food was returned at the conclusion of each interim blood collections for a fast of no more than 8 hours in duration.
Interim Blood Collection: All animals in Groups 1-6 had interim blood collected on Days −1, 3, 7, 14 & 21. Animals had whole blood for fasted serum collection. After collection animals would receive 0.5-1.0 mL lactated Ringer's, subcutaneously
Blood Collection: Whole blood for serum was collected by saphenous vein. Whole blood was collected into a serum separator with clot activator tube and processed into one (1) aliquot of serum per facility SOPs. All samples were be stored at nominally −70° C. until shipped to on dry ice.
Unscheduled Euthanasia: Terminal tissues were collected from moribund animals that are euthanized prior to their scheduled time point. If possible, tissues will be collected and stored from animals that are found dead.
Euthanasia: On Day 28, after a minimum 4 hours fast, animals were euthanized by CO₂ asphyxiation followed by thoracotomy and exsanguination.
Terminal Blood: Whole blood from exsanguination was collected into a serum separator with clot activator tube and processed into two (2) aliquot of serum per facility SOPs. All samples were stored at nominally −70° C. until shipped to on dry ice.
Phenylalanine (PHE) Levels: Serum samples were analyzed by the Pure Honey for PHE levels.
Activity Levels: Two (2) frozen liver samples were analyzed by Pure Honey for Activity levels.

### Results

The present study tested two codon optimized PAH sequences (i.e., mouse or human codon optimized PAH version 2 in combination with different promoters (i.e., VD promoter set or human AAT promoter. The specific elements contained in each tested ceDNA construct are summarized in Table 26.

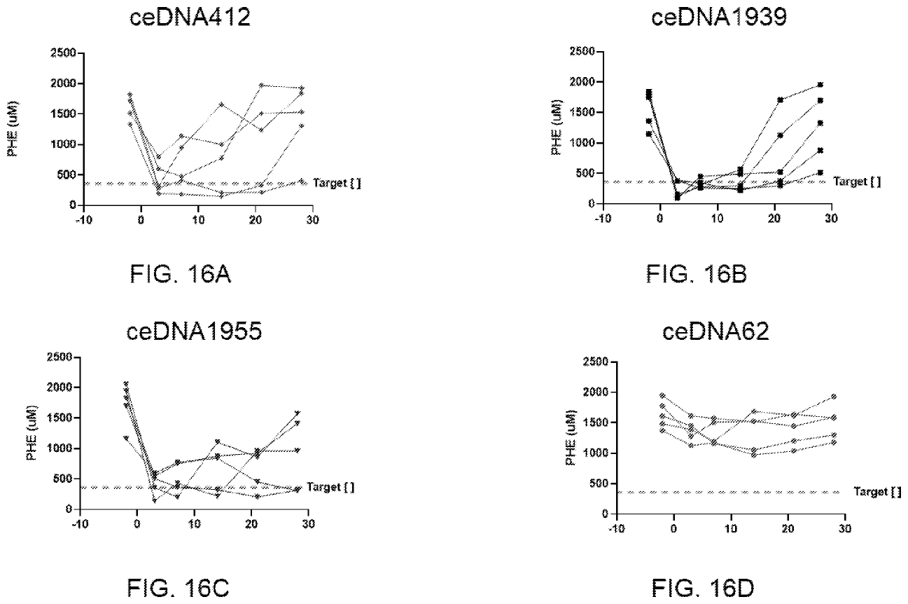

As shown in FIGS. 16A-16D, at a 0.5 μg dose, and as demonstrated in at least Example 9, ceDNA412 and ceDNA1939, both expressing the codon-optimized human PAH ORF version 2, showed acute correction of PHE levels (PHE μM) to below target concentration (less than 350 μM) in at least one out of total 5 mice within 7 days. As it was so observed in Example 9, ceDNA1939 again exhibited acute PHE correction in 4 out of total 5 mice within 7 days.

Surprisingly, ceDNA1955 and ceDNA62 that both expressed the codon-optimized mouse PAH ORF version 2, did not result in PHE correction in the mice that was superior to ceDNA412 and ceDNA1939. ceDNA1955, at best, can be said to result in PHE correction that was equivalent or slightly inferior to ceDNA1939 correction. ceDNA62 did not show any PHE correction altogether. ceDNA62 has the hAAT promoter but no enhancers like ceDNA1476 and ceDNA1479 have (studied in Example 8) and without CpG minimization like ceDNA1528 has (studied in Example 7). The lack of PHE correction in ceDNA62 illustrates the importance of enhancers and CpG minimization in improving the hAAT promoter.

### Example 11: Pharmacology Study to Evaluate Biochemical Correction of Phenylalanine Levels in PAHe^{nu2} Mice by Hydrodynamic Administration of ceDNA-Effect of Different Promoters and their Cis-Regulatory Elements, Introns, and UTRs on PHE Correction

ceDNA Constructs

This study tested ceDNA vectors having different promoter sets (i.e., hAAT promoter sets and TTR promoter sets) and their respective cis-regulatory elements, post-transcriptional regulatory elements, introns and UTRs and codon optimized human PAH sequences or ORFs (i.e., hPAH-r5-s29, hPAH-r5-s29::hIVS1B_33bpFlanks, or hPAH-r5-s29::mod-Intron_oIVS-v2_33bpFlanks). ceDNA vectors were prepared and purified as described above in Examples 1 and 5.

The nucleic acid sequence of ceDNA containing codon optimized human PAH version 2 (ceDNA “hPAH Codop2”) is shown herein as SEQ ID NO: 192, and includes the following elements: left-ITR_v1: spacer_left-ITR_v2.1: VD_Promoter Set (VD): PmeI_site: Modified_Minimum_Consensus_Kozak: hPAH_codop_ORF_v2: PacI_site: WPRE_3pUTR: bGH/spacer: spacer_right-ITR_v1: right-ITR_v1 (ceDNA412). ceDNA412 served as a control in this study described in Example 11.

The nucleic acid sequence of ceDNA containing codon optimized human PAH version 2 r5-s29 with an intron and an intron flanking region (hPAH-r5-s29::oIVS1B_33bpFlanks) is shown herein as SEQ ID NO: 570 and includes the TTR promoter and other enhancers (i.e. PromoterSet-1471), introns, UTRs, restriction endonuclease site, cis- and post-transcriptional regulatory elements (ceDNA2409).

The nucleic acid sequence of ceDNA containing codon optimized human PAH version 2 r5-s29 with an intron and an intron flanking region (hPAH-r5-s29::mod-Intron_oIVS-v2_33bpFlanks) is shown herein as SEQ ID NO: 571 and includes the TTR promoter and the same enhancers (i.e., PromoterSet-1471), introns, UTRs, restriction endonuclease site, cis- and post-transcriptional regulatory elements as in ceDNA2409 (ceDNA2410).

The nucleic acid sequence of ceDNA containing codon optimized human PAH version 2 r5-s29 with an intron and an intron flanking region (hPAH-r5-s29::oIVS1B_33bpFlanks) is shown herein as SEQ ID NO: 572 and includes the hAAT promoter and other enhancers (i.e., PromoterSet-1476), introns, UTRs, restriction endonuclease site, cis- and post-transcriptional regulatory elements (ceDNA2415).

The nucleic acid sequence of ceDNA containing codon optimized human PAH version 2 r5-s29 with an intron and an intron flanking region (hPAH-r5-s29::oIVS1B_33bpFlanks) is shown herein as SEQ ID NO: 573 and includes the hAAT promoter and other enhancers (i.e., PromoterSet-1479), introns, UTRs, restriction endonuclease site, cis- and post-transcriptional regulatory elements (ceDNA2418). ceDNA2418 differs from ceDNA2415 in terms of the number of copies and types of HNF binding sites.

The nucleic acid sequence of ceDNA containing codon optimized human PAH version 2 r5-s29 with an intron and an intron flanking region (hPAH-r5-s29::mod-Intron_oIVS-v2_33bpFlanks) is shown herein as SEQ ID NO: 574 and includes the hAAT promoter and the same enhancers (i.e., PromoterSet-1476), introns, UTRs, restriction endonuclease site, cis- and post-transcriptional regulatory elements as in ceDNA2415 (ceDNA2416).

The nucleic acid sequence of ceDNA containing codon optimized human PAH version 2 r5-s29 with an intron and an intron flanking region (hPAH-r5-s29::mod-Intron_oIVS-v2_33bpFlanks) is shown herein as SEQ ID NO: 575 and includes the hAAT promoter and the same enhancers (PromoterSet-1479, introns, UTRs, restriction endonuclease site, cis- and post-transcriptional regulatory elements as in ceDNA2418 (ceDNA2419). ceDNA2419 differs from ceDNA2416 in terms of the number of copies and types of HNF binding sites.

The nucleic acid sequence of ceDNA containing codon optimized human PAH version 2 r5-s29 without an intron and an intron flanking region (hPAH-r5-s29) is shown herein as SEQ ID NO: 576 and includes the TTR promoter and the same enhancers (i.e., PromoterSet-1471), introns, UTRs, restriction endonuclease site, cis- and post-transcriptional regulatory elements as in ceDNA2409 (ceDNA2420).

The features of the ceDNA vectors used in this study and as described above are summarized below in Table 29.

TABLE 29
Construct features of ceDNA vectors in Example 11 study

| ceDNA | Construct features |
|---|---|
| ceDNA412 | 1× VD_PromoterSet ∥ hPAH_codop_ORF_v2 |
| ceDNA2409 | PromoterSet-1471 ∥ TTR-MVM_v2-PmeI-Mod2-5pUTR ∥ |
| | hPAH-r5-s29::hIVS1B_33bpFlanks ∥ WPRE_3pUTR |
| ceDNA2410 | PromoterSet-1471 ∥ TTR-MVM_v2-PmeI-Mod2-5pUTR ∥ |
| | hPAH-r5-s29::mod-Intron_oIVS-v2_33bpFlanks ∥ WPRE_3pUTR |
| ceDNA2415 | PromoterSet-1476 ∥ hAAT-PmeI-Mod2-5pUTR ∥ hPAH-r5- |
| | s29::hIVS1B_33bpFlanks ∥ WPRE_3pUTR |
| ceDNA2418 | PromoterSet-1479 ∥ hAAT-PmeI-Mod2-5pUTR ∥ hPAH-r5- |
| | s29::hIVS1B_33bpFlanks ∥ WPRE_3pUTR |
| ceDNA2416 | PromoterSet-1476 ∥ hAAT-PmeI-Mod2-5pUTR ∥ hPAH-r5-s29:: |
| | mod-Intron_oIVS-v2_33bpFlanks ∥ WPRE_3pUTR |
| ceDNA2419 | PromoterSet-1479 ∥ hAAT-PmeI-Mod2-5pUTR ∥ hPAH-r5-s29:: |
| | mod-Intron_oIVS-v2_33bpFlanks ∥ WPRE_3pUTR |
| ceDNA2420 | PromoterSet-1471 ∥ TTR-MVM_v2-PmeI-Mod2-5pUTR ∥ |
| | hPAH-r5-s29 ∥ WPRE_3pUTR |

### Study Design

Each of the ceDNA PAH vectors (alone, without any LNP encapsulation) and the control were administered to mixed gender, age-matched PAHenu2 mice approximately 6-11 weeks old. The naked ceDNA vectors were dosed at 0.1 μg (only selected ones) or 0.5 μg per animal (5 animals per group) by hydrodynamic intravenous injection via lateral tail vein in a dose volume of 90-100 ml/kg on day 0. Day 28 was the terminal time point. The study design is as shown below in Table 30.

TABLE 30
Test material administration in Example 11 study

| | | | | Dose | | Dosing | |
|---|---|---|---|---|---|---|---|
| Group | No. of | | | Levels | | Regimen | Terminal |
| No. | Animals | Genotype | Test Material | (μg) | Dose Volume | ROA | Time Point |
| 1 | 5 | WT | PBS | NA | 90-100 ml/kg | ONCE ON | Day 28 |
| 2 | 5 | PAH^{enu2} | PBS | NA | (set volume) | Day 0 by | |
| 3 | 5 | MUT | ceDNA412 | 0.5 | | Hydrodynamic | |
| 4 | 5 | | ceDNA412 | 0.1 | | IV | |
| 5 | 5 | | ceDNA2409 | 0.5 | | | |
| 6 | 5 | | ceDNA2410 | 0.5 | | | |
| 7 | 5 | | ceDNA2410 | 0.1 | | | |
| 8 | 5 | | ceDNA2415 | 0.5 | | | |
| 9 | 5 | | ceDNA2415 | 0.1 | | | |
| 10 | 5 | | ceDNA2418 | 0.5 | | | |
| 11 | 5 | | ceDNA2418 | 0.1 | | | |
| 12 | 5 | | ceDNA2416 | 0.5 | | | |
| 13 | 5 | | ceDNA2419 | 0.5 | | | |
| 14 | 5 | | ceDNA2420 | 0.5 | | | |

No. = Number; IV = intravenous; ROA = route of administration; WT = wild type; MUT = homozygous mutant; min = minute; hr = hour

Blood was collected at interim and terminal time points as follows in Tables 31A and 31B, respectively.

TABLE 31A
Blood Collection (Interim) Example 11 study

| | |
|---|---|
| Group | Whole Blood Sample Collection Times |
| Number | Saphenous Only |
| | (Orbital by permission of Vet Staff) |
| | Serum^{a} |
| 1-14 | FASTED |
| | Day −6, 3, 7, 14, & 21 |
| Volume/ | ~50 μL whole blood |
| Portion | |
| Processing | One (1) aliquot |
| | (Pure Honey) |
| Storage | Frozen at nominally −70° C. |

^{a}Whole blood was collected into serum separator tubes, with clot activator

TABLE 31B
Terminal Blood and Tissue Collection

| | |
|---|---|
| Group | Sample Collection Times |
| | Terminal Whole Blood |
| | (cardiac) |
| Number | Serum^{a} |
| 1-14 | FASTED |
| | Day 28 |
| Portion | MOV |
| Processing/ | One (1) aliquot processed plasma |
| Storage | frozen at nominally −70° C. |

^{a}Whole blood was be collected into serum separator tubes, with clot activator,
MOV = maximum obtainable volume

### Study Details

Species (number, sex, age): 70+3 spare PAH^{enu2} Mutant (MUT) mice (mixed gender, ˜6-8 weeks old, age-matched at arrival); 5 Wild Type (WT); mixed gender, littermates; age-matched. Animals were ˜6-11 weeks of at dose initiation.
Cage Side Observations: Cage side observations were performed daily.
Clinical Observations: Clinical observations were performed ˜1, ˜5-6 and ˜24 hours post the Day 0 Test Material dose, as applicable for remaining groups.
Class of Compound: Recombinant DNA vector, ceDNA.
Body Weights: Body weights for all animals were recorded on Days −6, 0, 1, 2, 3, 7, 14, 21 & 28 (prior to euthanasia).
Dose Formulation: Test articles were supplied in a concentration stock. Stock was warmed to room temperature and diluted with the provided PBS immediately prior to use. Prepared materials may be stored at ˜4° C. if dosing is not performed immediately.
Dose Administration: Test Materials for Groups 1-14 were dosed on Day 0 by hydrodynamic IV administration, at a set volume per animal, 90-100 ml/kg (dependent on the lightest animal in the group) via lateral tail vein.
Fasting Prior to Blood Collection: All animals (all groups) were fasted for a minimum of 4 hours prior to all interim and terminal blood collections: Days −6, 3, 7, 14, 21 & 28.
Food was removed and bedding changed. Food was returned at the conclusion of each interim blood collections for a fast of no more than 8 hours in duration.
Interim Blood Collection: All animals in Groups 1-10 had interim blood collected on Days −6, 3, 7, 14 & 21. Animals had whole blood for fasted serum collection. After collection animals would receive 0.5-1.0 mL lactated Ringer's, subcutaneously
Blood Collection: Whole blood for serum was collected by saphenous vein. Whole blood was collected into a serum separator with clot activator tube and processed into one (1) aliquot of serum per facility SOPs. All samples were be stored at nominally −70° C. until shipped to on dry ice.
Unscheduled Euthanasia: Terminal tissues were collected from moribund animals that are euthanized prior to their scheduled time point. If possible, tissues will be collected and stored from animals that are found dead.
Euthanasia: On Day 28, after a minimum 4 hours fast, animals were euthanized by CO₂ asphyxiation followed by thoracotomy and exsanguination.
Terminal Blood: Whole blood from exsanguination was collected into a serum separator with clot activator tube and processed into two (2) aliquot of serum per facility SOPs. All samples were stored at nominally −70° C. until shipped to on dry ice.
Phenylalanine (PHE) Levels: Serum samples were analyzed by the Pure Honey for PHE levels.
Activity Levels: Two (2) frozen liver samples were analyzed by Pure Honey for Activity levels.

### Results

The present study tested two codon optimized PAH sequences (i.e., mouse or human codon optimized PAH version 2 in combination with different promoters (i.e., VD promoter set or human AAT promoter. The specific elements contained in each tested ceDNA construct are summarized in Table 26.

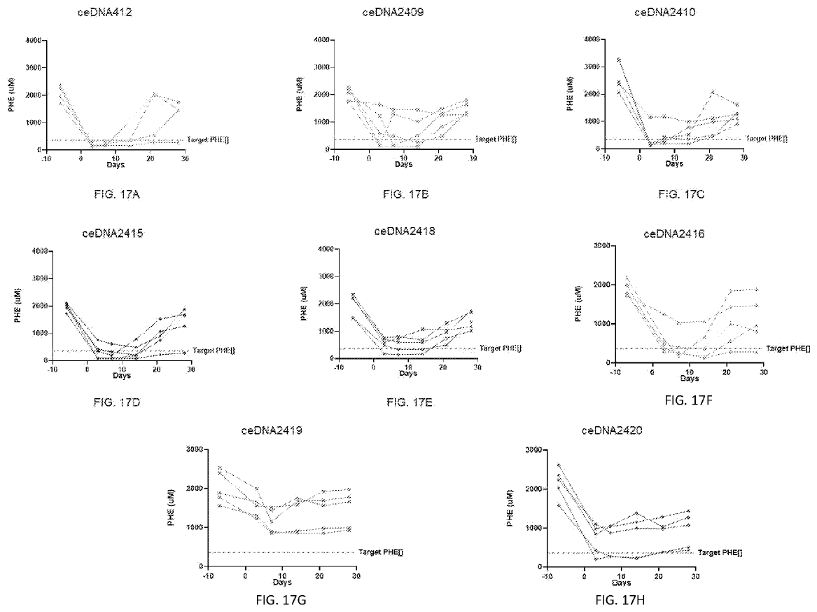

FIGS. 17A-17I show the results from the study. As shown in FIG. 17A, at a 0.5 μg dose, ceDNA412 showed acute correction of PHE levels (PHE μM) to below target concentration (less than 350 μM) in all 5 out of total 5 mice within 7 days. Notably, in one of the mice, the PHE correction was sustained through Day 28. Comparatively, in Examples 8 and 9, PHE corrections were observed in 4 out of total 5 mice for ceDNA412.

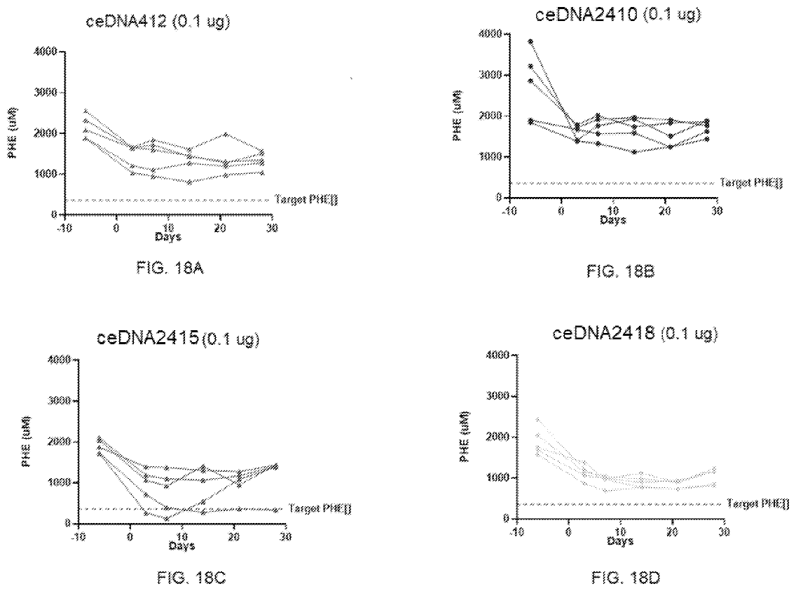

ceDNA2409, ceDNA2410, ceDNA2415, ceDNA2418, ceDNA2416, and ceDNA2420 each showed acute correction of PHE levels (PHE μM) to below target concentration (less than 350 μM) in at least 1 out of total 5 mice within 7 days. Of note, ceDNA2409, ceDNA2410, and ceDNA2415 each showed acute correction of PHE levels (PHE μM) to below target concentration (less than 350 μM) in 4 out of total 5 mice within 7 days, and in at least 1 out of total 5 mice within 3 days. Significantly, in one of the mice administered with ceDNA2415, the PHE correction was sustained until through Day 28. In other words, this study shows that at least ceDNA2415, which expresses the hPAH-r5-s29::mod-Intron_oIVS-v2_33bpFlanks ORF, the TTR promoter along with enhancers, introns, UTRs, restriction endonuclease site, cis- and post-transcriptional regulatory elements, is equivalent if not superior to ceDNA412 that possesses the VD promoter set and the codon optimized human PAH version 2 ORF. When the study was repeated at a lower dose of 0.1 μg as shown in FIGS. 18A-18E, remarkably ceDNA2415 showed acute correction of PHE levels (PHE μM) to below target concentration (less than 350 μM) in 2 out of total 5 mice within 14 days, and in 1 out of total 5 mice within 7 days.

When the number and types of HNF binding sites were varied, but all other structural elements including the promoter itself were kept the same, as in ceDNA2418 (5×HNF1) vis-à-vis ceDNA2415 (3×HNF1-4) and ceDNA2419 (5×HNF1) vis-à-vis ceDNA2416 (3×HNF1-4), the PHE level corrections appeared to be compromised. Similar observation was noted when the mice were dosed at 0.1 μg of ceDNA2418 (FIG. 18D) vis-à-vis 0.1 μg ceDNA2415 (FIG. 18C). These observations in Example 11 are consistent with the observation noted in Example 8 for ceDNA1476 and ceDNA1479.

Compared to ceDNA2409, ceDNA2410, ceDNA2415, and ceDNA2416, ceDNA2420 PHE correction levels were inferior with 3 mice not achieving the target level at any time point. Compared to ceDNA2409, ceDNA2410, ceDNA2415, and ceDNA2416, ceDNA2420 expresses the hPAH-r5-s29 ORF but without any intron and intron flanking region. This observation highlights the importance of introns and intron flanking regions in improving the hPAH-r5-s29 ORF.

### Example 12: Pharmacology Study to Evaluate Biochemical Correction of Phenylalanine Levels in PAHe^{nu2} Mice by Hydrodynamic Administration of ceDNA-Effect of Different Promoters and ceDNA Production Methods on PHE Correction

ceDNA Constructs

This study tested ceDNA vectors having different promoter sets and their respective cis-regulatory elements, post-transcriptional regulatory elements, introns and UTRs and codon optimized human PAH sequences or ORFs. With the exception of ceDNA412 which served as the control, ceDNA vectors in this study were prepared and purified as described above in International Patent Application No. PCT/US2019/14122.

The nucleic acid sequence of ceDNA containing codon optimized human PAH version 2 (ceDNA “hPAH Codop2”) is shown herein as SEQ ID NO: 192, and includes the following elements: left-ITR_v1: spacer_left-ITR_v2.1: VD_Promoter Set (VD): PmeI_site: Modified_Minimum_Consensus_Kozak: hPAH_codop_ORF_v2: PacI_site: WPRE_3pUTR: bGH/spacer: spacer_right-ITR_v1: right-ITR_v1 (ceDNA412). ceDNA412 served as a control in this study described in Example 12.

The nucleic acid sequence of ceDNA containing codon optimized human PAH version 2 (hPAH_codop_ORF_v2) is shown herein as SEQ ID NO: 577 and includes the VD promoter set, introns, UTRs, restriction endonuclease sites, cis- and post-transcriptional regulatory elements (ceDNA34).

The nucleic acid sequence of ceDNA containing codon optimized human PAH version 2 r5-s29 with an intron and an intron flanking region (hPAH-r5-s29::mod-Intron_oIVS-v2_33bpFlanks) is shown herein as SEQ ID NO: 581 and includes the hAAT promoter and other enhancers (i.e., PromoterSet-1476), introns, UTRs, restriction endonuclease site, cis- and post-transcriptional regulatory elements (ceDNA41).

The nucleic acid sequence of ceDNA containing codon optimized human PAH version 2 (hPAH_codop_ORF_v2) is shown herein as SEQ ID NO: 579 and includes the TTR promoter and other enhancers (i.e., HS-CRM8_FOXA_HNF4_consensus_v1), introns, UTRs, restriction endonuclease site, cis- and post-transcriptional regulatory elements (ceDNA36).

The nucleic acid sequence of ceDNA containing codon optimized human PAH version 2 r5-s29 with an intron and an intron flanking region (hPAH-r5-s29::oIVS1B_33bpFlanks) is shown herein as SEQ ID NO: 583 and includes the TTR promoter and other enhancers (i.e., HS-CRM8_FOXA_HNF4_consensus_v1), introns, UTRs, restriction endonuclease site, cis- and post-transcriptional regulatory elements (ceDNA43).

The nucleic acid sequence of ceDNA containing codon optimized human PAH version 2 r5-s29 with an intron and an intron flanking region (hPAH-r5-s29::oIVS1B_33bpFlanks) is shown herein as SEQ ID NO: 582 and includes the TTR promoter and other enhancers (i.e., 3×_HNF_FOXA_v1), introns, UTRs, restriction endonuclease site, cis- and post-transcriptional regulatory elements (ceDNA42).

The features of the ceDNA vectors used in this study and as described above are summarized below in Table 32.

TABLE 32
Construct features of ceDNA vectors in Example 12 study

| ceDNA | Construct features |
|---|---|
| ceDNA412 | 1× VD_PromoterSet ∥ hPAH_codop_ORF_v2 |
| ceDNA34 | 1× VD_PromoterSet ∥ PmeI-Mod2 ∥ |
| | hPAH_codop_ORF_v2 ∥ WPRE_3pUTR |
| ceDNA41 | PromoterSet-1476 ∥ hAAT-PmeI-Mod2-5pUTR ∥ hPAH-r5- |
| | s29::hIVS1B_33bpFlanks ∥ WPRE_3pUTR |
| ceDNA36 | HS-CRM_FOXA_HNF4_consensus_v1 ∥ |
| | TTRe-TTRm-MVM_v2-Mod2 ∥ |
| | hPAH_codop_ORF_v2 ∥ WPRE_3pUTR |
| ceDNA43 | HS-CRM_FOXA_HNF4_consensus_v1 ∥ |
| | TTRe-TTRm-MVM_v2-Mod2 ∥ hPAH-r5-s29:: |
| | hIVS1B_33bpFlanks ∥ WPRE_3pUTR |
| ceDNA42 | 3×_HNF4_FOXA_v1 ∥ TTRe-TTRm-MVM_v2-Mod2 ∥ |
| | hPAH-r5-s29::hIVS1B_33bpFlanks ∥ WPRE_3pUTR |

### Study Design

Each of the ceDNA PAH vectors (alone, without any LNP encapsulation) and the control were administered to mixed gender, age-matched PAHenu2 mice approximately 7-10 weeks old. The naked ceDNA vectors were dosed at 0.1 μg (only selected ones) or 0.5 μg per animal (5 animals per group) by hydrodynamic intravenous injection via lateral tail vein in a dose volume of 90-100 ml/kg on day 0. Day 28 was the terminal time point. The study design is as shown below in Table 33.

TABLE 33
Test material administration in Example 12 study

| | | | | Dose | | Dosing | |
|---|---|---|---|---|---|---|---|
| Group | No. of | | | Levels | | Regimen | Terminal |
| No. | Animals | Genotype | Test Material | (μg) | Dose Volume | ROA | Time Point |
| 1 | 5 | WT | PBS | NA | 90-100 ml/kg | ONCE ON | Day 28 |
| 2 | 5 | PAH^{enu2} | PBS | NA | (set volume) | Day 0 by | |
| 3 | 5 | MUT | ceDNA412 | 0.5 | | Hydrodynamic | |
| 4 | 5 | | ceDNA34 | 0.5 | | IV | |
| 5 | 5 | | ceDNA34 | 0.1 | | | |
| 6 | 5 | | ceDNA41 | 0.5 | | | |
| 7 | 5 | | ceDNA36 | 0.5 | | | |
| 8 | 5 | | ceDNA36 | 0.1 | | | |
| 9 | 5 | | ceDNA43 | 0.5 | | | |
| 10 | 5 | | ceDNA43 | 0.1 | | | |
| 11 | 5 | | ceDNA42 | 0.5 | | | |
| 12 | 5 | | ceDNA42 | 0.1 | | | |

No. = Number; IV = intravenous; ROA = route of administration; WT = wild type; MUT = homozygous mutant; min = minute; hr = hour

Blood was collected at interim and terminal time points as follows in Tables 34A and 34B, respectively.

TABLE 34A
Blood Collection (Interim) Example 12 study

| | |
|---|---|
| Group | Whole Blood Sample Collection Times |
| Number | Saphenous Only |
| | (Orbital by permission of Vet Staff) |
| | Serum^{a} |
| 1-12 | FASTED |
| | Day −4, 3, 7, 14, 21 & 28 |
| Volume/ | ~50 μL whole blood |
| Portion | |
| Processing | One (1) aliquot |
| | (Pure Honey) |
| Storage | Frozen at nominally −70° C. |

^{a}Whole blood was collected into serum separator tubes, with clot activator

TABLE 34B
Terminal Blood and Tissue Collection

| | |
|---|---|
| Group | Sample Collection Times |
| | Terminal Whole Blood |
| | (cardiac) |
| Number | Serum^{a} |
| 1-12 | FASTED |
| | Day 28 |
| Portion | MOV |
| Processing/ | One (1) aliquot processed plasma |
| Storage | frozen at nominally −70° C. |

^{a}Whole blood was be collected into serum separator tubes, with clot activator,
MOV = maximum obtainable volume

### Study Details

Species (number, sex, age): 60+5 spare PAH^{enu2} Mutant (MUT) mice (mixed gender, ˜6-8 weeks old, age-matched at arrival); 5 Wild Type (WT); mixed gender, littermates; age-matched. Animals were ˜7-10 weeks of at dose initiation.
Cage Side Observations: Cage side observations were performed daily.
Clinical Observations: Clinical observations were performed ˜1, ˜5-6 and ˜24 hours post the Day 0 Test Material dose, as applicable for remaining groups.
Class of Compound: Recombinant DNA vector, ceDNA.
Body Weights: Body weights for all animals were recorded on Days −4, 0, 1, 2, 3, 7, 14, 21 & 28 (prior to euthanasia).
Dose Formulation: Test articles were supplied in a concentration stock. Stock was warmed to room temperature and diluted with the provided PBS immediately prior to use. Prepared materials may be stored at ˜4° C. if dosing is not performed immediately.
Dose Administration: Test Materials for Groups 1-12 were dosed on Day 0 by hydrodynamic IV administration, at a set volume per animal, 90-100 ml/kg (dependent on the lightest animal in the group) via lateral tail vein.
Fasting Prior to Blood Collection: All animals (all groups) were fasted for a minimum of 4 hours prior to all interim and terminal blood collections: Days −4, 0, 1, 2, 3, 7, 14, 21 & 28.

Food was removed and bedding changed. Food was returned at the conclusion of each interim blood collections for a fast of no more than 8 hours in duration.
Interim Blood Collection: All animals in Groups 1-12 had interim blood collected on Days −4, 3, 7, 14 & 21. Animals had whole blood for fasted serum collection. After collection animals would receive 0.5-1.0 mL lactated Ringer's, subcutaneously
Blood Collection: Whole blood for serum was collected by saphenous vein. Whole blood was collected into a serum separator with clot activator tube and processed into one (1) aliquot of serum per facility SOPs. All samples were be stored at nominally −70° C. until shipped to on dry ice.
Unscheduled Euthanasia: Terminal tissues were collected from moribund animals that are euthanized prior to their scheduled time point. If possible, tissues will be collected and stored from animals that are found dead.
Euthanasia: On Day 28, after a minimum 4 hours fast, animals were euthanized by CO₂ asphyxiation followed by thoracotomy and exsanguination.
Terminal Blood: Whole blood from exsanguination was collected into a serum separator with clot activator tube and processed into two (2) aliquot of serum per facility SOPs. All samples were stored at nominally −70° C. until shipped to on dry ice.
Phenylalanine (PHE) Levels: Serum samples were analyzed by the Pure Honey for PHE levels.
Activity Levels: Two (2) frozen liver samples were analyzed by Pure Honey for Activity levels.

### Results

The present study tested two codon optimized PAH sequences (i.e., hPAH_codop_ORF_v2 and hPAH-r5-s29::hIVS1B_33bpFlanks) in combination with different promoters or promoter sets having at least promoters accompanied by enhancers (i.e., VD promoter set, PromoterSet-1476, and TTR promoter with either HS-CRM_FOXA_HNF4_consensus_v1 or 3×_HNF4_FOXA_v1 as one of the enhancers). The specific elements contained in each tested ceDNA construct are summarized in Table 32.

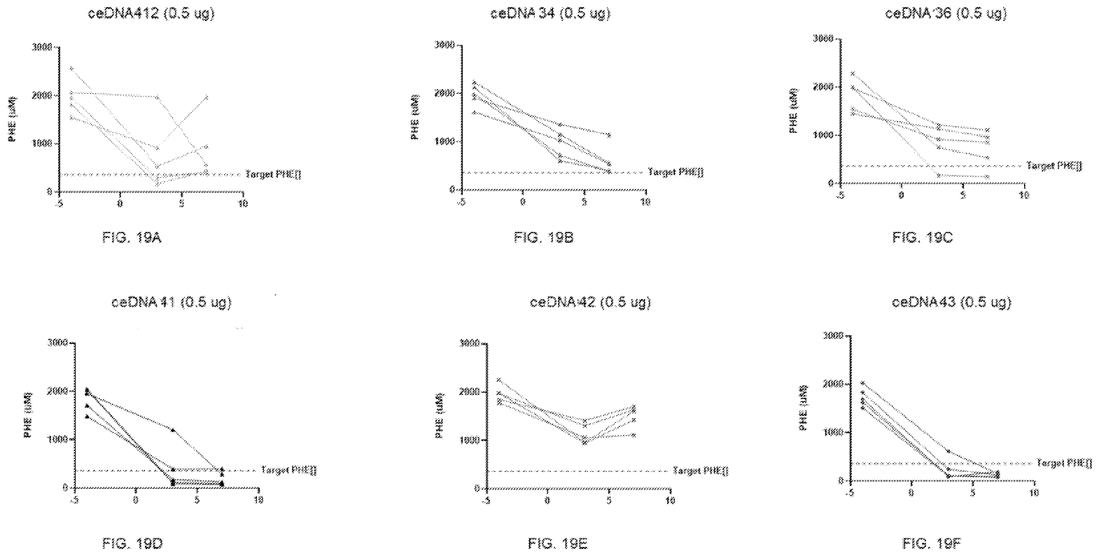

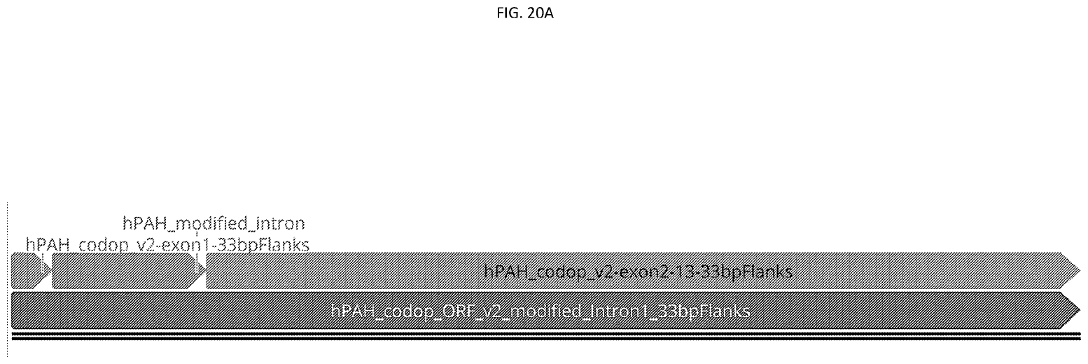

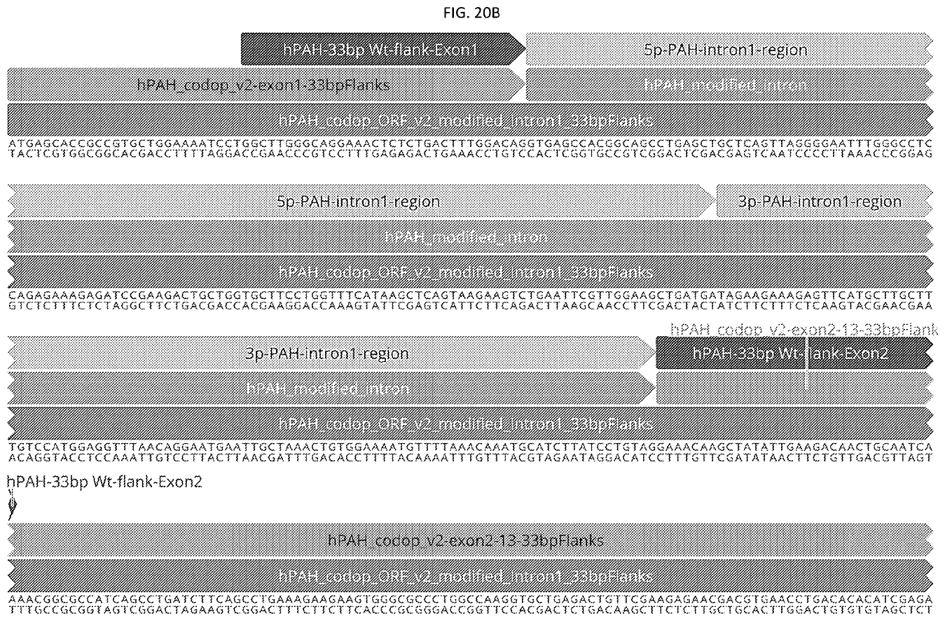

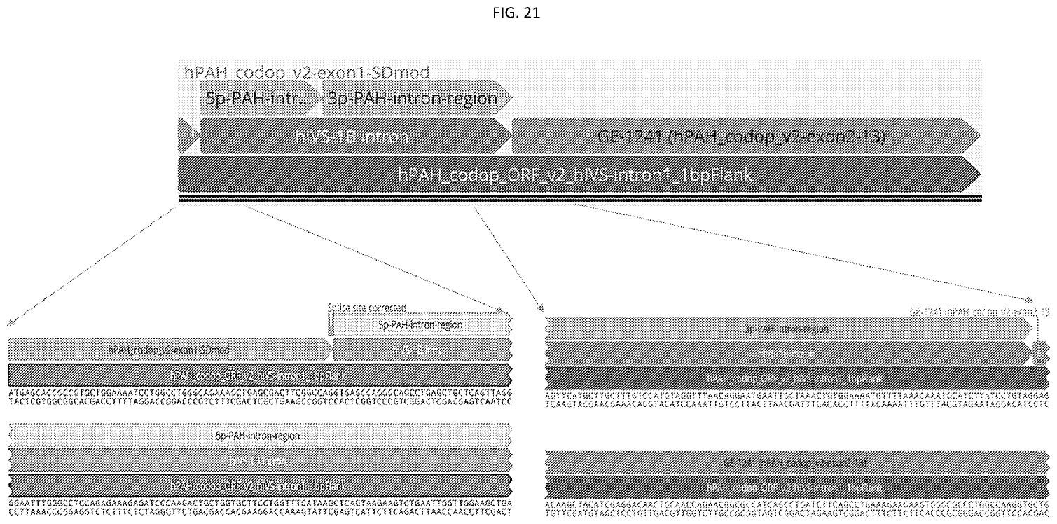

As shown in FIG. 19A and FIG. 19B, at a 0.5 μg dose, ceDNA412 and its corresponding synthetically produced counterpart vector, ceDNA34, showed equivalent PAH activity and PHE correction. At 0.5 μg dosing, other synthetically produced vectors, namely ceDNA36, ceDNA41, and ceDNA43 exhibited acute correction of PHE levels (PHE μM) to below target concentration (less than 350 μM) in at least 1 out of total 5 mice within 3 days. Notably, ceDNA41 and ceDNA43 outperformed control ceDNA412 in that up to 4 out of total 5 mice achieved PHE levels that are below the target concentration within 3 days of dosing.

ceDNA36 and ceDNA43 each had the TTR promoter in combination with the HS-CRM_FOXA_HNF4_consensus_v1 enhancer, but differ in the PAH sequences in that ceDNA43 contained the hPAH-r5-s29::hIVS1B_33bpFlanks PAH sequence which has a CpG content=0 while ceDNA41 contained the hPAH_codop_ORF_v2 PAH sequence (CpG content=77). The superior PHE correction in ceDNA43, as compared to ceDNA36, illustrates the importance of CpG minimization in the PAH sequence (see FIGS. 19C and 19E). Similarly, apart from different production methods, ceDNA41 and ceDNA412 also differ in the PAH sequences in that ceDNA41 contained the hPAH-r5-s29::hIVS1B_33bpFlanks PAH sequence whereas ceDNA412 contained the hPAH_codop_ORF_v2 PAH sequence. ceDNA41 exhibited superior PHE correction, in comparison to ceDNA412 (see FIGS. 19D and 19A).

Both of the vectors containing the TTR promoter in combination with HS-CRM_FOXA_HNF4_consensus_v1 as an enhancer, i.e., ceDNA43 and ceDNA36, showed PHE correction in at least 1 of total 5 mice within 3 days of dosing, with ceDNA43 achieving PHE correction in 4 of total 5 mice (see FIGS. 19B and 19E). Surprisingly, ceDNA42 which differs from ceDNA43 in that the 3×_HNF4_FOXA_v1 used in combination with the same TTR promoter contains 3 copies of HNF4 binding sites, did not achieve PHE correction in any mice (see FIG. 19D).

As can be seen from the 0.5 μg dosing studies, ceDNA43 that was synthetically produced, expressed the hPAH-r5-s29::hIVS1B_33bpFlanks PAH sequence which has a CpG content=0, and contained the TTR promoter in combination with HS-CRM_FOXA_HNF4_consensus_v1 as an enhancer, was the most potent vector among all the vectors studied.

Thus, the studies in Examples 6-12 have shown that a ceDNA construct (e.g., a ceDNA vector) comprising a PAH nucleic acid sequence that has been codon optimized can be thoughtfully combined with particular cis-acting elements (e.g., specific promoters, specific enhancers and specific promoter and enhancer combinations), that have been tested for optimal correction of phenylalanine level (e.g., expression and duration). Moreover, the Example 12 study demonstrates that a synthetically produced ceDNA construct having equivalent or more potent PHE corrections in PAH-deficient PAH^{enu2} mice.

### Nucleic Acid Sequences:

The nucleic acid sequence of ceDNA containing codon optimized human PAH version 2 (ceDNA “hPAH Codop2”) is shown below (ceDNA412). The promoter is shown underlined (SEQ ID NO:191) and the codon optimized PAH version 2 open reading frame (ORF) is shown double underlined (SEQ ID NO:382).

(SEQ ID NO: 192)
AAAGTAGCCGAAGATGACGGTTTGTCACATGGAGTTGGCA
GGATGTTTGATTAAAAACATAACAGGAAGAAAAATGCCCC
GCTGTGGGCGGACAAAATAGTTGGGAACTGGGAGGGGTGG
AAATGGAGTTTTTAAGGATTATTTAGGGAAGAGTGACAAA
ATAGATGGGAACTGGGTGTAGCGTCGTAAGCTAATACGAA
AATTAAAAATGACAAAATAGTTTGGAACTAGATTTCACTT
ATCTGGTTCGGATCTCCTAGGCCTGCAGGCAGCTGCGCGC
TCGCTCGCTCACTGAGGCCGCCCGGGCAAAGCCCGGGCGT
CGGGCGACCTTTGGTCGCCCGGCCTCAGTGAGCGAGCGAG
CGCGCAGAGAGGGAGTGGCCAACTCCATCACTAGGGGTTC
CTTGTAGTTAATGATTAACCCGCCATGCTACTTATCGCGG
CCGCCGGGGGAGGCTGCTGGTGAATATTAACCAAGGTCAC
CCCAGTTATCGGAGGAGCAAACAGGGGCTAAGTCCACACG
CGTGGTACCGTCTGTCTGCACATTTCGTAGAGCGAGTGTT
CCGATACTCTAATCTCCCTAGGCAAGGTTCATATTTGTGT
AGGTTACTTATTCTCCTTTTGTTGACTAAGTCAATAATCA
GAATCAGCAGGTTTGGAGTCAGCTTGGCAGGGATCAGCAG
CCTGGGTTGGAAGGAGGGGGTATAAAAGCCCCTTCACCAG
GAGAAGCCGTCACACAGATCCACAAGCTCCTGAAGAGGTA
AGGGTTTAAGGGATGGTTGGTTGGTGGGGTATTAATGTTT
AATTACCTGGAGCACCTGCCTGAAATCACTTTTTTTCAGG
TTGGTTTAAACCGCAGCCACCATGAGCACCGCCGTGCTGG
AAAATCCTGGCCTGGGCAGAAAGCTGAGCGACTTCGGCCA
AGAGACAAGCTACATCGAGGACAACTGCAACCAGAACGGC
GCCATCAGCCTGATCTTCAGCCTGAAAGAAGAAGTGGGCG
CCCTGGCCAAGGTGCTGAGACTGTTCGAAGAGAACGACGT
GAACCTGACACACATCGAGAGCAGACCCAGCAGACTGAAG
AAGGACGAGTACGAGTTCTTCACCCACCTGGACAAGCGGA
GCCTGCCTGCTCTGACCAACATCATCAAGATCCTGCGGCA
CGACATCGGCGCCACAGTGCACGAACTGAGCCGGGACAAG
AAAAAGGACACCGTGCCATGGTTCCCCAGAACCATCCAAG
AGCTGGACAGATTCGCCAACCAGATCCTGAGCTATGGCGC
CGAGCTGGACGCTGATCACCCTGGCTTTAAGGACCCCGTG
TACCGGGCCAGAAGAAAGCAGTTTGCCGATATCGCCTACA
ACTACCGGCACGGCCAGCCTATTCCTCGGGTCGAGTACAT
GGAAGAGGAAAAGAAAACCTGGGGCACCGTGTTCAAGACC
CTGAAGTCCCTGTACAAGACCCACGCCTGCTACGAGTACA
ACCACATCTTCCCACTGCTCGAAAAGTACTGCGGCTTCCA
CGAGGACAATATCCCTCAGCTTGAGGACGTGTCCCAGTTC
CTGCAGACCTGCACCGGCTTTAGACTGAGGCCAGTTGCCG
GACTGCTGAGCAGCAGAGATTTTCTCGGCGGCCTGGCCTT
CAGAGTGTTCCACTGTACCCAGTACATCAGACACGGCAGC
AAGCCCATGTACACCCCTGAGCCTGATATCTGCCACGAGC
TGCTGGGACATGTGCCCCTGTTCAGCGATAGAAGCTTCGC
CCAGTTCAGCCAAGAGATCGGACTGGCTTCTCTGGGAGCC
CCTGACGAGTACATTGAGAAGCTGGCCACCATCTACTGGT
TCACCGTGGAATTCGGCCTGTGCAAGCAGGGCGACAGCAT
CAAAGCTTATGGCGCTGGCCTGCTGTCTAGCTTCGGCGAG
CTGCAGTACTGTCTGAGCGAGAAGCCTAAGCTGCTGCCCC
TGGAACTGGAAAAGACCGCCATCCAGAACTACACCGTGAC
CGAGTTCCAGCCTCTGTACTACGTGGCCGAGAGCTTCAAC
GACGCCAAAGAAAAAGTGCGGAACTTCGCCGCCACCATTC
CTCGGCCTTTCAGCGTCAGATACGACCCCTACACACAGCG
GATCGAGGTGCTGGACAACACACAGCAGCTGAAAATTCTG
GCCGACTCCATCAACAGCGAGATCGGCATCCTGTGCAGCG
CCCTGCAGAAAATCAAGTGATAGTTAATTAAGAGCATCTT
ACCGCCATTTATTCCCATATTTGTTCTGTTTTTCTTGATT
TGGGTATACATTTAAATGTTAATAAAACAAAATGGTGGGG
CAATCATTTACATTTTTAGGGATATGTAATTACTAGTTCA
GGTGTATTGCCACAAGACAAACATGTTAAGAAACTTTCCC
GTTATTTACGCTCTGTTCCTGTTAATCAACCTCTGGATTA
CAAAATTTGTGAAAGATTGACTGATATTCTTAACTATGTT
GCTCCTTTTACGCTGTGTGGATATGCTGCTTTATAGCCTC
TGTATCTAGCTATTGCTTCCCGTACGGCTTTCGTTTTCTC
CTCCTTGTATAAATCCTGGTTGCTGTCTCTTTTAGAGGAG
TTGTGGCCCGTTGTCCGTCAACGTGGCGTGGTGTGCTCTG
TGTTTGCTGACGCAACCCCCACTGGCTGGGGCATTGCCAC
CACCTGTCAACTCCTTTCTGGGACTTTCGCTTTCCCCCTC
CCGATCGCCACGGCAGAACTCATCGCCGCCTGCCTTGCCC
GCTGCTGGACAGGGGCTAGGTTGCTGGGCACTGATAATTC
CGTGGTGTTGTCTGTGCCTTCTAGTTGCCAGCCATCTGTT
GTTTGCCCCTCCCCCGTGCCTTCCTTGACCCTGGAAGGTG
CCACTCCCACTGTCCTTTCCTAATAAAATGAGGAAATTGC
ATCGCATTGTCTGAGTAGGTGTCATTCTATTCTGGGGGGG
GGGTGGGGCAGGACAGCAAGGGGGAGGATTGGGAAGACAA
TAGCAGGCATGCTGGGGATGCGGTGGGCTCTATGGCTCTA
GAGCATGGCTACGTAGATAAGTAGCATGGCGGGTTAATCA
TTAACTACACCTGCAGGAGGAACCCCTAGTGATGGAGTTG
GCCACTCCCTCTCTGCGCGCTCGCTCGCTCACTGAGGCCG
GGCGACCAAAGGTCGCCCGACGCCCGGGCGGCCTCAGTGA
GCGAGCGAGCGCGCAGCTGCCTGCAGGGGCGCGCCTCGAG
CCATGGTGCTAGCAGCTGATGCATAGCATGCGGTACCGGG
AGATGGGGGAGGCTAACTGAAACACGGAAGGAGACAATAC
CGGAAGGAACCCGCGCTATGACGGCAATAAAAAGACAGAA
TAAAACGCACGGGTGTTGGGTCGTTTGTTCATAAACGCGG
GGTTCGGTCCCAGGGCTGGCACTCTGTCGATACCCCACCG
AGACCCCATTGGGACCAATACGCCCGCGTTTCTTCCTTTT
CCCCACCCCAACCCCCAAGTTCGGGTGAAGGCCCAGGGCT
CGCAGCCAACGTCGGGGCGGCAAGCCCTGCCATAGCCACT
ACGGGTACGTAGGCCAACCACTAGAACTATAGCTAGAGTC
CTGGGCGAACAAACGATGCTCGCCTTCCAGAAAACCGAGG
ATGCGAACCACTTCATCCGGGGTCAGCACCACCGGCAAGC
GCCGCGACGGCCGAGGTCTACCGATCTCCTGAAGCCAGGG
CAGATCCGTGCACAGCACCTTGCCGTAGAAGAACAGCAAG
GCCGCCAATGCCTGACGATGCGTGGAGACCGAAACCTTGC
GCTCGTTCGCCAGCCAGGACAGAAATGCCTCGACTTCGCT
GCTGCCCAAGGTTGCCGGGTGACGCACACCGTGGAAACGG
ATGAAGGCACGAACCCAGTTGACATAAGCCTGTTCGGTTC
GTAAACTGTAATGCAAGTAGCGTATGCGCTCACGCAACTG
GTCCAGAACCTTGACCGAACGCAGCGGTGGTAACGGCGCA
GTGGCGGTTTTCATGGCTTGTTATGACTGTTTTTTTGTAC
AGTCTATGCCTCGGGCATCCAAGCAGCAAGCGCGTTACGC
CGTGGGTCGATGTTTGATGTTATGGAGCAGCAACGATGTT
ACGCAGCAGCAACGATGTTACGCAGCAGGGCAGTCGCCCT
AAAACAAAGTTAGGTGGCTCAAGTATGGGCATCATTCGCA
CATGTAGGCTCGGCCCTGACCAAGTCAAATCCATGCGGGC
TGCTCTTGATCTTTTCGGTCGTGAGTTCGGAGACGTAGCC
ACCTACTCCCAACATCAGCCGGACTCCGATTACCTCGGGA
ACTTGCTCCGTAGTAAGACATTCATCGCGCTTGCTGCCTT
CGACCAAGAAGCGGTTGTTGGCGCTCTCGCGGCTTACGTT
CTGCCCAGGTTTGAGCAGCCGCGTAGTGAGATCTATATCT
ATGATCTCGCAGTCTCCGGCGAGCACCGGAGGCAGGGCAT
TGCCACCGCGCTCATCAATCTCCTCAAGCATGAGGCCAAC
GCGCTTGGTGCTTATGTGATCTACGTGCAAGCAGATTACG
GTGACGATCCCGCAGTGGCTCTCTATACAAAGTTGGGCAT
ACGGGAAGAAGTGATGCACTTTGATATCGACCCAAGTACC
GCCACCTAACAATTCGTTCAAGCCGAGATCGGCTTCCCGG
CCGCGGAGTTGTTCGGTAAATTGTCACAACGCCGCGAATA
TAGTCTTTACCATGCCCTTGGCCACGCCCCTCTTTAATAC
GACGGGCAATTTGCACTTCAGAAAATGAAGAGTTTGCTTT
AGCCATAACAAAAGTCCAGTATGCTTTTTCACAGCATAAC
TGGACTGATTTCAGTTTACAACTATTCTGTCTAGTTTAAG
ACTTTATTGTCATAGTTTAGATCTATTTTGTTCAGTTTAA
GACTTTATTGTCCGCCCACACCCGCTTACGCAGGGCATCC
ATTTATTACTCAACCGTAACCGATTTTGCCAGGTTACGCG
GCTGGTCTGCGGTGTGAAATACCGCACAGATGCGTAAGGA
GAAAATACCGCATCAGGCGCTCTTCCGCTTCCTCGCTCAC
TGACTCGCTGCGCTCGGTCGTTCGGCTGCGGCGAGCGGTA
TCAGCTCACTCAAAGGCGGTAATACGGTTATCCACAGAAT
CAGGGGATAACGCAGGAAAGAACATGTGAGCAAAAGGCCA
GCAAAAGGCCAGGAACCGTAAAAAGGCCGCGTTGCTGGCG
TTTTTCCATAGGCTCCGCCCCCCTGACGAGCATCACAAAA
ATCGACGCTCAAGTCAGAGGTGGCGAAACCCGACAGGACT
ATAAAGATACCAGGCGTTTCCCCCTGGAAGCTCCCTCGTG
CGCTCTCCTGTTCCGACCCTGCCGCTTACCGGATACCTGT
CCGCCTTTCTCCCTTCGGGAAGCGTGGCGCTTTCTCAATG
CTCACGCTGTAGGTATCTCAGTTCGGTGTAGGTCGTTCGC
TCCAAGCTGGGCTGTGTGCACGAACCCCCCGTTCAGCCCG
ACCGCTGCGCCTTATCCGGTAACTATCGTCTTGAGTCCAA
CCCGGTAAGACACGACTTATCGCCACTGGCAGCAGCCACT
GGTAACAGGATTAGCAGAGCGAGGTATGTAGGCGGTGCTA
CAGAGTTCTTGAAGTGGTGGCCTAACTACGGCTACACTAG
AAGGACAGTATTTGGTATCTGCGCTCTGCTGAAGCCAGTT
ACCTTCGGAAAAAGAGTTGGTAGCTCTTGATCCGGCAAAC
AAACCACCGCTGGTAGCGGTGGTTTTTTTGTTTGCAAGCA
GCAGATTACGCGCAGAAAAAAAGGATCTCAAGAAGATCCT
TTGATCTTTTCTACGGGGTCTGACGCTCAGTGGAACGAAA
ACTCACGTTAAGGGATTTTGGTCATGAGATTATCAAAAAG
GATCTTCACCTAGATCCTTTTAAATTAAAAATGAAGTTTT
AAATCAATCTAAAGTATATATGAGTAAACTTGGTCTGACA
GTTACCAATGCTTAATCAGTGAGGCACCTATCTCAGCGAT
CTGTCTATTTCGTTCATCCATAGTTGCCTGACTCCCCGTC
GTGTAGATAACTACGATACGGGAGGGCTTACCATCTGGCC
CCAGTGCTGCAATGATACCGCGAGACCCACGCTCACCGGC
TCCAGATTTATCAGCAATAAACCAGCCAGCCGGAAGGGCC
GAGCGCAGAAGTGGTCCTGCAACTTTATCCGCCTCCATCC
AGTCTATTAATTGTTGCCGGGAAGCTAGAGTAAGTAGTTC
GCCAGTTAATAGTTTGCGCAACGTTGTTGCCATTGCTACA
GGCATCGTGGTGTCACGCTCGTCGTTTGGTATGGCTTCAT
TCAGCTCCGGTTCCCAACGATCAAGGCGAGTTACATGATC
CCCCATGTTGTGCAAAAAAGCGGTTAGCTCCTTCGGTCCT
CCGATCGTTGTCAGAAGTAAGTTGGCCGCAGTGTTATCAC
TCATGGTTATGGCAGCACTGCATAATTCTCTTACTGTCAT
GCCATCCGTAAGATGCTTTTCTGTGACTGGTGAGTACTCA
ACCAAGTCATTCTGAGAATAGTGTATGCGGCGACCGAGTT
GCTCTTGCCCGGCGTCAATACGGGATAATACCGCGCCACA
TAGCAGAACTTTAAAAGTGCTCATCATTGGAAAACGTTCT
TCGGGGCGAAAACTCTCAAGGATCTTACCGCTGTTGAGAT
CCAGTTCGATGTAACCCACTCGTGCACCCAACTGATCTTC
AGCATCTTTTACTTTCACCAGCGTTTCTGGGTGAGCAAAA
ACAGGAAGGCAAAATGCCGCAAAAAAGGGAATAAGGGCGA
CACGGAAATGTTGAATACTCATACTCTTCCTTTTTCAATA
TTATTGAAGCATTTATCAGGGTTATTGTCTCATGAGCGGA
TACATATTTGAATGTATTTAGAAAAATAAACAAATAGGGG
TTCCGCGCACATTTCCCCGAAAAGTGCCACCTGAAATTGT
AAACGTTAATATTTTGTTAAAATTCGCGTTAAATTTTTGT
TAAATCAGCTCATTTTTTAACCAATAGGCCGAAATCGGCA
AAATCCCTTATAAATCAAAAGAATAGACCGAGATAGGGTT
GAGTGTTGTTCCAGTTTGGAACAAGAGTCCACTATTAAAG
AACGTGGACTCCAACGTCAAAGGGCGAAAAACCGTCTATC
AGGGCGATGGCCCACTACGTGAACCATCACCCTAATCAAG
TTTTTTGGGGTCGAGGTGCCGTAAAGCACTAAATCGGAAC
CCTAAAGGGAGCCCCCGATTTAGAGCTTGACGGGGAAAGC
CGGCGAACGTGGCGAGAAAGGAAGGGAAGAAAGCGAAAGG
AGCGGGCGCTAGGGCGCTGGCAAGTGTAGCGGTCACGCTG
CGCGTAACCACCACACCCGCCGCGCTTAATGCGCCGCTAC
AGGGCGCGTCCCATTCGCCATTCAGGCTGCAAATAAGCGT
TGATATTCAGTCAATTACAAACATTAATAACGAAGAGATG
ACAGAAAAATTTTCATTCTGTGACAGAGAA

The ceDNA construct above includes left-ITR_v1, spacer_left-ITR_v2.1, VD_PromoterSet, PmeI_site, Mod_Minimum_Consensus_Kozak, hPAH_codop_ORF_v2, PacI_site, WPRE_3pUTR, bGH, spacer_right-ITR_v1, right-ITR_v1

According to some embodiments, the nucleic acid sequence of ceDNA containing codon optimized human PAH version 2 (ceDNA “hPAH Codop2”) comprises SEQ ID NO: 192. According to some embodiments, the nucleic acid sequence of ceDNA containing codon optimized human PAH version 2 (ceDNA “hPAH Codop2”) is at least 85% identical to SEQ ID NO: 192. According to some embodiments, the nucleic acid sequence of ceDNA containing codon optimized human PAH version 2 (ceDNA “hPAH Codop2”) is at least 90% identical to SEQ ID NO: 192. According to some embodiments, the nucleic acid sequence of ceDNA containing codon optimized human PAH version 2 (ceDNA “hPAH Codop2”) is at least 91% identical to SEQ ID NO: 192. According to some embodiments, the nucleic acid sequence of ceDNA containing codon optimized human PAH version 2 (ceDNA “hPAH Codop2”) is at least 92% identical to SEQ ID NO: 192. According to some embodiments, the nucleic acid sequence of ceDNA containing codon optimized human PAH version 2 (ceDNA “hPAH Codop2”) is at least 93% identical to SEQ ID NO: 192. According to some embodiments, the nucleic acid sequence of ceDNA containing codon optimized human PAH version 2 (ceDNA “hPAH Codop2”) is at least 94% identical to SEQ ID NO: 192. According to some embodiments, the nucleic acid sequence of ceDNA containing codon optimized human PAH version 2 (ceDNA “hPAH Codop2”) is at least 95% identical to SEQ ID NO: 192. According to some embodiments, the nucleic acid sequence of ceDNA containing codon optimized human PAH version 2 (ceDNA “hPAH Codop2”) is at least 96% identical to SEQ ID NO: 192. According to some embodiments, the nucleic acid sequence of ceDNA containing codon optimized human PAH version 2 (ceDNA “hPAH Codop2”) is at least 97% identical to SEQ ID NO: 192. According to some embodiments, the nucleic acid sequence of ceDNA containing codon optimized human PAH version 2 (ceDNA “hPAH Codop2”) is at least 98% identical to SEQ ID NO: 192. According to some embodiments, the nucleic acid sequence of ceDNA containing codon optimized human PAH version 2 (ceDNA “hPAH Codop2”) is at least 99% identical to SEQ ID NO: 192. According to some embodiments, the nucleic acid sequence of ceDNA containing codon optimized human PAH version 2 (ceDNA “hPAH Codop2”) consists of SEQ ID NO: 192.

The nucleic acid sequence of ceDNA containing human PAH cDNA (ceDNA VD promoter linked to hPAH cDNA without codon optimization) is shown below. The promoter is shown underlined (SEQ ID NO:191) and the PAH open reading frame (ORF) is shown in double underline (SEQ ID NO:394; ceDNA802).

(SEQ ID NO: 193)
GGCCGGCCCCTGCAGGCAGCTGCGCGCTCGCTCGCTCACT
GAGGCCGCCCGGGCAAAGCCCGGGCGTCGGGCGACCTTTG
GTCGCCCGGCCTCAGTGAGCGAGCGAGCGCGCAGAGAGGG
AGTGGCCAACTCCATCACTAGGGGTTCCTTGTAGTTAATG
ATTAACCCGCCATGCTACTTATCTACGTAGCCATGCTCTA
GACGGGGGAGGCTGCTGGTGAATATTAACCAAGGTCACCC
CAGTTATCGGAGGAGCAAACAGGGGCTAAGTCCACACGCG
TGGTACCGTCTGTCTGCACATTTCGTAGAGCGAGTGTTCC
GATACTCTAATCTCCCTAGGCAAGGTTCATATTTGTGTAG
GTTACTTATTCTCCTTTTGTTGACTAAGTCAATAATCAGA
ATCAGCAGGTTTGGAGTCAGCTTGGCAGGGATCAGCAGCC
TGGGTTGGAAGGAGGGGGTATAAAAGCCCCTTCACCAGGA
GAAGCCGTCACACAGATCCACAAGCTCCTGAAGAGGTAAG
GGTTTAAGGGATGGTTGGTTGGTGGGGTATTAATGTTTAA
TTACCTGGAGCACCTGCCTGAAATCACTTTTTTTCAGGTT
GGTTTAAACGCCGCCACCATGTCCACTGCGGTCCTGGAAA
ACCCAGGCTTGGGCAGGAAACTCTCTGACTTTGGACAGGA
AACAAGCTATATTGAAGACAACTGCAATCAAAATGGTGCC
ATATCACTGATCTTCTCACTCAAAGAAGAAGTTGGTGCAT
TGGCCAAAGTATTGCGCTTATTTGAGGAGAATGATGTAAA
CCTGACCCACATTGAATCTAGACCTTCTCGTTTAAAGAAA
GATGAGTATGAATTTTTCACCCATTTGGATAAACGTAGCC
TGCCTGCTCTGACAAACATCATCAAGATCTTGAGGCATGA
CATTGGTGCCACTGTCCATGAGCTTTCACGAGATAAGAAG
AAAGACACAGTGCCCTGGTTCCCAAGAACCATTCAAGAGC
TGGACAGATTTGCCAATCAGATTCTCAGCTATGGAGCGGA
ACTGGATGCTGACCACCCTGGTTTTAAAGATCCTGTGTAC
CGTGCAAGACGGAAGCAGTTTGCTGACATTGCCTACAACT
ACCGCCATGGGCAGCCCATCCCTCGAGTGGAATACATGGA
GGAAGAAAAGAAAACATGGGGCACAGTGTTCAAGACTCTG
AAGTCCTTGTATAAAACCCATGCTTGCTATGAGTACAATC
ACATTTTTCCACTTCTTGAAAAGTACTGTGGCTTCCATGA
AGATAACATTCCCCAGCTGGAAGACGTTTCTCAGTTCCTG
CAGACTTGCACTGGTTTCCGCCTCCGACCTGTGGCTGGCC
TGCTTTCCTCTCGGGATTTCTTGGGTGGCCTGGCCTTCCG
AGTCTTCCACTGCACACAGTACATCAGACATGGATCCAAG
CCCATGTATACCCCCGAACCTGACATCTGCCATGAGCTGT
TGGGACATGTGCCCTTGTTTTCAGATCGCAGCTTTGCCCA
GTTTTCCCAGGAAATTGGCCTTGCCTCTCTGGGTGCACCT
GATGAATACATTGAAAAGCTCGCCACAATTTACTGGTTTA
CTGTGGAGTTTGGGCTCTGCAAACAAGGAGACTCCATAAA
GGCATATGGTGCTGGGCTCCTGTCATCCTTTGGTGAATTA
CAGTACTGCTTATCAGAGAAGCCAAAGCTTCTCCCCCTGG
AGCTGGAGAAGACAGCCATCCAAAATTACACTGTCACGGA
GTTCCAGCCCCTCTATTACGTGGCAGAGAGTTTTAATGAT
GCCAAGGAGAAAGTAAGGAACTTTGCTGCCACAATACCTC
GGCCCTTCTCAGTTCGCTACGACCCATACACCCAAAGGAT
TGAGGTCTTGGACAATACCCAGCAGCTTAAGATTTTGGCT
GATTCCATTAACAGTGAAATTGGAATCCTTTGCAGTGCCC
TCCAGAAAATAAAGTAATTAATTAAGAGCATCTTACCGCC
ATTTATTCCCATATTTGTTCTGTTTTTCTTGATTTGGGTA
TACATTTAAATGTTAATAAAACAAAATGGTGGGGCAATCA
TTTACATTTTTAGGGATATGTAATTACTAGTTCAGGTGTA
TTGCCACAAGACAAACATGTTAAGAAACTTTCCCGTTATT
TACGCTCTGTTCCTGTTAATCAACCTCTGGATTACAAAAT
TTGTGAAAGATTGACTGATATTCTTAACTATGTTGCTCCT
TTTACGCTGTGTGGATATGCTGCTTTATAGCCTCTGTATC
TAGCTATTGCTTCCCGTACGGCTTTCGTTTTCTCCTCCTT
GTATAAATCCTGGTTGCTGTCTCTTTTAGAGGAGTTGTGG
CCCGTTGTCCGTCAACGTGGCGTGGTGTGCTCTGTGTTTG
CTGACGCAACCCCCACTGGCTGGGGCATTGCCACCACCTG
TCAACTCCTTTCTGGGACTTTCGCTTTCCCCCTCCCGATC
GCCACGGCAGAACTCATCGCCGCCTGCCTTGCCCGCTGCT
GGACAGGGGCTAGGTTGCTGGGCACTGATAATTCCGTGGT
GTTGTCTGTGCCTTCTAGTTGCCAGCCATCTGTTGTTTGC
CCCTCCCCCGTGCCTTCCTTGACCCTGGAAGGTGCCACTC
CCACTGTCCTTTCCTAATAAAATGAGGAAATTGCATCGCA
TTGTCTGAGTAGGTGTCATTCTATTCTGGGGGGTGGGGTG
GGGCAGGACAGCAAGGGGGAGGATTGGGAAGACAATAGCA
GGCATGCTGGGGATGCGGTGGGCTCTATGGCTCTAGAGCA
TGGCTACGTAGATAAGTAGCATGGCGGGTTAATCATTAAC
TACACCTGCAGGAGGAACCCCTAGTGATGGAGTTGGCCAC
TCCCTCTCTGCGCGCTCGCTCGCTCACTGAGGCCGGGCGA
CCAAAGGTCGCCCGACGCCCGGGCGGCCTCAGTGAGCGAG
CGAGCGCGCAGCTGCCTGCAGGGGCGCGCCTCGAGGCATG
CGGTACCAAGCTTGTCGAGAAGTACTAGAGGATCATAATC
AGCCATACCACATTTGTAGAGGTTTTACTTGCTTTAAAAA
ACCTCCCACACCTCCCCCTGAACCTGAAACATAAAATGAA
TGCAATTGTTGTTGTTAACTTGTTTATTGCAGCTTATAAT
GGTTACAAATAAAGCAATAGCATCACAAATTTCACAAATA
AAGCATTTTTTTCACTGCATTCTAGTTGTGGTTTGTCCAA
ACTCATCAATGTATCTTATCATGTCTGGATCTGATCACTG
ATATCGCCTAGGAGATCCGAACCAGATAAGTGAAATCTAG
TTCCAAACTATTTTGTCATTTTTAATTTTCGTATTAGCTT
ACGACGCTACACCCAGTTCCCATCTATTTTGTCACTCTTC
CCTAAATAATCCTTAAAAACTCCATTTCCACCCCTCCCAG
TTCCCAACTATTTTGTCCGCCCACAGCGGGGCATTTTTCT
TCCTGTTATGTTTTTAATCAAACATCCTGCCAACTCCATG
TGACAAACCGTCATCTTCGGCTACTTTTTCTCTGTCACAG
AATGAAAATTTTTCTGTCATCTCTTCGTTATTAATGTTTG
TAATTGACTGAATATCAACGCTTATTTGCAGCCTGAATGG
CGAATGGGACGCGCCCTGTAGCGGCGCATTAAGCGCGGCG
GGTGTGGTGGTTACGCGCAGCGTGACCGCTACACTTGCCA
GCGCCCTAGCGCCCGCTCCTTTCGCTTTCTTCCCTTCCTT
TCTCGCCACGTTCGCCGGCTTTCCCCGTCAAGCTCTAAAT
CGGGGGCTCCCTTTAGGGTTCCGATTTAGTGCTTTACGGC
ACCTCGACCCCAAAAAACTTGATTAGGGTGATGGTTCACG
TAGTGGGCCATCGCCCTGATAGACGGTTTTTCGCCCTTTG
ACGTTGGAGTCCACGTTCTTTAATAGTGGACTCTTGTTCC
AAACTGGAACAACACTCAACCCTATCTCGGTCTATTCTTT
TGATTTATAAGGGATTTTGCCGATTTCGGCCTATTGGTTA
AAAAATGAGCTGATTTAACAAAAATTTAACGCGAATTTTA
ACAAAATATTAACGTTTACAATTTCAGGTGGCACTTTTCG
GGGAAATGTGCGCGGAACCCCTATTTGTTTATTTTTCTAA
ATACATTCAAATATGTATCCGCTCATGAGACAATAACCCT
GATAAATGCTTCAATAATATTGAAAAAGGAAGAGTATGAG
TATTCAACATTTCCGTGTCGCCCTTATTCCCTTTTTTGCG
GCATTTTGCCTTCCTGTTTTTGCTCACCCAGAAACGCTGG
TGAAAGTAAAAGATGCTGAAGATCAGTTGGGTGCACGAGT
GGGTTACATCGAACTGGATCTCAACAGCGGTAAGATCCTT
GAGAGTTTTCGCCCCGAAGAACGTTTTCCAATGATGAGCA
CTTTTAAAGTTCTGCTATGTGGCGCGGTATTATCCCGTAT
TGACGCCGGGCAAGAGCAACTCGGTCGCCGCATACACTAT
TCTCAGAATGACTTGGTTGAGTACTCACCAGTCACAGAAA
AGCATCTTACGGATGGCATGACAGTAAGAGAATTATGCAG
TGCTGCCATAACCATGAGTGATAACACTGCGGCCAACTTA
CTTCTGACAACGATCGGAGGACCGAAGGAGCTAACCGCTT
TTTTGCACAACATGGGGGATCATGTAACTCGCCTTGATCG
TTGGGAACCGGAGCTGAATGAAGCCATACCAAACGACGAG
CGTGACACCACGATGCCTGTAGCAATGGCAACAACGTTGC
GCAAACTATTAACTGGCGAACTACTTACTCTAGCTTCCCG
GCAACAATTAATAGACTGGATGGAGGCGGATAAAGTTGCA
GGACCACTTCTGCGCTCGGCCCTTCCGGCTGGCTGGTTTA
TTGCTGATAAATCTGGAGCCGGTGAGCGTGGGTCTCGCGG
TATCATTGCAGCACTGGGGCCAGATGGTAAGCCCTCCCGT
ATCGTAGTTATCTACACGACGGGGAGTCAGGCAACTATGG
ATGAACGAAATAGACAGATCGCTGAGATAGGTGCCTCACT
GATTAAGCATTGGTAACTGTCAGACCAAGTTTACTCATAT
ATACTTTAGATTGATTTAAAACTTCATTTTTAATTTAAAA
GGATCTAGGTGAAGATCCTTTTTGATAATCTCATGACCAA
AATCCCTTAACGTGAGTTTTCGTTCCACTGAGCGTCAGAC
CCCGTAGAAAAGATCAAAGGATCTTCTTGAGATCCTTTTT
TTCTGCGCGTAATCTGCTGCTTGCAAACAAAAAAACCACC
GCTACCAGCGGTGGTTTGTTTGCCGGATCAAGAGCTACCA
ACTCTTTTTCCGAAGGTAACTGGCTTCAGCAGAGCGCAGA
TACCAAATACTGTCCTTCTAGTGTAGCCGTAGTTAGGCCA
CCACTTCAAGAACTCTGTAGCACCGCCTACATACCTCGCT
CTGCTAATCCTGTTACCAGTGGCTGCTGCCAGTGGCGATA
AGTCGTGTCTTACCGGGTTGGACTCAAGACGATAGTTACC
GGATAAGGCGCAGCGGTCGGGCTGAACGGGGGGTTCGTGC
ACACAGCCCAGCTTGGAGCGAACGACCTACACCGAACTGA
GATACCTACAGCGTGAGCATTGAGAAAGCGCCACGCTTCC
CGAAGGGAGAAAGGCGGACAGGTATCCGGTAAGCGGCAGG
GTCGGAACAGGAGAGCGCACGAGGGAGCTTCCAGGGGGAA
ACGCCTGGTATCTTTATAGTCCTGTCGGGTTTCGCCACCT
CTGACTTGAGCGTCGATTTTTGTGATGCTCGTCAGGGGGG
CGGAGCCTATGGAAAAACGCCAGCAACGCGGCCTTTTTAC
GGTTCCTGGCCTTTTGCTGGCCTTTTGCTCACATGTTCTT
TCCTGCGTTATCCCCTGATTCTGTGGATAACCGTATTACC
GCCTTTGAGTGAGCTGATACCGCTCGCCGCAGCCGAACGA
CCGAGCGCAGCGAGTCAGTGAGCGAGGAAGCGGAAGAGCG
CCTGATGCGGTATTTTCTCCTTACGCATCTGTGCGGTATT
TCACACCGCAGACCAGCCGCGTAACCTGGCAAAATCGGTT
ACGGTTGAGTAATAAATGGATGCCCTGCGTAAGCGGGTGT
GGGCGGACAATAAAGTCTTAAACTGAACAAAATAGATCTA
AACTATGACAATAAAGTCTTAAACTAGACAGAATAGTTGT
AAACTGAAATCAGTCCAGTTATGCTGTGAAAAAGCATACT
GGACTTTTGTTATGGCTAAAGCAAACTCTTCATTTTCTGA
AGTGCAAATTGCCCGTCGTATTAAAGAGGGGCGTGGCCAA
GGGCATGGTAAAGACTATATTCGCGGCGTTGTGACAATTT
ACCGAACAACTCCGCGGCCGGGAAGCCGATCTCGGCTTGA
ACGAATTGTTAGGTGGCGGTACTTGGGTCGATATCAAAGT
GCATCACTTCTTCCCGTATGCCCAACTTTGTATAGAGAGC
CACTGCGGGATCGTCACCGTAATCTGCTTGCACGTAGATC
ACATAAGCACCAAGCGCGTTGGCCTCATGCTTGAGGAGAT
TGATGAGCGCGGTGGCAATGCCCTGCCTCCGGTGCTCGCC
GGAGACTGCGAGATCATAGATATAGATCTCACTACGCGGC
TGCTCAAACCTGGGCAGAACGTAAGCCGCGAGAGCGCCAA
CAACCGCTTCTTGGTCGAAGGCAGCAAGCGCGATGAATGT
CTTACTACGGAGCAAGTTCCCGAGGTAATCGGAGTCCGGC
TGATGTTGGGAGTAGGTGGCTACGTCTCCGAACTCACGAC
CGAAAAGATCAAGAGCAGCCCGCATGGATTTGACTTGGTC
AGGGCCGAGCCTACATGTGCGAATGATGCCCATACTTGAG
CCACCTAACTTTGTTTTAGGGCGACTGCCCTGCTGCGTAA
CATCGTTGCTGCTGCGTAACATCGTTGCTGCTCCATAACA
TCAAACATCGACCCACGGCGTAACGCGCTTGCTGCTTGGA
TGCCCGAGGCATAGACTGTACAAAAAAACAGTCATAACAA
GCCATGAAAACCGCCACTGCGCCGTTACCACCGCTGCGTT
CGGTCAAGGTTCTGGACCAGTTGCGTGAGCGCATACGCTA
CTTGCATTACAGTTTACGAACCGAACAGGCTTATGTCAAC
TGGGTTCGTGCCTTCATCCGTTTCCACGGTGTGCGTCACC
CGGCAACCTTGGGCAGCAGCGAAGTCGAGGCATTTCTGTC
CTGGCTGGCGAACGAGCGCAAGGTTTCGGTCTCCACGCAT
CGTCAGGCATTGGCGGCCTTGCTGTTCTTCTACGGCAAGG
TGCTGTGCACGGATCTGCCCTGGCTTCAGGAGATCGGAAG
ACCTCGGCCGTCGCGGCGCTTGCCGGTGGTGCTGACCCCG
GATGAAGTGGTTCGCATCCTCGGTTTTCTGGAAGGCGAGC
ATCGTTTGTTCGCCCAGGACTCTAGCTATAGTTCTAGTGG
TTGGCTACGTATACTCCGGAATATTAATAGATCATGGAGA
TAATTAAAATGATAACCATCTCGCAAATAAATAAGTATTT
TACTGTTTTCGTAACAGTTTTGTAATAAAAAAACCTATAA
ATATTCCGGATTATTCATACCGTCCCACCATCGGGCGCGG
ATCTCGGTCCGAAACCATGTCGTACTACCATCACCATCAC
CATCACGATTACGATATCCCAACGACCGAAAACCTGTATT
TTCAGGGCGCCATGGGATCC

SEQ ID NO: 193 includes the following elements. Left-ITR_v1: spacer_left-ITR_v1: VD_Promoter Set: PmeI_site: Consensus_Kozak: hPAH_cDNA_ORF_v3: PacI_site: WPRE_3pUTR: bGH: spacer_right-ITR_v1: right-ITR_v1.

According to some embodiments, the nucleic acid sequence of ceDNA containing human PAH cDNA (ceDNA VD promoter linked to hPAH cDNA without codon optimization) comprises SEQ ID NO: 193. According to some embodiments, the nucleic acid sequence of ceDNA containing human PAH cDNA (ceDNA VD promoter linked to hPAH cDNA without codon optimization) is at least 85% identical to SEQ ID NO: 193. According to some embodiments, the nucleic acid sequence of ceDNA containing human PAH cDNA (ceDNA VD promoter linked to hPAH cDNA without codon optimization) is at least 90% identical to SEQ ID NO: 193. According to some embodiments, the nucleic acid sequence of ceDNA containing human PAH cDNA (ceDNA VD promoter linked to hPAH cDNA without codon optimization) is at least 91% identical to SEQ ID NO: 193. According to some embodiments, the nucleic acid sequence of ceDNA containing human PAH cDNA (ceDNA VD promoter linked to hPAH cDNA without codon optimization) is at least 92% identical to SEQ ID NO: 193. According to some embodiments, the nucleic acid sequence of ceDNA containing human PAH cDNA (ceDNA VD promoter linked to hPAH cDNA without codon optimization) is at least 93% identical to SEQ ID NO: 193. According to some embodiments, the nucleic acid sequence of ceDNA containing human PAH cDNA (ceDNA VD promoter linked to hPAH cDNA without codon optimization) is at least 94% identical to SEQ ID NO: 193. According to some embodiments, the nucleic acid sequence of ceDNA containing human PAH cDNA (ceDNA VD promoter linked to hPAH cDNA without codon optimization) is at least 95% identical to SEQ ID NO: 193. According to some embodiments, the nucleic acid sequence of ceDNA containing human PAH cDNA (ceDNA VD promoter linked to hPAH cDNA without codon optimization) is at least 96% identical to SEQ ID NO: 193. According to some embodiments, the nucleic acid sequence of ceDNA containing human PAH cDNA (ceDNA VD promoter linked to hPAH cDNA without codon optimization) is at least 97% identical to SEQ ID NO: 193. According to some embodiments, the nucleic acid sequence of ceDNA containing human PAH cDNA (ceDNA VD promoter linked to hPAH cDNA without codon optimization) is at least 98% identical to SEQ ID NO: 193. According to some embodiments, the nucleic acid sequence of ceDNA containing human PAH cDNA (ceDNA VD promoter linked to hPAH cDNA without codon optimization) is at least 99% identical to SEQ ID NO: 193. According to some embodiments, the nucleic acid sequence of ceDNA containing human PAH cDNA (ceDNA VD promoter linked to hPAH cDNA without codon optimization) consists of SEQ ID NO: 193.

The nucleic acid sequence of ceDNA containing codon optimized human PAH version 2 (ceDNA “hPAH Codop2”) with specific cis-regulatory elements is shown below (ceDNA1132).

(SEQ ID NO: 194)
CCTGCAGGCAGCTGCGCGCTCGCTCGCTCACTGAGGCCGC
CCGGGCAAAGCCCGGGCGTCGGGCGACCTTTGGTCGCCCG
GCCTCAGTGAGCGAGCGAGCGCGCAGAGAGGGAGTGGCCA
ACTCCATCACTAGGGGTTCCTTGTAGTTAATGATTAACCC
GCCATGCTACTTATCGCGGCCGCGGGGGAGGCTGCTGGTG
AATATTAACCAAGGTCACCCCAGTTATCGGAGGAGCAAAC
AGGGGCTAAGTCCACCGGGGGAGGCTGCTGGTGAATATTA
ACCAAGGTCACCCCAGTTATCGGAGGAGCAAACAGGGGCT
AAGTCCACCGGGGGAGGCTGCTGGTGAATATTAACCAAGG
TCACCCCAGTTATCGGAGGAGCAAACAGGGGCTAAGTCCA
CGGTACCCACTGGGAGGATGTTGAGTAAGATGGAAAACTA
CTGATGACCCTTGCAGAGACAGAGTATTAGGACATGTTTG
AACAGGGGCCGGGCGATCAGCAGGTAGCTCTAGAGGATCC
CCGTCTGTCTGCACATTTCGTAGAGCGAGTGTTCCGATAC
TCTAATCTCCCTAGGCAAGGTTCATATTTGTGTAGGTTAC
TTATTCTCCTTTTGTTGACTAAGTCAATAATCAGAATCAG
CAGGTTTGGAGTCAGCTTGGCAGGGATCAGCAGCCTGGGT
TGGAAGGAGGGGGTATAAAAGCCCCTTCACCAGGAGAAGC
CGTCACACAGATCCACAAGCTCCTGAAGAGGTAAGGGTTT
AAGGGATGGTTGGTTGGTGGGGTATTAATGTTTAATTACC
TGGAGCACCTGCCTGAAATCACTTTTTTTCAGGTTGGGTT
TAAACCGCAGCCACCATGAGCACCGCCGTGCTGGAAAATC
CTGGCCTGGGCAGAAAGCTGAGCGACTTCGGCCAAGAGAC
AAGCTACATCGAGGACAACTGCAACCAGAACGGCGCCATC
AGCCTGATCTTCAGCCTGAAAGAAGAAGTGGGCGCCCTGG
CCAAGGTGCTGAGACTGTTCGAAGAGAACGACGTGAACCT
GACACACATCGAGAGCAGACCCAGCAGACTGAAGAAGGAC
GAGTACGAGTTCTTCACCCACCTGGACAAGCGGAGCCTGC
CTGCTCTGACCAACATCATCAAGATCCTGCGGCACGACAT
CGGCGCCACAGTGCACGAACTGAGCCGGGACAAGAAAAAG
GACACCGTGCCATGGTTCCCCAGAACCATCCAAGAGCTGG
ACAGATTCGCCAACCAGATCCTGAGCTATGGCGCCGAGCT
GGACGCTGATCACCCTGGCTTTAAGGACCCCGTGTACCGG
GCCAGAAGAAAGCAGTTTGCCGATATCGCCTACAACTACC
GGCACGGCCAGCCTATTCCTCGGGTCGAGTACATGGAAGA
GGAAAAGAAAACCTGGGGCACCGTGTTCAAGACCCTGAAG
TCCCTGTACAAGACCCACGCCTGCTACGAGTACAACCACA
TCTTCCCACTGCTCGAAAAGTACTGCGGCTTCCACGAGGA
CAATATCCCTCAGCTTGAGGACGTGTCCCAGTTCCTGCAG
ACCTGCACCGGCTTTAGACTGAGGCCAGTTGCCGGACTGC
TGAGCAGCAGAGATTTTCTCGGCGGCCTGGCCTTCAGAGT
GTTCCACTGTACCCAGTACATCAGACACGGCAGCAAGCCC
ATGTACACCCCTGAGCCTGATATCTGCCACGAGCTGCTGG
GACATGTGCCCCTGTTCAGCGATAGAAGCTTCGCCCAGTT
CAGCCAAGAGATCGGACTGGCTTCTCTGGGAGCCCCTGAC
GAGTACATTGAGAAGCTGGCCACCATCTACTGGTTCACCG
TGGAATTCGGCCTGTGCAAGCAGGGCGACAGCATCAAAGC
TTATGGCGCTGGCCTGCTGTCTAGCTTCGGCGAGCTGCAG
TACTGTCTGAGCGAGAAGCCTAAGCTGCTGCCCCTGGAAC
TGGAAAAGACCGCCATCCAGAACTACACCGTGACCGAGTT
CCAGCCTCTGTACTACGTGGCCGAGAGCTTCAACGACGCC
AAAGAAAAAGTGCGGAACTTCGCCGCCACCATTCCTCGGC
CTTTCAGCGTCAGATACGACCCCTACACACAGCGGATCGA
GGTGCTGGACAACACACAGCAGCTGAAAATTCTGGCCGAC
TCCATCAACAGCGAGATCGGCATCCTGTGCAGCGCCCTGC
AGAAAATCAAGTGATAGTTAATTAAGAGCATCTTACCGCC
ATTTATTCCCATATTTGTTCTGTTTTTCTTGATTTGGGTA
TACATTTAAATGTTAATAAAACAAAATGGTGGGGCAATCA
TTTACATTTTTAGGGATATGTAATTACTAGTTCAGGTGTA
TTGCCACAAGACAAACATGTTAAGAAACTTTCCCGTTATT
TACGCTCTGTTCCTGTTAATCAACCTCTGGATTACAAAAT
TTGTGAAAGATTGACTGATATTCTTAACTATGTTGCTCCT
TTTACGCTGTGTGGATATGCTGCTTTATAGCCTCTGTATC
TAGCTATTGCTTCCCGTACGGCTTTCGTTTTCTCCTCCTT
GTATAAATCCTGGTTGCTGTCTCTTTTAGAGGAGTTGTGG
CCCGTTGTCCGTCAACGTGGCGTGGTGTGCTCTGTGTTTG
CTGACGCAACCCCCACTGGCTGGGGCATTGCCACCACCTG
TCAACTCCTTTCTGGGACTTTCGCTTTCCCCCTCCCGATC
GCCACGGCAGAACTCATCGCCGCCTGCCTTGCCCGCTGCT
GGACAGGGGCTAGGTTGCTGGGCACTGATAATTCCGTGGT
GTTGTCTGTGCCTTCTAGTTGCCAGCCATCTGTTGTTTGC
CCCTCCCCCGTGCCTTCCTTGACCCTGGAAGGTGCCACTC
CCACTGTCCTTTCCTAATAAAATGAGGAAATTGCATCGCA
TTGTCTGAGTAGGTGTCATTCTATTCTGGGGGGTGGGGTG
GGGCAGGACAGCAAGGGGGAGGATTGGGAAGACAATAGCA
GGCATGCTGGGGATGCGGTGGGCTCTATGGCTCTAGAGCA
TGGCTACGTAGATAAGTAGCATGGCGGGTTAATCATTAAC
TACACCTGCAGGAGGAACCCCTAGTGATGGAGTTGGCCAC
TCCCTCTCTGCGCGCTCGCTCGCTCACTGAGGCCGGGCGA
CCAAAGGTCGCCCGACGCCCGGGCGGCCTCAGTGAGCGAG
CGAGCGCGCAGCTGCCTGCAGG

SEQ ID NO: 194 includes the following elements. Left-ITR_v1, spacer_left-ITR_v2.1, 3×SerpEnh-TTRe-TTRm, MVM_intron, PmeI_site, Mod_Minimum_Consensus_Kozak, hPAH_codop_ORF_v2, PacI_site, WPRE_3pUTR, bGH, spacer_right-ITR_v1, right-ITR_v1.

According to some embodiments, the nucleic acid sequence of ceDNA containing codon optimized human PAH version 2 (ceDNA “hPAH_codop_ORF_v2”) with specific cis-regulatory elements comprises SEQ ID NO: 194. According to some embodiments, the nucleic acid sequence of ceDNA containing codon optimized human PAH version 2 (ceDNA “hPAH_codop_ORF_v2”) with specific cis-regulatory elements is at least 85% identical to SEQ ID NO: 194. According to some embodiments, the nucleic acid sequence of ceDNA containing codon optimized human PAH version 2 (ceDNA “hPAH_codop_ORF_v2”) with specific cis-regulatory elements is at least 90% identical to SEQ ID NO: 194. According to some embodiments, the nucleic acid sequence of ceDNA containing codon optimized human PAH version 2 (ceDNA “hPAH_codop_ORF_v2”) with specific cis-regulatory elements is at least 91% identical to SEQ ID NO: 194. According to some embodiments, the nucleic acid sequence of ceDNA containing codon optimized human PAH version 2 (ceDNA “hPAH_codop_ORF_v2”) with specific cis-regulatory elements is at least 92% identical to SEQ ID NO: 194. According to some embodiments, the nucleic acid sequence of ceDNA containing codon optimized human PAH version 2 (ceDNA “hPAH_codop_ORF_v2”) with specific cis-regulatory elements is at least 93% identical to SEQ ID NO: 194. According to some embodiments, the nucleic acid sequence of ceDNA containing codon optimized human PAH version 2 (ceDNA “hPAH_codop_ORF_v2”) with specific cis-regulatory elements is at least 94% identical to SEQ ID NO: 194. According to some embodiments, the nucleic acid sequence of ceDNA containing codon optimized human PAH version 2 (ceDNA “hPAH_codop_ORF_v2”) with specific cis-regulatory elements is at least 95% identical to SEQ ID NO: 194. According to some embodiments, the nucleic acid sequence of ceDNA containing codon optimized human PAH version 2 (ceDNA “hPAH_codop_ORF_v2”) with specific cis-regulatory elements is at least 96% identical to SEQ ID NO: 194. According to some embodiments, the nucleic acid sequence of ceDNA containing codon optimized human PAH version 2 (ceDNA “hPAH_codop_ORF_v2”) with specific cis-regulatory elements is at least 97% identical to SEQ ID NO: 194. According to some embodiments, the nucleic acid sequence of ceDNA containing codon optimized human PAH version 2 (ceDNA “hPAH_codop_ORF_v2”) with specific cis-regulatory elements is at least 98% identical to SEQ ID NO: 194. According to some embodiments, the nucleic acid sequence of ceDNA containing codon optimized human PAH version 2 (ceDNA “hPAH_codop_ORF_v2”) with specific cis-regulatory elements is at least 99% identical to SEQ ID NO: 194. According to some embodiments, the nucleic acid sequence of ceDNA containing codon optimized human PAH version 2 (ceDNA “hPAH_codop_ORF_v2”) with specific cis-regulatory elements consists of SEQ ID NO: 194.

The nucleic acid sequence of ceDNA containing codon optimized human PAH version 2 (ceDNA “hPAH Codop2”) with a 29 amino acid deletion, with specific cis-regulatory elements is shown as SEQ ID NO: 195 (ceDNA1274).

SEQ ID NO: 195 includes the following elements. left-ITR_v1, spacer_left-ITR_v2.1, VD_PromoterSet, PmeI_site, Mod_Minimum_Consensus_Kozak, hPAH_codop_ORF_v2_delta1-29aa, PacI_site, WPRE_3pUTR, bGH, spacer_right-ITR_v1, right-ITR_v1.

According to some embodiments, the nucleic acid sequence of ceDNA containing codon optimized human PAH version 2 delta1-29aa (ceDNA “hPAH_codop_ORF_v2_delta1-29aa”) with specific cis-regulatory elements comprises SEQ ID NO: 195. According to some embodiments, the nucleic acid sequence of ceDNA containing codon optimized human PAH version 2 delta1-29aa (ceDNA “hPAH_codop_ORF_v2_delta1-29aa”) with specific cis-regulatory elements is at least 85% identical to SEQ ID NO: 195. According to some embodiments, the nucleic acid sequence of ceDNA containing codon optimized human PAH version 2 delta1-29aa (ceDNA “hPAH_codop_ORF_v2_delta1-29aa”) with specific cis-regulatory elements is at least 90% identical to SEQ ID NO: 195. According to some embodiments, the nucleic acid sequence of ceDNA containing codon optimized human PAH version 2 delta1-29aa (ceDNA “hPAH_codop_ORF_v2_delta1-29aa”) with specific cis-regulatory elements is at least 91% identical to SEQ ID NO: 195. According to some embodiments, the nucleic acid sequence of ceDNA containing codon optimized human PAH version 2 delta1-29aa (ceDNA “hPAH_codop_ORF_v2_delta1-29aa”) with specific cis-regulatory elements is at least 92% identical to SEQ ID NO: 195. According to some embodiments, the nucleic acid sequence of ceDNA containing codon optimized human PAH version 2 delta1-29aa (ceDNA “hPAH_codop_ORF_v2_delta1-29aa”) with specific cis-regulatory elements is at least 93% identical to SEQ ID NO: 195. According to some embodiments, the nucleic acid sequence of ceDNA containing codon optimized human PAH version 2 delta1-29aa (ceDNA “hPAH_codop_ORF_v2_delta1-29aa”) with specific cis-regulatory elements is at least 94% identical to SEQ ID NO: 195. According to some embodiments, the nucleic acid sequence of ceDNA containing codon optimized human PAH version 2 delta1-29aa (ceDNA “hPAH_codop_ORF_v2_delta1-29aa”) with specific cis-regulatory elements is at least 95% identical to SEQ ID NO: 195. According to some embodiments, the nucleic acid sequence of ceDNA containing codon optimized human PAH version 2 delta1-29aa (ceDNA “hPAH_codop_ORF_v2_delta1-29aa”) with specific cis-regulatory elements is at least 96% identical to SEQ ID NO: 195. According to some embodiments, the nucleic acid sequence of ceDNA containing codon optimized human PAH version 2 delta1-29aa (ceDNA “hPAH_codop_ORF_v2_delta1-29aa”) with specific cis-regulatory elements is at least 97% identical to SEQ ID NO: 195. According to some embodiments, the nucleic acid sequence of ceDNA containing codon optimized human PAH version 2 delta1-29aa (ceDNA “hPAH_codop_ORF_v2_delta1-29aa”) with specific cis-regulatory elements is at least 98% identical to SEQ ID NO: 195. According to some embodiments, the nucleic acid sequence of ceDNA containing codon optimized human PAH version 2 delta1-29aa (ceDNA “hPAH_codop_ORF_v2_delta1-29aa”) with specific cis-regulatory elements is at least 99% identical to SEQ ID NO: 195. According to some embodiments, the nucleic acid sequence of ceDNA containing codon optimized human PAH version 2 delta1-29aa (ceDNA “hPAH_codop_ORF_v2_delta1-29aa”) with specific cis-regulatory elements consists of SEQ ID NO: 195.

The nucleic acid sequence of ceDNA containing codon optimized human PAH version 2 r5-s29 (ceDNA “hPAH-r5-s29”), with specific cis-regulatory elements is shown as SEQ ID NO: 196 (ceDNA1414).

SEQ ID NO: 196 includes the following elements. left-ITR_v1, spacer_left-ITR_v2.1, 3×VanD_TTRe_PromoterSet, PmeI_site, Mod_Minimum_Consensus_Kozak, hPAH-r5-s29::hIVS1B, PacI_site, WPRE_3pUTR, bGH, spacer_right-ITR_v1, right-ITR_v1.

According to some embodiments, the nucleic acid sequence of ceDNA containing codon optimized human PAH version 2 r5-s29 (ceDNA “hPAH-r5-s29”) with specific cis-regulatory elements comprises SEQ ID NO: 196. According to some embodiments, the nucleic acid sequence of ceDNA containing codon optimized human PAH version 2 r5-s29 (ceDNA “hPAH-r5-s29”) with specific cis-regulatory elements is at least 85% identical to SEQ ID NO: 196. According to some embodiments, the nucleic acid sequence of ceDNA containing codon optimized human PAH version 2 r5-s29 (ceDNA “hPAH-r5-s29”) with specific cis-regulatory elements is at least 90% identical to SEQ ID NO: 196. According to some embodiments, the nucleic acid sequence of ceDNA containing codon optimized human PAH version 2 r5-s29 (ceDNA “hPAH-r5-s29”) with specific cis-regulatory elements is at least 91% identical to SEQ ID NO: 196. According to some embodiments, the nucleic acid sequence of ceDNA containing codon optimized human PAH version 2 r5-s29 (ceDNA “hPAH-r5-s29”) with specific cis-regulatory elements is at least 92% identical to SEQ ID NO: 196. According to some embodiments, the nucleic acid sequence of ceDNA containing codon optimized human PAH version 2 r5-s29 (ceDNA “hPAH-r5-s29”) with specific cis-regulatory elements is at least 93% identical to SEQ ID NO: 196. According to some embodiments, the nucleic acid sequence of ceDNA containing codon optimized human PAH version 2 r5-s29 (ceDNA “hPAH-r5-s29”) with specific cis-regulatory elements is at least 94% identical to SEQ ID NO: 196. According to some embodiments, the nucleic acid sequence of ceDNA containing codon optimized human PAH version 2 r5-s29 (ceDNA “hPAH-r5-s29”) with specific cis-regulatory elements is at least 95% identical to SEQ ID NO: 196. According to some embodiments, the nucleic acid sequence of ceDNA containing codon optimized human PAH version 2 r5-s29 (ceDNA “hPAH-r5-s29”) with specific cis-regulatory elements is at least 96% identical to SEQ ID NO: 196. According to some embodiments, the nucleic acid sequence of ceDNA containing codon optimized human PAH version 2 r5-s29 (ceDNA “hPAH-r5-s29”) with specific cis-regulatory elements is at least 97% identical to SEQ ID NO: 196. According to some embodiments, the nucleic acid sequence of ceDNA containing codon optimized human PAH version 2 r5-s29 (ceDNA “hPAH-r5-s29”) with specific cis-regulatory elements is at least 98% identical to SEQ ID NO: 196. According to some embodiments, the nucleic acid sequence of ceDNA containing codon optimized human PAH version 2 r5-s29 (ceDNA “hPAH-r5-s29”)] with specific cis-regulatory elements is at least 99% identical to SEQ ID NO: 196. According to some embodiments, the nucleic acid sequence of ceDNA containing codon optimized human PAH version 2 r5-s29 (ceDNA “hPAH-r5-s29”) with specific cis-regulatory elements consists of SEQ ID NO: 196.

The nucleic acid sequence of ceDNA containing codon optimized human PAH version 2 r5-s29 (ceDNA “hPAH-r5-s29”), with specific cis-regulatory elements is shown as SEQ ID NO: 197 (ceDNA1416).

SEQ ID NO: 197 includes the following elements. left-ITR_v1, spacer_left-ITR_v2.1, 3×VanD_TTRe_PromoterSet, PmeI_site, Mod_Minimum_Consensus_Kozak, hPAH-r5-s29::hIVS1B_33bpFlanks, PacI_site, WPRE_3pUTR, bGH, spacer_right-ITR_v1, right-ITR_v1.

According to some embodiments, the nucleic acid sequence of ceDNA containing codon optimized human PAH version 2 r5-s29 (ceDNA “hPAH-r5-s29”) with specific cis-regulatory elements comprises SEQ ID NO: 197. According to some embodiments, the nucleic acid sequence of ceDNA containing codon optimized human PAH version 2 r5-s29 (ceDNA “hPAH-r5-s29”) with specific cis-regulatory elements is at least 85% identical to SEQ ID NO: 197. According to some embodiments, the nucleic acid sequence of ceDNA containing codon optimized human PAH version 2 r5-s29 (ceDNA “hPAH-r5-s29”) with specific cis-regulatory elements is at least 90% identical to SEQ ID NO: 197. According to some embodiments, the nucleic acid sequence of ceDNA containing codon optimized human PAH version 2 r5-s29 (ceDNA “hPAH-r5-s29”) with specific cis-regulatory elements is at least 91% identical to SEQ ID NO: 197. According to some embodiments, the nucleic acid sequence of ceDNA containing codon optimized human PAH version 2 r5-s29 (ceDNA “hPAH-r5-s29”) with specific cis-regulatory elements is at least 92% identical to SEQ ID NO: 197. According to some embodiments, the nucleic acid sequence of ceDNA containing codon optimized human PAH version 2 r5-s29 (ceDNA “hPAH-r5-s29”) with specific cis-regulatory elements is at least 93% identical to SEQ ID NO: 197. According to some embodiments, the nucleic acid sequence of ceDNA containing codon optimized human PAH version 2 r5-s29 (ceDNA “hPAH-r5-s29”) with specific cis-regulatory elements is at least 94% identical to SEQ ID NO: 197. According to some embodiments, the nucleic acid sequence of ceDNA containing codon optimized human PAH version 2 r5-s29 (ceDNA “hPAH-r5-s29”) with specific cis-regulatory elements is at least 95% identical to SEQ ID NO: 197. According to some embodiments, the nucleic acid sequence of ceDNA containing codon optimized human PAH version 2 r5-s29 (ceDNA “hPAH-r5-s29”) with specific cis-regulatory elements is at least 96% identical to SEQ ID NO: 197. According to some embodiments, the nucleic acid sequence of ceDNA containing codon optimized human PAH version 2 r5-s29 (ceDNA “hPAH-r5-s29”) with specific cis-regulatory elements is at least 97% identical to SEQ ID NO: 197. According to some embodiments, the nucleic acid sequence of ceDNA containing codon optimized human PAH version 2 r5-s29 (ceDNA “hPAH-r5-s29”) with specific cis-regulatory elements is at least 98% identical to SEQ ID NO: 197. According to some embodiments, the nucleic acid sequence of ceDNA containing codon optimized human PAH version 2 r5-s29 (ceDNA “hPAH-r5-s29”) with specific cis-regulatory elements is at least 99% identical to SEQ ID NO: 197. According to some embodiments, the nucleic acid sequence of ceDNA containing codon optimized human PAH version 2 r5-s29 (ceDNA “hPAH-r5-s29”) with specific cis-regulatory elements consists of SEQ ID NO: 197. The nucleic acid sequence of ceDNA containing codon optimized human PAH version 2 r5-s29 (ceDNA “hPAH-r5-s29”), with specific cis-regulatory elements is shown as SEQ ID NO: 198(ceDNA1428).

SEQ ID NO: 198 includes the following elements. left-ITR_v1, spacer_left-ITR_v2.1, 3×VanD_TTRe_PromoterSet, PmeI_site, Mod_Minimum_Consensus_Kozak, hPAH-r5-s29::mod-Intron_oIVS-v2_33bpFlanks, PacI_site, WPRE_3pUTR, bGH, spacer_right-ITR_v1, right-ITR_v1.

According to some embodiments, the nucleic acid sequence of ceDNA containing codon optimized human PAH version 2 r5-s29 (ceDNA “hPAH-r5-s29”) with specific cis-regulatory elements comprises SEQ ID NO: 198. According to some embodiments, the nucleic acid sequence of ceDNA containing codon optimized human PAH version 2 r5-s29 (ceDNA “hPAH-r5-s29”) with specific cis-regulatory elements is at least 85% identical to SEQ ID NO: 198. According to some embodiments, the nucleic acid sequence of ceDNA containing codon optimized human PAH version 2 r5-s29 (ceDNA “hPAH-r5-s29”) with specific cis-regulatory elements is at least 90% identical to SEQ ID NO: 198. According to some embodiments, the nucleic acid sequence of ceDNA containing codon optimized human PAH version 2 r5-s29 (ceDNA “hPAH-r5-s29”) with specific cis-regulatory elements is at least 91% identical to SEQ ID NO: 198. According to some embodiments, the nucleic acid sequence of ceDNA containing codon optimized human PAH version 2 r5-s29 (ceDNA “hPAH-r5-s29”) with specific cis-regulatory elements is at least 92% identical to SEQ ID NO: 198. According to some embodiments, the nucleic acid sequence of ceDNA containing codon optimized human PAH version 2 r5-s29 (ceDNA “hPAH-r5-s29”) with specific cis-regulatory elements is at least 93% identical to SEQ ID NO: 198. According to some embodiments, the nucleic acid sequence of ceDNA containing codon optimized human PAH version 2 r5-s29 (ceDNA “hPAH-r5-s29”) with specific cis-regulatory elements is at least 94% identical to SEQ ID NO: 198. According to some embodiments, the nucleic acid sequence of ceDNA containing codon optimized human PAH version 2 r5-s29 (ceDNA “hPAH-r5-s29”) with specific cis-regulatory elements is at least 95% identical to SEQ ID NO: 198. According to some embodiments, the nucleic acid sequence of ceDNA containing codon optimized human PAH version 2 r5-s29 (ceDNA “hPAH-r5-s29”) with specific cis-regulatory elements is at least 96% identical to SEQ ID NO: 198. According to some embodiments, the nucleic acid sequence of ceDNA containing codon optimized human PAH version 2 r5-s29 (ceDNA “hPAH-r5-s29”) with specific cis-regulatory elements is at least 97% identical to SEQ ID NO: 198. According to some embodiments, the nucleic acid sequence of ceDNA containing codon optimized human PAH version 2 r5-s29 (ceDNA “hPAH-r5-s29”) with specific cis-regulatory elements is at least 98% identical to SEQ ID NO: 198. According to some embodiments, the nucleic acid sequence of ceDNA containing codon optimized human PAH version 2 r5-s29 (ceDNA “hPAH-r5-s29”) with specific cis-regulatory elements is at least 99% identical to SEQ ID NO: 198. According to some embodiments, the nucleic acid sequence of ceDNA containing codon optimized human PAH version 2 r5-s29 (ceDNA “hPAH-r5-s29”) with specific cis-regulatory elements consists of SEQ ID NO: 198.

The nucleic acid sequence of ceDNA containing codon optimized human PAH version 2 (ceDNA “hPAH_codop_ORF_v2”), with specific cis-regulatory elements is shown as SEQ ID NO: 199 (ceDNA1430).

SEQ ID NO: 199 includes the following elements. left-ITR_v1, spacer_left-ITR_v2.1, 3×VanD_TTRe_PromoterSet, PmeI_site, Mod_Minimum_Consensus_Kozak, hPAH_codop_ORF_v2_mIVS-intron1B_33bpFlanks, PacI_site, WPRE_3pUTR, bGH, spacer_right-ITR_v1, right-ITR_v1.

According to some embodiments, the nucleic acid sequence of ceDNA containing codon optimized human PAH version 2 (ceDNA “hPAH_codop_ORF_v2”) with specific cis-regulatory elements comprises SEQ ID NO: 199. According to some embodiments, the nucleic acid sequence of ceDNA containing codon optimized human PAH version 2 (ceDNA “hPAH_codop_ORF_v2”) with specific cis-regulatory elements is at least 85% identical to SEQ ID NO: 199. According to some embodiments, the nucleic acid sequence of ceDNA containing codon optimized human PAH version 2 (ceDNA “hPAH_codop_ORF_v2”) with specific cis-regulatory elements is at least 90% identical to SEQ ID NO: 199. According to some embodiments, the nucleic acid sequence of ceDNA containing codon optimized human PAH version 2 (ceDNA “hPAH_codop_ORF_v2”) with specific cis-regulatory elements is at least 91% identical to SEQ ID NO: 199. According to some embodiments, the nucleic acid sequence of ceDNA containing codon optimized human PAH version 2 (ceDNA “hPAH_codop_ORF_v2”) with specific cis-regulatory elements is at least 92% identical to SEQ ID NO: 199. According to some embodiments, the nucleic acid sequence of ceDNA containing codon optimized human PAH version 2 (ceDNA “hPAH_codop_ORF_v2”) with specific cis-regulatory elements is at least 93% identical to SEQ ID NO: 199. According to some embodiments, the nucleic acid sequence of ceDNA containing codon optimized human PAH version 2 (ceDNA “hPAH_codop_ORF_v2”) with specific cis-regulatory elements is at least 94% identical to SEQ ID NO: 199. According to some embodiments, the nucleic acid sequence of ceDNA containing codon optimized human PAH version 2 (ceDNA “hPAH_codop_ORF_v2”) with specific cis-regulatory elements is at least 95% identical to SEQ ID NO: 199. According to some embodiments, the nucleic acid sequence of ceDNA containing codon optimized human PAH version 2 (ceDNA “hPAH_codop_ORF_v2”) with specific cis-regulatory elements is at least 96% identical to SEQ ID NO: 199. According to some embodiments, the nucleic acid sequence of ceDNA containing codon optimized human PAH version 2 (ceDNA “hPAH_codop_ORF_v2”) with specific cis-regulatory elements is at least 97% identical to SEQ ID NO: 199. According to some embodiments, the nucleic acid sequence of ceDNA containing codon optimized human PAH version 2 (ceDNA “hPAH_codop_ORF_v2”) with specific cis-regulatory elements is at least 98% identical to SEQ ID NO: 199. According to some embodiments, the nucleic acid sequence of ceDNA containing codon optimized human PAH version 2 (ceDNA “hPAH_codop_ORF_v2”) with specific cis-regulatory elements is at least 99% identical to SEQ ID NO: 199. According to some embodiments, the nucleic acid sequence of ceDNA containing codon optimized human PAH version 2 (ceDNA “hPAH_codop_ORF_v2”) with specific cis-regulatory elements consists of SEQ ID NO: 199.

The nucleic acid sequence of ceDNA containing codon optimized human PAH version 2 (ceDNA “hPAH_codop_ORF_v2”), with specific cis-regulatory elements is shown as SEQ ID NO: 200 (ceDNA1432).

(SEQ ID NO: 200)
CCTGCAGGCAGCTGCGCGCTCGCTCGCTCACTGAGGCCGC
CCGGGCAAAGCCCGGGCGTCGGGCGACCTTTGGTCGCCCG
GCCTCAGTGAGCGAGCGAGCGCGCAGAGAGGGAGTGGCCA
ACTCCATCACTAGGGGTTCCTTGTAGTTAATGATTAACCC
GCCATGCTACTTATCGCGGCCGCGGGGGAGGCTGCTGGTG
AATATTAACCAAGGTCACCCCAGTTATCGGAGGAGCAAAC
AGGGGCTAAGTCCACCGGGGGAGGCTGCTGGTGAATATTA
ACCAAGGTCACCCCAGTTATCGGAGGAGCAAACAGGGGCT
AAGTCCACCGGGGGAGGCTGCTGGTGAATATTAACCAAGG
TCACCCCAGTTATCGGAGGAGCAAACAGGGGCTAAGTCCA
CGGTACCCACTGGGAGGATGTTGAGTAAGATGGAAAACTA
CTGATGACCCTTGCAGAGACAGAGTATTAGGACATGTTTG
AACAGGGGCCGGGCGATCAGCAGGTAGCTCTAGAGGATCC
CCGTCTGTCTGCACATTTCGTAGAGCGAGTGTTCCGATAC
TCTAATCTCCCTAGGCAAGGTTCATATTTGTGTAGGTTAC
TTATTCTCCTTTTGTTGACTAAGTCAATAATCAGAATCAG
CAGGTTTGGAGTCAGCTTGGCAGGGATCAGCAGCCTGGGT
TGGAAGGAGGGGGTATAAAAGCCCCTTCACCAGGAGAAGC
CGTCACACAGATCCACAAGCTCCTGAAGAGGTAAGGGTTT
AAGGGATGGTTGGTTGGTGGGGTATTAATGTTTAATTACC
TGGAGCACCTGCCTGAAATCACTTTTTTTCAGGTTGGGTT
TAAACCGCAGCCACCATGAGCACCGCCGTGCTGGAAAATC
CTGGCTTGGGCAGGAAACTCTCTGACTTTGGACAGGTGAG
CCACGGCAGCCTGAGCTGCTCAGTTAGGGGAATTTGGGCC
TCCAGAGAAAGAGATCCGAAGACTGCTGGTGCTTCCTGGT
TTCATAAGCTCAGTAAGAAGTCTGAATTCGTTGGAAGCTG
ATGATAGAAGAAAGAGTTCATGCTTGCTTTGTCCATGGAG
GTTTAACAGGAATGAATTGCTAAACTGTGGAAAATGTTTT
AAACAAATGCATCTTATCCTGTAGGAAACAAGCTATATTG
AAGACAACTGCAATCAAAACGGCGCCATCAGCCTGATCTT
CAGCCTGAAAGAAGAAGTGGGCGCCCTGGCCAAGGTGCTG
AGACTGTTCGAAGAGAACGACGTGAACCTGACACACATCG
AGAGCAGACCCAGCAGACTGAAGAAGGACGAGTACGAGTT
CTTCACCCACCTGGACAAGCGGAGCCTGCCTGCTCTGACC
AACATCATCAAGATCCTGCGGCACGACATCGGCGCCACAG
TGCACGAACTGAGCCGGGACAAGAAAAAGGACACCGTGCC
ATGGTTCCCCAGAACCATCCAAGAGCTGGACAGATTCGCC
AACCAGATCCTGAGCTATGGCGCCGAGCTGGACGCTGATC
ACCCTGGCTTTAAGGACCCCGTGTACCGGGCCAGAAGAAA
GCAGTTTGCCGATATCGCCTACAACTACCGGCACGGCCAG
CCTATTCCTCGGGTCGAGTACATGGAAGAGGAAAAGAAAA
CCTGGGGCACCGTGTTCAAGACCCTGAAGTCCCTGTACAA
GACCCACGCCTGCTACGAGTACAACCACATCTTCCCACTG
CTCGAAAAGTACTGCGGCTTCCACGAGGACAATATCCCTC
AGCTTGAGGACGTGTCCCAGTTCCTGCAGACCTGCACCGG
CTTTAGACTGAGGCCAGTTGCCGGACTGCTGAGCAGCAGA
GATTTTCTCGGCGGCCTGGCCTTCAGAGTGTTCCACTGTA
CCCAGTACATCAGACACGGCAGCAAGCCCATGTACACCCC
TGAGCCTGATATCTGCCACGAGCTGCTGGGACATGTGCCC
CTGTTCAGCGATAGAAGCTTCGCCCAGTTCAGCCAAGAGA
TCGGACTGGCTTCTCTGGGAGCCCCTGACGAGTACATTGA
GAAGCTGGCCACCATCTACTGGTTCACCGTGGAATTCGGC
CTGTGCAAGCAGGGCGACAGCATCAAAGCTTATGGCGCTG
GCCTGCTGTCTAGCTTCGGCGAGCTGCAGTACTGTCTGAG
CGAGAAGCCTAAGCTGCTGCCCCTGGAACTGGAAAAGACC
GCCATCCAGAACTACACCGTGACCGAGTTCCAGCCTCTGT
ACTACGTGGCCGAGAGCTTCAACGACGCCAAAGAAAAAGT
GCGGAACTTCGCCGCCACCATTCCTCGGCCTTTCAGCGTC
AGATACGACCCCTACACACAGCGGATCGAGGTGCTGGACA
ACACACAGCAGCTGAAAATTCTGGCCGACTCCATCAACAG
CGAGATCGGCATCCTGTGCAGCGCCCTGCAGAAAATCAAG
TGAtagTTAATTAAGAGCATCTTACCGCCATTTATTCCCA
TATTTGTTCTGTTTTTCTTGATTTGGGTATACATTTAAAT
GTTAATAAAACAAAATGGTGGGGCAATCATTTACATTTTT
AGGGATATGTAATTACTAGTTCAGGTGTATTGCCACAAGA
CAAACATGTTAAGAAACTTTCCCGTTATTTACGCTCTGTT
CCTGTTAATCAACCTCTGGATTACAAAATTTGTGAAAGAT
TGACTGATATTCTTAACTATGTTGCTCCTTTTACGCTGTG
TGGATATGCTGCTTTATAGCCTCTGTATCTAGCTATTGCT
TCCCGTACGGCTTTCGTTTTCTCCTCCTTGTATAAATCCT
GGTTGCTGTCTCTTTTAGAGGAGTTGTGGCCCGTTGTCCG
TCAACGTGGCGTGGTGTGCTCTGTGTTTGCTGACGCAACC
CCCACTGGCTGGGGCATTGCCACCACCTGTCAACTCCTTT
CTGGGACTTTCGCTTTCCCCCTCCCGATCGCCACGGCAGA
ACTCATCGCCGCCTGCCTTGCCCGCTGCTGGACAGGGGCT
AGGTTGCTGGGCACTGATAATTCCGTGGTGTTGTCTGTGC
CTTCTAGTTGCCAGCCATCTGTTGTTTGCCCCTCCCCCGT
GCCTTCCTTGACCCTGGAAGGTGCCACTCCCACTGTCCTT
TCCTAATAAAATGAGGAAATTGCATCGCATTGTCTGAGTA
GGTGTCATTCTATTCTGGGGGGTGGGGTGGGGCAGGACAG
CAAGGGGGAGGATTGGGAAGACAATAGCAGGCATGCTGGG
GATGCGGTGGGCTCTATGGCTCTAGAGCATGGCTACGTAG
ATAAGTAGCATGGCGGGTTAATCATTAACTACACCTGCAG
GAGGAACCCCTAGTGATGGAGTTGGCCACTCCCTCTCTGC
GCGCTCGCTCGCTCACTGAGGCCGGGCGACCAAAGGTCGC
CCGACGCCCGGGCGGCCTCAGTGAGCGAGCGAGCGCGCAG
CTGCCTGCAGG

SEQ ID NO: 200 includes the following elements. left-ITR_v1, spacer_left-ITR_v2.1, 3×VanD_TTRe_PromoterSet, PmeI_site, Mod_Minimum_Consensus_Kozak, hPAH_codop_ORF_v2_modified_Intron1_33bpFlanks, PacI_site, WPRE_3pUTR, bGH, spacer_right-ITR_v1, right-ITR_v1.

According to some embodiments, the nucleic acid sequence of ceDNA containing codon optimized human PAH version 2 (ceDNA “hPAH_codop_ORF_v2”) with specific cis-regulatory elements comprises SEQ ID NO: 200. According to some embodiments, the nucleic acid sequence of ceDNA containing codon optimized human PAH version 2 (ceDNA “hPAH_codop_ORF_v2”) with specific cis-regulatory elements is at least 85% identical to SEQ ID NO: 200. According to some embodiments, the nucleic acid sequence of ceDNA containing codon optimized human PAH version 2 (ceDNA “hPAH_codop_ORF_v2”) with specific cis-regulatory elements is at least 90% identical to SEQ ID NO: 200. According to some embodiments, the nucleic acid sequence of ceDNA containing codon optimized human PAH version 2 (ceDNA “hPAH_codop_ORF_v2”) with specific cis-regulatory elements is at least 91% identical to SEQ ID NO: 200. According to some embodiments, the nucleic acid sequence of ceDNA containing codon optimized human PAH version 2 (ceDNA “hPAH_codop_ORF_v2”) with specific cis-regulatory elements is at least 92% identical to SEQ ID NO: 200. According to some embodiments, the nucleic acid sequence of ceDNA containing codon optimized human PAH version 2 (ceDNA “hPAH_codop_ORF_v2”) with specific cis-regulatory elements is at least 93% identical to SEQ ID NO: 200. According to some embodiments, the nucleic acid sequence of ceDNA containing codon optimized human PAH version 2 (ceDNA “hPAH_codop_ORF_v2”) with specific cis-regulatory elements is at least 94% identical to SEQ ID NO: 200. According to some embodiments, the nucleic acid sequence of ceDNA containing codon optimized human PAH version 2 (ceDNA “hPAH_codop_ORF_v2”) with specific cis-regulatory elements is at least 95% identical to SEQ ID NO: 200. According to some embodiments, the nucleic acid sequence of ceDNA containing codon optimized human PAH version 2 (ceDNA “hPAH_codop_ORF_v2”) with specific cis-regulatory elements is at least 96% identical to SEQ ID NO: 200. According to some embodiments, the nucleic acid sequence of ceDNA containing codon optimized human PAH version 2 (ceDNA “hPAH_codop_ORF_v2”) with specific cis-regulatory elements is at least 97% identical to SEQ ID NO: 200. According to some embodiments, the nucleic acid sequence of ceDNA containing codon optimized human PAH version 2 (ceDNA “hPAH_codop_ORF_v2”) with specific cis-regulatory elements is at least 98% identical to SEQ ID NO: 200. According to some embodiments, the nucleic acid sequence of ceDNA containing codon optimized human PAH version 2 (ceDNA “hPAH_codop_ORF_v2”) with specific cis-regulatory elements is at least 99% identical to SEQ ID NO: 200. According to some embodiments, the nucleic acid sequence of ceDNA containing codon optimized human PAH version 2 (ceDNA “hPAH_codop_ORF_v2”) with specific cis-regulatory elements consists of SEQ ID NO: 200.

The nucleic acid sequence of ceDNA containing codon optimized human PAH version 2 (ceDNA “hPAH_codop_ORF_v2”), with specific cis-regulatory elements is shown as SEQ ID NO: 201 (ceDNA1436).

SEQ ID NO: 202 includes the following elements. left-ITR_v1, spacer_left-ITR_v2.1, hAAT(979)_PromoterSet, PmeI_site, Mod_Minimum_Consensus_Kozak, hPAH_codop_ORF_v2, PacI_site, WPRE_3pUTR, bGH, spacer_right-ITR_v1, right-ITR_v1.

According to some embodiments, the nucleic acid sequence of ceDNA containing codon optimized human PAH version 2 (ceDNA “hPAH_codop_ORF_v2”) with specific cis-regulatory elements comprises SEQ ID NO: 201. According to some embodiments, the nucleic acid sequence of ceDNA containing codon optimized human PAH version 2 (ceDNA “hPAH_codop_ORF_v2”) with specific cis-regulatory elements is at least 85% identical to SEQ ID NO: 201. According to some embodiments, the nucleic acid sequence of ceDNA containing codon optimized human PAH version 2 (ceDNA “hPAH_codop_ORF_v2”) with specific cis-regulatory elements is at least 90% identical to SEQ ID NO: 201. According to some embodiments, the nucleic acid sequence of ceDNA containing codon optimized human PAH version 2 (ceDNA “hPAH_codop_ORF_v2”) with specific cis-regulatory elements is at least 91% identical to SEQ ID NO: 201. According to some embodiments, the nucleic acid sequence of ceDNA containing codon optimized human PAH version 2 (ceDNA “hPAH_codop_ORF_v2”) with specific cis-regulatory elements is at least 92% identical to SEQ ID NO: 201. According to some embodiments, the nucleic acid sequence of ceDNA containing codon optimized human PAH version 2 (ceDNA “hPAH_codop_ORF_v2”) with specific cis-regulatory elements is at least 93% identical to SEQ ID NO: 201. According to some embodiments, the nucleic acid sequence of ceDNA containing codon optimized human PAH version 2 (ceDNA “hPAH_codop_ORF_v2”) with specific cis-regulatory elements is at least 94% identical to SEQ ID NO: 201. According to some embodiments, the nucleic acid sequence of ceDNA containing codon optimized human PAH version 2 (ceDNA “hPAH_codop_ORF_v2”) with specific cis-regulatory elements is at least 95% identical to SEQ ID NO: 201. According to some embodiments, the nucleic acid sequence of ceDNA containing codon optimized human PAH version 2 (ceDNA “hPAH_codop_ORF_v2”) with specific cis-regulatory elements is at least 96% identical to SEQ ID NO: 201. According to some embodiments, the nucleic acid sequence of ceDNA containing codon optimized human PAH version 2 (ceDNA “hPAH_codop_ORF_v2”) with specific cis-regulatory elements is at least 97% identical to SEQ ID NO: 201. According to some embodiments, the nucleic acid sequence of ceDNA containing codon optimized human PAH version 2 (ceDNA “hPAH_codop_ORF_v2”) with specific cis-regulatory elements is at least 98% identical to SEQ ID NO: 201. According to some embodiments, the nucleic acid sequence of ceDNA containing codon optimized human PAH version 2 (ceDNA “hPAH_codop_ORF_v2”) with specific cis-regulatory elements is at least 99% identical to SEQ ID NO: 201. According to some embodiments, the nucleic acid sequence of ceDNA containing codon optimized human PAH version 2 (ceDNA “hPAH_codop_ORF_v2”) with specific cis-regulatory elements consists of SEQ ID NO: 201.

The nucleic acid sequence of ceDNA containing codon optimized human PAH version 2 (ceDNA “hPAH_codop_ORF_v2”), with specific cis-regulatory elements is shown as SEQ ID NO: 202 (ceDNA1458).

SEQ ID NO: 202 includes the following elements. left-ITR_v1, spacer_left-ITR_v2.1, VD_PromoterSet, PmeI_site, Mod_Minimum_Consensus_Kozak, hPAH_codop_ORF_v2, PacI_site, HBBv2_3pUTR, SV40_polyA, spacer_right-ITR_v1, right-ITR_v1.

According to some embodiments, the nucleic acid sequence of ceDNA containing codon optimized human PAH version 2 (ceDNA “hPAH_codop_ORF_v2”) with specific cis-regulatory elements comprises SEQ ID NO: 202. According to some embodiments, the nucleic acid sequence of ceDNA containing codon optimized human PAH version 2 (ceDNA “hPAH_codop_ORF_v2”) with specific cis-regulatory elements is at least 85% identical to SEQ ID NO: 202. According to some embodiments, the nucleic acid sequence of ceDNA containing codon optimized human PAH version 2 (ceDNA “hPAH_codop_ORF_v2”) with specific cis-regulatory elements is at least 90% identical to SEQ ID NO: 202. According to some embodiments, the nucleic acid sequence of ceDNA containing codon optimized human PAH version 2 (ceDNA “hPAH_codop_ORF_v2”) with specific cis-regulatory elements is at least 91% identical to SEQ ID NO: 202. According to some embodiments, the nucleic acid sequence of ceDNA containing codon optimized human PAH version 2 (ceDNA “hPAH_codop_ORF_v2”) with specific cis-regulatory elements is at least 92% identical to SEQ ID NO: 202. According to some embodiments, the nucleic acid sequence of ceDNA containing codon optimized human PAH version 2 (ceDNA “hPAH_codop_ORF_v2”) with specific cis-regulatory elements is at least 93% identical to SEQ ID NO: 202. According to some embodiments, the nucleic acid sequence of ceDNA containing codon optimized human PAH version 2 (ceDNA “hPAH_codop_ORF_v2”) with specific cis-regulatory elements is at least 94% identical to SEQ ID NO: 202. According to some embodiments, the nucleic acid sequence of ceDNA containing codon optimized human PAH version 2 (ceDNA “hPAH_codop_ORF_v2”) with specific cis-regulatory elements is at least 95% identical to SEQ ID NO: 202. According to some embodiments, the nucleic acid sequence of ceDNA containing codon optimized human PAH version 2 (ceDNA “hPAH_codop_ORF_v2”) with specific cis-regulatory elements is at least 96% identical to SEQ ID NO: 202. According to some embodiments, the nucleic acid sequence of ceDNA containing codon optimized human PAH version 2 (ceDNA “hPAH_codop_ORF_v2”) with specific cis-regulatory elements is at least 97% identical to SEQ ID NO: 202. According to some embodiments, the nucleic acid sequence of ceDNA containing codon optimized human PAH version 2 (ceDNA “hPAH_codop_ORF_v2”) with specific cis-regulatory elements is at least 98% identical to SEQ ID NO: 202. According to some embodiments, the nucleic acid sequence of ceDNA containing codon optimized human PAH version 2 (ceDNA “hPAH_codop_ORF_v2”) with specific cis-regulatory elements is at least 99% identical to SEQ ID NO: 202. According to some embodiments, the nucleic acid sequence of ceDNA containing codon optimized human PAH version 2 (ceDNA “hPAH_codop_ORF_v2”) with specific cis-regulatory elements consists of SEQ ID NO: 202.

The nucleic acid sequence of ceDNA containing codon optimized human PAH version 2 (ceDNA “hPAH_codop_ORF_v2”), with specific cis-regulatory elements is shown as SEQ ID NO: 203 (ceDNA1459).

SEQ ID NO: 203 includes the following elements. 1 left-ITR_v1, spacer_left-ITR_v2.1, VD_PromoterSet, PmeI_site, Mod_Minimum_Consensus_Kozak, hPAH_codop_ORF_v2, PacI_site, HBBv3_3pUTR, SV40_polyA, spacer_right-ITR_v1, right-ITR_v1.

According to some embodiments, the nucleic acid sequence of ceDNA containing codon optimized human PAH version 2 (ceDNA “hPAH_codop_ORF_v2”) with specific cis-regulatory elements comprises SEQ ID NO: 203. According to some embodiments, the nucleic acid sequence of ceDNA containing codon optimized human PAH version 2 (ceDNA “hPAH_codop_ORF_v2”) with specific cis-regulatory elements is at least 85% identical to SEQ ID NO: 203. According to some embodiments, the nucleic acid sequence of ceDNA containing codon optimized human PAH version 2 (ceDNA “hPAH_codop_ORF_v2”) with specific cis-regulatory elements is at least 90% identical to SEQ ID NO: 203. According to some embodiments, the nucleic acid sequence of ceDNA containing codon optimized human PAH version 2 (ceDNA “hPAH_codop_ORF_v2”) with specific cis-regulatory elements is at least 91% identical to SEQ ID NO: 203. According to some embodiments, the nucleic acid sequence of ceDNA containing codon optimized human PAH version 2 (ceDNA “hPAH_codop_ORF_v2”) with specific cis-regulatory elements is at least 92% identical to SEQ ID NO: 203. According to some embodiments, the nucleic acid sequence of ceDNA containing codon optimized human PAH version 2 (ceDNA “hPAH_codop_ORF_v2”) with specific cis-regulatory elements is at least 93% identical to SEQ ID NO: 203. According to some embodiments, the nucleic acid sequence of ceDNA containing codon optimized human PAH version 2 (ceDNA “hPAH_codop_ORF_v2”) with specific cis-regulatory elements is at least 94% identical to SEQ ID NO: 203. According to some embodiments, the nucleic acid sequence of ceDNA containing codon optimized human PAH version 2 (ceDNA “hPAH_codop_ORF_v2”) with specific cis-regulatory elements is at least 95% identical to SEQ ID NO: 203. According to some embodiments, the nucleic acid sequence of ceDNA containing codon optimized human PAH version 2 (ceDNA “hPAH_codop_ORF_v2”) with specific cis-regulatory elements is at least 96% identical to SEQ ID NO: 203. According to some embodiments, the nucleic acid sequence of ceDNA containing codon optimized human PAH version 2 (ceDNA “hPAH_codop_ORF_v2”) with specific cis-regulatory elements is at least 97% identical to SEQ ID NO: 203. According to some embodiments, the nucleic acid sequence of ceDNA containing codon optimized human PAH version 2 (ceDNA “hPAH_codop_ORF_v2”) with specific cis-regulatory elements is at least 98% identical to SEQ ID NO: 203. According to some embodiments, the nucleic acid sequence of ceDNA containing codon optimized human PAH version 2 (ceDNA “hPAH_codop_ORF_v2”) with specific cis-regulatory elements is at least 99% identical to SEQ ID NO: 203. According to some embodiments, the nucleic acid sequence of ceDNA containing codon optimized human PAH version 2 (ceDNA “hPAH_codop_ORF_v2”) with specific cis-regulatory elements consists of SEQ ID NO: 203.

The nucleic acid sequence of ceDNA containing codon optimized human PAH version 2 (ceDNA “hPAH_codop_ORF_v2”), with specific cis-regulatory elements is shown as SEQ ID NO: 204 (ceDNA1464).

SEQ ID NO: 204 includes the following elements. left-ITR_v1, spacer_left-ITR_v2.1, VD_PromoterSet, PmeI_site, Mod_Minimum_Consensus_Kozak, hPAH_codop_ORF_v2, PacI_site, WPRE_3pUTR, SV40_polyA, spacer_right-ITR_v1, right-ITR_v1.

According to some embodiments, the nucleic acid sequence of ceDNA containing codon optimized human PAH version 2 (ceDNA “hPAH_codop_ORF_v2”) with specific cis-regulatory elements comprises SEQ ID NO: 204. According to some embodiments, the nucleic acid sequence of ceDNA containing codon optimized human PAH version 2 (ceDNA “hPAH_codop_ORF_v2”) with specific cis-regulatory elements is at least 85% identical to SEQ ID NO: 204. According to some embodiments, the nucleic acid sequence of ceDNA containing codon optimized human PAH version 2 (ceDNA “hPAH_codop_ORF_v2”) with specific cis-regulatory elements is at least 90% identical to SEQ ID NO: 204. According to some embodiments, the nucleic acid sequence of ceDNA containing codon optimized human PAH version 2 (ceDNA “hPAH_codop_ORF_v2”) with specific cis-regulatory elements is at least 91% identical to SEQ ID NO: 204. According to some embodiments, the nucleic acid sequence of ceDNA containing codon optimized human PAH version 2 (ceDNA “hPAH_codop_ORF_v2”) with specific cis-regulatory elements is at least 92% identical to SEQ ID NO: 204. According to some embodiments, the nucleic acid sequence of ceDNA containing codon optimized human PAH version 2 (ceDNA “hPAH_codop_ORF_v2”) with specific cis-regulatory elements is at least 93% identical to SEQ ID NO: 204. According to some embodiments, the nucleic acid sequence of ceDNA containing codon optimized human PAH version 2 (ceDNA “hPAH_codop_ORF_v2”) with specific cis-regulatory elements is at least 94% identical to SEQ ID NO: 204. According to some embodiments, the nucleic acid sequence of ceDNA containing codon optimized human PAH version 2 (ceDNA “hPAH_codop_ORF_v2”) with specific cis-regulatory elements is at least 95% identical to SEQ ID NO: 204. According to some embodiments, the nucleic acid sequence of ceDNA containing codon optimized human PAH version 2 (ceDNA “hPAH_codop_ORF_v2”) with specific cis-regulatory elements is at least 96% identical to SEQ ID NO: 204. According to some embodiments, the nucleic acid sequence of ceDNA containing codon optimized human PAH version 2 (ceDNA “hPAH_codop_ORF_v2”) with specific cis-regulatory elements is at least 97% identical to SEQ ID NO: 204. According to some embodiments, the nucleic acid sequence of ceDNA containing codon optimized human PAH version 2 (ceDNA “hPAH_codop_ORF_v2”) with specific cis-regulatory elements is at least 98% identical to SEQ ID NO: 204. According to some embodiments, the nucleic acid sequence of ceDNA containing codon optimized human PAH version 2 (ceDNA “hPAH_codop_ORF_v2”) with specific cis-regulatory elements is at least 99% identical to SEQ ID NO: 204. According to some embodiments, the nucleic acid sequence of ceDNA containing codon optimized human PAH version 2 (ceDNA “hPAH_codop_ORF_v2”) with specific cis-regulatory elements consists of SEQ ID NO: 204.

The nucleic acid sequence of ceDNA containing codon optimized human PAH version 2 (ceDNA “hPAH_codop_ORF_v2”), with specific cis-regulatory elements is shown as SEQ ID NO: 205 (ceDNA1466).

SEQ ID NO: 205 includes the following elements. left-ITR_v1, spacer_left-ITR_v2.1, VD_PromoterSet, PmeI_site, Mod_Minimum_Consensus_Kozak, hPAH_codop_ORF_v2, PacI_site, WPRE_3pUTR, HBBv2_3pUTR, spacer_right-ITR_v1, right-ITR_v1.

According to some embodiments, the nucleic acid sequence of ceDNA containing codon optimized human PAH version 2 (ceDNA “hPAH_codop_ORF_v2”) with specific cis-regulatory elements comprises SEQ ID NO: 205. According to some embodiments, the nucleic acid sequence of ceDNA containing codon optimized human PAH version 2 (ceDNA “hPAH_codop_ORF_v2”) with specific cis-regulatory elements is at least 85% identical to SEQ ID NO: 205. According to some embodiments, the nucleic acid sequence of ceDNA containing codon optimized human PAH version 2 (ceDNA “hPAH_codop_ORF_v2”) with specific cis-regulatory elements is at least 90% identical to SEQ ID NO: 205. According to some embodiments, the nucleic acid sequence of ceDNA containing codon optimized human PAH version 2 (ceDNA “hPAH_codop_ORF_v2”) with specific cis-regulatory elements is at least 91% identical to SEQ ID NO: 205. According to some embodiments, the nucleic acid sequence of ceDNA containing codon optimized human PAH version 2 (ceDNA “hPAH_codop_ORF_v2”) with specific cis-regulatory elements is at least 92% identical to SEQ ID NO: 205. According to some embodiments, the nucleic acid sequence of ceDNA containing codon optimized human PAH version 2 (ceDNA “hPAH_codop_ORF_v2”) with specific cis-regulatory elements is at least 93% identical to SEQ ID NO: 205. According to some embodiments, the nucleic acid sequence of ceDNA containing codon optimized human PAH version 2 (ceDNA “hPAH_codop_ORF_v2”) with specific cis-regulatory elements is at least 94% identical to SEQ ID NO: 205. According to some embodiments, the nucleic acid sequence of ceDNA containing codon optimized human PAH version 2 (ceDNA “hPAH_codop_ORF_v2”) with specific cis-regulatory elements is at least 95% identical to SEQ ID NO: 205. According to some embodiments, the nucleic acid sequence of ceDNA containing codon optimized human PAH version 2 (ceDNA “hPAH_codop_ORF_v2”) with specific cis-regulatory elements is at least 96% identical to SEQ ID NO: 205. According to some embodiments, the nucleic acid sequence of ceDNA containing codon optimized human PAH version 2 (ceDNA “hPAH_codop_ORF_v2”) with specific cis-regulatory elements is at least 97% identical to SEQ ID NO: 205. According to some embodiments, the nucleic acid sequence of ceDNA containing codon optimized human PAH version 2 (ceDNA “hPAH_codop_ORF_v2”) with specific cis-regulatory elements is at least 98% identical to SEQ ID NO: 205. According to some embodiments, the nucleic acid sequence of ceDNA containing codon optimized human PAH version 2 (ceDNA “hPAH_codop_ORF_v2”) with specific cis-regulatory elements is at least 99% identical to SEQ ID NO: 205. According to some embodiments, the nucleic acid sequence of ceDNA containing codon optimized human PAH version 2 (ceDNA “hPAH_codop_ORF_v2”) with specific cis-regulatory elements consists of SEQ ID NO: 205.

The nucleic acid sequence of ceDNA containing codon optimized human PAH version 2 (ceDNA “hPAH_codop_ORF_v2”), with specific cis-regulatory elements is shown as SEQ ID NO: 206 (ceDNA1471).

SEQ ID NO: 206 includes the following elements. left-ITR_v1, spacer_left-ITR_v2.1, 3×HNF1-4_ProEnh_10mer, BamHI_site, TTR_liver_specific_Promoter, MVM_intron, PmeI_site, Mod_Minimum_Consensus_Kozak, hPAH_codop_ORF_v2, PacI_site, WPRE_3pUTR, bGH, spacer_right-ITR_v1, right-ITR_v1.

According to some embodiments, the nucleic acid sequence of ceDNA containing codon optimized human PAH version 2 (ceDNA “hPAH_codop_ORF_v2”) with specific cis-regulatory elements comprises SEQ ID NO: 206. According to some embodiments, the nucleic acid sequence of ceDNA containing codon optimized human PAH version 2 (ceDNA “hPAH_codop_ORF_v2”) with specific cis-regulatory elements is at least 85% identical to SEQ ID NO: 206. According to some embodiments, the nucleic acid sequence of ceDNA containing codon optimized human PAH version 2 (ceDNA “hPAH_codop_ORF_v2”) with specific cis-regulatory elements is at least 90% identical to SEQ ID NO: 206. According to some embodiments, the nucleic acid sequence of ceDNA containing codon optimized human PAH version 2 (ceDNA “hPAH_codop_ORF_v2”) with specific cis-regulatory elements is at least 91% identical to SEQ ID NO: 206. According to some embodiments, the nucleic acid sequence of ceDNA containing codon optimized human PAH version 2 (ceDNA “hPAH_codop_ORF_v2”) with specific cis-regulatory elements is at least 92% identical to SEQ ID NO: 206. According to some embodiments, the nucleic acid sequence of ceDNA containing codon optimized human PAH version 2 (ceDNA “hPAH_codop_ORF_v2”) with specific cis-regulatory elements is at least 93% identical to SEQ ID NO: 206. According to some embodiments, the nucleic acid sequence of ceDNA containing codon optimized human PAH version 2 (ceDNA “hPAH_codop_ORF_v2”) with specific cis-regulatory elements is at least 94% identical to SEQ ID NO: 206. According to some embodiments, the nucleic acid sequence of ceDNA containing codon optimized human PAH version 2 (ceDNA “hPAH_codop_ORF_v2”) with specific cis-regulatory elements is at least 95% identical to SEQ ID NO: 206. According to some embodiments, the nucleic acid sequence of ceDNA containing codon optimized human PAH version 2 (ceDNA “hPAH_codop_ORF_v2”) with specific cis-regulatory elements is at least 96% identical to SEQ ID NO: 206. According to some embodiments, the nucleic acid sequence of ceDNA containing codon optimized human PAH version 2 (ceDNA “hPAH_codop_ORF_v2”) with specific cis-regulatory elements is at least 97% identical to SEQ ID NO: 206. According to some embodiments, the nucleic acid sequence of ceDNA containing codon optimized human PAH version 2 (ceDNA “hPAH_codop_ORF_v2”) with specific cis-regulatory elements is at least 98% identical to SEQ ID NO: 206. According to some embodiments, the nucleic acid sequence of ceDNA containing codon optimized human PAH version 2 (ceDNA “hPAH_codop_ORF_v2”) with specific cis-regulatory elements is at least 99% identical to SEQ ID NO: 206. According to some embodiments, the nucleic acid sequence of ceDNA containing codon optimized human PAH version 2 (ceDNA “hPAH_codop_ORF_v2”) with specific cis-regulatory elements consists of SEQ ID NO: 206.

The nucleic acid sequence of ceDNA containing codon optimized human PAH version 2 (ceDNA “hPAH_codop_ORF_v2”), with specific cis-regulatory elements is shown as SEQ ID NO: 207 (ceDNA1472).

SEQ ID NO: 207 includes the following elements. left-ITR_v1, spacer_left-ITR_v2.1, 3×HNF1-4_ProEnh_10mer, HS-CRM8_SERP_Enhancer_nospacer, HS-CRM8_SERP_Enhancer_nospacer, BamHI_site, TTR_liver_specific_Promoter, MVM_intron, PmeI_site, Mod_Minimum_Consensus_Kozak, hPAH_codop_ORF_v2, PacI_site, WPRE_3pUTR, bGH, spacer_right-ITR_v1, right-ITR_v1.

According to some embodiments, the nucleic acid sequence of ceDNA containing codon optimized human PAH version 2 (ceDNA “hPAH_codop_ORF_v2”) with specific cis-regulatory elements comprises SEQ ID NO: 207. According to some embodiments, the nucleic acid sequence of ceDNA containing codon optimized human PAH version 2 (ceDNA “hPAH_codop_ORF_v2”) with specific cis-regulatory elements is at least 85% identical to SEQ ID NO: 207. According to some embodiments, the nucleic acid sequence of ceDNA containing codon optimized human PAH version 2 (ceDNA “hPAH_codop_ORF_v2”) with specific cis-regulatory elements is at least 90% identical to SEQ ID NO: 207. According to some embodiments, the nucleic acid sequence of ceDNA containing codon optimized human PAH version 2 (ceDNA “hPAH_codop_ORF_v2”) with specific cis-regulatory elements is at least 91% identical to SEQ ID NO: 207. According to some embodiments, the nucleic acid sequence of ceDNA containing codon optimized human PAH version 2 (ceDNA “hPAH_codop_ORF_v2”) with specific cis-regulatory elements is at least 92% identical to SEQ ID NO: 207. According to some embodiments, the nucleic acid sequence of ceDNA containing codon optimized human PAH version 2 (ceDNA “hPAH_codop_ORF_v2”) with specific cis-regulatory elements is at least 93% identical to SEQ ID NO: 207. According to some embodiments, the nucleic acid sequence of ceDNA containing codon optimized human PAH version 2 (ceDNA “hPAH_codop_ORF_v2”) with specific cis-regulatory elements is at least 94% identical to SEQ ID NO: 207. According to some embodiments, the nucleic acid sequence of ceDNA containing codon optimized human PAH version 2 (ceDNA “hPAH_codop_ORF_v2”) with specific cis-regulatory elements is at least 95% identical to SEQ ID NO: 207. According to some embodiments, the nucleic acid sequence of ceDNA containing codon optimized human PAH version 2 (ceDNA “hPAH_codop_ORF_v2”) with specific cis-regulatory elements is at least 96% identical to SEQ ID NO: 207. According to some embodiments, the nucleic acid sequence of ceDNA containing codon optimized human PAH version 2 (ceDNA “hPAH_codop_ORF_v2”) with specific cis-regulatory elements is at least 97% identical to SEQ ID NO: 207. According to some embodiments, the nucleic acid sequence of ceDNA containing codon optimized human PAH version 2 (ceDNA “hPAH_codop_ORF_v2”) with specific cis-regulatory elements is at least 98% identical to SEQ ID NO: 207. According to some embodiments, the nucleic acid sequence of ceDNA containing codon optimized human PAH version 2 (ceDNA “hPAH_codop_ORF_v2”) with specific cis-regulatory elements is at least 99% identical to SEQ ID NO: 207. According to some embodiments, the nucleic acid sequence of ceDNA containing codon optimized human PAH version 2 (ceDNA “hPAH_codop_ORF_v2”) with specific cis-regulatory elements consists of SEQ ID NO: 207.

The nucleic acid sequence of ceDNA containing codon optimized human PAH version 2 (ceDNA “hPAH_codop_ORF_v2”), with specific cis-regulatory elements is shown as SEQ ID NO: 208 (ceDNA1473).

SEQ ID NO: 208 includes the following elements. left-ITR_v1, spacer_left-ITR_v2.1, CpGfree20mer_1, 5×HNF1_ProEnh_10mer, BamHI_site, TTR_liver_specific_Promoter, MVM_intron, PmeI_site, Mod_Minimum_Consensus_Kozak, hPAH_codop_ORF_v2, PacI_site, WPRE_3pUTR, bGH, spacer_right-ITR_v1, right-ITR_v1.

According to some embodiments, the nucleic acid sequence of ceDNA containing codon optimized human PAH version 2 (ceDNA “hPAH_codop_ORF_v2”) with specific cis-elements comprises SEQ ID NO: 208. According to some embodiments, the nucleic acid sequence of ceDNA containing codon optimized human PAH version 2 (ceDNA “hPAH_codop_ORF_v2”) with specific cis-regulatory elements is at least 85% identical to SEQ ID NO: 208. According to some embodiments, the nucleic acid sequence of ceDNA containing codon optimized human PAH version 2 (ceDNA “hPAH_codop_ORF_v2”) with specific cis-regulatory elements is at least 90% identical to SEQ ID NO: 208. According to some embodiments, the nucleic acid sequence of ceDNA containing codon optimized human PAH version 2 (ceDNA “hPAH_codop_ORF_v2”) with specific cis-regulatory elements is at least 91% identical to SEQ ID NO: 2082. According to some embodiments, the nucleic acid sequence of ceDNA containing codon optimized human PAH version 2 (ceDNA “hPAH_codop_ORF_v2”) with specific cis-regulatory elements is at least 92% identical to SEQ ID NO: 208. According to some embodiments, the nucleic acid sequence of ceDNA containing codon optimized human PAH version 2 (ceDNA “hPAH_codop_ORF_v2”) with specific cis-regulatory elements is at least 93% identical to SEQ ID NO: 208. According to some embodiments, the nucleic acid sequence of ceDNA containing codon optimized human PAH version 2 (ceDNA “hPAH_codop_ORF_v2”) with specific cis-regulatory elements is at least 94% identical to SEQ ID NO: 208. According to some embodiments, the nucleic acid sequence of ceDNA containing codon optimized human PAH version 2 (ceDNA “hPAH_codop_ORF_v2”) with specific cis-regulatory elements is at least 95% identical to SEQ ID NO: 208. According to some embodiments, the nucleic acid sequence of ceDNA containing codon optimized human PAH version 2 (ceDNA “hPAH_codop_ORF_v2”) with specific cis-regulatory elements is at least 96% identical to SEQ ID NO: 208. According to some embodiments, the nucleic acid sequence of ceDNA containing codon optimized human PAH version 2 (ceDNA “hPAH_codop_ORF_v2”) with specific cis-regulatory elements is at least 97% identical to SEQ ID NO: 208. According to some embodiments, the nucleic acid sequence of ceDNA containing codon optimized human PAH version 2 (ceDNA “hPAH_codop_ORF_v2”) with specific cis-regulatory elements is at least 98% identical to SEQ ID NO: 208. According to some embodiments, the nucleic acid sequence of ceDNA containing codon optimized human PAH version 2 (ceDNA “hPAH_codop_ORF_v2”) with specific cis-regulatory elements is at least 99% identical to SEQ ID NO: 208. According to some embodiments, the nucleic acid sequence of ceDNA containing codon optimized human PAH version 2 (ceDNA “hPAH_codop_ORF_v2”) with specific cis-regulatory elements consists of SEQ ID NO: 208.

The nucleic acid sequence of ceDNA containing codon optimized human PAH version 2 (ceDNA “hPAH_codop_ORF_v2”), with specific cis-regulatory elements is shown as SEQ ID NO: 209 (ceDNA1474).

SEQ ID NO: 209 includes the following elements. left-ITR_v1, spacer_left-ITR_v2.1, CpGfree20mer_1, 5×HNF1_ProEnh_10mer, 3×VanD_TTRe_PromoterSet_v2, PmeI_site, Mod_Minimum_Consensus_Kozak, hPAH_codop_ORF_v2, PacI_site, WPRE_3pUTR, bGH, spacer_right-ITR_v1, right-ITR_v1.

According to some embodiments, the nucleic acid sequence of ceDNA containing codon optimized human PAH version 2 (ceDNA “hPAH_codop_ORF_v2”) with specific cis-regulatory elements comprises SEQ ID NO: 209. According to some embodiments, the nucleic acid sequence of ceDNA containing codon optimized human PAH version 2 (ceDNA “hPAH_codop_ORF_v2”) with specific cis-regulatory elements is at least 85% identical to SEQ ID NO: 209. According to some embodiments, the nucleic acid sequence of ceDNA containing codon optimized human PAH version 2 (ceDNA “hPAH_codop_ORF_v2”) with specific cis-regulatory elements is at least 90% identical to SEQ ID NO: 209. According to some embodiments, the nucleic acid sequence of ceDNA containing codon optimized human PAH version 2 (ceDNA “hPAH_codop_ORF_v2”) with specific cis-regulatory elements is at least 91% identical to SEQ ID NO: 209. According to some embodiments, the nucleic acid sequence of ceDNA containing codon optimized human PAH version 2 (ceDNA “hPAH_codop_ORF_v2”) with specific cis-regulatory elements is at least 92% identical to SEQ ID NO: 209. According to some embodiments, the nucleic acid sequence of ceDNA containing codon optimized human PAH version 2 (ceDNA “hPAH_codop_ORF_v2”) with specific cis-regulatory elements is at least 93% identical to SEQ ID NO: 209. According to some embodiments, the nucleic acid sequence of ceDNA containing codon optimized human PAH version 2 (ceDNA “hPAH_codop_ORF_v2”) with specific cis-regulatory elements is at least 94% identical to SEQ ID NO: 209. According to some embodiments, the nucleic acid sequence of ceDNA containing codon optimized human PAH version 2 (ceDNA “hPAH_codop_ORF_v2”) with specific cis-regulatory elements is at least 95% identical to SEQ ID NO: 209. According to some embodiments, the nucleic acid sequence of ceDNA containing codon optimized human PAH version 2 (ceDNA “hPAH_codop_ORF_v2”) with specific cis-regulatory elements is at least 96% identical to SEQ ID NO: 209. According to some embodiments, the nucleic acid sequence of ceDNA containing codon optimized human PAH version 2 (ceDNA “hPAH_codop_ORF_v2”) with specific cis-regulatory elements is at least 97% identical to SEQ ID NO: 209. According to some embodiments, the nucleic acid sequence of ceDNA containing codon optimized human PAH version 2 (ceDNA “hPAH_codop_ORF_v2”) with specific cis-regulatory elements is at least 98% identical to SEQ ID NO: 209. According to some embodiments, the nucleic acid sequence of ceDNA containing codon optimized human PAH version 2 (ceDNA “hPAH_codop_ORF_v2”) with specific cis-regulatory elements is at least 99% identical to SEQ ID NO: 209. According to some embodiments, the nucleic acid sequence of ceDNA containing codon optimized human PAH version 2 (ceDNA “hPAH_codop_ORF_v2”) with specific cis-regulatory elements consists of SEQ ID NO: 209.

The nucleic acid sequence of ceDNA containing codon optimized human PAH version 2 (ceDNA “hPAH_codop_ORF_v2”), with specific cis-regulatory elements is shown as SEQ ID NO: 210 (ceDNA1527).

SEQ ID NO: 210 includes the following elements. left-ITR_v1, spacer_left-ITR_v2.1, 3×VanD_TTRe_PromoterSet_v2, PmeI_site, Mod_Minimum_Consensus_Kozak, hPAH_codop_ORF_v2, PacI_site, WPRE_3pUTR, bGH, spacer_right-ITR_v1, right-ITR_v1.

According to some embodiments, the nucleic acid sequence of ceDNA containing codon optimized human PAH version 2 (ceDNA “hPAH_codop_ORF_v2”) with specific cis-regulatory elements comprises SEQ ID NO: 210. According to some embodiments, the nucleic acid sequence of ceDNA containing codon optimized human PAH version 2 (ceDNA “hPAH_codop_ORF_v2”) with specific cis-regulatory elements is at least 85% identical to SEQ ID NO: 210. According to some embodiments, the nucleic acid sequence of ceDNA containing codon optimized human PAH version 2 (ceDNA “hPAH_codop_ORF_v2”) with specific cis-regulatory elements is at least 90% identical to SEQ ID NO: 210. According to some embodiments, the nucleic acid sequence of ceDNA containing codon optimized human PAH version 2 (ceDNA “hPAH_codop_ORF_v2”) with specific cis-regulatory elements is at least 91% identical to SEQ ID NO: 210. According to some embodiments, the nucleic acid sequence of ceDNA containing codon optimized human PAH version 2 (ceDNA “hPAH_codop_ORF_v2”) with specific cis-regulatory elements is at least 92% identical to SEQ ID NO: 210. According to some embodiments, the nucleic acid sequence of ceDNA containing codon optimized human PAH version 2 (ceDNA “hPAH_codop_ORF_v2”) with specific cis-regulatory elements is at least 93% identical to SEQ ID NO: 210. According to some embodiments, the nucleic acid sequence of ceDNA containing codon optimized human PAH version 2 (ceDNA “hPAH_codop_ORF_v2”) with specific cis-regulatory elements is at least 94% identical to SEQ ID NO: 210. According to some embodiments, the nucleic acid sequence of ceDNA containing codon optimized human PAH version 2 (ceDNA “hPAH_codop_ORF_v2”) with specific cis-regulatory elements is at least 95% identical to SEQ ID NO: 210. According to some embodiments, the nucleic acid sequence of ceDNA containing codon optimized human PAH version 2 (ceDNA “hPAH_codop_ORF_v2”) with specific cis-regulatory elements is at least 96% identical to SEQ ID NO: 210. According to some embodiments, the nucleic acid sequence of ceDNA containing codon optimized human PAH version 2 (ceDNA “hPAH_codop_ORF_v2”) with specific cis-regulatory elements is at least 97% identical to SEQ ID NO: 210. According to some embodiments, the nucleic acid sequence of ceDNA containing codon optimized human PAH version 2 (ceDNA “hPAH_codop_ORF_v2”) with specific cis-regulatory elements is at least 98% identical to SEQ ID NO: 210. According to some embodiments, the nucleic acid sequence of ceDNA containing codon optimized human PAH version 2 (ceDNA “hPAH_codop_ORF_v2”) with specific cis-regulatory elements is at least 99% identical to SEQ ID NO: 210. According to some embodiments, the nucleic acid sequence of ceDNA containing codon optimized human PAH version 2 (ceDNA “hPAH_codop_ORF_v2”) with specific cis-regulatory elements consists of SEQ ID NO: 210.

The nucleic acid sequence of ceDNA containing codon optimized human PAH version 2 (ceDNA “hPAH_codop_ORF_v2”), with specific cis-regulatory elements is shown as SEQ ID NO: 211 (ceDNA1528).

SEQ ID NO: 211 includes the following elements. left-ITR_v1, spacer_left-ITR_v2.1, CpGmin_hAAT_Promoter_Set, PmeI_site, Mod_Minimum_Consensus_Kozak, hPAH_codop_ORF_v2, PacI_site, WPRE_3pUTR, bGH, spacer_right-ITR_v1, right-ITR_v1.

According to some embodiments, the nucleic acid sequence of ceDNA containing codon optimized human PAH version 2 (ceDNA “hPAH_codop_ORF_v2”) with specific cis-regulatory elements comprises SEQ ID NO: 211. According to some embodiments, the nucleic acid sequence of ceDNA containing codon optimized human PAH version 2 (ceDNA “hPAH_codop_ORF_v2”) with specific cis-regulatory elements is at least 85% identical to SEQ ID NO: 211. According to some embodiments, the nucleic acid sequence of ceDNA containing codon optimized human PAH version 2 (ceDNA “hPAH_codop_ORF_v2”) with specific cis-regulatory elements is at least 90% identical to SEQ ID NO: 211. According to some embodiments, the nucleic acid sequence of ceDNA containing codon optimized human PAH version 2 (ceDNA “hPAH_codop_ORF_v2”) with specific cis-regulatory elements is at least 91% identical to SEQ ID NO: 211. According to some embodiments, the nucleic acid sequence of ceDNA containing codon optimized human PAH version 2 (ceDNA “hPAH_codop_ORF_v2”) with specific cis-regulatory elements is at least 92% identical to SEQ ID NO: 211. According to some embodiments, the nucleic acid sequence of ceDNA containing codon optimized human PAH version 2 (ceDNA “hPAH_codop_ORF_v2”) with specific cis-regulatory elements is at least 93% identical to SEQ ID NO: 211. According to some embodiments, the nucleic acid sequence of ceDNA containing codon optimized human PAH version 2 (ceDNA “hPAH_codop_ORF_v2”) with specific cis-regulatory elements is at least 94% identical to SEQ ID NO: 211. According to some embodiments, the nucleic acid sequence of ceDNA containing codon optimized human PAH version 2 (ceDNA “hPAH_codop_ORF_v2”) with specific cis-regulatory elements is at least 95% identical to SEQ ID NO: 211. According to some embodiments, the nucleic acid sequence of ceDNA containing codon optimized human PAH version 2 (ceDNA “hPAH_codop_ORF_v2”) with specific cis-regulatory elements is at least 96% identical to SEQ ID NO: 211. According to some embodiments, the nucleic acid sequence of ceDNA containing codon optimized human PAH version 2 (ceDNA “hPAH_codop_ORF_v2”) with specific cis-regulatory elements is at least 97% identical to SEQ ID NO: 211. According to some embodiments, the nucleic acid sequence of ceDNA containing codon optimized human PAH version 2 (ceDNA “hPAH_codop_ORF_v2”) with specific cis-regulatory elements is at least 98% identical to SEQ ID NO: 211. According to some embodiments, the nucleic acid sequence of ceDNA containing codon optimized human PAH version 2 (ceDNA “hPAH_codop_ORF_v2”) with specific cis-regulatory elements is at least 99% identical to SEQ ID NO: 211. According to some embodiments, the nucleic acid sequence of ceDNA containing codon optimized human PAH version 2 (ceDNA “hPAH_codop_ORF_v2”) with specific cis-regulatory elements consists of SEQ ID NO: 211.

The nucleic acid sequence of ceDNA containing codon optimized human PAH version 2 r3-s34 (ceDNA “hPAH-r3-s34), with specific cis-regulatory elements is shown as SEQ ID NO: 212.(ceDNA1529).

SEQ ID NO: 212 includes the following elements. left-ITR_v1, spacer_left-ITR_v2, HS-CRM8_SERP_Enhancer_nospacer, HS-CRM8_SERP_Enhancer_nospacer, HS-CRM8_SERP_Enhancer_nospacer, BamHI_site, TTR-promoter-d5pUTR, MVM_intron, PmeI_site, Mod_Minimum_Consensus_Kozak, hPAH-r3-s34, PacI_site, WPRE_3pUTR, bGH, spacer_right-ITR_v1, right-ITR_v1.

According to some embodiments, the nucleic acid sequence of ceDNA containing codon optimized human PAH version 2 r3-s34 (ceDNA “hPAH-r3-s34”) with specific cis-regulatory elements comprises SEQ ID NO: 212. According to some embodiments, the nucleic acid sequence of ceDNA containing codon optimized human PAH version 2 r3-s34 (ceDNA “hPAH-r3-s34”) with specific cis-regulatory elements is at least 85% identical to SEQ ID NO: 212. According to some embodiments, the nucleic acid sequence of ceDNA containing codon optimized human PAH version 2 r3-s34 (ceDNA “hPAH-r3-s34”) with specific cis-regulatory elements is at least 90% identical to SEQ ID NO: 212. According to some embodiments, the nucleic acid sequence of ceDNA containing codon optimized human PAH version 2 r3-s34 (ceDNA “hPAH-r3-s34”) with specific cis-regulatory elements is at least 91% identical to SEQ ID NO: 212. According to some embodiments, the nucleic acid sequence of ceDNA containing codon optimized human PAH version 2 r3-s34 (ceDNA “hPAH-r3-s34”) with specific cis-regulatory elements is at least 92% identical to SEQ ID NO: 212. According to some embodiments, the nucleic acid sequence of ceDNA containing codon optimized human PAH version 2 r3-s34 (ceDNA “hPAH-r3-s34”) with specific cis-regulatory elements is at least 93% identical to SEQ ID NO: 212. According to some embodiments, the nucleic acid sequence of ceDNA containing codon optimized human PAH version 2 r3-s34 (ceDNA “hPAH-r3-s34”) with specific cis-regulatory elements is at least 94% identical to SEQ ID NO: 212. According to some embodiments, the nucleic acid sequence of ceDNA containing codon optimized human PAH version 2 r5-s29 (ceDNA “hPAH-r3-s34”) with specific cis-regulatory elements is at least 95% identical to SEQ ID NO: 212. According to some embodiments, the nucleic acid sequence of ceDNA containing codon optimized human PAH version 2 r3-s34 (ceDNA “hPAH-r3-s34”) with specific cis-regulatory elements is at least 96% identical to SEQ ID NO: 212. According to some embodiments, the nucleic acid sequence of ceDNA containing codon optimized human PAH version 2 r3-s34 (ceDNA “hPAH-r3-s34”) with specific cis-regulatory elements is at least 97% identical to SEQ ID NO: 212. According to some embodiments, the nucleic acid sequence of ceDNA containing codon optimized human PAH version 2 r3-s34 (ceDNA “hPAH-r3-s34”) with specific cis-regulatory elements is at least 98% identical to SEQ ID NO: 212. According to some embodiments, the nucleic acid sequence of ceDNA containing codon optimized human PAH version 2 r3-s34 (ceDNA “hPAH-r3-s34”) with specific cis-regulatory elements is at least 99% identical to SEQ ID NO: 212. According to some embodiments, the nucleic acid sequence of ceDNA containing codon optimized human PAH version 2 r3-s34 (ceDNA “hPAH-r3-s34”) with specific cis-regulatory elements consists of SEQ ID NO: 212.

The nucleic acid sequence of ceDNA containing codon optimized human PAH version 2 r5-s29 (ceDNA “hPAH-r5-s29”), with specific cis-regulatory elements is shown as SEQ ID NO: 213 (ceDNA1530).

SEQ ID NO: 213 includes the following elements: left-ITR_v1, spacer_left-ITR_v2, HS-CRM8_SERP_Enhancer_nospacer, HS-CRM8_SERP_Enhancer_nospacer, HS-CRM8_SERP_Enhancer_nospacer, BamHI_site, TTR-promoter-d5pUTR, MVM_intron, PmeI_site, Mod_Minimum_Consensus_Kozak, hPAH-r5-s29, PacI_site, WPRE_3pUTR, bGH, spacer_right-ITR_v1, right-ITR_v1.

According to some embodiments, the nucleic acid sequence of ceDNA containing codon optimized human PAH version 2 r5-s29 (ceDNA “hPAH-r5-s29”) with specific cis-regulatory elements comprises SEQ ID NO: 213. According to some embodiments, the nucleic acid sequence of ceDNA containing codon optimized human PAH version 2 r5-s29 (ceDNA “hPAH-r5-s29”) with specific cis-regulatory elements is at least 85% identical to SEQ ID NO: 213. According to some embodiments, the nucleic acid sequence of ceDNA containing codon optimized human PAH version 2 r5-s29 (ceDNA “hPAH-r5-s29”) with specific cis-regulatory elements is at least 90% identical to SEQ ID NO: 213. According to some embodiments, the nucleic acid sequence of ceDNA containing codon optimized human PAH version 2 r5-s29 (ceDNA “hPAH-r5-s29”) with specific cis-regulatory elements is at least 91% identical to SEQ ID NO: 213. According to some embodiments, the nucleic acid sequence of ceDNA containing codon optimized human PAH version 2 r5-s29 (ceDNA “hPAH-r5-s29”) with specific cis-regulatory elements is at least 92% identical to SEQ ID NO: 213. According to some embodiments, the nucleic acid sequence of ceDNA containing codon optimized human PAH version 2 r5-s29 (ceDNA “hPAH-r5-s29”) with specific cis-regulatory elements is at least 93% identical to SEQ ID NO: 213. According to some embodiments, the nucleic acid sequence of ceDNA containing codon optimized human PAH version 2 r5-s29 (ceDNA “hPAH-r5-s29”) with specific cis-regulatory elements is at least 94% identical to SEQ ID NO: 213. According to some embodiments, the nucleic acid sequence of ceDNA containing codon optimized human PAH version 2 r5-s29 (ceDNA “hPAH-r5-s29”) with specific cis-regulatory elements is at least 95% identical to SEQ ID NO: 213. According to some embodiments, the nucleic acid sequence of ceDNA containing codon optimized human PAH version 2 r5-s29 (ceDNA “hPAH-r5-s29”) with specific cis-regulatory elements is at least 96% identical to SEQ ID NO: 213. According to some embodiments, the nucleic acid sequence of ceDNA containing codon optimized human PAH version 2 r5-s29 (ceDNA “hPAH-r5-s29”) with specific cis-regulatory elements is at least 97% identical to SEQ ID NO: 213. According to some embodiments, the nucleic acid sequence of ceDNA containing codon optimized human PAH version 2 r5-s29 (ceDNA “hPAH-r5-s29”) with specific cis-regulatory elements is at least 98% identical to SEQ ID NO: 213. According to some embodiments, the nucleic acid sequence of ceDNA containing codon optimized human PAH version 2 r5-s29 (ceDNA “hPAH-r5-s29”) with specific cis-regulatory elements is at least 99% identical to SEQ ID NO: 213. According to some embodiments, the nucleic acid sequence of ceDNA containing codon optimized human PAH version 2 r5-s29 (ceDNA “hPAH-r5-s29”) with specific cis-regulatory elements consists of SEQ ID NO: 213.

Additional full-length ceDNA PAH construct sequences are set forth in Table 35. In some embodiments, the nucleic acid sequence of the full-length ceDNA PAH construct sequence is at least 90% identical to any sequence set forth in Table 35. In some embodiments, the nucleic acid sequence of the full-length ceDNA PAH construct sequence is at least 95% identical to any sequence set forth in Table 35. In some embodiments, the nucleic acid sequence of the full-length ceDNA PAH construct sequence is at least 96% identical to any sequence set forth in Table 35. In some embodiments, the nucleic acid sequence of the full-length ceDNA PAH construct sequence is at least 97% identical to any sequence set forth in Table 35. In some embodiments, the nucleic acid sequence of the full-length ceDNA PAH construct sequence is at least 98% identical to any sequence set forth in Table 35. In some embodiments, the nucleic acid sequence of the full-length ceDNA PAH construct sequence is at least 99% identical to any sequence set forth in Table 35. In some embodiments, the nucleic acid sequence of the full-length ceDNA PAH construct sequence comprises any sequence set forth in Table 35. In some embodiments, the nucleic acid sequence of the full-length ceDNA PAH construct sequence consists of any sequence set forth in Table 35.

TABLE 35
Additonal Full-length ceDNA
PAH Construct Sequences

| | Sequence |
|---|---|
| Registry ID | Identifier |
| ceDNA1137 | 541 |
| ceDNA1146 | 542 |
| ceDNA1279 | 543 |
| ceDNA1285 | 544 |
| ceDNA1475 | 545 |
| ceDNA1476 | 546 |
| ceDNA1477 | 547 |
| ceDNA1478 | 548 |
| ceDNA1479 | 549 |
| ceDNA1480 | 550 |
| ceDNA1497 | 551 |
| ceDNA1498 | 552 |
| ceDNA1499 | 553 |
| ceDNA1500 | 554 |
| ceDNA1501 | 555 |
| ceDNA1502 | 556 |
| ceDNA1503 | 557 |
| ceDNA1504 | 558 |
| ceDNA1505 | 559 |
| ceDNA1531 | 560 |
| ceDNA1532 | 561 |
| ceDNA1939 | 562 |
| ceDNA1940 | 563 |
| ceDNA1941 | 564 |
| ceDNA1942 | 565 |
| ceDNA1943 | 566 |
| ceDNA1945 | 567 |
| ceDNA1955 | 568 |
| ceDNA62 | 569 |
| ceDNA2409 | 570 |
| ceDNA2410 | 571 |
| ceDNA2415 | 572 |
| ceDNA2418 | 573 |
| ceDNA2416 | 574 |
| ceDNA2419 | 575 |
| ceDNA2420 | 576 |
| ceDNA34 | 577 |
| ceDNA35 | 578 |
| ceDNA36 | 579 |
| ceDNA37 | 580 |
| ceDNA41 | 581 |
| ceDNA42 | 582 |
| ceDNA43 | 583 |
| ceDNA44 | 584 |

### REFERENCES

All publications and references, including but not limited to patents and patent applications, cited in this specification and Examples herein are incorporated by reference in their entirety as if each individual publication or reference were specifically and individually indicated to be incorporated by reference herein as being fully set forth. Any patent application to which this application claims priority is also incorporated by reference herein in the manner described above for publications and references.

1. A closed-ended DNA (ceDNA) vector comprising:
at least one nucleic acid sequence that encodes at least one phenylalanine hydroxylase (PAH) protein, wherein the at least one nucleic acid sequence is selected from a sequence having at least 90% identity to any of the sequences listed in Table 1A, wherein the at least one nucleic acid sequence is codon optimized, and wherein the at least one nucleic acid sequence is located between flanking inverted terminal repeats (ITRs); and
a promoter operatively linked to the least one nucleotide sequence that encodes the at least one PAH protein, wherein the promoter is selected from the group consisting of the VD promoter, the human alpha 1-antitrypsin (hAAT) promoter (including the hAAT(979) promoter and the CpGmin_hAAT promoter) and the transthyretin (TTR) liver specific promoter.

2. The ceDNA vector of claim 1, wherein the at least one nucleic acid sequence encoding the at least one PAH protein is selected from a sequence having at least 95% identity to any one of the sequences set forth in Table 1A.

3. A closed-ended DNA (ceDNA) vector comprising:
a nucleic acid sequence that encodes at least one PAH protein, wherein the nucleic acid sequence is selected from a sequence having at least 95% identity to any of the sequences listed in Table 1A, wherein the at least one nucleic acid sequence is located between flanking inverted terminal repeats (ITRs); and
a promoter operatively linked to the nucleic acid sequence that encodes the at least one PAH protein, wherein the promoter is selected from the group consisting of the VD promoter, the human alpha 1-antitrypsin (hAAT) promoter and the transthyretin (TTR) liver specific promoter.

4. A closed-ended DNA (ceDNA) vector comprising:
a nucleic acid sequence that encodes at least one PAH protein, wherein the nucleic acid sequence is selected from a sequence having at least 96% identity to any of the sequences listed in Table 1A, wherein the at least one nucleic acid sequence is located between flanking inverted terminal repeats (ITRs); and
a promoter operatively linked to the nucleic acid sequence that encodes the at least one PAH protein, wherein the promoter is selected from the group consisting of the VD promoter, the human alpha 1-antitrypsin (hAAT) promoter and the transthyretin (TTR) liver specific promoter.

5. A closed-ended DNA (ceDNA) vector comprising:
a nucleic acid sequence that encodes at least one PAH protein, wherein the nucleic acid sequence is selected from a sequence having at least 97% identity to any of the sequences listed in Table 1A, wherein the at least one nucleic acid sequence is located between flanking inverted terminal repeats (ITRs); and
a promoter operatively linked to the nucleic acid sequence that encodes the at least one PAH protein, wherein the promoter is selected from the group consisting of the VD promoter, the human alpha 1-antitrypsin (hAAT) promoter and the transthyretin (TTR) liver specific promoter.

6. A closed-ended DNA (ceDNA) vector comprising:
a nucleic acid sequence that encodes at least one PAH protein, wherein the nucleic acid sequence is selected from a sequence having at least 98% identity to any of the sequences listed in Table 1A, wherein the at least one nucleic acid sequence is located between flanking inverted terminal repeats (ITRs); and
a promoter operatively linked to the nucleic acid sequence that encodes the at least one PAH protein, wherein the promoter is selected from the group consisting of the VD promoter, the human alpha 1-antitrypsin (hAAT) promoter and the transthyretin (TTR) liver specific promoter.

7. A closed-ended DNA (ceDNA) vector comprising:
a nucleic acid sequence that encodes at least one PAH protein, wherein the nucleic acid sequence is selected from a sequence having at least 99% identity to any of the sequences listed in Table 1A, wherein the at least one nucleic acid sequence is located between flanking inverted terminal repeats (ITRs); and
a promoter operatively linked to the nucleic acid sequence that encodes the at least one PAH protein, wherein the promoter is selected from the group consisting of the VD promoter, the human alpha 1-antitrypsin (hAAT) promoter and the transthyretin (TTR) liver specific promoter.

8. The ceDNA vector of any one of claims 1-7, wherein the at least one nucleic acid sequence encoding the at least one PAH protein is a sequence having at least 98% identity to the sequence set forth as SEQ ID NO:382.

9. The ceDNA vector of any one of claims 1-7, wherein the at least one nucleic acid sequence encoding the at least one PAH protein is a sequence having at least 99% identity to the sequence set forth as SEQ ID NO:382.

10. The ceDNA vector of any one of claims 1-7, wherein the at least one nucleic acid sequence encoding the at least one PAH protein is set forth as SEQ ID NO:382.

11. The ceDNA vector of any one of claims 1-7, wherein the at least one nucleic acid sequence encoding the at least one PAH protein is a sequence having at least 99% identity to the sequence set forth as SEQ ID NO:425.

12. The ceDNA vector of any one of claims 1-7, wherein the at least one nucleic acid sequence encoding the at least one PAH protein is set forth as SEQ ID NO:425.

13. The ceDNA vector of any one of claims 1-7, wherein the at least one nucleic acid sequence encoding the at least one PAH protein is a sequence having at least 99% identity to the sequence set forth as SEQ ID NO:431.

14. The ceDNA vector of any one of claims 1-7, wherein the at least one nucleic acid sequence encoding the at least one PAH protein is set forth as SEQ ID NO:431.

15. The ceDNA vector of any one of claims 1-7, wherein the at least one nucleic acid sequence encoding the at least one PAH protein is a sequence having at least 99% identity to the sequence set forth as SEQ ID NO:435.

16. The ceDNA vector of any one of claims 1-7, wherein the at least one nucleic acid sequence encoding the at least one PAH protein is set forth as SEQ ID NO:435.

17. The ceDNA vector of any one of claims 1-7, wherein the promoter comprises a nucleic acid sequence having at least 85% identity to SEQ ID NO: 191.

18. The ceDNA vector of any one of claims 1-7, wherein the promoter comprises a nucleic acid sequence having at least 98% identity to SEQ ID NO: 443.

19. The ceDNA vector of any one of claims 1-7, wherein the promoter comprises a nucleic acid sequence having at least 99% identity to SEQ ID NO: 444.

20. The ceDNA vector of any one of claims 1-7, wherein the promoter comprises a nucleic acid sequence having at least 99% identity to SEQ ID NO: 445.

21. The ceDNA vector of any one of claims 1-7, wherein the promoter comprises a nucleic acid sequence having at least 99% identity to SEQ ID NO: 446.

22. The ceDNA vector of any one of claims 1-7, wherein the promoter comprises a nucleic acid sequence having at least 96% identity to SEQ ID NO: 447.

23. The ceDNA vector of any of claims 1 to 22, wherein the ceDNA vector further comprises an enhancer.

24. The ceDNA vector of claim 23, wherein the enhancer is selected from the group consisting of a serpin enhancer, a 3×HNF1-4_ProEnh_10mer, and a 5×HNF1_ProEnh_10mer.

25. The ceDNA vector of claim 23, wherein the enhancer comprises a nucleic acid sequence having at least 85% identity to SEQ ID NO: 450.

26. The ceDNA vector of claim 23, wherein the enhancer comprises a nucleic acid sequence having at least 85% identity to SEQ ID NO: 586.

27. The ceDNA vector of claim 23, wherein the enhancer comprises a nucleic acid sequence having at least 85% identity to SEQ ID NO: 587.

28. The ceDNA vector of claim any one of claims 1-7, wherein the promoter is a promoter set that comprises a nucleic acid sequence having at least 85% identity to SEQ ID NO: 462.

29. The ceDNA vector of claim any one of claims 1-7, wherein the promoter is a promoter set that comprises a nucleic acid sequence having at least 85% identity to SEQ ID NO: 467.

30. The ceDNA vector of claim any one of claims 1-7, wherein the promoter is a promoter set that comprises a nucleic acid sequence having at least 85% identity to SEQ ID NO: 470.

31. The ceDNA vector of claim any one of claims 1-7, wherein the promoter is a promoter set that comprises a nucleic acid sequence having at least 90% identity to SEQ ID NO: 470.

32. The ceDNA vector of claim any one of claims 1-7, wherein the promoter is a promoter set that comprises a nucleic acid sequence having at least 95% identity to SEQ ID NO: 470.

33. The ceDNA vector of any of claims 1 to 32, wherein the ceDNA vector further comprises one or more introns.

34. The ceDNA vector of claim 33, wherein the one or more introns is the minute virus of mice (MVM).

35. The ceDNA vector of any of claims 1 to 34, wherein the ceDNA vector comprises a 3′ untranslated region (3′ UTR).

36. The ceDNA vector of any of claims 1 to 35, wherein the ceDNA vector comprises at least one polyA sequence.

37. The ceDNA vector of any one of claims 1-7, wherein the VD promoter comprises a SERP enhancer.

38. The ceDNA vector of any one of claims 1-7, wherein the VD promoter comprises a 3×SERP enhancer.

39. The ceDNA vector of any one of claims 1-7, wherein the promoter is the TTR liver promoter and the ceDNA further comprises an MVM intron.

40. The ceDNA vector of any one of claims 1-39, wherein the ceDNA vector comprises a nucleic acid sequence that is at least 90% identical to a nucleic acid sequence selected from the group consisting of: SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 570, SEQ ID NO: 571, SEQ ID NO: 572, SEQ ID NO:573, SEQ ID NO: 574, SEQ ID NO: 575, SEQ ID NO: 576, SEQ ID NO: 577, SEQ ID NO: 578, SEQ ID NO: 579, SEQ ID NO:580, SEQ ID NO: 581, SEQ ID NO: 582, SEQ ID NO:583, and SEQ ID NO:584.

41. The ceDNA vector of any one of claims 1-40, wherein at least one nucleic acid sequence is cDNA for PAH.

42. The ceDNA vector of any one of claims 1-41, wherein at least one ITR comprises a functional terminal resolution site (TRS) and a Rep binding site.

43. The ceDNA vector of any one of claims 1-42, wherein one or both of the ITRs are from a virus selected from a parvovirus, a dependovirus, and an adeno-associated virus (AAV).

44. The ceDNA vector of any one of claims 1-43, wherein the flanking ITRs are symmetric or asymmetric.

45. The ceDNA vector of claim 44, wherein the flanking ITRs are symmetrical or substantially symmetrical.

46. The ceDNA vector of claim 45, wherein the flanking ITRs are asymmetric.

47. The ceDNA vector of any one of claims 1-46, wherein one or both of the ITRs are wild type, or wherein both of the ITRs are wild-type.

48. The ceDNA vector of any one of claims 1-47, wherein both of the ITRs are wild-type of the same AAV.

49. The ceDNA vector of claim 48, wherein both of the ITRs are wild-type of AAV2.

50. The ceDNA vector of any one of claims 1-49, wherein the flanking ITRs are from different viral serotypes.

51. The ceDNA vector of any one of claims 1-50, wherein the flanking ITRs are from a pair of viral serotypes shown in Table 2.

52. The ceDNA vector of any one of claims 1-51, wherein one or both of the ITRs comprises a sequence selected from the sequences in Table 3, Table 5A, Table 5B, or Table 6.

53. The ceDNA vector of any one of claims 1-52, wherein at least one of the ITRs is altered from a wild-type AAV ITR sequence by a deletion, addition, or substitution that affects the overall three-dimensional conformation of the ITR.

54. The ceDNA vector of any one of claims 1-53, wherein one or both of the ITRs are derived from an AAV serotype selected from AAV1, AAV2, AAV3, AAV4, AAV5, AAV6, AAV7, AAV8, AAV9, AAV10, AAV11, and AAV12.

55. The ceDNA vector of any one of claims 1-54, wherein one or both of the ITRs are synthetic.

56. The ceDNA vector of any one of claims 1-55, wherein one or both of the ITRs is not a wild type ITR, or wherein both of the ITRs are not wild-type.

57. The ceDNA vector of any one of claims 1-56, wherein one or both of the ITRs is modified by a deletion, insertion, and/or substitution in at least one of the ITR regions selected from A, A′, B, B′, C, C′, D, and D′.

58. The ceDNA vector of claim 57, wherein the deletion, insertion, and/or substitution results in the deletion of all or part of a stem-loop structure normally formed by the A, A′, B, B′ C, or C′ regions.

59. The ceDNA vector of any one of claims 1-58, wherein one or both of the ITRs are modified by a deletion, insertion, and/or substitution that results in the deletion of all or part of a stem-loop structure normally formed by the B and B′ regions.

60. The ceDNA vector of any one of claims 1-59, wherein one or both of the ITRs are modified by a deletion, insertion, and/or substitution that results in the deletion of all or part of a stem-loop structure normally formed by the C and C′ regions.

61. The ceDNA vector of any one of claims 1-60, wherein one or both of the ITRs are modified by a deletion, insertion, and/or substitution that results in the deletion of part of a stem-loop structure normally formed by the B and B′ regions and/or part of a stem-loop structure normally formed by the C and C′ regions.

62. The ceDNA vector of any one of claims 1-61, wherein one or both of the ITRs comprise a single stem-loop structure in the region that normally comprises a first stem-loop structure formed by the B and B′ regions and a second stem-loop structure formed by the C and C′ regions.

63. The ceDNA vector of any one of claims 1-62, wherein one or both of the ITRs comprise a single stem and two loops in the region that normally comprises a first stem-loop structure formed by the B and B′ regions and a second stem-loop structure formed by the C and C′ regions.

64. The ceDNA vector of any one of claims 1-63, wherein one or both of the ITRs comprise a single stem and a single loop in the region that normally comprises a first stem-loop structure formed by the B and B′ regions and a second stem-loop structure formed by the C and C′ regions.

65. The ceDNA vector of any one of claims 1-64, wherein both ITRs are altered in a manner that results in an overall three-dimensional symmetry when the ITRs are inverted relative to each other.

66. The ceDNA vector of any one of claims 1-65, wherein the ceDNA vector comprises a nucleic acid sequence that is at least 90% identical to SEQ ID NO: 382, SEQ ID NO:383, SEQ ID NO: 384, SEQ ID NO: 385 or SEQ ID NO:386.

67. The ceDNA vector of any one of claims 1-65, wherein the ceDNA vector comprises a nucleic acid sequence that is at least 95% identical to SEQ ID NO: 382, SEQ ID NO:383, SEQ ID NO: 384, SEQ ID NO: 385 or SEQ ID NO:386.

68. The ceDNA vector of any one of claims 1-65, wherein the ceDNA vector comprises a nucleic acid sequence that is at least 96% identical to SEQ ID NO: 382, SEQ ID NO:383, SEQ ID NO: 384, SEQ ID NO: 385 or SEQ ID NO:386.

69. The ceDNA vector of any one of claims 1-65, wherein the ceDNA vector comprises a nucleic acid sequence that is at least 97% identical to SEQ ID NO: 382, SEQ ID NO:383, SEQ ID NO: 384, SEQ ID NO: 385 or SEQ ID NO:386.

70. The ceDNA vector of any one of claims 1-65, wherein the ceDNA vector comprises a nucleic acid sequence that is at least 98% identical to SEQ ID NO: 382, SEQ ID NO:383, SEQ ID NO: 384, SEQ ID NO: 385 or SEQ ID NO:386.

71. The ceDNA vector of any one of claims 1-65, wherein the ceDNA vector comprises a nucleic acid sequence that is at least 99% identical to SEQ ID NO: 382, SEQ ID NO:383, SEQ ID NO: 384, SEQ ID NO: 385 or SEQ ID NO:386.

72. A method of expressing a PAH protein in a cell, the method comprising contacting the cell with the ceDNA vector of any one of claims 1-71.

73. The method of claim 72, wherein the cell is a photoreceptor cell or a retinal pigment epithelial (RPE) cell.

74. The method of claim 72 or 73, wherein the cell in in vitro or in vivo.

75. A method of treating a subject with phenylketonuria (PKU), the method comprising administering to the subject a ceDNA vector of any one of claims 1-71.

76. The method of claim 75, wherein the least one nucleic acid sequence that encodes at least one PAH protein is selected from a sequence having at least 90% identity with any of the sequences set forth in Table 1A.

77. The method of any one of claims 72-76, wherein the subject exhibits at least about a 50% decrease in level of serum phenylalanine as compared to a level of serum phenylalanine in the subject prior to administration.

78. The method of any one of claims 72-77, wherein the subject exhibits at least about a 10% increase in PAH activity after administration as compared to a level of PAH activity prior to administration.

79. The method of any one of claims 72-78, wherein the ceDNA vector is formulated in lipid nanoparticles.

80. The method of any one of claims 72-79, wherein the ceDNA vector is administered intravenously.

81. The method of any one of claims 72-79, wherein the ceDNA vector is administered intramuscularly.

82. The method of any one of claims 72-79, wherein the ceDNA vector is administered by infusion.

83. A pharmaceutical composition comprising the ceDNA vector of any one of claims 1-71.

84. A composition comprising a ceDNA vector of any of claims 1-71 and a lipid.

85. The composition of claim 84, wherein the lipid is a lipid nanoparticle (LNP).

86. A kit comprising the ceDNA vector of any one of claims 1-71, the pharmaceutical composition of claim 83, or the composition of claim 84 or 85.
